SEQUENCE LISTING (393 sequences)

SEQ ID NO: 1 - protein, 337 aa, Homo sapiens
MNSTCIEEQHDLDHYLFPIVYIFVIIVSIPANIGSLCVSFLQAKKESELGIYLFSLSLSDLLYALTLPLWIDYTWNKDNWTFSPALCKGSAFLMYMNFYSSTAFLTCIAVDRYLAVVYPLKFFFLRTRRFALMVSLSIWILETIFNAVMLWEDETVVEYCDAEKSNFTLCYDKYPLEKWQINLNLFRTCTGYAIPLVTILICNRKVYQAVRHNKATENKEKKRIIKLLVSITVTFVLCFTPFHVMLLIRCILEHAVNFEDHSNSGKRTYTMYRITVALTSLNCVADPILYCFVTETGRYDMWNILKFCTGRCNTSQRQRKRILSVSTKDTMELEVLE

SEQ ID NO: 2 - protein, 470 aa, Homo sapiens
MGWAAFNLSNVTGNSRILCSVYCNGSQITGSSNITVYRLPERVELAPLPPWQPVGQNFTLRCQVEDGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSHFVPVFVAVLLTLGVVTIVLALMYVFREHQRSGSYHVREESTYLPLTSMQPTEAMGEEPSRAE

SEQ ID NO: 3 - protein, 488 aa, Homo sapiens
MSTPGVNSSASLSPDRLNSPVTIPAVMFIFGVVGNLVAIVVLCKSRKEQKETTFYTLVCGLAVTDLLGTLLVSPVTIATYMKGQWPGGQPLCEYSTFILLFFSLSGLSIICAMSVERYLAINHAYFYSHYVDKRLAGLTLFAVYASNVLFCALPNMGLGSSRLQYPDTWCFIDWTTNVTAHAAYSYMYAGFSSFLILATVLCNVLVCGALLRMHRQFMRRTSLGTEQHHAAAAASVASRGHPAASPALPRLSDFRRRRSFRRIAGAEIQMVILLIATSLVVLICSIPLVVRVFVNQLYQPSLEREVSKNPDLQAIRIASVNPILDPWIYILLRKTVLSKAIEKIKCLFCRIGGSRRERSGQHCSDSQRTSSAMSGHSRSFISRELKEISSTSQTLLPDLSLPDLSENGLGGRNLLPGVPGMGLAQEDTTSLRTLRISETSDSSQGQDSESVLLVDEAGGSGRAGPAPKGSSLQVTFPSETLNLSEKCI

SEQ ID NO: 4 - protein, 256 aa, Homo sapiens
MRPAALRGALLGCLCLALLCLGGADKRLRDNHEWKKLIMVQHWPETVCEKIQNDCRDPPDYWTIHGLWPDKSEGCNRSWPFNLEEIKDLLPEMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 5 - protein, 184 aa, Artificial Sequence
  Description of Artificial Sequence: Synthetic polypeptide
EGCNRSWPFNLEEIKDLLPEMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 6 - protein, 164 aa, Artificial Sequence
  Description of Artificial Sequence: Synthetic polypeptide
MRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 7-208 - skipped (202 entries)

SEQ ID NO: 209 - protein, 8 aa, Artificial Sequence
  Description of Artificial Sequence: Synthetic peptide
GFTFSTYG

SEQ ID NO: 210 - protein, 8 aa, Artificial Sequence
  Description of Artificial Sequence: Synthetic peptide
ISGTGRTT

SEQ ID NO: 211 - protein, 14 aa, Artificial Sequence
  Description of Artificial Sequence: Synthetic peptide
TKERGDYYYGVFDY

SEQ ID NO: 212 - protein, 6 aa, Artificial Sequence
  Description of Artificial Sequence: Synthetic peptide
QTISSW

SEQ ID NO: 213 - protein, 3 aa, Artificial Sequence
  Description of Artificial Sequence: Synthetic peptide
AAS

SEQ ID NO: 214 - protein, 8 aa, Artificial Sequence
  Description of Artificial Sequence: Synthetic peptide
QQYHRSWT

SEQ ID NO: 215 - protein, 121 aa, Artificial Sequence
  Description of Artificial Sequence: Synthetic polypeptide
EVQLLESGGGLVQPGKSLRLSCAVSGFTFSTYGMNWVRQAPGKGLEWVSSISGTGRTTYHADSVQGRFTVSRDNSKNILYLQMNSLRADDTAVYFCTKERGDYYYGVFDYWGQGTLVTVSS

SEQ ID NO: 216 - protein, 106 aa, Artificial Sequence
  Description of Artificial Sequence: Synthetic polypeptide
DIQMTQSPSTLSASVGDRVTITCRASQTISSWLAWYQQTPEKAPKLLIYAASNLQSGVPSRFSGSGSGTEFTLTISSLQPDDFATYYCQQYHRSWTFGQGTKVEIT

SEQ ID NO: 217 - protein, 8 aa, Artificial Sequence
  Description of Artificial Sequence: Synthetic peptide
GFTFSSYW

SEQ ID NO: 218 - protein, 8 aa, Artificial Sequence
  Description of Artificial Sequence: Synthetic peptide
IKEDGSEK

SEQ ID NO: 219 - protein, 15 aa, Artificial Sequence
  Description of Artificial Sequence: Synthetic peptide
AREDYDSYYKYGMDV

SEQ ID NO: 220 - protein, 12 aa, Artificial Sequence
  Description of Artificial Sequence: Synthetic peptide
QSILYSSNNKNY

SEQ ID NO: 221 - protein, 3 aa, Artificial Sequence
  Description of Artificial Sequence: Synthetic peptide
WAS

SEQ ID NO: 222 - protein, 9 aa, Artificial Sequence
  Description of Artificial Sequence: Synthetic peptide
QQYYSTPFT

SEQ ID NO: 223 - protein, 122 aa, Artificial Sequence
  Description of Artificial Sequence: Synthetic polypeptide
EVQLVESGGGLVQPGGSLRLSCAVSGFTFSSYWMSWVRQAPGKGLEWVANIKEDGSEKNYVDSVKGRFTLSSDNAKNSLYLQMNSLRAEDTAVYYCAREDYDSYYKYGMDVWGQGTAVIVSS

SEQ ID NO: 224 - protein, 113 aa, Artificial Sequence
  Description of Artificial Sequence: Synthetic polypeptide
DIVMTQSPDSLAVSLGERATINCKSSQSILYSSNNKNYLAWYQQKPGQPPKLLIYWASTRESGVPDRFSGSGSGTDFTLTISSLQAEDVSVYYCQQYYSTPFTFGPGTKVDIK

SEQ ID NO: 225 - protein, 8 aa, Artificial Sequence
  Description of Artificial Sequence: Synthetic peptide
GGSFTGFY

SEQ ID NO: 226 - protein, 7 aa, Artificial Sequence
  Description of Artificial Sequence: Synthetic peptide
INHRGNT

SEQ ID NO: 227 - protein, 13 aa, Artificial Sequence
  Description of Artificial Sequence: Synthetic peptide
ASPFYDFWSGSDY

SEQ ID NO: 228 - protein, 11 aa, Artificial Sequence
  Description of Artificial Sequence: Synthetic peptide
QSLVHSDGNTY

SEQ ID NO: 229 - protein, 3 aa, Artificial Sequence
  Description of Artificial Sequence: Synthetic peptide
KIS

SEQ ID NO: 230 - protein, 9 aa, Artificial Sequence
  Description of Artificial Sequence: Synthetic peptide
MQATQFPLT

SEQ ID NO: 231 - protein, 119 aa, Artificial Sequence
  Description of Artificial Sequence: Synthetic polypeptide
QVQLQQWGAGLLKPSETLSLTCAVYGGSFTGFYWSWIRQPPGKGLEWIGEINHRGNTNYNPSLKSRVTMSVDTSKNQFSLNMISVTAADTAMYFCASPFYDFWSGSDYWGQGTLVTVSS

SEQ ID NO: 232 - protein, 112 aa, Artificial Sequence
  Description of Artificial Sequence: Synthetic polypeptide
DIMLTQTPLTSPVTLGQPASISCKSSQSLVHSDGNTYLSWLQQRPGQPPRLLFYKISNRFSGVPDRFSGSGAGTDFTLKISRVEAEDVGVYYCMQATQFPLTFGGGTKVEIK

SEQ ID NO: 233 - protein, 10 aa, Artificial Sequence
  Description of Artificial Sequence: Synthetic peptide
  MOD_RES (3)..(3): Pro, Ser, Asp, Gln or Asn
  MOD_RES (5)..(5): Thr or Arg
  MOD_RES (6)..(6): Asn, Thr, Tyr or His
GYXFXXYGIS

SEQ ID NO: 234 - protein, 17 aa, Artificial Sequence
  Description of Artificial Sequence: Synthetic peptide
  MOD_RES (4)..(4): Thr, Pro, Ser or Ala
  MOD_RES (8)..(8): Asn, Gly, Val, Lys or Ala
  MOD_RES (9)..(9): Thr or Lys
  MOD_RES (10)..(10): His or Asn
  MOD_RES (13)..(13): Gln or Arg
  MOD_RES (14)..(14): Lys or Met
  MOD_RES (15)..(15): Leu or His
WISXYNGXXXYAXXXQG

SEQ ID NO: 235 - protein, 15 aa, Artificial Sequence
  Description of Artificial Sequence: Synthetic peptide
  MOD_RES (8)..(8): Ser or Ala
  MOD_RES (9)..(9): Tyr or Pro
  MOD_RES (15)..(15): Val, Ala or Gly
ENYYGSGXXRGGMDX

SEQ ID NO: 236 - protein, 10 aa, Artificial Sequence
  Description of Artificial Sequence: Synthetic peptide
GYDFTYYGIS

SEQ ID NO: 237 - protein, 17 aa, Artificial Sequence
  Description of Artificial Sequence: Synthetic peptide
WISTYNGNTHYARMLQG

SEQ ID NO: 238 - protein, 15 aa, Artificial Sequence
  Description of Artificial Sequence: Synthetic peptide
ENYYGSGAYRGGMDV

SEQ ID NO: 239 - protein, 11 aa, Artificial Sequence
  Description of Artificial Sequence: Synthetic peptide
RASQSVSSYLA

SEQ ID NO: 240 - protein, 7 aa, Artificial Sequence
  Description of Artificial Sequence: Synthetic peptide
DASNRAT

SEQ ID NO: 241 - protein, 9 aa, Artificial Sequence
  Description of Artificial Sequence: Synthetic peptide
QQRSNWPWT

SEQ ID NO: 242 - protein, 124 aa, Artificial Sequence
  Description of Artificial Sequence: Synthetic polypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 243 - protein, 107 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 244 - protein, 453 aa, Artificial Sequence
  Description of Artificial Sequence: Synthetic polypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 245 - protein, 214 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 7 aa, Artificial Sequence
  Description of Artificial Sequence: Synthetic peptide
SRSYYWG

SEQ ID NO: 247 - protein, 16 aa, Artificial Sequence
  Description of Artificial Sequence: Synthetic peptide
SIYYNGRTYYNPSLKS

SEQ ID NO: 248 - protein, 11 aa, Artificial Sequence
  Description of Artificial Sequence: Synthetic peptide
EDYGDYGAFDI

SEQ ID NO: 249 - protein, 11 aa, Artificial Sequence
  Description of Artificial Sequence: Synthetic peptide
RASQGISSALA

SEQ ID NO: 250 - protein, 7 aa, Artificial Sequence
  Description of Artificial Sequence: Synthetic peptide
DASSLES

SEQ ID NO: 251 - protein, 9 aa, Artificial Sequence
  Description of Artificial Sequence: Synthetic peptide
QQFNSYPLT

SEQ ID NO: 252 - protein, 121 aa, Artificial Sequence
  Description of Artificial Sequence: Synthetic polypeptide
QLQLQESGPGLVKPSETLSLTCTVSGGSISSRSYYWGWIRQPPGKGLEWIGSIYYNGRTYYNPSLKSRVTISVDTSKNQFSLKLSSVTAADTAVYYCAREDYGDYGAFDIWGQGTMVTVSS

SEQ ID NO: 253 - protein, 107 aa, Artificial Sequence [sequence omitted]

SEQ ID NO: 254 - protein, 7 aa, Artificial Sequence
  Description of Artificial Sequence: Synthetic peptide
TSNMGVV

SEQ ID NO: 255 - protein, 16 aa, Artificial Sequence
  Description of Artificial Sequence: Synthetic peptide
HILWDDREYSNPALKS

SEQ ID NO: 256 - protein, 14 aa, Artificial Sequence
  Description of Artificial Sequence: Synthetic peptide
MSRNYYGSSYVMDY

SEQ ID NO: 257 - protein, 10 aa, Artificial Sequence
  Description of Artificial Sequence: Synthetic peptide
SASSSVNYMH

SEQ ID NO: 258 - protein, 7 aa, Artificial Sequence
  Description of Artificial Sequence: Synthetic peptide
STSNLAS

SEQ ID NO: 259 - protein, 8 aa, Artificial Sequence
  Description of Artificial Sequence: Synthetic peptide
HQWNNYGT

SEQ ID NO: 260 - protein, 124 aa, Artificial Sequence
  Description of Artificial Sequence: Synthetic polypeptide
QVTLKESGPALVKPTQTLTLTCTFSGFSLSTSNMGVVWIRQPPGKALEWLAHILWDDREYSNPALKSRLTISKDTSKNQVVLTMTNMDPVDTATYYCARMSRNYYGSSYVMDYWGQGTLVTVSS

SEQ ID NO: 261 - protein, 106 aa, Artificial Sequence
  Description of Artificial Sequence: Synthetic polypeptide
DIQLTQSPSFLSASVGDRVTITCSASSSVNYMHWYQQKPGKAPKLLIYSTSNLASGVPSRFSGSGSGTEFTLTISSLQPEDFATYYCHQWNNYGTFGQGTKVEIKR

SEQ ID NO: 262 - protein, 5 aa, Artificial Sequence
  Description of Artificial Sequence: Synthetic peptide
LYGMN

SEQ ID NO: 263 - protein, 5 aa, Artificial Sequence
  Description of Artificial Sequence: Synthetic peptide
NYGMN

SEQ ID NO: 264 - protein, 17 aa, Artificial Sequence
  Description of Artificial Sequence: Synthetic peptide
WINTYTGEPTYADDFKG

SEQ ID NO: 265 - protein, 10 aa, Artificial Sequence
  Description of Artificial Sequence: Synthetic peptide
DTAMDYAMAY

SEQ ID NO: 266 - protein, 13 aa, Artificial Sequence
  Description of Artificial Sequence: Synthetic peptide
DYGKYGDYYAMDY

SEQ ID NO: 267 - protein, 16 aa, Artificial Sequence
  Description of Artificial Sequence: Synthetic peptide
KSSQNIVHSDGNTYLE

SEQ ID NO: 268 - protein, 16 aa, Artificial Sequence
  Description of Artificial Sequence: Synthetic peptide
RSSQSIVHSNGNTYLD

SEQ ID NO: 269 - protein, 7 aa, Artificial Sequence
  Description of Artificial Sequence: Synthetic peptide
KVSNRFS

SEQ ID NO: 270 - protein, 9 aa, Artificial Sequence
  Description of Artificial Sequence: Synthetic peptide
FQGSHVPLT

SEQ ID NO: 271 - protein, 119 aa, Artificial Sequence
  Description of Artificial Sequence: Synthetic polypeptide
QVQLVQSGSELKKPGASVKVSCKASGYTFTLYGMNWVRQAPGQGLEWMGWINTYTGEPTYADDFKGRFVFSLDTSVSTAYLQISSLKAEDTAVYYCARDTAMDYAMAYWGQGTLVTVSS

SEQ ID NO: 272 - protein, 119 aa, Artificial Sequence
  Description of Artificial Sequence: Synthetic polypeptide
QVQLVQSGSELKKPGASVKVSCKASGYTFTLYGMNWVKQAPGKGLKWMGWINTYTGEPTYADDFKGRFVFSLDTSVSTAYLQISSLKAEDTAVYFCARDTAMDYAMAYWGQGTLVTVSS

SEQ ID NO: 273 - protein, 122 aa, Artificial Sequence
  Description of Artificial Sequence: Synthetic polypeptide
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARDYGKYGDYYAMDYWGQGTLVTVSS

SEQ ID NO: 274 - protein, 122 aa, Artificial Sequence
  Description of Artificial Sequence: Synthetic polypeptide
QVQLVQSGSELKKPGASVKVSCKASGYTFTNYGMNWVRQAPGKGLKWMGWINTYTGEPTYADDFKGRFVFSLDTSVSTAYLQISSLKAEDTAVYFCARDYGKYGDYYAMDYWGQGTLVTVSS

SEQ ID NO: 275 - protein, 113 aa, Artificial Sequence
  Description of Artificial Sequence: Synthetic polypeptide
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGGGTKVEIKR

SEQ ID NO: 276 - protein, 113 aa, Artificial Sequence
  Description of Artificial Sequence: Synthetic polypeptide
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGQGTKVEIKR

SEQ ID NO: 277 - protein, 113 aa, Artificial Sequence
  Description of Artificial Sequence: Synthetic polypeptide
DVVMTQTPLSLPVTPGEPASISCKSSQNIVHSDGNTYLEWYLQKPGQSPQLLIYKVSNRFSGVPDRFSGSGSGTDFTLKISRVEAEDLGVYYCFQGSHVPLTFGGGTKVEIKR

SEQ ID NO: 278 - protein, 113 aa, Artificial Sequence
  Description of Artificial Sequence: Synthetic polypeptide
DVVMTQTPLSLPVSLGDQASISCKSSQNIVHSDGNTYLEWYLQKPGQSPKVLIYKVSNRFSGVPDRFSGSGSGTDFTLKISRVEAEDLGVYYCFQGSHVPLTFGGGTKVEIKR

SEQ ID NO: 279 - protein, 113 aa, Artificial Sequence
  Description of Artificial Sequence: Synthetic polypeptide
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGGGTKVEIKR

SEQ ID NO: 280 - protein, 113 aa, Artificial Sequence
  Description of Artificial Sequence: Synthetic polypeptide
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGQGTKVEIKR

SEQ ID NO: 281 - protein, 113 aa, Artificial Sequence
  Description of Artificial Sequence: Synthetic polypeptide
DVVMTQTPLSLPVTPGEPASISCRSSQSIVHSNGNTYLDWYLQKPGQSPQLLIYKVSNRFSGVPDRFSGSGSGTDFTLKISRVEAEDLGVYYCFQGSHVPLTFGGGTKVEIKR

SEQ ID NO: 282 - protein, 113 aa, Artificial Sequence
  Description of Artificial Sequence: Synthetic polypeptide
DVVMTQTPLSLPVSLGDQASISCRSSQSIVHSNGNTYLDWYLQKPGQSPKVLIYKVSNRFSGVPDRFSGSGSGTDFTLKINRVEAEDLGVYFCFQGSHVPLTFGGGTKLEIKR

SEQ ID NO: 283 - protein, 10 aa, Artificial Sequence
  Description of Artificial Sequence: Synthetic peptide
GYTFTSSWMH

SEQ ID NO: 284 - protein, 8 aa, Artificial Sequence
  Description of Artificial Sequence: Synthetic peptide
IHPNSGGT

SEQ ID NO: 285 - protein, 14 aa, Artificial Sequence
  Description of Artificial Sequence: Synthetic peptide
ARGDYYGYVSWFAY

SEQ ID NO: 286 - protein, 6 aa, Artificial Sequence
  Description of Artificial Sequence: Synthetic peptide
QNINVL

SEQ ID NO: 287 - protein, 3 aa, Artificial Sequence
  Description of Artificial Sequence: Synthetic peptide
KAS

SEQ ID NO: 288 - protein, 9 aa, Artificial Sequence
  Description of Artificial Sequence: Synthetic peptide
QQGQSYPYT

SEQ ID NO: 289 - protein, 119 aa, Artificial Sequence
  Description of Artificial Sequence: Synthetic polypeptide
QVQLQQPGSVLVRPGASVKVSCKASGYTFTSSWMHWAKQRPGQGLEWIGEIHPNSGGTNYNEKFKGKATVDTSSSTAYVDLSSLTSEDSAVYYCARGDYYGYVSWFAYWGQGTLVTVSS

SEQ ID NO: 290 - protein, 121 aa, Artificial Sequence
  Description of Artificial Sequence: Synthetic polypeptide
QVQLVQSGAEVKKPGASVKVSCKASGYTFTSSWMHWARQAPGQGLEWIGEIHPNSGGTNYAQKFQGRATLTVDTSSSTAYMELSRLRSDDTAVYYCARGDYYGYVSWFAYWGQGTLVTVSS

SEQ ID NO: 291 - protein, 121 aa, Artificial Sequence
  Description of Artificial Sequence: Synthetic polypeptide
QVQLVQSGAEVKKPGASVKVSCKASGYTFTSSWMHWARQAPGQGLEWIGEIHPNSGGTNYAQKFQGRATMTVDTSISTAYMELSRLRSDDTAVYYCARGDYYGYVSWFAYWGQGTLVTVSS

SEQ ID NO: 292 - protein, 121 aa, Artificial Sequence
  Description of Artificial Sequence: Synthetic polypeptide
QVQLVQSGAEVKKPGASVKVSCKASGYTFTSSWMHWARQAPGQGLEWIGEIHPNSGGTNYAQKFQGRVTMTVDTSISTAYMELSRLRSDDTAVYYCARGDYYGYVSWFAYWGQGTLVTVSS

SEQ ID NO: 293 - protein, 121 aa, Artificial Sequence
  Description of Artificial Sequence: Synthetic polypeptide
QVQLVQSGAEVKKPGASVKVSCKASGYTFTSSWMHWARQAPGQGLEWMGEIHPNSGGTNYAQKFQGRVTMTVDTSISTAYMELSRLRSDDTAVYYCARGDYYGYVSWFAYWGQGTLVTVSS

SEQ ID NO: 294 - protein, 107 aa, Artificial Sequence
  Description of Artificial Sequence: Synthetic polypeptide
DIQMNQSPSSLSASLGDTITITCHASQNINVLLSWYQQKPGNIPKLLIYKASNLHTGVPSRFSGSGSGTGFTFTISSLQPEDIATYYCQQGQSYPYTFGGGTKLEIK

SEQ ID NO: 295 - protein, 107 aa, Artificial Sequence
  Description of Artificial Sequence: Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 296 - protein, 107 aa, Artificial Sequence
  Description of Artificial Sequence: Synthetic polypeptide
DIQMTQSPSSLSASVGDRVTITCQASQNINVLLNWYQQKPGKAPKLLIYKASNLHTGVPSRFSGSGSGTDFTFTISSLQPEDIATYYCQQGQSYPYTFGQGTKLEIK

SEQ ID NO: 297 - protein, 107 aa, Artificial Sequence
  Description of Artificial Sequence: Synthetic polypeptide
DIQMNQSPSSLSASVGDRVTITCQASQNINVLLSWYQQKPGKAPKLLIYKASNLHTGVPSRFSGSGSGTDFTFTISSLQPEDIATYYCQQGQSYPYTFGQGTKLEIK

SEQ ID NO: 298 - protein, 10 aa, Artificial Sequence
  Description of Artificial Sequence: Synthetic peptide
GYTFTSYDIN

SEQ ID NO: 299 - protein, 10 aa, Artificial Sequence
  Description of Artificial Sequence: Synthetic peptide
  MOD_RES (8)..(8): Asn or Tyr
WLNPNSGXTG

SEQ ID NO: 300 - protein, 10 aa, Artificial Sequence
  Description of Artificial Sequence: Synthetic peptide
EVPETAAFEY

SEQ ID NO: 301 - protein, 14 aa, Artificial Sequence
  Description of Artificial Sequence: Synthetic peptide
  MOD_RES (10)..(10): Gly or Ala
  MOD_RES (11)..(11): Leu, Ser or Gln
  MOD_RES (14)..(14): His or Leu
TSSSSDIGAXXGVX

SEQ ID NO: 302 - protein, 7 aa, Artificial Sequence
  Description of Artificial Sequence: Synthetic peptide
GYYNRPS

SEQ ID NO: 303 - protein, 10 aa, Artificial Sequence
  Description of Artificial Sequence: Synthetic peptide
  MOD_RES (3)..(3): Tyr, Trp or Phe
QSXDGTLSAL

SEQ ID NO: 304 - protein, 119 aa, Artificial Sequence
  Description of Artificial Sequence: Synthetic polypeptide
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 305 - protein, 111 aa, Artificial Sequence
  Description of Artificial Sequence: Synthetic polypeptide
  MOD_RES (32)..(32): Any amino acid
  MOD_RES (33)..(33): Any amino acid
  MOD_RES (36)..(36): Any amino acid
  MOD_RES (93)..(93): Any amino acid
QSVLTQPPSVSGAPGQRVTISCTSSSSDIGAXXGVXWYQQLPGTAPKLLIEGYYNRPSGVPDRFSGSKSGTSASLTITGLLPEDEGDYYCQSXDGTLSALFGGGTKLTVLG

SEQ ID NO: 306 - protein, 119 aa, Artificial Sequence; identical to SEQ ID NO: 304
  Description of Artificial Sequence: Synthetic polypeptide

SEQ ID NO: 307 - protein, 111 aa, Artificial Sequence
  Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 308 - protein, 119 aa, Artificial Sequence
  Description of Artificial Sequence: Synthetic polypeptide
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 309 - protein, 111 aa, Artificial Sequence
  Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 310 - protein, 119 aa, Artificial Sequence; identical to SEQ ID NO: 304
  Description of Artificial Sequence: Synthetic polypeptide

SEQ ID NO: 311 - protein, 111 aa, Artificial Sequence
  Description of Artificial Sequence: Synthetic polypeptide
QSVLTQPPSVSGAPGQRVTISCTSSSSDIGAALGVHWYQQLPGTAPKLLIEGYYNRPSGVPDRFSGSKSGTSASLTITGLLPEDEGDYYCQSWDGTLSALFGGGTKLTVLG

SEQ ID NO: 312 - protein, 119 aa, Artificial Sequence; identical to SEQ ID NO: 304
  Description of Artificial Sequence: Synthetic polypeptide

SEQ ID NO: 313 - protein, 111 aa, Artificial Sequence
  Description of Artificial Sequence: Synthetic polypeptide
QSVLTQPPSVSGAPGQRVTISCTSSSSDIGAGSGVHWYQQLPGTAPKLLIEGYYNRPSGVPDRFSGSKSGTSASLTITGLLPEDEGDYYCQSWDGTLSALFGGGTKLTVLG

SEQ ID NO: 314 - protein, 119 aa, Artificial Sequence; identical to SEQ ID NO: 304
  Description of Artificial Sequence: Synthetic polypeptide

SEQ ID NO: 315 - protein, 111 aa, Artificial Sequence
  Description of Artificial Sequence: Synthetic polypeptide
QSVLTQPPSVSGAPGQRVTISCTSSSSDIGAGQGVHWYQQLPGTAPKLLIEGYYNRPSGVPDRFSGSKSGTSASLTITGLLPEDEGDYYCQSWDGTLSALFGGGTKLTVLG

SEQ ID NO: 316 - protein, 119 aa, Artificial Sequence; identical to SEQ ID NO: 304
  Description of Artificial Sequence: Synthetic polypeptide

SEQ ID NO: 317 - protein, 111 aa, Artificial Sequence
  Description of Artificial Sequence: Synthetic polypeptide
QSVLTQPPSVSGAPGQRVTISCTSSSSDIGAGLGVLWYQQLPGTAPKLLIEGYYNRPSGVPDRFSGSKSGTSASLTITGLLPEDEGDYYCQSWDGTLSALFGGGTKLTVLG

SEQ ID NO: 318 - protein, 119 aa, Artificial Sequence; identical to SEQ ID NO: 308
  Description of Artificial Sequence: Synthetic polypeptide

SEQ ID NO: 319 - protein, 111 aa, Artificial Sequence; identical to SEQ ID NO: 307
  Description of Artificial Sequence: Synthetic polypeptide

SEQ ID NO: 320 - protein, 119 aa, Artificial Sequence; identical to SEQ ID NO: 308
  Description of Artificial Sequence: Synthetic polypeptide

SEQ ID NO: 321 - protein, 111 aa, Artificial Sequence; identical to SEQ ID NO: 313
  Description of Artificial Sequence: Synthetic polypeptide

SEQ ID NO: 322 - protein, 119 aa, Artificial Sequence; identical to SEQ ID NO: 308
  Description of Artificial Sequence: Synthetic polypeptide

SEQ ID NO: 323 - protein, 111 aa, Artificial Sequence; identical to SEQ ID NO: 315
  Description of Artificial Sequence: Synthetic polypeptide

SEQ ID NO: 324 - protein, 119 aa, Artificial Sequence; identical to SEQ ID NO: 308
  Description of Artificial Sequence: Synthetic polypeptide

SEQ ID NO: 325 - protein, 111 aa, Artificial Sequence; identical to SEQ ID NO: 317
  Description of Artificial Sequence: Synthetic polypeptide

SEQ ID NO: 326 - protein, 119 aa, Artificial Sequence; identical to SEQ ID NO: 308
  Description of Artificial Sequence: Synthetic polypeptide

SEQ ID NO: 327 - protein, 111 aa, Artificial Sequence
  Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSFDGTLSALFGGGTKLTVLG

SEQ ID NO: 328 - protein, 5 aa, Artificial Sequence
  Description of Artificial Sequence: Synthetic peptide
SYFWS

SEQ ID NO: 329 - protein, 16 aa, Artificial Sequence
  Description of Artificial Sequence: Synthetic peptide
YIYYSGNTKYNPSLKS

SEQ ID NO: 330 - protein, 10 aa, Artificial Sequence
  Description of Artificial Sequence: Synthetic peptide
ETGSYYGFDY

SEQ ID NO: 331 - protein, 11 aa, Artificial Sequence
  Description of Artificial Sequence: Synthetic peptide
RASQSINNYLN

SEQ ID NO: 332 - protein, 7 aa, Artificial Sequence
  Description of Artificial Sequence: Synthetic peptide
AASSLQS

SEQ ID NO: 333 - protein, 9 aa, Artificial Sequence
  Description of Artificial Sequence: Synthetic peptide
QQSYSTPRT

SEQ ID NO: 334 - protein, 118 aa, Artificial Sequence
  Description of Artificial Sequence: Synthetic polypeptide
QVQLQESGPGLVKPSETLSLTCTVSGGSISSYFWSWIRQPPGKGLEWIGYIYYSGNTKYNPSLKSRVTISIDTSKNQFSLKLSSVTAADTAVYYCARETGSYYGFDYWGQGTLVTVSS

SEQ ID NO: 335 - protein, 107 aa, Artificial Sequence
  Description of Artificial Sequence: Synthetic polypeptide
DIQMTQSPSSLSASVGDRVTITCRASQSINNYLNWYQQRPGKAPKLLIYAASSLQSGVPSRFSGSGSGTDFTLTISSLQPGDFATYYCQQSYSTPRTFGQGTKLEIK

SEQ ID NO: 336 - protein, 5 aa, Artificial Sequence
  Description of Artificial Sequence: Synthetic peptide
GYYWN

SEQ ID NO: 337 - protein, 16 aa, Artificial Sequence
  Description of Artificial Sequence: Synthetic peptide
EINHAGNTNYNPSLKS

SEQ ID NO: 338 - protein, 12 aa, Artificial Sequence
  Description of Artificial Sequence: Synthetic peptide
GYCRSTTCYFDY

SEQ ID NO: 339 - protein, 12 aa, Artificial Sequence
  Description of Artificial Sequence: Synthetic peptide
RASQSVRSSYLA

SEQ ID NO: 340 - protein, 7 aa, Artificial Sequence
  Description of Artificial Sequence: Synthetic peptide
GASSRAT

SEQ ID NO: 341 - protein, 8 aa, Artificial Sequence
  Description of Artificial Sequence: Synthetic peptide
QQYGSSPT

SEQ ID NO: 342 - protein, 120 aa, Artificial Sequence
  Description of Artificial Sequence: Synthetic polypeptide
QVQLQQWGAGLLKPSETLSLTCAVHGGSFSGYYWNWIRQPPGKGLEWIGEINHAGNTNYNPSLKSRVTISLDTSKNQFSLTLTSVTAADTAVYYCARGYCRSTTCYFDYWGQGTLVTVSS

SEQ ID NO: 343 - protein, 107 aa, Artificial Sequence
  Description of Artificial Sequence: Synthetic polypeptide
EIVLTQSPGTLSLSPGERATLSCRASQSVRSSYLAWYQQKPGQAPRLLIYGASSRATGIPDRFSGSGSGTDFTLTISRLEPEDFAVYYCQQYGSSPTFGQGTRLEIK

SEQ ID NO: 344 - protein, 116 aa, Artificial Sequence
  Description of Artificial Sequence: Synthetic polypeptide
EVQLQQSGAELVKPGASVKLSCTASGFDIQDTYMHWVKQRPEQGLEWIGRIDPASGHTKYDPKFQVKATITTDTSSNTAYLQLSSLTSEDTAVYYCSRSGGLPDVWGAGTTVTVSS

SEQ ID NO: 345 - protein, 106 aa, Artificial Sequence
  Description of Artificial Sequence: Synthetic polypeptide
QIVLSQSPAILSASPGEKVTMTCRASSSVSYMYWYQQKPGSSPKPWIYATSNLASGVPDRFSGSGSGTSYSLTISRVEAEDAATYYCQQWSGNPRTFGGGTKLEIK

SEQ ID NO: 346 - protein, 10 aa, Artificial Sequence
  Description of Artificial Sequence: Synthetic peptide
GFDIQDTYMH

SEQ ID NO: 347 - protein, 17 aa, Artificial Sequence
  Description of Artificial Sequence: Synthetic peptide
RIDPASGHTKYDPKFQV

SEQ ID NO: 348 - protein, 7 aa, Artificial Sequence
  Description of Artificial Sequence: Synthetic peptide
SGGLPDV

SEQ ID NO: 349 - protein, 10 aa, Artificial Sequence
  Description of Artificial Sequence: Synthetic peptide
RASSSVSYMY

SEQ ID NO: 350 - protein, 7 aa, Artificial Sequence
  Description of Artificial Sequence: Synthetic peptide
ATSNLAS

SEQ ID NO: 351 - protein, 9 aa, Artificial Sequence
  Description of Artificial Sequence: Synthetic peptide
QQWSGNPRT

SEQ ID NO: 352 - protein, 116 aa, Artificial Sequence
  Description of Artificial Sequence: Synthetic polypeptide
QVQLVQSGAEVKKPGASVKLSCKASGFDIQDTYMHWVRQAPGQGLEWMGRIDPASGHTKYDPKFQVRVTMTTDTSTSTVYMELSSLRSEDTAVYYCSRSGGLPDVWGQGTTVTVSS

SEQ ID NO: 353 - protein, 106 aa, Artificial Sequence
  Description of Artificial Sequence: Synthetic polypeptide
EIVLTQSPGTLSLSPGERVTMSCRASSSVSYMYWYQQKPGQAPRPWIYATSNLASGVPDRFSGSGSGTDYTLTISRLEPEDFAVYYCQQWSGNPRTFGGGTKLEIK

SEQ ID NO: 354 - protein, 116 aa, Artificial Sequence
  Description of Artificial Sequence: Synthetic polypeptide
QVQLVQSGAEVKKPGASVKLSCKASGFDIQDTYMHWVRQAPGQGLEWMGRIDPASGHTKYDPKFQVRVTMTRDTSTSTVYMELSSLRSEDTAVYYCSRSGGLPDVWGQGTTVTVSS

SEQ ID NO: 355 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRTFGGGTKLEIK

SEQ ID NO: 356 - protein, 116 aa, Artificial Sequence
  Description of Artificial Sequence: Synthetic polypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDVWGQGTTVTVSS

SEQ ID NO: 357 - protein, 106 aa, Artificial Sequence
  Description of Artificial Sequence: Synthetic polypeptide
EIVLTQSPGTLSLSPGERATLSCRASSSVSYMYWYQQKPGQAPRLLIYATSNLASGVPDRFSGSGSGTDYTLTISRLEPEDFAVYYCQQWSGNPRTFGGGTKLEIK

SEQ ID NO: 358 - protein, 121 aa, Artificial Sequence
  Description of Artificial Sequence: Synthetic polypeptide
EVMLVESGGGLVKPGGSLKLSCAASGFTFTNYAMSWVRQTPEKRLEWVATITSGGSYIYYLDSVKGRFTISRDNAKSTLYLQMSSLRSEDTAIYNCARRKDGNYYYAMDYWGQGTSVTVSS

SEQ ID NO: 359 - protein, 121 aa, Artificial Sequence
  Description of Artificial Sequence: Synthetic polypeptide
EVMLVESGGGLVKPGGSLKLSCAASGFTFTNYAMSWVRQTPEKRLEWVATITSGGSYIYYLDSVKGRFTISRDNAKSTLYLQMSSLRSEDTAIYYCARRKDGNYYYAMDYWGQGTSVTVSS

SEQ ID NO: 360 - protein, 121 aa, Artificial Sequence
  Description of Artificial Sequence: Synthetic polypeptide
EVQLVESGGGLVKPGGSLRLSCAASGFTFTNYAMSWVRQAPGQRLEWVSTITSGGSYIYYLDSVKGRFTISRDNAKSTLYLQMNSLRAEDTAVYNCARRKDGNYYYAMDYWGQGTTVTVSS

SEQ ID NO: 361 - protein, 121 aa, Artificial Sequence
  Description of Artificial Sequence: Synthetic polypeptide
EVQLVESGGGLVKPGGSLRLSCAASGFTFTNYAMSWVRQAPGQRLEWVSTITSGGSYIYYLDSVKGRFTISRDNAKSTLYLQMNSLRAEDTAVYYCARRKDGNYYYAMDYWGQGTTVTVSS

SEQ ID NO: 362 - protein, 121 aa, Artificial Sequence
  Description of Artificial Sequence: Synthetic polypeptide
EVQLLESGGGLVQPGRSLRLSCAASGFTFTNYAMSWVRQAPGQRLEWLATITSGGSYIYYLDSVKGRFTISRDNSKSTLYLQMGSLRAEDMAVYNCARRKDGNYYYAMDYWGQGTTVTVSS

SEQ ID NO: 363 - protein, 121 aa, Artificial Sequence
  Description of Artificial Sequence: Synthetic polypeptide
EVQLLESGGGLVQPGRSLRLSCAASGFTFTNYAMSWVRQAPGQRLEWLATITSGGSYIYYLDSVKGRFTISRDNSKSTLYLQMGSLRAEDMAVYYCARRKDGNYYYAMDYWGQGTTVTVSS

SEQ ID NO: 364 - protein, 121 aa, Artificial Sequence
  Description of Artificial Sequence: Synthetic polypeptide
QVQLVESGGGLIQPGGSLRLSCAASGFTFTNYAMSWVRQARGQRLEWVSTITSGGSYIYYLDSVKGRFTISRDNSKSTLYMELSSLRSEDTAVYNCARRKDGNYYYAMDYWGQGTTVTVSS

SEQ ID NO: 365 - protein, 121 aa, Artificial Sequence
  Description of Artificial Sequence: Synthetic polypeptide
QVQLVESGGGLIQPGGSLRLSCAASGFTFTNYAMSWVRQARGQRLEWVSTITSGGSYIYYLDSVKGRFTISRDNSKSTLYMELSSLRSEDTAVYYCARRKDGNYYYAMDYWGQGTTVTVSS

SEQ ID NO: 366 - protein, 121 aa, Artificial Sequence
  Description of Artificial Sequence: Synthetic polypeptide
QVQLVQSGSELKKPGASVKVSCKASGFTFTNYAMSWVRQAPGKRLEWVSTITSGGSYIYYLDSVKGRFTISRENAKSTLYLQMNSLRTEDTALYNCARRKDGNYYYAMDYWGQGTTVTVSS

SEQ ID NO: 367 - protein, 121 aa, Artificial Sequence
  Description of Artificial Sequence: Synthetic polypeptide
QVQLVQSGSELKKPGASVKVSCKASGFTFTNYAMSWVRQAPGKRLEWVATITSGGSYIYYLDSVKGRFTISRENAKSTLYLQMNSLRTEDTALYYCARRKDGNYYYAMDYWGQGTTVTVSS

SEQ ID NO: 368 - protein, 121 aa, Artificial Sequence
  Description of Artificial Sequence: Synthetic polypeptide
EVQLLQSGAEVKKPGASVKVSCKASGFTFTNYAMSWVRQAPGQRLEWVATITSGGSYIYYLDSVKGRFTISRDNAKSTLHLQMNSLRAEDTAVYNCARRKDGNYYYAMDYWGQGTTVTVSS

SEQ ID NO: 369 - protein, 121 aa, Artificial Sequence
  Description of Artificial Sequence: Synthetic polypeptide
EVQLLQSGAEVKKPGASVKVSCKASGFTFTNYAMSWVRQAPGQRLEWVATITSGGSYIYYLDSVKGRFTISRDNAKSTLHLQMNSLRAEDTAIYYCARRKDGNYYYAMDYWGQGTTVTVSS

SEQ ID NO: 370 - protein, 121 aa, Artificial Sequence
  Description of Artificial Sequence: Synthetic polypeptide
EVMLLQSGAEVKKPGASVKVSCKASGFTFTNYAMSWVRQAPGQRLEWVATITSGGSYIYYLDSVKGRFTISRDNAKSTLHLQMNSLRAEDTAVYYCARRKDGNYYYAMDYWGQGTTVTVSS

SEQ ID NO: 371 - protein, 111 aa, Artificial Sequence
  Description of Artificial Sequence: Synthetic polypeptide
DIVLTQSPASLAVSLGQRATISCRASESVDSYGNSFIHWYQQKAGQPPKLLIYRASNLESGIPARFSGSGSRTDFTLTINPVEADDVATYYCQQSYEDPWTFGGGTKLEIK

SEQ ID NO: 372 - protein, 111 aa, Artificial Sequence
  Description of Artificial Sequence: Synthetic polypeptide
  MOD_RES (108)..(108): Any amino acid
DIVLTQSPATLSLSPGERATLSCRASESVDSYGNSFIHWYQQKPGQPPKLLIYRASNLESGIPARFSGSGSRTDFTLTISSLEPEDFAVYYCQQSYEDPWTFGGGTKXEIK

SEQ ID NO: 373 - protein, 111 aa, Artificial Sequence
  Description of Artificial Sequence: Synthetic polypeptide
  MOD_RES (108)..(108): Any amino acid
DIVLTQSPSSLSASVGDRVTITCRASESVDSYGNSFIHWYQQKPGQPPKLLIYRASNLESGIPARFSGSGSRTDFTLTISSLQPEDFATYYCQQSYEDPWTFGGGTKXEIK

SEQ ID NO: 374 - protein, 111 aa, Artificial Sequence
  Description of Artificial Sequence: Synthetic polypeptide
  MOD_RES (108)..(108): Any amino acid
DIVLTQSPDFQSVTPKEKVTITCRASESVDSYGNSFIHWYQQKPGQPPKLLIYRASNLESGIPARFSGSGSRTDFTLTISSLEAEDAATYYCQQSYEDPWTFGGGTKXEIK

SEQ ID NO: 375 - protein, 111 aa, Artificial Sequence
  Description of Artificial Sequence: Synthetic polypeptide
  MOD_RES (108)..(108): Any amino acid
DIVLTQTPLSLSVTPGQPASISCRASESVDSYGNSFIHWYQQKPGQPPKLLIYRASNLESGIPARFSGSGSRTDFTLKISRVEAEDVGVYYCQQSYEDPWTFGGGTKXEIK

SEQ ID NO: 376 - protein, 5 aa, Artificial Sequence
  Description of Artificial Sequence: Synthetic peptide
TYGMS

SEQ ID NO: 377 - protein, 17 aa, Artificial Sequence
  Description of Artificial Sequence: Synthetic peptide
WMNTYSGVTTYADDFKG

SEQ ID NO: 378 - protein, 13 aa, Artificial Sequence
  Description of Artificial Sequence: Synthetic peptide
EGYVFDDYYATDY

SEQ ID NO: 379 - protein, 16 aa, Artificial Sequence
  Description of Artificial Sequence: Synthetic peptide
RSSQNIVHSDGNTYLE

SEQ ID NO: 380 - protein, 7 aa, Artificial Sequence
  Description of Artificial Sequence: Synthetic peptide
KVSNRFS

SEQ ID NO: 381 - protein, 9 aa, Artificial Sequence
  Description of Artificial Sequence: Synthetic peptide
FQGSHVPLT

SEQ ID NO: 382 - protein, 122 aa, Artificial Sequence
  Description of Artificial Sequence: Synthetic polypeptide
QIQLVQSGPELKKPGETVKISCKASGYTFTTYGMSWVKQAPGKGLKWMGWMNTYSGVTTYADDFKGRFAFSLETSASTAYMQIDNLKNEDTATYFCAREGYVFDDYYATDYWGQGTSVTVSS

SEQ ID NO: 383 - protein, 112 aa, Artificial Sequence
  Description of Artificial Sequence: Synthetic polypeptide
DVLMTQTPLSLPVSLGDQASISCRSSQNIVHSDGNTYLEWYLQKPGQSPKLLIYKVSNRFSGVPDRFSGSGSGTDFTLKISRVEAEDLGIYYCFQGSHVPLTFGAGTKLELK

SEQ ID NO: 384 - protein, 5 aa, Artificial Sequence
  Description of Artificial Sequence: Synthetic peptide
KYDIN

SEQ ID NO: 385 - protein, 17 aa, Artificial Sequence
  Description of Artificial Sequence: Synthetic peptide
WIFPGDGRTDYNEKFKG

SEQ ID NO: 386 - protein, 7 aa, Artificial Sequence
  Description of Artificial Sequence: Synthetic peptide
YGPAMDY

SEQ ID NO: 387 - protein, 16 aa, Artificial Sequence
  Description of Artificial Sequence: Synthetic peptide
RSSQTIVHSNGDTYLD

SEQ ID NO: 388 - protein, 7 aa, Artificial Sequence
  Description of Artificial Sequence: Synthetic peptide
KVSNRFS

SEQ ID NO: 389 - protein, 9 aa, Artificial Sequence
  Description of Artificial Sequence: Synthetic peptide
FQGSHVPYT

SEQ ID NO: 390 - protein, 135 aa, Artificial Sequence
  Description of Artificial Sequence: Synthetic polypeptide
MGWSWVFLFLLSVTAGVHSQVHLQQSGPELVKPGASVKLSCKASGYTFTKYDINWVRQRPEQGLEWIGWIFPGDGRTDYNEKFKGKATLTTDKSSSTAYMEVSRLTSEDSAVYFCARYGPAMDYWGQGTSVTVAS

SEQ ID NO: 391 - protein, 131 aa, Artificial Sequence
  Description of Artificial Sequence: Synthetic polypeptide
MKLPVRLLVLMFWIPASSSDVLMTQTPLSLPVSLGDQASISCRSSQTIVHSNGDTYLDWFLQKPGQSPKLLIYKVSNRFSGVPDRFSGSGSGTDFTLKISRVEAEDLGVYYCFQGSHVPYTFGGGTKLEIK

SEQ ID NO: 392 - protein, 4 aa, Homo sapiens
DEAD

SEQ ID NO: 393 - protein, 4 aa, Homo sapiens
KDEL

(19) United States
(12) Patent Application Publication
(10) Pub. No.: US 2023/0020356 A1
(43) Pub. Date: Jan. 19, 2023

(54) METHODS OF STRATIFYING AND TREATING A SUB-POPULATION OF INFLAMMATORY BOWEL DISEASE PATIENTS

(71) Applicant: Cedars-Sinai Medical Center, Los Angeles, CA (US)

(72) Inventors: Rebecca Gonsky, Los Angeles, CA (US); Stephan R. Targan, Santa Monica, CA (US)

(21) Appl. No.: 17/334,109

(22) Filed: May 28, 2021

Related U.S. Application Data
(63) Continuation of application No. PCT/US19/63770, filed on Nov. 27, 2019.
(60) Provisional application No. 62/849,622, filed on May 17, 2019.
(60) Provisional application No. 62/820,061, filed on Mar. 18, 2019.
(60) Provisional application No. 62/773,020, filed on Nov. 29, 2018.

(51) Int. Cl. C12Q 1/6883

(52) U.S. Cl. CPC C12Q 1/6883; C12Q 2600/158; C12Q 2600/112

(57) ABSTRACT

Described herein are methods and systems for identifying subpopulations of patients having Crohn's disease, including populations at risk of developing structuring or other severe disease, and populations susceptible to success or failure with surgical intervention. Further provided are therapies useful for treating subpopulations of patients having Crohn's disease.

### CROSS-REFERENCE

This application is a continuation of International Application No. PCT/US19/63770, filed Nov. 27, 2019, which claims the benefit of U.S. Provisional Application Ser. No. 62/773,020, filed Nov. 29, 2018, U.S. Provisional Application No. 62/820,061, filed Mar. 18, 2019, and U.S. Provisional Application No. 62/849,622, filed May 17, 2019, each of which is incorporated herein by reference in its entirety.

### STATEMENT REGARDING FEDERALLY-SPONSORED RESEARCH OR DEVELOPMENT

This invention was made with government support under Grant No. DK043211, RR033176-01, DK062413-18, awarded by National Institutes of Health. The government has certain rights in the invention.

### SEQUENCE LISTING

This application incorporates by reference in its entirety the Computer Readable Form (“CRF”) of a Sequence Listing in ASCII text format submitted via EFS-Web. The Sequence Listing text file submitted via EFS-Web is entitled “56884-745.301_SL.txt,” was created on Aug. 24, 2021 and is 156,673 bytes in size.

### BACKGROUND OF THE INVENTION

Inflammatory bowel disease (IBD) is a pathobiologically heterogeneous disease. Defining distinct disease populations is critical for improved prognostic accuracy, targeted therapeutics and biomarker discovery.

### SUMMARY

Aspects disclosed herein provide methods for processing or analyzing a biological sample from a subject, comprising: (a) obtaining the biological sample comprising gene expression products, wherein the subject has or is suspected of having Crohn's Disease (CD); (b) subjecting the biological sample to an assay to yield a data set including data corresponding to gene expression product levels; (c) in a programmed computer, inputting said data including said gene expression product levels from (b) to a trained algorithm to generate a classification of said sample as positive for a CD-PBmu subtype based detection of an expression profile comprising an increase in the gene expression levels compared to a reference expression profile, wherein the trained algorithm is trained with a plurality of training samples, and wherein said biological sample is independent of said plurality of training samples; and (d) electronically outputting a report that identifies the classification of the biological sample as positive for the CD-PBmu subtype. In some embodiments, the gene expression product comprises RNA. In some embodiments, the assay comprises using one or more of a microarray, sequencing, and qPCR. In some embodiments, the trained algorithm is trained with one or more datasets of gene expression product levels obtained from the plurality of training samples. In some embodiments, the gene expression products are expressed from genes comprising two or more of A disintegrin and metalloproteinase with thrombospondin motifs 1 (ADAMTS1), Neutrophil gelatinase-associated lipocalin (LCN2), Disintegrin and metalloproteinase domain-containing protein 28 (ADAM28), Tryptase beta-2 (TPSB2), peptidylprolyl isomerase A pseudogene 30 (PPIAP30), glutamine-fructose-6-phosphate transaminase 2 (GFPT2), KIT proto-oncogene, receptor tyrosine kinase (KIT), phospholipid transfer protein (PLTP), major facilitator superfamily domain containing 2A (MFSD2A), interleukin 22 (IL22), LIM and cysteine rich domains 1 (LMCD1), interleukin 6 (IL6), TBC1 domain family member 9 (TBC1D9), ChaC glutathione specific gamma-glutamylcyclotransferase 1 (CHAC1), selenoprotein P (SEPP1), superoxide dismutase 3 (SOD3), RAB13, member RAS oncogene family (RAB13), lysozyme (LYZ), carboxypeptidase A3 (CPA3), serine dehydratase (SDS), dual specificity tyrosine phosphorylation regulated kinase 3 (DYRK3), DAB adaptor protein 2 (DAB2), TBC1 domain family member 8 (TBC1D8), crystallin alpha B (CRYAB), TBC1 domain family member 3 (TBC1D3), leucine rich repeat containing 32 (LRRC32), serpin family G member 1 (SERPING1), ubiquitin D (UBD), fatty acid binding protein 1 (FABP1), spleen associated tyrosine kinase (SYK), aldolase, fructose-bisphosphate B (ALDOB), semaphorin 6B (SEMA6B), NANOG neighbor homeobox (NANOGNB), dermatan sulfate epimerase (DSE), formyl peptide receptor 3 (FPR3), tenascin XB (TNXB), olfactory receptor family 4 subfamily A member 5 (OR4A5), decorin (DCN), carbohydrate sulfotransferase 15 (CHST15), ADAM like decysin 1 (ADAMDEC1), histidine decarboxylase (HDC), RRAD, Ras related glycolysis inhibitor and calcium channel regulator (RRAD), complement C1s (C1S), or phospholipase A2 group IIA (PLA2G2A), or a combination thereof. In some embodiments, the gene expression products are expressed from genes comprising two or more of ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, or UBD, or a combination thereof. In some embodiments, the increase in the gene expression product levels is at least 2-fold greater than in the reference expression profile. In some embodiments, the reference expression profile comprises expression levels of the one or more genes of one or more subjects that do not have CD. In some embodiments, the biological sample comprises a blood sample or is purified from a blood sample of the subject. In some embodiments, methods further comprise treating the subject by administering to the subject a therapeutic agent. In some embodiments, methods further comprise optimizing a therapeutic regimen of the subject comprising increasing or decreasing a dosage amount of a therapeutic agent administered to the subject for the treatment of the CD, based on the CD-PBmu subtype.

Aspects disclosed herein provide methods of treating Crohn's disease (CD) in a subject, the method comprising administering to the subject a therapeutically effective amount of a therapeutic agent, provided the subject is identified as having a CD-PBmu subtype by: (a) detecting an expression profile comprising an increase in a level of expression of one or more genes in the biological sample, relative to a reference expression profile; and (b) identifying the subject as having a CD-PBmu subtype based upon the expression profile that is detected in (b). In some embodiments, the one or more genes comprises ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPING1, UBD, FABP1, SYK, ALDOB, SEMA6B, NANOGNB, DSE, FPR3, TNXB, OR4A5, DCN, CHST15, ADAMDEC1, HDC, RRAD, C1S, or PLA2G2A, or a combination thereof. In some embodiments, the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, or UBD, or a combination thereof. In some embodiments, the one or more genes comprises at least 10 of the one or more genes. In some embodiments, the one or more genes comprises between about 10-27 of the one or more genes. In some embodiments, the increase in the level of expression of the one or more genes in the biological sample is at least 2-fold greater than in the reference expression profile. In some embodiments, the reference expression profile comprises expression levels of the one or more genes of one or more subjects that do not have CD. In some embodiments, detecting the expression profile comprises detecting the increase in the level of expression of the one or more genes by: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes.

Aspects disclosed herein provide methods of determining a Crohn's Disease (CD) subtype in a subject having CD, the method comprising: (a) measuring a level of expression of one or more genes from Table 1A in a biological sample obtained from a subject having CD; (b) detecting an expression profile comprising an increase in the level of expression of the one or more genes in the biological sample, relative to a reference expression profile; and (c) identifying the subject as having a CD-PBmu subtype based upon the expression profile that is detected in (b). In some embodiments, provided that the one or more genes comprises ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPING1, UBD, FABP1, SYK, ALDOB, SEMA6B, NANOGNB, DSE, FPR3, TNXB, OR4A5, DCN, CHST15, ADAMDEC1, HDC, RRAD, C1S, or PLA2G2A or a combination thereof. In some embodiments, the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, or UBD, or a combination thereof. In some embodiments, the one or more genes comprises at least 10 of the one or more genes. In some embodiments, the increase in the level of expression of the one or more genes in the biological sample is at least 2-fold greater than in the reference expression profile. In some embodiments, the reference expression profile comprises expression levels of the one or more genes of one or more subjects that do not have CD. In some embodiments, measuring a level of expression of one or more genes comprises utilizing an assay selected from the group consisting of an RNA sequencing method, a microarray method, and quantitative polymerase chain reaction (qPCR). In some embodiments, measuring a level of expression of one or more genes comprises: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes. In some embodiments, methods further comprise treating the subject by administering a therapeutic agent to the subject. In some embodiments, methods further comprise optimizing a therapeutic regimen of the subject comprising increasing or decreasing a dosage amount of a therapeutic agent administered to the subject for the treatment of the CD, based on the CD-PBmu subtype. In some embodiments, provided the biological sample comprises a blood sample or is purified from a blood sample of the subject.

In one aspect, the present application provides methods and systems for identifying sub-populations of Crohn's Disease (CD) patients based on transcriptomic profiling and/or monocyte profiling. Patients having a mucosal-like expression profile may be characterized as having a CD-PBmu subtype, which in some cases is associated with an altered composition of T-cell subsets, clinical disease severity markers, and decreased pro-inflammatory gene expression following surgery. Patients having a particular monocyte expression profile may be characterized as having a more severe form of CD; may be more susceptible to failure with anti-TNF, 6-mercaptopurine, or methotrexate; may be IgG ASCA positive; may have a CD-PBmu subtype, or any combination thereof. Patients having a CD-PBmu and/or monocyte subtype may be specifically treated and monitored using the methods provided herein.

In one aspect, provided herein is a method of determining a Crohn's Disease (CD) subtype status in a subject having CD, wherein the status comprises distinguishing a CD PBmucosal (CD-PBmu) subtype from a non-CD-PBmu subtype, the method comprising: detecting expression of one or more genes from Table 1A in a biological sample from the subject to obtain an expression profile comprising the expression levels of each of the one or more genes in the biological sample, and determining the CD subtype status of the subject based upon the expression profile, wherein an increased level of expression in the one or more genes as compared to a reference expression profile indicates status of CD-PBmu subtype as distinguished from a non-CD-PBmu subtype. In some embodiments, the one or more genes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 65, 70, 75, 80, 85, 90, 100, 110, 120, 140, 160, 180, or 200 genes. In some embodiments, the one or more genes comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or all of the genes in Table 4. In some embodiments, the one or more genes comprises ADAMTS1. In some embodiments, the one or more genes comprises LCN2. In some embodiments, the one or more genes comprises ADAM28. In some embodiments, the one or more genes comprises TPSB2. In some embodiments, the one or more genes comprises PPIAP30. In some embodiments, the one or more genes comprises GFPT2. In some embodiments, the one or more genes comprises KIT. In some embodiments, the one or more genes comprises PLTP. In some embodiments, the one or more genes comprises MFSD2A. In some embodiments, the one or more genes comprises IL22. In some embodiments, the one or more genes comprises LMCD1. In some embodiments, the one or more genes comprises IL6. In some embodiments, the one or more genes comprises TBC1D9. In some embodiments, the one or more genes comprises CHAC1. In some embodiments, the one or more genes comprises SEPP1. In some embodiments, the one or more genes comprises SOD3. In some embodiments, the one or more genes comprises RAB13. In some embodiments, the one or more genes comprises LYZ. In some embodiments, the one or more genes comprises CPA3. In some embodiments, the one or more genes comprises SDS. In some embodiments, the one or more genes comprises DYRK3. In some embodiments, the one or more genes comprises DAB2. In some embodiments, the one or more genes comprises TBC1D8. In some embodiments, the one or more genes comprises CRYAB. In some embodiments, the one or more genes comprises TBC1D3. In some embodiments, the one or more genes comprises LRRC32. In some embodiments, the one or more genes comprises SERPING1. In some embodiments, the one or more genes comprises UBD. In some embodiments, the one or more genes comprises FABP1. In some embodiments, the one or more genes comprises SYK. In some embodiments, the one or more genes comprises ALDOB. In some embodiments, the one or more genes comprises SEMA6B. In some embodiments, the one or more genes comprises NANOGNB. In some embodiments, the one or more genes comprises DSE. In some embodiments, the one or more genes comprises FPR3. In some embodiments, the one or more genes comprises TNXB. In some embodiments, the one or more genes comprises OR4A5. In some embodiments, the one or more genes comprises DCN. In some embodiments, the one or more genes comprises CHST15. In some embodiments, the one or more genes comprises ADAMDEC1. In some embodiments, the one or more genes comprises HDC. In some embodiments, the one or more genes comprises RRAD. In some embodiments, the one or more genes comprises C1S. In some embodiments, the one or more genes comprises PLA2G2A. In some embodiments, the expression of at least one of the one or more genes in the biological sample is at least 2-fold greater than in the reference expression profile. In some embodiments, the reference expression profile comprises expression levels of the one or more genes of one or more subjects who do not have IBD. In some embodiments, detecting expression of the one or more genes comprises a RNA sequencing method. In some embodiments, detecting expression of the one or more genes comprises a microarray method. In some embodiments, detecting expression of the one or more genes comprises hybridization of a nucleic acid primer and/or probe to the biological sample, wherein the nucleic acid primer and/or probe comprises at least about 10 contiguous nucleobases of one of the one or more genes from Table 1A. In some embodiments, the reference expression profile is stored in a database. In some embodiments, the method further comprises treating the subject with a therapeutic agent. In some embodiments, the therapeutic agent comprises a protein, peptide, nucleic acid, or compound that targets a molecule in a pathway of one or more genes of Table 4. In some embodiments, the biological sample comprises a blood sample or is purified from a blood sample of the subject. In some embodiments, the subject is less than 18 years of age. In some embodiments, the subject is 18 years of age or older. In some embodiments, the subject is not responsive to anti-TNFα therapy. In some embodiments, the subject has or is susceptible to having structuring disease. In some embodiments, the subject has or is susceptible to having increased length of bowel resection.

In another aspect, provided is a method for processing or analyzing a biological sample from a subject, comprising: (a) obtaining the biological sample comprising gene expression products, wherein the subject has or is suspected of having Crohn's Disease (CD); (b) subjecting the biological sample to an assay by sequencing, array hybridization, and/or nucleic acid amplification to yield a data set including data corresponding to gene expression product levels; (c) in a programmed computer, inputting said data including said gene expression product levels from (b) to a trained algorithm to generate a classification of said sample as positive or negative for a CD subtype, wherein the trained algorithm is trained with a plurality of training samples, and wherein said biological sample is independent of said plurality of training samples; and (d) electronically outputting a report that identifies the classification of the biological sample as positive or negative for the CD subtype. In some embodiments, the sample is classified at an accuracy of at least about 90%, 91%, 92%, 93%, 94%, 95%, 96%, 97%, 98%, or 99%. In some embodiments, the gene expression product comprises ribonucleic acid. In some embodiments, the assay comprises using one or more of the following: microarray, sequencing, SAGE, blotting, reverse transcription, and quantitative polymerase chain reaction (PCR). In some embodiments, the trained algorithm is trained with one or more datasets of gene expression product levels obtained from the plurality of training samples. In some embodiments, the gene expression products comprise ADAMTS1. In some embodiments, the gene expression products comprise LCN2. In some embodiments, the gene expression products comprise ADAM28. In some embodiments, the gene expression products comprise TPSB2. In some embodiments, the gene expression products comprise PPIAP30. In some embodiments, the gene expression products comprise GFPT2. In some embodiments, the gene expression products comprise KIT. In some embodiments, the gene expression products comprise PLTP. In some embodiments, the gene expression products comprise MFSD2A. In some embodiments, the gene expression products comprise IL22. In some embodiments, the gene expression products comprise LMCD1. In some embodiments, the gene expression products comprise IL6. In some embodiments, the gene expression products comprise TBC1D9. In some embodiments, the gene expression products comprise CHAC1. In some embodiments, the gene expression products comprise SEPP1. In some embodiments, the gene expression products comprise SOD3. In some embodiments, the gene expression products comprise RAB13. In some embodiments, the gene expression products comprise LYZ. In some embodiments, the gene expression products comprise CPA3. In some embodiments, the gene expression products comprise SDS. In some embodiments, the gene expression products comprise DYRK3. In some embodiments, the gene expression products comprise DAB2. In some embodiments, the gene expression products comprise TBC1D8. In some embodiments, the gene expression products comprise CRYAB. In some embodiments, the gene expression products comprise TBC1D3. In some embodiments, the gene expression products comprise LRRC32. In some embodiments, the gene expression products comprise SERPING1. In some embodiments, the gene expression products comprise UBD. In some embodiments, the gene expression products comprise FABP1. In some embodiments, the gene expression products comprise SYK. In some embodiments, the gene expression products comprise ALDOB. In some embodiments, the gene expression products comprise SEMA6B. In some embodiments, the gene expression products comprise NANOGNB. In some embodiments, the gene expression products comprise DSE. In some embodiments, the gene expression products comprise FPR3. In some embodiments, the gene expression products comprise TNXB. In some embodiments, the gene expression products comprise OR4A5. In some embodiments, the gene expression products comprise DCN. In some embodiments, the gene expression products comprise CHST15. In some embodiments, the gene expression products comprise ADAMDEC1. In some embodiments, the gene expression products comprise HDC. In some embodiments, the gene expression products comprise RRAD. In some embodiments, the gene expression products comprise C1S. In some embodiments, the gene expression products comprise PLA2G2A.

Further provided herein is a composition comprising at least 10 but less than 100 contiguous nucleobases of a gene of Table 1A or its complement, and a detectable label.

Further provided herein is a panel of biomarker nucleic acids comprising at least 10 but less than 100 contiguous nucleobases of a plurality of genes, the plurality of genes comprising two or more genes from Table 1A.

Further provided herein is a composition comprising an agent that modulates expression and/or activity of a molecule in a pathway of one or more genes selected from Table 4.

Further provided herein is a method comprising treating a subject with a therapeutic agent that targets a molecule in a pathway of one or more genes selected from Table 4, provided that the subject is determined to have a CD-PBmu subtype as described in any method above. In some embodiments, the therapeutic agent comprises a peptide, nucleic acid, compound, or a combination thereof.

Further provided is a method comprising determining an increase or decrease in expression of a gene effectuated by a therapeutic agent in a subject, the method comprising detecting expression of the gene after administration of the therapeutic agent to the subject, wherein the gene is selected from Table 1A. In some embodiments, the therapeutic agent modulates activity and/or expression of a molecule in a pathway of one or more genes selected from Table 4. In some embodiments, the expression is detected using the method of any above method.

Further provided is a method of determining a Crohn's Disease (CD) subtype status in a subject having CD, wherein the status comprises distinguishing a first CD monocyte subtype from a second CD monocyte subtype, the method comprising: detecting expression of one or more genes from Table 7A in a monocyte sample from the subject to obtain an expression profile comprising the expression levels of each of the one or more genes in the monocyte sample, and determining the CD subtype status of the subject by comparison of the expression profile of the subject to a reference expression profile. In some embodiments, the one or more genes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 65, 70, 75, 80, 85, 90, 100, 110, 120, 140, 160, 180, or 200 genes. In some embodiments, the reference expression profile comprises an expression profile of a first CD monocyte subtype. In some embodiments, if the expression of at least one of the one or more genes in the monocyte sample is at least 2-fold greater than in the reference expression profile, the subject has the second CD monocyte subtype. In some embodiments, if the expression of at least one of the one or more genes in the monocyte sample is at least 2-fold less than in the reference expression profile, the subject has the second CD monocyte subtype. In some embodiments, the reference expression profile comprises an expression profile of a second CD monocyte subtype. In some embodiments, if the expression of at least one of the one or more genes in the monocyte sample is at least 2-fold greater than in the reference expression profile, the subject has the first CD monocyte subtype. In some embodiments, if the expression of at least one of the one or more genes in the monocyte sample is at least 2-fold less than in the reference expression profile, the subject has the first CD monocyte subtype. In some embodiments, the reference expression profile comprises an expression profile comprising expression levels of the one or more genes of a subject who does not have IBD. In some embodiments, the expression of at least one of the one or more genes in the monocyte sample is at least 2-fold greater than in the reference expression profile, the subject has the first CD monocyte subtype. In some embodiments, if the expression of at least one of the one or more genes in the monocyte sample is at least 2-fold greater than in the reference expression profile, the subject has the second CD monocyte subtype. In some embodiments, if the expression of at least one of the one or more genes in the monocyte sample is at least 2-fold less than in the reference expression profile, the subject has the first CD monocyte subtype. In some embodiments, if the expression of at least one of the one or more genes in the monocyte sample is at least 2-fold less than in the reference expression profile, the subject has the second CD monocyte subtype. In some embodiments, detecting expression of the one or more genes comprises a RNA sequencing method. In some embodiments, detecting expression of the one or more genes comprises a microarray method. In some embodiments, detecting expression of the one or more genes comprises hybridization of a nucleic acid primer and/or probe to the biological sample, wherein the nucleic acid primer and/or probe comprises at least about 10 contiguous nucleobases of one of the one or more genes from Table 7A. In some embodiments, the reference expression profile is stored in a database. In some embodiments, the method further comprises treating the subject with a therapeutic agent. In some embodiments, the therapeutic agent comprises a protein, peptide, nucleic acid, or compound that targets a molecule in a pathway of one or more genes of Table 7B. In some embodiments, the therapeutic agent targets a kinase from the group comprising: DNAPK, CDK4, ERK1, HIPK2, CDC2, MAPK1, MAPK3, ERK2, CSNK2A1, CK2ALPHA, JNK1, CDK1, MAPK14, PKR, and CDK2. In some embodiments, the subject is less than 18 years of age. the subject is 18 years of age or older. In some embodiments, the subject is not responsive to anti-TNFα therapy. In some embodiments, the subject has or is susceptible to having structuring disease. In some embodiments, the subject has or is susceptible to having increased length of bowel resection.

Further provided is a method for processing or analyzing a biological sample from a subject, comprising: (a) obtaining the biological sample comprising gene expression products, wherein the subject has or is suspected of having Crohn's Disease (CD); (b) subjecting the biological sample to an assay by sequencing, array hybridization, and/or nucleic acid amplification to yield a data set including data corresponding to gene expression product levels; (c) in a programmed computer, inputting said data including said gene expression product levels from (b) to a trained algorithm to generate a classification of said sample as positive or negative for a CD subtype, wherein the trained algorithm is trained with a plurality of training samples, and wherein said biological sample is independent of said plurality of training samples; and (d) electronically outputting a report that identifies the classification of the biological sample as positive or negative for the CD subtype. In some embodiments, the sample is classified at an accuracy of at least about 90%, 91%, 92%, 93%, 94%, 95%, 96%, 97%, 98%, or 99%. In some embodiments, the gene expression product comprises ribonucleic acid. In some embodiments, the assay comprises using one or more of the following: microarray, sequencing, SAGE, blotting, reverse transcription, and quantitative polymerase chain reaction (PCR). In some embodiments, the trained algorithm is trained with one or more datasets of gene expression product levels obtained from the plurality of training samples. In some embodiments, the gene expression products comprise a gene selected from Table 7A.

Further provided is a composition comprising at least 10 but less than 100 contiguous nucleobases of a gene of Table 7A or its complement, and a detectable label.

A panel of biomarker nucleic acids comprising at least 10 but less than 100 contiguous nucleobases of a plurality of genes, the plurality of genes comprising two or more genes from Table 7A.

Further provided is a composition comprising an agent that modulates expression and/or activity of a molecule in a pathway of one or more genes selected from Table 7B.

Further provided is a method comprising treating a subject with a therapeutic agent that targets a molecule in a pathway of one or more genes selected from Table 7B, provided that the subject is determined to have a monocyte 2 subtype as described in any previous embodiment.

Further provided is a method comprising treating a subject with a therapeutic agent that targets a molecule in a pathway of one or more genes selected from Table 7B, provided that the subject is determined to have a monocyte 1 subtype as described in any of previous embodiment. In some embodiments, the therapeutic agent comprises a peptide, nucleic acid, compound, or a combination thereof.

Further provided is a method comprising determining an increase or decrease in expression of a gene effectuated by a therapeutic agent in a subject, the method comprising detecting expression of the gene after administration of the therapeutic agent to the subject, wherein the gene is selected from Table 7A. In some embodiments, the therapeutic agent modulates activity and/or expression of a molecule in a pathway of one or more genes selected from Table 7B. In some embodiments, the expression is detected using the method described herein.

Further provided is a method of treating or preventing a disease or condition in a subject, the method comprising administering a modulator of the activity or expression of one or more gene from Table 4 to the subject, provided the subject has a first CD subtype characterized by an increased expression in one or more of the genes of Table 1A as compared to the expression of the one or more genes in a reference subject having a second CD subtype. In some embodiments, the disease or condition is inflammatory bowel disease (IBD). In some embodiments, the IBD comprises Crohn's disease and/or ulcerative colitis. In some embodiments, the method further comprises determining the CD subtype of the subject. In some embodiments, determining the CD subtype of the subject comprises determining the expression level of the one or more genes of Table 1A. In some embodiments, determining the CD subtype of the subject comprises performing RNA sequencing.

Further provided is a method of treating or preventing a disease or condition in a subject, the method comprising administering a modulator of the activity or expression of one or more kinases selected from the group consisting of DNAPK, CDK4, ERK1, HIPK2, CDC2, MAPK1, MAPK3, ERK2, CSNK2A1, CK2ALPHA, JNK1, CDK1, MAPK14, PKR, and CDK2, to the subject, provided the subject has a first CD subtype characterized by an increased expression in one or more of the genes of Table 7A as compared to the expression of the one or more genes in a reference subject having a second CD subtype. In some embodiments, the disease or condition is inflammatory bowel disease (IBD). the IBD comprises Crohn's disease and/or ulcerative colitis. In some embodiments, the method further comprises determining the CD subtype of the subject. In some embodiments, determining the CD subtype of the subject comprises determining the expression level of the one or more genes of Table 7A. determining the CD subtype of the subject comprises performing RNA sequencing.

Aspects disclosed herein provide methods of monitoring a treatment of Crohn's disease (CD) in a subject, the method comprising: (a) detecting an expression profile comprising an increase in a level of expression of one or more genes in the biological sample, relative to a reference expression profile; (b) identifying the subject as having a CD-PBmu subtype based upon the expression profile that is detected in (b); and (c) increasing or decreasing a dosage amount of a therapeutic agent that is administered to the subject to treat the CD, based on the CD-PBmu subtype. In some embodiments, the one or more genes comprises ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPING1, UBD, FABP1, SYK, ALDOB, SEMA6B, NANOGNB, DSE, FPR3, TNXB, OR4A5, DCN, CHST15, ADAMDEC1, HDC, RRAD, C1S, or PLA2G2A, or a combination thereof. In some embodiments, the one or more genes comprises at least 10 of the one or more genes. In some embodiments, the one or more genes comprises between about 10-27 of the one or more genes. In some embodiments, the one or more genes comprises 44 of the one or more genes. In some embodiments, the one or more genes comprises ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPING1, UBD, FABP1, SYK, ALDOB, SEMA6B, NANOGNB, DSE, FPR3, TNXB, OR4A5, DCN, CHST15, ADAMDEC1, HDC, RRAD, C1S, and PLA2G2A. In some embodiments, the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, or UBD, or a combination thereof. In some embodiments, the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, and UBD. In some embodiments, the increase in the level of expression of the one or more genes in the biological sample is at least 2-fold greater than in the reference expression profile. In some embodiments, the reference expression profile comprises expression levels of the one or more genes of one or more subjects that do not have CD. In some embodiments, detecting the expression profile comprises detecting the increase in the level of expression of the one or more genes by: (a) contacting the biological sample with a nucleic acid primer and/or detectable nucleic acid probe; and (b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes.

### BRIEF DESCRIPTION OF THE DRAWINGS

FIG. 1A shows differential gene expression and clustering defining a PBmu transcriptomic signature

FIG. 1B shows clustering defining a PBmu transcriptomic signature. Gene expression from a classified CD-PBT clustered tightly with non-IBD subjects and gene expression drifted from a peripheral toward a mucosal profile, classified as CD-PBmu(cosal).

FIG. 1C shows pathways enriched in the CD-PBmu subtype as compared to CD-PBT subtype.

FIG. 1D shows cell population differences in CD-PBT vs CD-PBmu mimic differential RNA expression. LPT refers to mucosal lamina propria T cells.

FIG. 1E shows that the CD-PBmu subtype is enriched in NKT and Tgd T cells and depleted in CD4/CD8 T cells subsets.

FIG. 1F shows that the CD-PBmu expression signature can stratify CD patients who failed on anti-TNF alpha therapy based on whole blood analysis.

FIG. 2 shows NKT cell subset is enriched in CD-PBmu and associated with structuring disease. T-cell subset composition was analyzed using xCell and Bindea bioinformatics tools.

FIG. 3 shows CD4+ memory cells are depleted in CD-PBmu and associated with increased length of bowel resection.

FIG. 4A shows T cell expression changes following surgery in CD-PBmu, but not PBT, subtype.

FIG. 4B shows the CD-PBmu gene signature reverts to that observed for CD-PBT following surgery.

FIG. 5A shows PBmu 44 biomarker signature colocalized with ileal biopsy samples from studies of treatment naive pediatric Crohn's patients.

FIG. 5B shows pathways enriched in the CD-PBmu 44 biomarker signature.

FIG. 5C shows that PBmu 44 biomarker signature is associated with expression of kinases provided.

FIG. 6 shows that for CD-PBmu patients, depletion of CD4+ and CD8+ memory T cells is associated with post-op recurrence of disease.

FIG. 7A-7B show that 44 Biomarker expression gene panel correlates PB-mu enriched NKT and depleted CD4+ memory T cell subsets. FIG. 7A shows enrichment of NKT cells and negative enrichment of CD4+ T cells by cellular enrichment score. FIG. 7B shows enrichment of gene markers of NKT cells and negative enrichment of gene markers of CD4+ T cells.

FIG. 8 shows clustering of CD monocytes to reveal two signatures: monocyte 1 subtype and monocyte 2 subtype.

FIG. 9 shows differential gene expression in monocyte 1 substype versus monocyte 2 subtype.

FIG. 10A shows differentially expressed genes (DEG) in PBmu as compared to PBT in a genome wide association study (GWAS).

FIG. 10B shows enriched pathways that overlap with the GWAS DEG in CD-PBmu.

### DETAILED DESCRIPTION OF THE INVENTION

The present disclosure provides methods and systems for characterizing and treating patients having Crohn's disease (CD). In particular embodiments, a CD patient is characterized as having or not having a mucosal-like CD expression signature (CD-PBmu) by transcriptomic profiling. Patients having the CD-PBmu profile may be suitable for subtype-specific treatment, including administration with a therapeutic agent that targets a biomolecule provided in Table 1A or 3, or a biomolecule in a biological pathway of a biomolecule provided in Table 1A or 3. In some embodiments, a CD patient is characterized as having or not having a particular monocyte profile, monocyte 2 subtype. Patients having the monocyte 2 subtype may have or become susceptive to having a more severe disease phenotype. As a non-limiting example, the subject with the monocyte 2 subtype has or is likely to fail treatment with anti-TNF, 6-mercaptopurine, and/or methotrexate. Patients having the particular monocyte profile may be suitable for subtype-specific treatment, including administration with a therapeutic agent that targets a biomolecule provided in Table 7A or 7B, or a biomolecule in a biological pathway of a biomolecule provided in Table 7A or 7B. In some cases, a subject may be treated with a modulator of a kinase selected from DNAPK, CDK4, ERK1, HIPK2, CDC2, MAPK1, MAPK3, ERK2, CSNK2A1, CK2ALPHA, JNK1, CDK1, MAPK14, PKR, and CDK2.

Further provided herein are methods and systems for characterizing and treating a patient having CD, wherein the patient is characterized as having or not having a CD-PBmu subtype, and having or not having a monocyte 2 subtype. The non-CD-PBmut subtype may be a PBT subtype. The non-monocyte 2 subtype may be a monocyte 1 subtype. The subtype characterization may be determined sequentially or concurrently. In some cases, a patient having a CD-PBmu subtype and monocyte 2 subtype is treated with a therapeutic agent that targets a biomolecule provided in Table 1A, 4, 7A, 7B, DNAPK, CDK4, ERK1, HIPK2, CDC2, MAPK1, MAPK3, ERK2, CSNK2A1, CK2ALPHA, JNK1, CDK1, MAPK14, PKR, or CDK2. In some cases, a patient having a CD-PBmu subtype and monocyte 2 subtype is not treated with anti-TNF, 6-mercaptopurine, or methotrexate. In some cases, a patient having a CD-PBmu subtype and monocyte 1 subtype is treated with a therapeutic agent that targets a biomolecule provided in Table 1A, 4, 7A, 7B, DNAPK, CDK4, ERK1, HIPK2, CDC2, MAPK1, MAPK3, ERK2, CSNK2A1, CK2ALPHA, JNK1, CDK1, MAPK14, PKR, or CDK2. In some cases, a patient having a CD-PBmu subtype and monocyte 1 subtype is not treated with anti-TNF, 6-mercaptopurine, or methotrexate. In some cases, a patient having a CD-PBT subtype and monocyte 2 subtype is treated with a therapeutic agent that targets a biomolecule provided in Table 1A, 4, 7A, 7B, DNAPK, CDK4, ERK1, HIPK2, CDC2, MAPK1, MAPK3, ERK2, CSNK2A1, CK2ALPHA, JNK1, CDK1, MAPK14, PKR, or CDK2. In some cases, a patient having a CD-PBT subtype and monocyte 2 subtype is not treated with anti-TNF, 6-mercaptopurine, or methotrexate. In some cases, a patient having a CD-PBT subtype and monocyte 1 subtype is treated with a therapeutic agent that targets a biomolecule provided in Table 1A, 4, 7A, 7B, DNAPK, CDK4, ERK1, HIPK2, CDC2, MAPK1, MAPK3, ERK2, CSNK2A1, CK2ALPHA, JNK1, CDK1, MAPK14, PKR, or CDK2. In some cases, a patient having a CD-PBT subtype and monocyte 1 subtype is not treated with anti-TNF, 6-mercaptopurine, or methotrexate.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215(3):403-10; Nucleic Acids Res. 1997 Sep. 1; 25(17):3389-402). Percent homology of sequences can be determined using the most recent version of BLAST, as of the filing date of this application. Percent identity of sequences can be determined using the most recent version of BLAST, as of the filing date of this application.

### Transcriptomic Signature and Profiling

In one aspect, provided herein are transcriptomic signatures associated with a subtype of IBD, including CD. In some cases, the transcriptomic signature comprises one or more genes of Table 1A. In some cases, the transcriptomic signature comprises about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Table 1A. In some cases, the transcriptomic signature comprises genes 1-44 of Table 1A. In some cases, the transcriptomic signature comprises genes 1-117 of Table 1A.

TABLE 1A
Exemplary Biomarkers of a Transcriptomic Signature.

| No | Biomarker | Name | EntrezID | Accession | UGCluster | Ensembl |
|---|---|---|---|---|---|---|
| 1 | ADAM28 | ADAM metallopeptidase domain 28 | 10863 | NM_001304351 | Hs.174030 | ENSG00000042980 |
| 2 | ADAMDEC1 | ADAM-like, decysin 1 | 27299 | NM_001145271 | Hs.521459 | ENSG00000134028 |
| 3 | ADAMTS1 | ADAM metallopeptidase with | 9510 | NM_006988 | Hs.643357 | ENSG00000154734 |
| | | thrombospondin type 1 motif, 1 | | | | |
| 4 | ALDOB | aldolase B, fructose-bisphosphate | 229 | NM_000035 | Hs.530274 | ENSG00000136872 |
| 5 | C1S | complement component 1, s | 716 | NM_001734 | Hs.458355 | ENSG00000182326 |
| | | subcomponent | | | | |
| 6 | CHAC1 | ChaC glutathione-specific gamma- | 79094 | NM_001142776 | Hs.155569 | ENSG00000128965 |
| | | glutamylcyclotransferase 1 | | | | |
| 7 | CHST15 | carbohydrate (N-acetylgalactosamine | 51363 | NM_00127076 | Hs.287537 | ENSG00000182022 |
| | | 4-sulfate 6-O) sulfotransferase 15 | | | | |
| 8 | CPA3 | carboxypeptidase A3 (mast cell) | 1359 | NM_001870 | Hs.646 | ENSG00000163751 |
| 9 | CRYAB | crystallin, alpha B | 1410 | NM_001289807 | Hs.53454 | ENSG00000109846 |
| 10 | DAB2 | Dab, mitogen-responsive | 1601 | NM_001244871 | Hs.696631 | ENSG00000153071 |
| | | phosphoprotein, homolog 2 | | | | |
| | | (Drosophila) | | | | |
| 11 | DCN | decorin | 1634 | NM_001920 | Hs.156316 | ENSG00000011465 |
| 12 | DSE | dermatan sulfate epimerase | 29940 | NM_001080976 | Hs.458358 | ENSG00000111817 |
| 13 | DYRK3 | dual-specificity tyrosine-(Y)- | 8444 | NM_001004023 | Hs.164267 | ENSG00000143479 |
| | | phosphorylation regulated kinase 3 | | | | |
| 14 | FABP1 | fatty acid binding protein 1, liver | 2168 | NM_001443 | Hs.380135 | ENSG00000163586 |
| 15 | FPR3 | formyl peptide receptor 3 | 2359 | NM_002030 | Hs.445466 | ENSG00000187474 |
| 16 | GFPT2 | glutamine-fructose-6-phosphate | 9945 | NM-005110 | Hs.696497 | ENSG00000131459 |
| | | transaminase 2 | | | | |
| 17 | HDC | histidine decarboxylase | 3067 | NM_001306146 | Hs.1481 | ENSG00000140287 |
| 18 | IL22 | interleukin 22 | 50616 | NM_020525 | Hs.287369 | ENSG00000127318 |
| 19 | IL6 | interleukin 6 | 3569 | NM_000600 | Hs.654458 | ENSG00000136244 |
| 20 | KIT | v-kit Hardy-Zuckerman 4 feline | 3815 | NM_000222 | Hs.479754 | ENSG00000157404 |
| | | sarcoma viral oncogene homolog | | | | |
| 21 | LCN2 | lipocalin 2 | 3934 | NM_005564 | Hs.204238 | ENSG00000148346 |
| 22 | LMCD1 | LIM and cysteine-rich domains 1 | 29995 | NM_001278233 | Hs.475353 | ENSG00000071282 |
| 23 | LRRC32 | leucine rich repeat containing 32 | 2615 | NM_001128922 | Hs.151641 | ENSG00000137507 |
| 24 | LYZ | lysozyme | 4069 | NM_000239 | Hs.524579 | ENSG00000090382 |
| 25 | MFSD2A | major facilitator superfamily | 84879 | NM_001136493 | Hs.655177 | ENSG00000168389 |
| | | domain containing 2A | | | | |
| 26 | NANOGNB | NANOG neighbor homeobox | 360030 | NM_001145465 | Hs.558004 | ENSG00000205857 |
| 27 | OR4A5 | olfactory receptor, family 4, | 81318 | NM_001005272 | Hs.554531 | ENSG00000221840 |
| | | subfamily A, member 5 | | | | |
| 28 | PLA2G2A | phospholipase A2, group IIA | 5320 | NM_000300 | Hs.466804 | ENSG00000188257 |
| | | (platelets, synovial fluid) | | | | |
| 29 | PLTP | phospholipid transfer protein | 5360 | NM_001242920 | Hs.439312 | ENSG00000100979 |
| 30 | PPIAP30 | peptidylprolyl isomerase A | 100192204 | NR_036506 | Hs.714691 | |
| | | (cyclophilin A) pseudogene 30 | | | | |
| 31 | RAB13 | RAB13, member RAS oncogene | 5872 | NM_001272038 | Hs.151536 | ENSG00000143545 |
| | | family | | | | |
| 32 | RRAD | Ras-related associated with | 6236 | NM_001128850 | Hs.1027 | ENSG00000166592 |
| | | diabetes | | | | |
| 33 | SDS | serine dehydratase | 10993 | NM_006843 | Hs.439023 | ENSG00000135094 |
| 34 | SEMA6B | sema domain, transmembrane | 10501 | NM_020241 | Hs.465642 | ENSG00000167680 |
| | | domain (TM), and cytoplasmic | | | | |
| | | domain, (semaphorin) 6B | | | | |
| 35 | SEPP1 | selenoprotein P, plasma, 1 | 6414 | NM_001085486 | Hs.275775 | ENSG00000250722 |
| 36 | SERPING1 | serpin peptidase inhibitor, clade G | 710 | NM_000062 | Hs.384598 | ENSG00000149131 |
| | | (C1 inhibitor), member 1 | | | | |
| 37 | SOD3 | superoxide dismutase 3, | 6649 | NM_003102 | Hs.2420 | ENSG00000109610 |
| | | extracellular | | | | |
| 38 | SYK | spleen tyrosine kinase | 6850 | NM_001135052 | Hs.371720 | ENSG00000165025 |
| 39 | TBC1D3 | TBC1 domain family, member 3 | 729873 | NM_001123391 | Hs.454716 | ENSG00000274611 |
| 40 | TBC1D8 | TBC1 domain family, member 8 | 11138 | NM_001102426 | Hs.442657 | ENSG00000204634 |
| | | (with GRAM domain) | | | | |
| 41 | TBC1D9 | TBC1 domain family, member 9 | 23158 | NM_015130 | Hs.480819 | ENSG00000109436 |
| | | (with GRAM domain) | | | | |
| 42 | TNXB | tenascin XB | 7148 | NM_019105 | Hs.485104 | ENSG00000168477 |
| 43 | TPSB2 | tryptase beta 2 (gene/pseudogene) | 64499 | NM_024164 | Hs.405479 | ENSG00000197253 |
| 44 | UBD | ubiquitin D | 10537 | NM_006398 | Hs.44532 | ENSG00000213886 |
| 45 | ABI3BP | ABI family, member 3 (NESH) | 25890 | NM_015429 | Hs.477015 | ENSG00000154175 |
| | | binding protein | | | | |
| 46 | ANKRD20A3 | ankyrin repeat domain 20 family, | 441425 | NM_001012419 | Hs.632663 | ENSG00000276203 |
| | | member A3 | | | | |
| 47 | APOC1P1 | apolipoprotein C-I pseudogene 1 | 342 | NR_028412 | Hs.110675 | ENSG00000214855 |
| 48 | AQP7P3 | aquaporin 7 pseudogene 3 | 441432 | NR_026558 | Hs.743215 | |
| 49 | C11orf96 | chromosome 11 open reading | 387763 | NM_001145033 | Hs.530443 | ENSG00000187479 |
| | | frame 96 | | | | |
| 50 | C1QB | complement component 1, q | 713 | NM_000491 | Hs.8986 | ENSG00000173369 |
| | | subcomponent, B chain | | | | |
| 51 | C1QC | complement component 1, q | 714 | NM_001114101 | Hs.467753 | ENSG00000159189 |
| | | subcomponent, C chain | | | | |
| 52 | C2orf27A | chromosome 2 open reading | 29798 | NM_013310 | Hs.635289 | |
| | | frame 27A | | | | |
| 53 | C8orf4 | chromosome 8 open reading frame 4 | 56892 | NM_020130 | Hs.591849 | ENSG00000176907 |
| 54 | CKB | creatine kinase, brain | 1152 | NM_001823 | Hs.173724 | ENSG00000166165 |
| 55 | CLDN10 | claudin 10 | 9071 | NM_001160100 | Hs.534377 | ENSG00000134873 |
| 56 | CLEC3B | C-type lectin domain family 3, | 7123 | NM_001308394 | Hs.476092 | ENSG00000163815 |
| | | member B | | | | |
| 57 | CLIC4 | chloride intracellular channel 4 | 25932 | NM_013943 | Hs.440544 | ENSG00000169504 |
| 58 | COL1A1 | collagen, type I, alpha 1 | 1277 | NM_000088 | Hs.172928 | ENSG00000108821 |
| 59 | COL1A2 | collagen, type I, alpha 2 | 1278 | NM_000089 | Hs.489142 | ENSG00000164692 |
| 60 | COL5A1 | collagen, type V, alpha 1 | 1289 | NM_000093 | Hs.210283 | ENSG00000130635 |
| 61 | CXCL13 | chemokine (C-X-C motif) ligand 13 | 10563 | NM_006419 | Hs.100431 | ENSG00000156234 |
| 62 | CYCSP52 | cytochrome c, somatic | 360155 | NR_001560 | Hs.491808 | ENSG00000235700 |
| | | pseudogene 52 | | | | |
| 63 | FAM138D | family with sequence similarity 138, | 677784 | NR_026823 | Hs.722487 | ENSG00000249054 |
| | | member D | | | | |
| 64 | FAM182B | family with sequence similarity 182, | 728882 | NR_026714 | Hs.682103 | ENSG00000175170 |
| | | member B | | | | |
| 65 | FAM222A | family with sequence similarity 222, | 84915 | NM_032829 | Hs.661785 | ENSG00000139438 |
| | | member A | | | | |
| 66 | FAM231A | family with sequence similarity 231, | 729574 | NM_001282321 | | ENSG00000237847 |
| | | member A | | | | |
| 67 | FAM27A | | | | | |
| 68 | FSTL1 | follistatin-like 1 | 11167 | NM_007085 | Hs.269512 | ENSG00000163430 |
| 69 | GAS7 | growth arrest-specific 7 | 8522 | NM_001130831 | Hs.462214 | ENSG00000007237 |
| 70 | GEM | GTP binding protein overexpressed | 2669 | NM_005261 | Hs.654463 | ENSG00000164949 |
| | | in skeletal muscle | | | | |
| 71 | GOLGA6L5P | golgin A6 family-like 5, pseudogene | 374650 | NM_198079 | Hs.454625 | |
| 72 | GPNMB | glycoprotein (transmembrane) nmb | 10457 | NM_001005340 | Hs.190495 | ENSG00000136235 |
| 73 | GYPE | glycophorin E (MNS blood group) | 2996 | NM_002102 | Hs.654368 | ENSG00000197465 |
| 74 | HNRNPA1P33 | heterogeneous nuclear | 728643 | NR_003277 | Hs.711067 | ENSG00000213412 |
| | | ribonucleoprotein A1 pseudogene 33 | | | | |
| 75 | HSPA2 | heat shock 70 kDa protein 2 | 3306 | NM_021979 | Hs.432648 | ENSG00000126803 |
| 76 | HSPB6 | heat shock protein, alpha- | 126393 | NM_144617 | Hs.534538 | ENSG00000004776 |
| | | crystallin-related, B6 | | | | |
| 77 | KGFLP2 | keratinocyte growth factor- | 654466 | NM_001039113 | Hs.536967 | |
| | | like protein 2 | | | | |
| 78 | KRT20 | keratin 20, type I | 54474 | NM_019010 | Hs.84905 | ENSG00000171431 |
| 79 | LIMS3L | LIM and senescent cell | 100288695 | NM_001205288 | Hs.535619 | ENSG00000256671 |
| | | antigen-like domains 3-like | | | | |
| 80 | LINC00348 | long intergenic non-protein | 100885781 | NR_047699 | Hs.372660 | ENSG00000226846 |
| | | coding RNA 348 | | | | |
| 81 | LINC00700 | long intergenic non-protein | 282980 | NR_040253 | Hs.576810 | ENSG00000234962 |
| | | coding RNA 700 | | | | |
| 82 | LINC00857 | long intergenic non-protein | 439990 | NR_038464 | Hs.365566 | ENSG00000237523 |
| | | coding RNA 857 | | | | |
| 83 | LINC01189 | long intergenic non-protein | 643648 | NR_046203 | Hs.640178 | |
| | | coding RNA 1189 | | | | |
| 84 | LOC100129138 | THAP domain containing, apoptosis | 100129138 | NR_033990 | Hs.514487 | ENSG00000215869 |
| | | associated protein 3 pseudogene | | | | |
| 85 | LOC100507006 | uncharacterized LOC100507006 | 100507006 | NR_120420 | Hs.442789 | |
| 86 | LOC100508046 | uncharacterized LOC100508046 | 100508046 | NR_110505 | Hs.433218 | ENSG00000275563 |
| 87 | LOC101927123 | uncharacterized LOC101927123 | 101927123 | NR_110147 | Hs.526761 | ENSG00000244215 |
| 88 | LOC101927905 | uncharacterized LOC101927905 | 101927905 | NR_120454 | Hs.621425 | ENSG00000215241 |
| 89 | LOC101928163 | uncharacterized LOC101928163 | 101928163 | NR_110799 | Hs.588761 | |
| 90 | LOC102724034 | uncharacterized LOC102724034 | 102724034 | NR_120378 | Hs.694638 | |
| 91 | LOC642426 | uncharacterized LOC642426 | 642426 | NR_046104 | Hs.578301 | ENSG00000257504 |
| 92 | LOC645166 | lymphocyte-specific protein 1 | 645166 | NR_027354 | Hs.744183 | ENSG00000232527 |
| | | pseudogene | | | | |
| 93 | LOC646736 | uncharacterized LOC646736 | 646736 | NR_046102 | Hs.712836 | |
| 94 | MIR663A | microRNA 663a | 724033 | NR_030386 | | ENSG00000273684 |
| 95 | MLLT10P1 | myeloid/lymphoid or mixed- | 140678 | NR_045115 | Hs.653099 | |
| | | lineage leukemia; translocated to, | | | | |
| | | 10 pseudogene 1 | | | | |
| 96 | MMP19 | matrix metallopeptidase 19 | 4327 | NM_001032360 | Hs.591033 | ENSG00000123342 |
| 97 | NCOR1P1 | nuclear receptor corepressor 1 | 149934 | NM_001039379 | Hs.711274 | ENSG00000240108 |
| | | pseudogene 1 | | | | |
| 98 | PGM5-AS1 | PGM5 antisense RNA 1 | 572558 | NR_015423 | Hs.552819 | ENSG00000224958 |
| 99 | PHLDB1 | pleckstrin homology-like domain, | 23187 | NM_001144758 | Hs.504062 | ENSG00000019144 |
| | | family B, member 1 | | | | |
| 100 | PMP22 | peripheral myelin protein 22 | 5376 | NM_000304 | Hs.372031 | ENSG00000109099 |
| 101 | PTENP1-AS | PTENP1 antisense RNA | 101243555 | NR_103745 | Hs.598470 | ENSG00000281128 |
| 102 | REG3A | regenerating islet-derived 3 alpha | 5068 | NM_002580 | Hs.567312 | ENSG00000172016 |
| 103 | RPSAP9 | ribosomal protein SA pseudogene 9 | 653162 | NR_026890 | Hs.655646 | ENSG00000234618 |
| 104 | SEPSECS-AS1 | SEPSECS antisense RNA 1 | 285540 | NR_037934 | Hs.732278 | |
| | | (head to head) | | | | |
| 105 | SEPT14 | | | | | |
| 106 | SLC9B1 | solute carrier family 9, subfamily B | 150159 | NM_001100874 | Hs.666728 | ENSG00000164037 |
| | | (NHA1, cation proton antiporter 1), | | | | |
| | | member 1 | | | | |
| 107 | SLCO4A1 | solute carrier organic anion | 28231 | NM_016354 | Hs.235782 | ENSG00000101187 |
| | | transporter family, member 4A1 | | | | |
| 108 | SMOX | spermine oxidase | 54498 | NM_001270691 | Hs.433337 | ENSG00000088826 |
| 109 | SPARCL1 | SPARC-like 1 (hevin) | 8404 | NM_001128310 | Hs.62886 | ENSG00000152583 |
| 110 | SRC | SRC proto-oncogene, non-receptor | 6714 | NM_005417 | Hs.195659 | ENSG00000197122 |
| | | tyrosine kinase | | | | |
| 111 | ST13P4 | suppression of tumorigenicity 13 | 145165 | NM_153290 | Hs.511834 | |
| | | (colon carcinoma) (Hsp70 | | | | |
| | | interacting protein) pseudogene 4 | | | | |
| 112 | TCF21 | transcription factor 21 | 6943 | NM_003206 | Hs.78061 | ENSG00000118526 |
| 113 | TCF4 | transcription factor 4 | 6925 | NM_001083962 | Hs.605153 | ENSG00000196628 |
| 114 | TMEM45B | transmembrane protein 45B | 120224 | NM_138788 | Hs.504301 | ENSG00000151715 |
| 115 | UBE2Q2L | ubiquitin-conjugating enzyme E2Q | 100505679 | NM_001243531 | Hs.726826 | ENSG00000259511 |
| | | family member 2-like | | | | |
| 116 | UBTFL1 | upstream binding transcription factor, | 642623 | NM_001143975 | Hs.719885 | ENSG00000255009 |
| | | RNA polymerase I-like 1 | | | | |
| 117 | ZNF582-AS1 | ZNF582 antisense RNA 1 | 386758 | NR_037159 | Hs.549564 | ENSG00000267454 |
| | | (head to head) | | | | |
| 118 | ADM | adrenomedullin | 133 | NM_001124 | Hs.441047 | ENSG00000148926 |
| 119 | ANPEP | alanyl (membrane) aminopeptidase | 290 | NM_001150 | Hs.1239 | ENSG00000166825 |
| 120 | AOAH-IT1 | AOAH intronic transcript 1 | 100874264 | NR_046764 | Hs.690994 | ENSG00000230539 |
| 121 | ASB2 | ankyrin repeat and SOCS box | 51676 | NM_001202429 | Hs.510327 | ENSG00000100628 |
| | | containing 2 | | | | |
| 122 | ATP5J2-PTCD1 | ATP5J2-PTCD1 readthrough | 100526740 | NM_001198879 | Hs.632313 | ENSG00000248919 |
| 123 | BASP1 | brain abundant, membrane | 10409 | NM_001271606 | Hs.201641 | ENSG00000176788 |
| | | attached signal protein 1 | | | | |
| 124 | CCL11 | chemokine (C-C motif) ligand 11 | 6356 | NM_002986 | Hs.54460 | ENSG00000172156 |
| 125 | CD68 | CD68 molecule | 968 | NM_001040059 | Hs.647419 | ENSG00000129226 |
| 126 | CSF2RB | colony stimulating factor 2 receptor, | 1439 | NM_000395 | Hs.592192 | ENSG00000100368 |
| | | beta, low-affinity (granulocyte- | | | | |
| | | macrophage) | | | | |
| 127 | CTAGE8 | CTAGE family, member 8 | 100142659 | NM_001278507 | Hs.661442 | ENSG00000244693 |
| 128 | CTGF | connective tissue growth factor | 1490 | NM_001901 | Hs.410037 | ENSG00000118523 |
| 129 | CXCL1 | chemokine (C-X-C motif) ligand 1 | 2919 | NM_001511 | Hs.789 | ENSG00000163739 |
| | | (melanoma growth stimulating | | | | |
| | | activity, alpha) | | | | |
| 130 | CXCL3 | chemokine (C-X-C motif) ligand 3 | 2921 | NM_002090 | Hs.89690 | ENSG00000163734 |
| 131 | DEFA5 | defensin, alpha 5, Paneth cell- | 1670 | NM_021010 | Hs.655233 | ENSG00000164816 |
| | | specific | | | | |
| 132 | DEFA6 | defensin, alpha 6, Paneth cell- | 1671 | NM_001926 | Hs.711 | ENSG00000164822 |
| | | specific | | | | |
| 133 | DERL3 | derlin 3 | 91319 | NM_001002862 | Hs.593679 | ENSG00000099958 |
| 134 | DNASE1L3 | deoxyribonuclease I-like 3 | 1776 | NM_001256560 | Hs.476453 | ENSG00000163687 |
| 135 | DOK3 | docking protein 3 | 79930 | NM_001144875 | Hs.720849 | ENSG00000146094 |
| 136 | EGR2 | early growth response 2 | 1959 | NM_000399 | Hs.1395 | ENSG00000122877 |
| 137 | EGR3 | early growth response 3 | 1960 | NM_001199880 | Hs.534313 | ENSG00000179388 |
| 138 | EMP1 | epithelial membrane protein 1 | 2012 | NM_001423 | Hs.719042 | ENSG00000134531 |
| 139 | EPAS1 | endothelial PAS domain protein 1 | 2034 | NM_001430 | Hs.468410 | ENSG00000116016 |
| 140 | FAM138A | family with sequence similarity 138, | 645520 | NR_026818 | Hs.569137 | ENSG00000237613 |
| | | member A | | | | |
| 141 | FAM138F | family with sequence similarity 138, | 641702 | NR_026820 | Hs.569137 | ENSG00000282591 |
| | | member F | | | | |
| 142 | FAM157B | family with sequence similarity 157, | 100132403 | NM_001145249 | Hs.741123 | |
| | | member B | | | | |
| 143 | FDCSP | follicular dendritic cell secreted | 260436 | NM_152997 | Hs.733448 | ENSG00000181617 |
| | | protein | | | | |
| 144 | FOSL1 | FOS-like antigen 1 | 8061 | NM_001300844 | Hs.283565 | ENSG00000175592 |
| 145 | FSCN1 | fascin actin-bundling protein 1 | 6624 | NM_003088 | Hs.118400 | ENSG00000075618 |
| 146 | FTH1P3 | ferritin, heavy polypeptide 1 | 2498 | NR_002201 | Hs.658438 | |
| | | pseudogene 3 | | | | |
| 147 | GAS6 | growth arrest-specific 6 | 2621 | NM_000820 | Hs.646346 | ENSG00000183087 |
| 148 | GATA2 | GATA binding protein 2 | 2624 | NM_001145661 | Hs.367725 | ENSG00000179348 |
| 149 | GPX3 | glutathione peroxidase 3 | 2878 | NM_002084 | Hs.386793 | ENSG00000211445 |
| 150 | HES1 | hes family bHLH transcription | 3280 | NM_005524 | Hs.250666 | ENSG00000114315 |
| | | factor 1 | | | | |
| 151 | HES4 | hes family bHLH transcription | 57801 | NM_001142467 | Hs.154029 | ENSG00000188290 |
| | | factor 4 | | | | |
| 152 | HLA-L | major histocompatibility complex, | 3139 | NR_027822 | Hs.656020 | ENSG00000243753 |
| | | class I, L (pseudogene) | | | | |
| 153 | IGFBP7 | insulin-like growth factor | 3490 | NM_001253835 | Hs.479808 | ENSG00000163453 |
| | | binding protein 7 | | | | |
| 154 | IL1RN | interleukin 1 receptor antagonist | 3557 | NM_000577 | Hs.81134 | ENSG00000136689 |
| 155 | IL21R-AS1 | IL21R antisense RNA 1 | 283888 | NR_037158 | Hs.660935 | ENSG00000259954 |
| 156 | LINC01194 | long intergenic non-protein | 404663 | NR_033383 | Hs.552273 | |
| | | coding RNA 1194 | | | | |
| 157 | LOC100240735 | uncharacterized LOC100240735 | 100240735 | NR_026658 | Hs.635297 | ENSG00000250654 |
| 158 | LOC101927817 | uncharacterized LOC101927817 | 101927817 | NR_110931 | Hs.667942 | |
| 159 | LOC284801 | | | | | |
| 160 | LOC285740 | uncharacterized LOC285740 | 285740 | NR_027113 | Hs.432656 | ENSG00000235740 |
| 161 | LOC441242 | uncharacterized LOC441242 | 441242 | NM_001013464 | Hs.373941 | ENSG00000272693 |
| 162 | LOC644172 | mitogen-activated protein kinase 8 | 644172 | NR_026901 | Hs.448859 | |
| | | interacting protein 1 pseudogene | | | | |
| 163 | MAFF | v-maf avian musculoaponeurotic | 23764 | NM_001161572 | Hs.517617 | ENSG00000185022 |
| | | fibrosarcoma oncogene homolog F | | | | |
| 164 | MARCKS | myristoylated alanine-rich | 4082 | NM_002356 | Hs.519909 | ENSG00000277443 |
| | | protein kinase C substrate | | | | |
| 165 | MCTP1 | multiple C2 domains, | 79772 | NM_001002796 | Hs.591248 | ENSG00000175471 |
| | | transmembrane 1 | | | | |
| 166 | MGP | matrix Gla protein | 4256 | NM_000900 | Hs.365706 | ENSG00000111341 |
| 167 | MIR548I1 | microRNA 548i-1 | 100302204 | NR_031687 | | ENSG00000221737 |
| 168 | MIR663B | microRNA 663b | 100313824 | NR_031608 | | ENSG00000221288 |
| 169 | MMP9 | matrix metallopeptidase 9 | 4318 | NM_004994 | Hs.297413 | ENSG00000100985 |
| 170 | MT1G | metallothionein 1G | 4495 | NM_001301267 | Hs.433391 | ENSG00000125144 |
| 171 | NPIPB9 | nuclear pore complex interacting | 100507607 | NM_001287250 | Hs.710214 | ENSG00000196993 |
| | | protein family, member B9 | | | | |
| 172 | NUCB1-AS1 | NUCB1 antisense RNA 1 | 100874085 | NR_046633 | Hs.569933 | ENSG00000235191 |
| 173 | OR4F21 | olfactory receptor, family 4, | 441308 | NM_001005504 | Hs.690459 | ENSG00000176269 |
| | | subfamily F, member 21 | | | | |
| 174 | PHACTR1 | phosphatase and actin regulator 1 | 221692 | NM_001242648 | Hs.436996 | ENSG00000112137 |
| 175 | PLEKHA4 | pleckstrin homology domain | 57664 | NM_001161354 | Hs.9469 | ENSG00000105559 |
| | | containing, family A | | | | |
| | | (phosphoinositide binding specific) | | | | |
| | | member 4 | | | | |
| 176 | PLGLB1 | plasminogen-like B1 | 5343 | NM_001032392 | Hs.652169 | ENSG00000183281 |
| 177 | POC1B-GALNT4 | POC1B-GALNT4 readthrough | 100528030 | NM_001199781 | Hs.25130 | ENSG00000259075 |
| 178 | PRKX-AS1 | PRKX antisense RNA 1 | 100873944 | NR_046643 | | ENSG00000236188 |
| 179 | PTGS2 | prostaglandin-endoperoxide | 5743 | NM_000963 | Hs.196384 | ENSG00000073756 |
| | | synthase 2 (prostaglandin G/H | | | | |
| | | synthase and cyclooxygenase) | | | | |
| 180 | RAB20 | RAB20, member RAS oncogene | 55647 | NM_017817 | Hs.743563 | ENSG00000139832 |
| | | family | | | | |
| 181 | REG1A | regenerating islet-derived 1 alpha | 5967 | NM_002909 | Hs.49407 | ENSG00000115386 |
| 182 | RNASE1 | ribonuclease, RNase A family, | 6035 | NM_002933 | Hs.78224 | ENSG00000129538 |
| | | 1 (pancreatic) | | | | |
| 183 | SDC4 | syndecan 4 | 6385 | NM_002999 | Hs.632267 | ENSG00000124145 |
| 184 | SEPT10 | | | | | |
| 185 | SIRPA | signal-regulatory protein alpha | 140885 | NM_001040022 | Hs.581021 | ENSG00000198053 |
| 186 | SNAI1 | snail family zinc finger 1 | 6615 | NM_005985 | Hs.48029 | ENSG00000124216 |
| 187 | SPARC | secreted protein, acidic, cysteine- | 6678 | NM_001309443 | Hs.111779 | ENSG00000113140 |
| | | rich (osteonectin) | | | | |
| 188 | SPHK1 | sphingosine kinase 1 | 8877 | NM_001142601 | Hs.68061 | ENSG00000176170 |
| 189 | SPINK4 | serine peptidase inhibitor, Kazal | 27290 | NM_014471 | Hs.555934 | ENSG00000122711 |
| | | type 4 | | | | |
| 190 | STAB1 | stabilin 1 | 23166 | NM_015136 | Hs.301989 | ENSG00000010327 |
| 191 | TMEM114 | transmembrane protein 114 | 283953 | NM_001146336 | Hs.150849 | ENSG00000232258 |
| 192 | TNFAIP2 | tumor necrosis factor, alpha- | 7127 | NM_006291 | Hs.525607 | ENSG00000185215 |
| | | induced protein 2 | | | | |
| 193 | TNFRSF12A | tumor necrosis factor receptor | 51330 | NM_016639 | Hs.355899 | ENSG00000006327 |
| | | superfamily, member 12A | | | | |
| 194 | TNFRSF13B | tumor necrosis factor receptor | 23495 | NM_012452 | Hs.158341 | ENSG00000240505 |
| | | superfamily, member 13B | | | | |
| 195 | TPSAB1 | tryptase alpha/beta 1 | 7177 | NM_003294 | Hs.405479 | ENSG00000172236 |
| 196 | TREM1 | triggering receptor expressed on | 54210 | NM_001242589 | Hs.283022 | ENSG00000124731 |
| | | myeloid cells 1 | | | | |
| 197 | TUBB6 | tubulin, beta 6 class V | 84617 | NM_001303524 | Hs.193491 | ENSG00000176014 |
| 198 | UGT2B10 | UDP glucuronosyltransferase 2 | 7365 | NM_001075 | Hs.201634 | ENSG00000109181 |
| | | family, polypeptide B10 | | | | |
| 199 | UPK3B | uroplakin 3B | 80761 | NM_030570 | Hs.488861 | ENSG00000243566 |
| 200 | VEGFA | vascular endothelial growth factor A | 7422 | NM_001025366 | Hs.73793 | ENSG00000112715 |

Further provided are methods and compositions for characterizing a subtype of Crohn's Disease (CD) in a subject. A non-limiting subtype is CD-PBmu, which is associated with a mucosal-like expression profile. In some cases, the CD-PBmu subtype is associated with an altered composition of T-cell subsets, clinical disease severity markers, and decreased pro-inflammatory gene expression following surgery. The characterization methods provided include diagnosing the presence or absence of a CD subtype, prognosing whether a subject is predisposed to developing a particular CD subtype, prognosing a response of a patient with a particular CD subtype to a therapeutic treatment, and monitoring CD treatment.

In some embodiments, the methods involve detecting in a biological sample from a subject expression levels of one or more genes of a transcriptomic signature to obtain an expression profile comprising the expression levels of each of the one or more genes in the signature. In some embodiments, the transcriptomic signature comprises one or more biomarkers listed in Table 1A. In some embodiments, the transcriptomic signature comprises any combination of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 5, 60, 65, 70, 75, 80, 90, 100, or more of the genes of Table 1A. In some cases, the transcriptomic signature comprises genes 1-44 of Table 1A. In some cases, the transcriptomic signature comprises genes 1-117 of Table 1A.

In some embodiments, gene expression profiling may be used as a research tool to identify new markers for diagnosis and/or classification of an IBD disease or condition, to monitor the effect of drugs or candidate drugs on biological samples and/or patients, to uncover new pathways for IBD treatment, or any combination thereof.

In some embodiments, the transcriptomic signature comprises ADAMTS1. In some embodiments, the transcriptomic signature comprises LCN2. In some embodiments, the transcriptomic signature comprises ADAM28. In some embodiments, the transcriptomic signature comprises TPSB2. In some embodiments, the transcriptomic signature comprises PPIAP30. In some embodiments, the transcriptomic signature comprises GFPT2. In some embodiments, the transcriptomic signature comprises KIT. In some embodiments, the transcriptomic signature comprises PLTP. In some embodiments, the transcriptomic signature comprises MFSD2A. In some embodiments, the transcriptomic signature comprises IL22. In some embodiments, the transcriptomic signature comprises LMCD1. In some embodiments, the transcriptomic signature comprises IL6. In some embodiments, the transcriptomic signature comprises TBC1D9. In some embodiments, the transcriptomic signature comprises CHAC1. In some embodiments, the transcriptomic signature comprises SEPP1. In some embodiments, the transcriptomic signature comprises SOD3. In some embodiments, the transcriptomic signature comprises RAB13. In some embodiments, the transcriptomic signature comprises LYZ. In some embodiments, the transcriptomic signature comprises CPA3. In some embodiments, the transcriptomic signature comprises SDS. In some embodiments, the transcriptomic signature comprises DYRK3. In some embodiments, the transcriptomic signature comprises DAB2. In some embodiments, the transcriptomic signature comprises TBC1D8. In some embodiments, the transcriptomic signature comprises CRYAB. In some embodiments, the transcriptomic signature comprises TBC1D3. In some embodiments, the transcriptomic signature comprises LRRC32. In some embodiments, the transcriptomic signature comprises SERPING1. In some embodiments, the transcriptomic signature comprises UBD. In some embodiments, the transcriptomic signature comprises FABP1. In some embodiments, the transcriptomic signature comprises SYK. In some embodiments, the transcriptomic signature comprises ALDOB. In some embodiments, the transcriptomic signature comprises SEMA6B. In some embodiments, the transcriptomic signature comprises NANOGNB. In some embodiments, the transcriptomic signature comprises DSE. In some embodiments, the transcriptomic signature comprises FPR3. In some embodiments, the transcriptomic signature comprises TNXB. In some embodiments, the transcriptomic signature comprises OR4A5. In some embodiments, the transcriptomic signature comprises DCN. In some embodiments, the transcriptomic signature comprises CHST15. In some embodiments, the transcriptomic signature comprises ADAMDEC1. In some embodiments, the transcriptomic signature comprises HDC. In some embodiments, the transcriptomic signature comprises RRAD. In some embodiments, the transcriptomic signature comprises C1S. In some embodiments, the transcriptomic signature comprises PLA2G2A. In some embodiments, the transcriptomic signature comprises CYCSP52. In some embodiments, the transcriptomic signature comprises C11orf96. In some embodiments, the transcriptomic signature comprises SEPSECS-AS1. In some embodiments, the transcriptomic signature comprises C1QC. In some embodiments, the transcriptomic signature comprises SLC9B1. In some embodiments, the transcriptomic signature comprises MLLT10P1. In some embodiments, the transcriptomic signature comprises LOC102724034. In some embodiments, the transcriptomic signature comprises SMOX. In some embodiments, the transcriptomic signature comprises CKB. In some embodiments, the transcriptomic signature comprises NCOR1P1. In some embodiments, the transcriptomic signature comprises LOC646736. In some embodiments, the transcriptomic signature comprises CLEC3B. In some embodiments, the transcriptomic signature comprises SLCO4A1. In some embodiments, the transcriptomic signature comprises APOC1P1. In some embodiments, the transcriptomic signature comprises KGFLP2. In some embodiments, the transcriptomic signature comprises ABI3BP. In some embodiments, the transcriptomic signature comprises LINC01189. In some embodiments, the transcriptomic signature comprises SEPT14. In some embodiments, the transcriptomic signature comprises FSTL1. In some embodiments, the transcriptomic signature comprises GEM. In some embodiments, the transcriptomic signature comprises FAM27A. In some embodiments, the transcriptomic signature comprises PTENP1-AS. In some embodiments, the transcriptomic signature comprises LIMS3L. In some embodiments, the transcriptomic signature comprises ST13P4. In some embodiments, the transcriptomic signature comprises C1QB. In some embodiments, the transcriptomic signature comprises HNRNPA1P33. In some embodiments, the transcriptomic signature comprises MIR663A. In some embodiments, the transcriptomic signature comprises LOC101927123. In some embodiments, the transcriptomic signature comprises C2orf27A. In some embodiments, the transcriptomic signature comprises LOC645166. In some embodiments, the transcriptomic signature comprises ZNF582-AS1. In some embodiments, the transcriptomic signature comprises HSPA2. In some embodiments, the transcriptomic signature comprises COL1A1. In some embodiments, the transcriptomic signature comprises COL5A1. In some embodiments, the transcriptomic signature comprises GOLGA6L5P. In some embodiments, the transcriptomic signature comprises PGM5-AS1. In some embodiments, the transcriptomic signature comprises CLDN10. In some embodiments, the transcriptomic signature comprises UBE2Q2L. In some embodiments, the transcriptomic signature comprises LOC100129138. In some embodiments, the transcriptomic signature comprises COL1A2. In some embodiments, the transcriptomic signature comprises SPARCL1. In some embodiments, the transcriptomic signature comprises FAM222A. In some embodiments, the transcriptomic signature comprises LINC00857. In some embodiments, the transcriptomic signature comprises CLIC4. In some embodiments, the transcriptomic signature comprises FAM182B. In some embodiments, the transcriptomic signature comprises LOC642426. In some embodiments, the transcriptomic signature comprises GYPE. In some embodiments, the transcriptomic signature comprises C8orf4. In some embodiments, the transcriptomic signature comprises RPSAP9. In some embodiments, the transcriptomic signature comprises FAM231A. In some embodiments, the transcriptomic signature comprises LINC00700. In some embodiments, the transcriptomic signature comprises ANKRD20A3. In some embodiments, the transcriptomic signature comprises FAM138D. In some embodiments, the transcriptomic signature comprises KRT20. In some embodiments, the transcriptomic signature comprises UBTFL1. In some embodiments, the transcriptomic signature comprises GAS7. In some embodiments, the transcriptomic signature comprises GPNMB. In some embodiments, the transcriptomic signature comprises TCF4. In some embodiments, the transcriptomic signature comprises LINC00348. In some embodiments, the transcriptomic signature comprises SRC. In some embodiments, the transcriptomic signature comprises HSPB6. In some embodiments, the transcriptomic signature comprises LOC100507006. In some embodiments, the transcriptomic signature comprises TCF21. In some embodiments, the transcriptomic signature comprises TMEM45B. In some embodiments, the transcriptomic signature comprises LOC101927905. In some embodiments, the transcriptomic signature comprises CXCL13. In some embodiments, the transcriptomic signature comprises AQP7P3. In some embodiments, the transcriptomic signature comprises PMP22. In some embodiments, the transcriptomic signature comprises LOC101928163. In some embodiments, the transcriptomic signature comprises REG3A. In some embodiments, the transcriptomic signature comprises MMP19. In some embodiments, the transcriptomic signature comprises PHLDB1. In some embodiments, the transcriptomic signature comprises LOC100508046. In some embodiments, the transcriptomic signature comprises SPINK4. In some embodiments, the transcriptomic signature comprises HES4. In some embodiments, the transcriptomic signature comprises TREM1. In some embodiments, the transcriptomic signature comprises TNFRSF12A. In some embodiments, the transcriptomic signature comprises PRKX-AS1. In some embodiments, the transcriptomic signature comprises PLGLB1. In some embodiments, the transcriptomic signature comprises SNAI1. In some embodiments, the transcriptomic signature comprises NUCB1-AS1. In some embodiments, the transcriptomic signature comprises BASP1. In some embodiments, the transcriptomic signature comprises MGP. In some embodiments, the transcriptomic signature comprises ANPEP. In some embodiments, the transcriptomic signature comprises PHACTR1. In some embodiments, the transcriptomic signature comprises ADM. In some embodiments, the transcriptomic signature comprises DEFA6. In some embodiments, the transcriptomic signature comprises VEGFA. In some embodiments, the transcriptomic signature comprises EGR2. In some embodiments, the transcriptomic signature comprises DEFA5. In some embodiments, the transcriptomic signature comprises CXCL3. In some embodiments, the transcriptomic signature comprises SDC4. In some embodiments, the transcriptomic signature comprises TPSAB 1. In some embodiments, the transcriptomic signature comprises CD68. In some embodiments, the transcriptomic signature comprises EPAS1. In some embodiments, the transcriptomic signature comprises MARCKS. In some embodiments, the transcriptomic signature comprises TNFAIP2. In some embodiments, the transcriptomic signature comprises MIR663B. In some embodiments, the transcriptomic signature comprises TMEM114. In some embodiments, the transcriptomic signature comprises SIRPA. In some embodiments, the transcriptomic signature comprises GAS6. In some embodiments, the transcriptomic signature comprises IGFBP7. In some embodiments, the transcriptomic signature comprises ASB2. In some embodiments, the transcriptomic signature comprises HES1. In some embodiments, the transcriptomic signature comprises LOC284801. In some embodiments, the transcriptomic signature comprises TNFRSF13B. In some embodiments, the transcriptomic signature comprises MIR548I1. In some embodiments, the transcriptomic signature comprises DERL3. In some embodiments, the transcriptomic signature comprises SPARC. In some embodiments, the transcriptomic signature comprises EMP1. In some embodiments, the transcriptomic signature comprises LOC100240735. In some embodiments, the transcriptomic signature comprises LOC101927817. In some embodiments, the transcriptomic signature comprises STAB1. In some embodiments, the transcriptomic signature comprises UPK3B. In some embodiments, the transcriptomic signature comprises RAB20. In some embodiments, the transcriptomic signature comprises MMP9. In some embodiments, the transcriptomic signature comprises MT1G. In some embodiments, the transcriptomic signature comprises POC1B-GALNT4. In some embodiments, the transcriptomic signature comprises CSF2RB. In some embodiments, the transcriptomic signature comprises IL1RN. In some embodiments, the transcriptomic signature comprises PLEKHA4. In some embodiments, the transcriptomic signature comprises LOC644172. In some embodiments, the transcriptomic signature comprises MAFF. In some embodiments, the transcriptomic signature comprises FDCSP. In some embodiments, the transcriptomic signature comprises DNASE1L3. In some embodiments, the transcriptomic signature comprises PTGS2. In some embodiments, the transcriptomic signature comprises TUBB6. In some embodiments, the transcriptomic signature comprises LINC01194. In some embodiments, the transcriptomic signature comprises CTAGE8. In some embodiments, the transcriptomic signature comprises REG1A. In some embodiments, the transcriptomic signature comprises ATP5J2-PTCD1. In some embodiments, the transcriptomic signature comprises DOK3. In some embodiments, the transcriptomic signature comprises EGR3. In some embodiments, the transcriptomic signature comprises AOAH-IT1. In some embodiments, the transcriptomic signature comprises RNASE1. In some embodiments, the transcriptomic signature comprises CCL11. In some embodiments, the transcriptomic signature comprises OR4F21. In some embodiments, the transcriptomic signature comprises FAM157B. In some embodiments, the transcriptomic signature comprises GATA2. In some embodiments, the transcriptomic signature comprises CTGF. In some embodiments, the transcriptomic signature comprises CXCL1. In some embodiments, the transcriptomic signature comprises GPX3. In some embodiments, the transcriptomic signature comprises FAM138A. In some embodiments, the transcriptomic signature comprises FAM138F. In some embodiments, the transcriptomic signature comprises FOSL1. In some embodiments, the transcriptomic signature comprises FSCN1. In some embodiments, the transcriptomic signature comprises FTH1P3. In some embodiments, the transcriptomic signature comprises SPHK1. In some embodiments, the transcriptomic signature comprises LOC441242. In some embodiments, the transcriptomic signature comprises UGT2B10. In some embodiments, the transcriptomic signature comprises MCTP1. In some embodiments, the transcriptomic signature comprises IL21R-AS1. In some embodiments, the transcriptomic signature comprises LOC285740. In some embodiments, the transcriptomic signature comprises HLA-L. In some embodiments, the transcriptomic signature comprises NPIPB9. In some embodiments, the transcriptomic signature comprises SEPT10.

The expression profile of a transcriptomic signature in a subject may be determined by analyzing genetic material obtained from a subject. The subject may be human. In some embodiments, the genetic material is obtained from a subject having an inflammatory disease, such as inflammatory bowel disease, or specifically, Crohn's Disease. Although the methods described herein are generally referenced for use with Crohn's Disease patients, in some cases the methods and transcriptomic signatures are applicable to other inflammatory diseases, including, ulcerative colitis.

In some embodiments, the genetic material is obtained from blood, serum, plasma, sweat, hair, tears, urine, or tissue. Techniques for obtaining samples from a subject include, for example, obtaining samples by a mouth swab or a mouth wash, drawing blood, and obtaining a biopsy. In some cases, the genetic material is obtained from a biopsy, e.g., from the intestinal track of the subject. Isolating components of fluid or tissue samples (e.g., cells or RNA or DNA) may be accomplished using a variety of techniques. After the sample is obtained, it may be further processed to enrich for or purify genomic material.

In some embodiments, the expression level of a biomarker in a sample from a subject is compared to a reference expression level. In some cases, the reference expression level is from a subject that does not comprise IBD. In some cases, the reference expression level is from a subject that comprises a non-PBmu subtype of CD. In some cases, the reference expression level is from a subject that comprises a CD-PBmu subtype. In some cases, a patient having a CD-PBmu subtype has an expression level of one or more biomarkers at least 1.5-fold, 2-fold, 2.5-fold, 3-fold, 3.5-fold, 4-fold, 4.5-fold, or 5-fold greater than the expression level of the one or more biomarkers in a reference subject (e.g., a subject who does not have IBD or has a non-PBmu CD subtype). Table 1B provides non-limiting examples of increased expression fold of biomarkers in a CD-PBmu subject as compared to a subject who does not have IBD (NL) or has a PBT CD subtype.

TABLE 1B
Increased Expression of Biomarkers in CD-PBmu Subject

| | | Fold-change | Fold change |
|---|---|---|---|
| No. | Biomarker | PBmu vs PBT | PBmu vs NL |
| 1 | ADAM28 | 2.43 | |
| 2 | ADAMDEC1 | 6.76 | 4.658 |
| 3 | ADAMTS1 | 2.22 | 2.273 |
| 4 | ALDOB | 5.32 | 5.686 |
| 5 | C1S | 5.42 | 2.923 |
| 6 | CHAC1 | 4.65 | 3.857 |
| 7 | CHST15 | 3.18 | 2.211 |
| 8 | CPA3 | 5.19 | 5.849 |
| 9 | CRYAB | 6.32 | 5.2 |
| 10 | DAB2 | 2.29 | |
| 11 | DCN | 8.23 | 7.66 |
| 12 | DSE | 2.01 | 2.04 |
| 13 | DYRK3 | 3.79 | 3.357 |
| 14 | FABP1 | 6.38 | 3.571 |
| 15 | FPR3 | 4.35 | 4.133 |
| 16 | GFPT2 | 2.69 | |
| 17 | HDC | 5.99 | 5.357 |
| 18 | IL22 | 4.37 | |
| 19 | IL6 | 4.78 | 4.756 |
| 20 | KIT | 2.36 | 2.167 |
| 21 | LCN2 | 4.56 | |
| 22 | LMCD1 | 3.12 | 2.636 |
| 23 | LRRC32 | 2.83 | 2.267 |
| 24 | LYZ | 2.07 | 1.842 |
| 25 | MFSD2A | 3.13 | 2.611 |
| 26 | NANOGNB | 5.73 | 5.22 |
| 27 | OR4A5 | 11.69 | 6.429 |
| 28 | PLA2G2A | 7.93 | 3.429 |
| 29 | PLTP | 2.51 | |
| 30 | PPIAP30 | 3.01 | 3.258 |
| 31 | RAB13 | 2.09 | 1.787 |
| 32 | RRAD | 6.91 | 3.425 |
| 33 | SDS | 3.87 | 5 |
| 34 | SEMA6B | 5.8 | 3.714 |
| 35 | SEPP1 | 2.84 | 2.333 |
| 36 | SERPING1 | 4.12 | 4.343 |
| 37 | SOD3 | 5.11 | 3.929 |
| 38 | SYK | 2.34 | 1.761 |
| 39 | TBC1D3 | 11.52 | 5.867 |
| 40 | TBC1D8 | 2.01 | 2 |
| 41 | TBC1D9 | 2.26 | 1.859 |
| 42 | TNXB | 2.79 | 2.295 |
| 43 | TPSB2 | 4.12 | 3.5 |
| 44 | UBD | 6.82 | 4 |
| 45 | ABI3BP | 2.54 | 3.818 |
| 46 | ANKRD20A3 | 5.07 | 4.409 |
| 47 | APOC1P1 | 3.24 | 4.442 |
| 48 | AQP7P3 | 16.67 | 13.553 |
| 49 | C11orf96 | 3.99 | 3.621 |
| 50 | C1QB | 4.66 | 3.71 |
| 51 | C1QC | 4.19 | 6.14 |
| 52 | C2orf27A | 4.86 | 3.095 |
| 53 | C8orf4 | 8.42 | 6.176 |
| 54 | CKB | 3.13 | 1.867 |
| 55 | CLDN10 | 2.86 | 2.873 |
| 56 | CLEC3B | 4.85 | 2.55 |
| 57 | CLIC4 | 2.19 | 1.714 |
| 58 | COL1A1 | 5.31 | 4.028 |
| 59 | COL1A2 | 5.99 | 5.172 |
| 60 | COL5A1 | 2.69 | 2.529 |
| 61 | CXCL13 | 8.3 | 6.038 |
| 62 | CYCSP52 | 3.89 | 3.6 |
| 63 | FAM138D | 3.64 | 3.281 |
| 64 | FAM182B | 14.52 | 10.833 |
| 65 | FAM222A | 2.67 | 1.725 |
| 66 | FAM231A | 2.11 | 1.842 |
| 67 | FAM27A | 9.15 | 4.829 |
| 68 | FSTL1 | 4.4 | 4.824 |
| 69 | GAS7 | 2.21 | 1.591 |
| 70 | GEM | 4.97 | 5.542 |
| 71 | GOLGA6L5P | 3.44 | 2.067 |
| 72 | GPNMB | 6.33 | 4.59 |
| 73 | GYPE | 4.27 | 4.963 |
| 74 | HNRNPA1P33 | 7.75 | 3.278 |
| 75 | HSPA2 | 3.24 | 3.222 |
| 76 | HSPB6 | 6.69 | 4.386 |
| 77 | KGFLP2 | 2.6 | 2.083 |
| 78 | KRT20 | 7.48 | 5 |
| 79 | LIMS3L | 2.07 | 2 |
| 80 | LINC00348 | 3.85 | 2.932 |
| 81 | LINC00700 | 3.68 | 2.879 |
| 82 | LINC00857 | 2.26 | 1.907 |
| 83 | LINC01189 | 6.85 | 5.931 |
| 84 | LOC100129138 | 3.63 | 3.73 |
| 85 | LOC100507006 | 2.14 | 1.372 |
| 86 | LOC100508046 | 16.42 | 12.727 |
| 87 | LOC101927123 | 6.44 | |
| 88 | LOC101927905 | 3.39 | 2.864 |
| 89 | LOC101928163 | 5.48 | 4.151 |
| 90 | LOC102724034 | 2.85 | 1.8 |
| 91 | LOC642426 | 8.09 | 8.542 |
| 92 | LOC645166 | 4.71 | 6.258 |
| 93 | LOC646736 | 3.18 | 4.136 |
| 94 | MIR663A | 24.45 | 17.565 |
| 95 | MLLT10P1 | 2.37 | 3.687 |
| 96 | MMP19 | 7.06 | 4.066 |
| 97 | NCOR1P1 | 7.48 | 10.482 |
| 98 | PGM5-AS1 | 13.24 | 10.532 |
| 99 | PHLDB1 | 2.95 | 2.286 |
| 100 | PMP22 | 7.75 | 3.793 |
| 101 | PTENP1-AS | 5.08 | 5.882 |
| 102 | REG3A | 9.48 | 5.172 |
| 103 | RPSAP9 | 4.16 | 3.734 |
| 104 | SEPSECS-AS1 | 2.6 | |
| 105 | SEPT14 | 4.4 | |
| 106 | SLC9B1 | 2.43 | 2.175 |
| 107 | SLCO4A1 | 2.78 | 2.684 |
| 108 | SMOX | 3.01 | 2.229 |
| 109 | SPARCL1 | 5.83 | 4.561 |
| 110 | SRC | 2.42 | 2.418 |
| 111 | ST13P4 | 5.79 | 5.857 |
| 112 | TCF21 | 8.89 | 8.125 |
| 113 | TCF4 | 2.67 | 2.5 |
| 114 | TMEM45B | 2.05 | 1.585 |
| 115 | UBE2Q2L | 3.7 | 2.33 |
| 116 | UBTFL1 | 16.01 | 9.495 |
| 117 | ZNF582-AS1 | 2.43 | 1.766 |
| 118 | ADM | 3.54 | 3.296 |
| 119 | ANPEP | 2.77 | 2.262 |
| 120 | AOAH-IT1 | 5.73 | 3.767 |
| 121 | ASB2 | 2.16 | 1.629 |
| 122 | ATP5J2-PTCD1 | 8.72 | 2.679 |
| 123 | BASP1 | 2.38 | 1.976 |
| 124 | CCL11 | 7.01 | 4.242 |
| 125 | CD68 | 2.07 | 1.656 |
| 126 | CSF2RB | 2.58 | 2.061 |
| 127 | CTAGE8 | 3.47 | 2.03 |
| 128 | CTGF | 6.8 | 6.25 |
| 129 | CXCL1 | 9.88 | 8.571 |
| 130 | CXCL3 | 4.64 | 5.6 |
| 131 | DEFA5 | 5.86 | 5.161 |
| 132 | DEFA6 | 5.05 | 3.667 |
| 133 | DERL3 | 2.1 | 2.054 |
| 134 | DNASE1L3 | 5.79 | 3.167 |
| 135 | DOK3 | 3.05 | 2.118 |
| 136 | EGR2 | 2.12 | 3.57 |
| 137 | EGR3 | 3.37 | 4.522 |
| 138 | EMP1 | 3.72 | 3.056 |
| 139 | EPAS1 | 2.26 | 2.611 |
| 140 | FAM138A | 5.18 | 3.225 |
| 141 | FAM138F | 5.18 | 3.225 |
| 142 | FAM157B | 3.21 | 4.365 |
| 143 | FDCSP | 5.69 | 3.333 |
| 144 | FOSL1 | 3.85 | 3.851 |
| 145 | FSCN1 | 2.65 | 2.902 |
| 146 | FTH1P3 | 3.3 | 2.75 |
| 147 | GAS6 | 2.24 | 2.315 |
| 148 | GATA2 | 3.44 | 3.667 |
| 149 | GPX3 | 2.01 | 1.92 |
| 150 | HES1 | 4.07 | 4.9 |
| 151 | HES4 | 2.62 | 3.667 |
| 152 | HLA-L | 2.06 | 2.014 |
| 153 | IGFBP7 | 2.98 | 2.068 |
| 154 | IL1RN | 2.99 | 2.598 |
| 155 | IL21R-AS1 | 2.27 | 2.828 |
| 156 | LINC01194 | 6.64 | 2.952 |
| 157 | LOC100240735 | 2.26 | |
| 158 | LOC101927817 | 2.05 | 2.297 |
| 159 | LOC284801 | 2.66 | 4.337 |
| 160 | LOC285740 | 2.22 | 2.321 |
| 161 | LOC441242 | 2.11 | 1.901 |
| 162 | LOC644172 | 9.36 | 7.364 |
| 163 | MAFF | 2.04 | 2.345 |
| 164 | MARCKS | 2.36 | 2.637 |
| 165 | MCTP1 | 2.43 | 2.116 |
| 166 | MGP | 2.66 | 2.081 |
| 167 | MIR548I1 | 6.27 | 5.586 |
| 168 | MIR663B | 15.79 | 30.76 |
| 169 | MMP9 | 5.7 | 4.091 |
| 170 | MT1G | 7.38 | |
| 171 | NPIPB9 | 2.9 | 3.075 |
| 172 | NUCB1-AS1 | 4.88 | 4.429 |
| 173 | OR4F21 | 12.9 | 8.358 |
| 174 | PHACTR1 | 2.26 | 2.211 |
| 175 | PLEKHA4 | 2.88 | 2.278 |
| 176 | PLGLB1 | 2.42 | 2.678 |
| 177 | POC1B-GALNT4 | 6.4 | 5.075 |
| 178 | PRKX-AS1 | 2.53 | 1.952 |
| 179 | PTGS2 | 3.37 | 5.259 |
| 180 | RAB20 | 2.32 | 2.349 |
| 181 | REGI1A | 6.54 | 6.818 |
| 182 | RNASE1 | 7.97 | 3.263 |
| 183 | SDC4 | 2.02 | 2.281 |
| 184 | SEPT10 | 2.38 | |
| 185 | SIRPA | 2.57 | 1.9 |
| 186 | SNAI1 | 2.82 | 3.238 |
| 187 | SPARC | 2.61 | 2.013 |
| 188 | SPHK1 | 4.35 | 3.226 |
| 189 | SPINK4 | 4.27 | |
| 190 | STAB1 | 3.03 | 2.145 |
| 191 | TMEM114 | 5.7 | 2.976 |
| 192 | TNFAIP2 | 2.68 | 2.376 |
| 193 | TNFRSF12A | 3.31 | 4.062 |
| 194 | TNFRSF13B | 3.17 | 2.316 |
| 195 | TPSAB1 | 3.89 | 3.667 |
| 196 | TREM1 | 2.72 | |
| 197 | TUBB6 | 2.55 | 2.039 |
| 198 | UGT2B10 | 11.04 | 10.69 |
| 199 | UPK3B | 2.08 | |
| 200 | VEGFA | 2.58 | 2.531 |

In embodiments where more than one biomarker is detected, the differences in expression between a patient having a CD-PBmu subtype and a reference subject (e.g., non-IBD subject or subject with CD PBT) may be different for each marker, e.g., each of the biomarkers detected is at least about 1.1, about 1.2, about 1.3, about 1.4, about 1.5, about 1.6, about 1.7, about 1.8, about 1.9, about 2, about 3, about 4, about 5, about 6, about 7, about 8, about 9, about 10, or about 15 fold up-modulated as compared to the expression level of the respective biomarker in the reference non-CD-PBmu sample. In some cases, at least about 80%, 85%, 90%, 95%, 96%, 97%, 98%, or 99% of the biomarkers detected in a transcriptomic signature is at least about 1.1, about 1.2, about 1.3, about 1.4, about 1.5, about 1.6, about 1.7, about 1.8, about 1.9, about 2, about 3, about 4, about 5, about 6, about 7, about 8, about 9, about 10, or about 15 fold up-modulated as compared to the expression level of the respective biomarker in the reference non-CD-PBmu sample.

### Monocyte Signature and Profiling

In one aspect, provided herein are monocyte signatures associated with a subtype of IBD, including CD. In some cases, the monocyte signature comprises one or more genes of Table 7A. In some cases, the monocyte signature comprises about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Table 7A.

Further provided are methods and compositions for characterizing a subtype of Crohn's Disease (CD) in a subject. Non-limiting examples of subtypes are monocyte 2 subtype and monocyte 1 subtype. The characterization methods provided include diagnosing the presence or absence of a CD subtype, prognosing whether a subject is predisposed to developing a particular CD subtype, prognosing a response of a patient with a particular CD subtype to a therapeutic treatment, and monitoring CD treatment.

In some embodiments, the methods involve detecting in a biological sample comprising monocytes from a subject expression levels of one or more genes of a monocyte signature to obtain an expression profile comprising the expression levels of each of the one or more genes in the signature. In some embodiments, the monocyte signature comprises one or more biomarkers listed in Table 7A. In some embodiments, the monocyte signature comprises any combination of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Table 7A.

The expression profile of a monocyte signature in a subject may be determined by analyzing monocytes of a subject. The subject may be human. In some embodiments, the monocytes are obtained from a subject having an inflammatory disease, such as inflammatory bowel disease, or specifically, Crohn's Disease. Although the methods described herein are generally referenced for use with Crohn's Disease patients, in some cases the methods and monocyte signatures are applicable to other inflammatory diseases, including, ulcerative colitis.

In some embodiments, the expression level of a biomarker in a sample from a subject is compared to a reference expression level. In some cases, the reference expression level is from a subject that does not comprise IBD. In some cases, the reference expression level is from a subject that comprises a monocyte 1 subtype of CD. In some cases, the reference expression level is from a subject that comprises a monocyte 2 subtype of CD. In some cases, a patient having a monocyte 2 subtype has an expression level of one or more biomarkers at least 1.5-fold, 2-fold, 2.5-fold, 3-fold, 3.5-fold, 4-fold, 4.5-fold, or 5-fold greater than the expression level of the one or more biomarkers in a reference subject (e.g., a subject who has a monocyte 1 subtype). In some cases, a patient having a monocyte 1 subtype has an expression level of one or more biomarkers at least 1.5-fold, 2-fold, 2.5-fold, 3-fold, 3.5-fold, 4-fold, 4.5-fold, or 5-fold greater than the expression level of the one or more biomarkers in a reference subject (e.g., a subject who has a monocyte 2 subtype). Table 7A provides non-limiting examples of expression fold of biomarkers in a monocyte 1 subtype as compared to a monocyte 2 subtype.

### Expression and RNA Sequencing Methods

Any suitable method can be utilized to assess (directly or indirectly) the level of expression of a biomarker in a sample. Non-limiting examples of such methods include analyzing the sample using nucleic acid hybridization methods, nucleic acid reverse transcription methods, nucleic acid amplification methods, array analysis, and combinations thereof. In some embodiments, the level of expression of a biomarker in a sample is determined by detecting a transcribed polynucleotide, or portion thereof, e.g., mRNA, or cDNA, of the biomarker gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, quantitative PCR analysis, RNase protection assays, Northern blotting and in situ hybridization. Other suitable systems for RNA sample analysis include microarray analysis (e.g., using Affymetrix's microarray system or Illumina's BeadArray Technology).

Isolated RNA can be used in hybridization or amplification assays that include, but are not limited to, Southern or Northern analyses, polymerase chain reaction (PCR) analyses and probe arrays. An exemplary method for the determination of RNA levels involves contacting RNA with a nucleic acid molecule (e.g., probe) that can hybridize to the biomarker mRNA. The nucleic acid molecule can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the biomarker genomic DNA. In some embodiments, the RNA is immobilized on a solid surface and contacted with a probe, for example by running the isolated RNA on an agarose gel and transferring the RNA from the gel to a membrane, such as nitrocellulose. In some embodiments, the probe(s) are immobilized on a solid surface, for example, in an Affymetrix gene chip array, and the probe(s) are contacted with RNA.

The level of expression of the biomarker in a sample can also be determined using methods that involve the use of nucleic acid amplification and/or reverse transcriptase, e.g., by RT-PCR, ligase chain reaction, self-sustained sequence replication, transcriptional amplification system, Q-Beta Replicase, rolling circle replication or any other nucleic acid amplification method, followed by the detection of the amplified molecules. These approaches may be useful for the detection of nucleic acid molecules if such molecules are present in very low numbers. In some embodiments, the level of expression of the biomarker is determined by quantitative fluorogenic RT-PCR (e.g., the TaqMan™ System). Such methods may utilize pairs of oligonucleotide primers that are specific for the biomarker.

In some embodiments, biomarker expression is determined by sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

The expression levels of biomarker RNA can be monitored using a membrane blot (such as used in hybridization analysis such as Northern, Southern, dot, and the like), microwells, sample tubes, gels, beads, fibers, or any solid support comprising bound nucleic acids. The determination of biomarker expression level may also comprise using nucleic acid probes in solution.

In some embodiments, microarrays are used to detect the level of expression of a biomarker. DNA microarrays provide one method for the simultaneous measurement of the expression levels of large numbers of genes. Each array consists of a reproducible pattern of capture probes attached to a solid support. Labeled nucleic acid is hybridized to complementary probes on the array and then detected, e.g., by laser scanning Hybridization intensities for each probe on the array are determined and converted to a quantitative value representing relative gene expression levels. High-density oligonucleotide arrays may be useful for determining the gene expression profile for a large number of RNA's in a sample.

Expression of a biomarker can also be assessed at the protein level, using a detection reagent that detects the protein product encoded by the mRNA of the biomarker, directly or indirectly. For example, if an antibody reagent is available that binds specifically to a biomarker protein product to be detected, then such an antibody reagent can be used to detect the expression of the biomarker in a sample from the subject, using techniques, such as immunohistochemistry, ELISA, FACS analysis, and the like.

Other methods for detecting the biomarker at the protein level include methods such as electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), enzyme-linked immunosorbent assays (ELISAs), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

In some instances, a method of detecting an expression profile in a subject comprises contacting nucleic acids from a sample of the subject with a nucleic acid polymer that hybridizes to a region of a biomarker nucleic acid sequence. Hybridization may occur at standard hybridization temperatures, e.g., between about 35° C. and about 65° C. in a standard PCR buffer. In some cases, the biomarker nucleic acid sequence is a sequence comprising at least about 30, 40, 50, 60, 70, 80, 90, or 100 nucleobases of a biomarker listed in Table 1A or Table 7A. The nucleic acid polymer can comprise an oligonucleotide of at least or about 5, 10, 15, 20, 25, 30, 35, 40, 45, 50, 75, 100 or more nucleobases in length and sufficient to specifically hybridize to a biomarker of Table 1A or Table 7A. In some instances, the nucleic acid polymer comprises between about 10 and about 100 nucleobases, between about 10 and about 75 nucleobases, between about 10 and about 50 nucleobases, between about 15 and about 100 nucleobases, between about 15 and about 75 nucleobases, between about 15 and about 50 nucleobases, between about 20 and about 100 nucleobases, between about 20 and about 75 nucleobases, between about 20 and about 50 nucleobases, between about 25 and about 100 nucleobases, between about 25 and about 75 nucleobases, or between about 25 and about 50 nucleobases.

Provided herein is a nucleic acid polymer that specifically hybridizes to ADAMTS1. Provided herein is a nucleic acid polymer that specifically hybridizes to LCN2. Provided herein is a nucleic acid polymer that specifically hybridizes to ADAM28. Provided herein is a nucleic acid polymer that specifically hybridizes to TPSB2. Provided herein is a nucleic acid polymer that specifically hybridizes to PPIAP30. Provided herein is a nucleic acid polymer that specifically hybridizes to GFPT2. Provided herein is a nucleic acid polymer that specifically hybridizes to KIT. Provided herein is a nucleic acid polymer that specifically hybridizes to PLTP. Provided herein is a nucleic acid polymer that specifically hybridizes to MFSD2A. Provided herein is a nucleic acid polymer that specifically hybridizes to IL22. Provided herein is a nucleic acid polymer that specifically hybridizes to LMCD1. Provided herein is a nucleic acid polymer that specifically hybridizes to IL6. Provided herein is a nucleic acid polymer that specifically hybridizes to TBC1D9. Provided herein is a nucleic acid polymer that specifically hybridizes to CHAC1. Provided herein is a nucleic acid polymer that specifically hybridizes to SEPP1. Provided herein is a nucleic acid polymer that specifically hybridizes to SOD3. Provided herein is a nucleic acid polymer that specifically hybridizes to RAB13. Provided herein is a nucleic acid polymer that specifically hybridizes to LYZ. Provided herein is a nucleic acid polymer that specifically hybridizes to CPA3. Provided herein is a nucleic acid polymer that specifically hybridizes to SDS. Provided herein is a nucleic acid polymer that specifically hybridizes to DYRK3. Provided herein is a nucleic acid polymer that specifically hybridizes to DAB2. Provided herein is a nucleic acid polymer that specifically hybridizes to TBC1D8. Provided herein is a nucleic acid polymer that specifically hybridizes to CRYAB. Provided herein is a nucleic acid polymer that specifically hybridizes to TBC1D3. Provided herein is a nucleic acid polymer that specifically hybridizes to LRRC32. Provided herein is a nucleic acid polymer that specifically hybridizes to SERPING1. Provided herein is a nucleic acid polymer that specifically hybridizes to UBD. Provided herein is a nucleic acid polymer that specifically hybridizes to FABP1. Provided herein is a nucleic acid polymer that specifically hybridizes to SYK. Provided herein is a nucleic acid polymer that specifically hybridizes to ALDOB. Provided herein is a nucleic acid polymer that specifically hybridizes to SEMA6B. Provided herein is a nucleic acid polymer that specifically hybridizes to NANOGNB. Provided herein is a nucleic acid polymer that specifically hybridizes to DSE. Provided herein is a nucleic acid polymer that specifically hybridizes to FPR3. Provided herein is a nucleic acid polymer that specifically hybridizes to TNXB. Provided herein is a nucleic acid polymer that specifically hybridizes to OR4A5. Provided herein is a nucleic acid polymer that specifically hybridizes to DCN. Provided herein is a nucleic acid polymer that specifically hybridizes to CHST15. Provided herein is a nucleic acid polymer that specifically hybridizes to ADAMDEC1. Provided herein is a nucleic acid polymer that specifically hybridizes to HDC. Provided herein is a nucleic acid polymer that specifically hybridizes to RRAD. Provided herein is a nucleic acid polymer that specifically hybridizes to C1S. Provided herein is a nucleic acid polymer that specifically hybridizes to PLA2G2A. Provided herein is a nucleic acid polymer that specifically hybridizes to CYCSP52. Provided herein is a nucleic acid polymer that specifically hybridizes to C11orf96. Provided herein is a nucleic acid polymer that specifically hybridizes to SEPSECS-AS1. Provided herein is a nucleic acid polymer that specifically hybridizes to C1QC. Provided herein is a nucleic acid polymer that specifically hybridizes to SLC9B1. Provided herein is a nucleic acid polymer that specifically hybridizes to MLLT10P1. Provided herein is a nucleic acid polymer that specifically hybridizes to LOC102724034. Provided herein is a nucleic acid polymer that specifically hybridizes to SMOX. Provided herein is a nucleic acid polymer that specifically hybridizes to CKB. Provided herein is a nucleic acid polymer that specifically hybridizes to NCOR1P1. Provided herein is a nucleic acid polymer that specifically hybridizes to LOC646736. Provided herein is a nucleic acid polymer that specifically hybridizes to CLEC3B. Provided herein is a nucleic acid polymer that specifically hybridizes to SLCO4A1. Provided herein is a nucleic acid polymer that specifically hybridizes to APOC1P1. Provided herein is a nucleic acid polymer that specifically hybridizes to KGFLP2. Provided herein is a nucleic acid polymer that specifically hybridizes to ABI3BP. Provided herein is a nucleic acid polymer that specifically hybridizes to LINC01189. Provided herein is a nucleic acid polymer that specifically hybridizes to SEPT14. Provided herein is a nucleic acid polymer that specifically hybridizes to FSTL1. Provided herein is a nucleic acid polymer that specifically hybridizes to GEM. Provided herein is a nucleic acid polymer that specifically hybridizes to FAM27A. Provided herein is a nucleic acid polymer that specifically hybridizes to PTENP1-AS. Provided herein is a nucleic acid polymer that specifically hybridizes to LIMS3L. Provided herein is a nucleic acid polymer that specifically hybridizes to ST13P4. Provided herein is a nucleic acid polymer that specifically hybridizes to C1QB. Provided herein is a nucleic acid polymer that specifically hybridizes to HNRNPA1P33. Provided herein is a nucleic acid polymer that specifically hybridizes to MIR663A. Provided herein is a nucleic acid polymer that specifically hybridizes to LOC101927123. Provided herein is a nucleic acid polymer that specifically hybridizes to C2orf27A. Provided herein is a nucleic acid polymer that specifically hybridizes to LOC645166. Provided herein is a nucleic acid polymer that specifically hybridizes to ZNF582-AS1. Provided herein is a nucleic acid polymer that specifically hybridizes to HSPA2. Provided herein is a nucleic acid polymer that specifically hybridizes to COL1A1. Provided herein is a nucleic acid polymer that specifically hybridizes to COL5A1. Provided herein is a nucleic acid polymer that specifically hybridizes to GOLGA6L5P. Provided herein is a nucleic acid polymer that specifically hybridizes to PGM5-AS1. Provided herein is a nucleic acid polymer that specifically hybridizes to CLDN10. Provided herein is a nucleic acid polymer that specifically hybridizes to UBE2Q2L. Provided herein is a nucleic acid polymer that specifically hybridizes to LOC100129138. Provided herein is a nucleic acid polymer that specifically hybridizes to COL1A2. Provided herein is a nucleic acid polymer that specifically hybridizes to SPARCL1. Provided herein is a nucleic acid polymer that specifically hybridizes to FAM222A. Provided herein is a nucleic acid polymer that specifically hybridizes to LINC00857. Provided herein is a nucleic acid polymer that specifically hybridizes to CLIC4. Provided herein is a nucleic acid polymer that specifically hybridizes to FAM182B. Provided herein is a nucleic acid polymer that specifically hybridizes to LOC642426. Provided herein is a nucleic acid polymer that specifically hybridizes to GYPE. Provided herein is a nucleic acid polymer that specifically hybridizes to C8orf4. Provided herein is a nucleic acid polymer that specifically hybridizes to RPSAP9. Provided herein is a nucleic acid polymer that specifically hybridizes to FAM231A. Provided herein is a nucleic acid polymer that specifically hybridizes to LINC00700. Provided herein is a nucleic acid polymer that specifically hybridizes to ANKRD20A3. Provided herein is a nucleic acid polymer that specifically hybridizes to FAM138D. Provided herein is a nucleic acid polymer that specifically hybridizes to KRT20. Provided herein is a nucleic acid polymer that specifically hybridizes to UBTFL1. Provided herein is a nucleic acid polymer that specifically hybridizes to GAS7. Provided herein is a nucleic acid polymer that specifically hybridizes to GPNMB. Provided herein is a nucleic acid polymer that specifically hybridizes to TCF4. Provided herein is a nucleic acid polymer that specifically hybridizes to LINC00348. Provided herein is a nucleic acid polymer that specifically hybridizes to SRC. Provided herein is a nucleic acid polymer that specifically hybridizes to HSPB6. Provided herein is a nucleic acid polymer that specifically hybridizes to LOC100507006. Provided herein is a nucleic acid polymer that specifically hybridizes to TCF21. Provided herein is a nucleic acid polymer that specifically hybridizes to TMEM45B. Provided herein is a nucleic acid polymer that specifically hybridizes to LOC101927905. Provided herein is a nucleic acid polymer that specifically hybridizes to CXCL13. Provided herein is a nucleic acid polymer that specifically hybridizes to AQP7P3. Provided herein is a nucleic acid polymer that specifically hybridizes to PMP22. Provided herein is a nucleic acid polymer that specifically hybridizes to LOC101928163. Provided herein is a nucleic acid polymer that specifically hybridizes to REG3A. Provided herein is a nucleic acid polymer that specifically hybridizes to MMP19. Provided herein is a nucleic acid polymer that specifically hybridizes to PHLDB1. Provided herein is a nucleic acid polymer that specifically hybridizes to LOC100508046. Provided herein is a nucleic acid polymer that specifically hybridizes to SPINK4. Provided herein is a nucleic acid polymer that specifically hybridizes to HES4. Provided herein is a nucleic acid polymer that specifically hybridizes to TREM1. Provided herein is a nucleic acid polymer that specifically hybridizes to TNFRSF12A. Provided herein is a nucleic acid polymer that specifically hybridizes to PRKX-AS1. Provided herein is a nucleic acid polymer that specifically hybridizes to PLGLB1. Provided herein is a nucleic acid polymer that specifically hybridizes to SNAI1. Provided herein is a nucleic acid polymer that specifically hybridizes to NUCB1-AS1. Provided herein is a nucleic acid polymer that specifically hybridizes to BASP1. Provided herein is a nucleic acid polymer that specifically hybridizes to MGP. Provided herein is a nucleic acid polymer that specifically hybridizes to ANPEP. Provided herein is a nucleic acid polymer that specifically hybridizes to PHACTR1. Provided herein is a nucleic acid polymer that specifically hybridizes to ADM. Provided herein is a nucleic acid polymer that specifically hybridizes to DEFA6. Provided herein is a nucleic acid polymer that specifically hybridizes to VEGFA. Provided herein is a nucleic acid polymer that specifically hybridizes to EGR2. Provided herein is a nucleic acid polymer that specifically hybridizes to DEFA5. Provided herein is a nucleic acid polymer that specifically hybridizes to CXCL3. Provided herein is a nucleic acid polymer that specifically hybridizes to SDC4. Provided herein is a nucleic acid polymer that specifically hybridizes to TPSAB 1. Provided herein is a nucleic acid polymer that specifically hybridizes to CD68. Provided herein is a nucleic acid polymer that specifically hybridizes to EPAS1. Provided herein is a nucleic acid polymer that specifically hybridizes to MARCKS. Provided herein is a nucleic acid polymer that specifically hybridizes to TNFAIP2. Provided herein is a nucleic acid polymer that specifically hybridizes to MIR663B. Provided herein is a nucleic acid polymer that specifically hybridizes to TMEM114. Provided herein is a nucleic acid polymer that specifically hybridizes to SIRPA. Provided herein is a nucleic acid polymer that specifically hybridizes to GAS6. Provided herein is a nucleic acid polymer that specifically hybridizes to IGFBP7. Provided herein is a nucleic acid polymer that specifically hybridizes to ASB2. Provided herein is a nucleic acid polymer that specifically hybridizes to HES1. Provided herein is a nucleic acid polymer that specifically hybridizes to LOC284801. Provided herein is a nucleic acid polymer that specifically hybridizes to TNFRSF13B. Provided herein is a nucleic acid polymer that specifically hybridizes to MIR548I1. Provided herein is a nucleic acid polymer that specifically hybridizes to DERL3. Provided herein is a nucleic acid polymer that specifically hybridizes to SPARC. Provided herein is a nucleic acid polymer that specifically hybridizes to EMP1. Provided herein is a nucleic acid polymer that specifically hybridizes to LOC100240735. Provided herein is a nucleic acid polymer that specifically hybridizes to LOC101927817. Provided herein is a nucleic acid polymer that specifically hybridizes to STAB 1. Provided herein is a nucleic acid polymer that specifically hybridizes to UPK3B. Provided herein is a nucleic acid polymer that specifically hybridizes to RAB20. Provided herein is a nucleic acid polymer that specifically hybridizes to MMP9. Provided herein is a nucleic acid polymer that specifically hybridizes to MT1G. Provided herein is a nucleic acid polymer that specifically hybridizes to POC1B-GALNT4. Provided herein is a nucleic acid polymer that specifically hybridizes to CSF2RB. Provided herein is a nucleic acid polymer that specifically hybridizes to IL1RN. Provided herein is a nucleic acid polymer that specifically hybridizes to PLEKHA4. Provided herein is a nucleic acid polymer that specifically hybridizes to LOC644172. Provided herein is a nucleic acid polymer that specifically hybridizes to MAFF. Provided herein is a nucleic acid polymer that specifically hybridizes to FDCSP. Provided herein is a nucleic acid polymer that specifically hybridizes to DNASE1L3. Provided herein is a nucleic acid polymer that specifically hybridizes to PTGS2. Provided herein is a nucleic acid polymer that specifically hybridizes to TUBB6. Provided herein is a nucleic acid polymer that specifically hybridizes to LINC01194. Provided herein is a nucleic acid polymer that specifically hybridizes to CTAGE8. Provided herein is a nucleic acid polymer that specifically hybridizes to REG1A. Provided herein is a nucleic acid polymer that specifically hybridizes to ATP5J2-PTCD1. Provided herein is a nucleic acid polymer that specifically hybridizes to DOK3. Provided herein is a nucleic acid polymer that specifically hybridizes to EGR3. Provided herein is a nucleic acid polymer that specifically hybridizes to AOAH-IT1. Provided herein is a nucleic acid polymer that specifically hybridizes to RNASE1. Provided herein is a nucleic acid polymer that specifically hybridizes to CCL11. Provided herein is a nucleic acid polymer that specifically hybridizes to OR4F21. Provided herein is a nucleic acid polymer that specifically hybridizes to FAM157B. Provided herein is a nucleic acid polymer that specifically hybridizes to GATA2. Provided herein is a nucleic acid polymer that specifically hybridizes to CTGF. Provided herein is a nucleic acid polymer that specifically hybridizes to CXCL1. Provided herein is a nucleic acid polymer that specifically hybridizes to GPX3. Provided herein is a nucleic acid polymer that specifically hybridizes to FAM138A. Provided herein is a nucleic acid polymer that specifically hybridizes to FAM138F. Provided herein is a nucleic acid polymer that specifically hybridizes to FOSL1. Provided herein is a nucleic acid polymer that specifically hybridizes to FSCN1. Provided herein is a nucleic acid polymer that specifically hybridizes to FTH1P3. Provided herein is a nucleic acid polymer that specifically hybridizes to SPHK1. Provided herein is a nucleic acid polymer that specifically hybridizes to LOC441242. Provided herein is a nucleic acid polymer that specifically hybridizes to UGT2B10. Provided herein is a nucleic acid polymer that specifically hybridizes to MCTP1. Provided herein is a nucleic acid polymer that specifically hybridizes to IL21R-AS1. Provided herein is a nucleic acid polymer that specifically hybridizes to LOC285740. Provided herein is a nucleic acid polymer that specifically hybridizes to HLA-L. Provided herein is a nucleic acid polymer that specifically hybridizes to NPIPB9. Provided herein is a nucleic acid polymer that specifically hybridizes to SEPT10.

Nucleic acid polymers include primers useful for amplifying a nucleic acid of biomarker provided in Table 1A or Table 5. For example, for use in an amplification assay such as qPCR. Nucleic acid polymers also include probes comprising a detectable label for detecting and/or quantifying a biomarker of Table 1A or Table 5. In some cases the probes are reporters that comprise a dye label on one end and a quencher on the other end. When the probes are hybridized to a biomarker nucleic acid, an added DNA polymerase may cleave those hybridized probes, separating the reporter dye from the quencher, and thus increasing fluorescence by the reporter. In some cases, provided is a probe comprising a nucleic acid polymer described herein.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 34-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some instances, primers and/or probes described herein for hybridization to a biomarker of Table 1A or Table 7A are used in an amplification reaction. In some instances, the amplification reaction is qPCR. An exemplary qPCR is a method employing a TaqMan™ assay.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In one aspect, the methods provided herein for determining an expression profile in a subject comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

In an exemplary qPCR assay, the nucleic acid sample is combined with primers and probes specific for a biomarker nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a biomarker nucleic acid.

### Profile Analysis

The expression profile of a patient sample (test sample) may be compared to a reference sample, e.g., a sample from a subject who does not have IBD such as CD (normal sample), or a sample from a subject who has a non-CD-PBmu subtype. In some cases, a normal sample is that which is or is expected to be free of IBD disease or condition, or a sample that would test negative for any IBD disease or condition. The reference sample may be assayed at the same time, or at a different time from the test sample. In some cases, the expression profile of a reference sample is obtained and stored in a database for comparison to the test sample.

The results of an assay on the test sample may be compared to the results of the same assay on a reference sample. In some cases the results of the assay on the normal sample are from a database. In some cases, the results of the assay on the normal sample are a known or generally accepted value by those skilled in the art. In some cases the comparison is qualitative. In other cases the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, gene product expression levels, gene product expression level changes, alternative exon usage, changes in alternative exon usage, protein levels, DNA polymorphisms, coy number variations, indications of the presence or absence of one or more DNA markers or regions, and/or nucleic acid sequences.

In some embodiments, the gene expression profile of a test sample is evaluated using methods for correlating gene product expression levels with a specific phenotype of CD, such as the CD-PBmu subtype described herein. In some cases, a specified statistical confidence level may be determined in order to provide a diagnostic confidence level. For example, it may be determined that a confidence level of greater than 90% may be a useful predictor of CD-PBmu. In other embodiments, more or less stringent confidence levels may be chosen. For example, a confidence level of approximately 70%, 75%, 80%, 85%, 90%, 95%, 97.5%, 99%, 99.5%, or 99.9% may be chosen as a useful phenotypic predictor. The confidence level provided may in some cases be related to the quality of the sample, the quality of the data, the quality of the analysis, the specific methods used, and the number of gene expression products analyzed. The specified confidence level for providing a diagnosis may be chosen on the basis of the expected number of false positives or false negatives and/or cost. Methods for choosing parameters for achieving a specified confidence level or for identifying markers with diagnostic power include but are not limited to Receiver Operator Curve analysis (ROC), binormal ROC, principal component analysis, partial least squares analysis, singular value decomposition, least absolute shrinkage and selection operator analysis, least angle regression, and the threshold gradient directed regularization method.

Raw gene expression level data may in some cases be improved through the application of algorithms designed to normalize and or improve the reliability of the data. In some embodiments of the present invention the data analysis requires a computer or other device, machine or apparatus for application of the various algorithms described herein due to the large number of individual data points that are processed. A “machine learning algorithm” refers to a computational-based prediction methodology, also known as a “classifier”, employed for characterizing a gene expression profile. The signals corresponding to certain expression levels, which are obtained by, e.g., microarray-based hybridization assays or sequencing, are typically subjected to the algorithm in order to classify the expression profile. Supervised learning generally involves “training” a classifier to recognize the distinctions among classes and then “testing” the accuracy of the classifier on an independent test set. For test samples the classifier can be used to predict the class in which the samples belong.

In some cases, the robust multi-array Average (RMA) method may be used to normalize the raw data. The RMA method begins by computing background-corrected intensities for each matched cell on a number of microarrays. The background corrected values are restricted to positive values as described by Irizarry et al. Biostatistics 2003 Apr. 4 (2): 249-64. The back-ground corrected, log-transformed, matched intensity on each microarray is then normalized using the quantile normalization method in which for each input array and each probe expression value, the array percentile probe value is replaced with the average of all array percentile points, this method is more completely described by Bolstad et al. Bioinformatics 2003. Following quantile normalization, the normalized data may then be fit to a linear model to obtain an expression measure for each probe on each microarray. Tukey's median polish algorithm (Tukey, J. W., Exploratory Data Analysis. 1977) may then be used to determine the log-scale expression level for the normalized probe set data.

Data may further be filtered to remove data that may be considered suspect. In some embodiments, data deriving from microarray probes that have fewer than about 4, 5, 6, 7 or 8 guanosine+cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+cytosine nucleotides may be considered unreliable due to their aberrant hybridization propensity or secondary structure issues.

In some cases, unreliable probe sets may be selected for exclusion from data analysis by ranking probe-set reliability against a series of reference datasets. For example, RefSeq or Ensembl (EMBL) are considered very high quality reference datasets. Data from probe sets matching RefSeq or Ensembl sequences may in some cases be specifically included in microarray analysis experiments due to their expected high reliability. Similarly data from probe-sets matching less reliable reference datasets may be excluded from further analysis, or considered on a case by case basis for inclusion. In some cases, the Ensembl high throughput cDNA (HTC) and/or mRNA reference datasets may be used to determine the probe-set reliability separately or together. In other cases, probe-set reliability may be ranked. For example, probes and/or probe-sets that match perfectly to all reference datasets such as for example RefSeq, HTC, and mRNA, may be ranked as most reliable (1). Furthermore, probes and/or probe-sets that match two out of three reference datasets may be ranked as next most reliable (2), probes and/or probe-sets that match one out of three reference datasets may be ranked next (3) and probes and/or probe sets that match no reference datasets may be ranked last (4). Probes and or probe-sets may then be included or excluded from analysis based on their ranking. For example, one may choose to include data from category 1, 2, 3, and 4 probe-sets; category 1, 2, and 3 probe-sets; category 1 and 2 probe-sets; or category 1 probe-sets for further analysis. In another example, probe-sets may be ranked by the number of base pair mismatches to reference dataset entries. It is understood that there are many methods understood in the art for assessing the reliability of a given probe and/or probe-set for molecular profiling and the methods of the present invention encompass any of these methods and combinations thereof.

The results of the expression profile may be analyzed to classify a subject as having or lacking an IBD disease or condition, such as a CD-PBmu subtype. In some cases, a diagnostic result may indicate a certain molecular pathway involved in the IBD disease or condition, or a certain grade or stage of a particular IBD disease or condition. In some cases, a diagnostic result may inform an appropriate therapeutic intervention, such as a specific drug regimen like a molecule that targets a biomolecule in a pathway of any biomarker in Table 1A or 3, or a surgical intervention. In some cases, a diagnostic result indicates suitability or non-suitability of a patient for treatment with anti-TNFα.

In some embodiments, results are classified using a trained algorithm. Trained algorithms include algorithms that have been developed using a reference set of samples with a known IBD phenotype, such as PBT and CD-PBmu. Algorithms suitable for categorization of samples include but are not limited to k-nearest neighbor algorithms, concept vector algorithms, naive bayesian algorithms, neural network algorithms, hidden markov model algorithms, genetic algorithms, and mutual information feature selection algorithms or any combination thereof. In some cases, trained algorithms may incorporate data other than gene expression such as DNA polymorphism data, sequencing data, scoring or diagnosis by cytologists or pathologists, information provided by the pre-classifier algorithm, or information about the medical history of the subject.

### Compositions and Methods of Treatment

Provided herein are compositions and methods of treating an individual having an inflammatory disease or condition. Non-limiting examples of inflammatory diseases include diseases of the gastrointestinal tract, liver, and/or gallbladder, including Crohn's disease (CD) and ulcerative colitis, systemic lupus erythematosus (SLE), and rheumatoid arthritis. Compositions include any therapeutic agent that modulates expression and/or activity of a biomolecule in a pathway of one or more markers in Table 4. In some embodiments, the therapeutic agent is am odulator of Adenylate cyclase type 7 (ADCY7), G protein-coupled receptor 65 (GPR65), intercellular adhesion molecule 3 (ICAM3), interferon gamma (IFNGMitogen-activated protein kinase kinase kinase kinase 4 (MAP4K4), E2 receptor EP4 subtype (PTGER4), Receptor-interacting serine/threonine-protein kinase 2 (RIPK2), Ribonuclease T2 (RNASET2), or Tumor necrosis factor ligand superfamily member 15 (TNFSF15). In some implementations, the therapeutic agent is administered to a patient determined to have a CD-PBmu subtype as determined by a method provided herein.

In certain embodiments, described herein are methods for evaluating an effect of a treatment described herein. In some instances, the treatment comprises administration with a therapeutic agent provided herein, and optionally one or more additional therapeutic agents. In some instances, the treatment is monitored by evaluating the gene expression profile of a subject for expression of one or more genes in Table 1A or Table 7A. The gene expression profile may be determined prior to and/or after administration of a therapeutic agent. Gene expression profiling may also be used to ascertain the potential efficacy of a specific therapeutic intervention prior to administering to a subject.

In some embodiments, a therapeutic agent modulates expression and/or activity of ADAMTS1. In some embodiments, a therapeutic agent modulates expression and/or activity of LCN2. In some embodiments, a therapeutic agent modulates expression and/or activity of ADAM28. In some embodiments, a therapeutic agent modulates expression and/or activity of TPSB2. In some embodiments, a therapeutic agent modulates expression and/or activity of PPIAP30. In some embodiments, a therapeutic agent modulates expression and/or activity of GFPT2. In some embodiments, a therapeutic agent modulates expression and/or activity of KIT. In some embodiments, a therapeutic agent modulates expression and/or activity of PLTP. In some embodiments, a therapeutic agent modulates expression and/or activity of MFSD2A. In some embodiments, a therapeutic agent modulates expression and/or activity of IL22. In some embodiments, a therapeutic agent modulates expression and/or activity of LMCD1. In some embodiments, a therapeutic agent modulates expression and/or activity of IL6. In some embodiments, a therapeutic agent modulates expression and/or activity of TBC1D9. In some embodiments, a therapeutic agent modulates expression and/or activity of CHAC1. In some embodiments, a therapeutic agent modulates expression and/or activity of SEPP1. In some embodiments, a therapeutic agent modulates expression and/or activity of SOD3. In some embodiments, a therapeutic agent modulates expression and/or activity of RAB13. In some embodiments, a therapeutic agent modulates expression and/or activity of LYZ. In some embodiments, a therapeutic agent modulates expression and/or activity of CPA3. In some embodiments, a therapeutic agent modulates expression and/or activity of SDS. In some embodiments, a therapeutic agent modulates expression and/or activity of DYRK3. In some embodiments, a therapeutic agent modulates expression and/or activity of DAB2. In some embodiments, a therapeutic agent modulates expression and/or activity of TBC1D8. In some embodiments, a therapeutic agent modulates expression and/or activity of CRYAB. In some embodiments, a therapeutic agent modulates expression and/or activity of TBC1D3. In some embodiments, a therapeutic agent modulates expression and/or activity of LRRC32. In some embodiments, a therapeutic agent modulates expression and/or activity of SERPING1. In some embodiments, a therapeutic agent modulates expression and/or activity of UBD. In some embodiments, a therapeutic agent modulates expression and/or activity of FABP1. In some embodiments, a therapeutic agent modulates expression and/or activity of SYK. In some embodiments, a therapeutic agent modulates expression and/or activity of ALDOB. In some embodiments, a therapeutic agent modulates expression and/or activity of SEMA6B. In some embodiments, a therapeutic agent modulates expression and/or activity of NANOGNB. In some embodiments, a therapeutic agent modulates expression and/or activity of DSE. In some embodiments, a therapeutic agent modulates expression and/or activity of FPR3. In some embodiments, a therapeutic agent modulates expression and/or activity of TNXB. In some embodiments, a therapeutic agent modulates expression and/or activity of OR4A5. In some embodiments, a therapeutic agent modulates expression and/or activity of DCN. In some embodiments, a therapeutic agent modulates expression and/or activity of CHST15. In some embodiments, a therapeutic agent modulates expression and/or activity of ADAMDEC1. In some embodiments, a therapeutic agent modulates expression and/or activity of HDC. In some embodiments, a therapeutic agent modulates expression and/or activity of RRAD. In some embodiments, a therapeutic agent modulates expression and/or activity of C1S. In some embodiments, a therapeutic agent modulates expression and/or activity of PLA2G2A. In some embodiments, a therapeutic agent modulates expression and/or activity of CYCSP52. In some embodiments, a therapeutic agent modulates expression and/or activity of C11orf96. In some embodiments, a therapeutic agent modulates expression and/or activity of SEPSECS-AS1. In some embodiments, a therapeutic agent modulates expression and/or activity of C1QC. In some embodiments, a therapeutic agent modulates expression and/or activity of SLC9B1. In some embodiments, a therapeutic agent modulates expression and/or activity of MLLT10P1. In some embodiments, a therapeutic agent modulates expression and/or activity of LOC102724034. In some embodiments, a therapeutic agent modulates expression and/or activity of SMOX. In some embodiments, a therapeutic agent modulates expression and/or activity of CKB. In some embodiments, a therapeutic agent modulates expression and/or activity of NCOR1P1. In some embodiments, a therapeutic agent modulates expression and/or activity of LOC646736. In some embodiments, a therapeutic agent modulates expression and/or activity of CLEC3B. In some embodiments, a therapeutic agent modulates expression and/or activity of SLCO4A1. In some embodiments, a therapeutic agent modulates expression and/or activity of APOC1P1. In some embodiments, a therapeutic agent modulates expression and/or activity of KGFLP2. In some embodiments, a therapeutic agent modulates expression and/or activity of ABI3BP. In some embodiments, a therapeutic agent modulates expression and/or activity of LINC01189. In some embodiments, a therapeutic agent modulates expression and/or activity of SEPT14. In some embodiments, a therapeutic agent modulates expression and/or activity of FSTL1. In some embodiments, a therapeutic agent modulates expression and/or activity of GEM. In some embodiments, a therapeutic agent modulates expression and/or activity of FAM27A. In some embodiments, a therapeutic agent modulates expression and/or activity of PTENP1-AS. In some embodiments, a therapeutic agent modulates expression and/or activity of LIMS3L. In some embodiments, a therapeutic agent modulates expression and/or activity of ST13P4. In some embodiments, a therapeutic agent modulates expression and/or activity of C1QB. In some embodiments, a therapeutic agent modulates expression and/or activity of HNRNPA1P33. In some embodiments, a therapeutic agent modulates expression and/or activity of MIR663A. In some embodiments, a therapeutic agent modulates expression and/or activity of LOC101927123. In some embodiments, a therapeutic agent modulates expression and/or activity of C2orf27A. In some embodiments, a therapeutic agent modulates expression and/or activity of LOC645166. In some embodiments, a therapeutic agent modulates expression and/or activity of ZNF582-AS1. In some embodiments, a therapeutic agent modulates expression and/or activity of HSPA2. In some embodiments, a therapeutic agent modulates expression and/or activity of COL1A1. In some embodiments, a therapeutic agent modulates expression and/or activity of COL5A1. In some embodiments, a therapeutic agent modulates expression and/or activity of GOLGA6L5P. In some embodiments, a therapeutic agent modulates expression and/or activity of PGM5-AS1. In some embodiments, a therapeutic agent modulates expression and/or activity of CLDN10. In some embodiments, a therapeutic agent modulates expression and/or activity of UBE2Q2L. In some embodiments, a therapeutic agent modulates expression and/or activity of LOC100129138. In some embodiments, a therapeutic agent modulates expression and/or activity of COL1A2. In some embodiments, a therapeutic agent modulates expression and/or activity of SPARCL1. In some embodiments, a therapeutic agent modulates expression and/or activity of FAM222A. In some embodiments, a therapeutic agent modulates expression and/or activity of LINC00857. In some embodiments, a therapeutic agent modulates expression and/or activity of CLIC4. In some embodiments, a therapeutic agent modulates expression and/or activity of FAM182B. In some embodiments, a therapeutic agent modulates expression and/or activity of LOC642426. In some embodiments, a therapeutic agent modulates expression and/or activity of GYPE. In some embodiments, a therapeutic agent modulates expression and/or activity of C8orf4. In some embodiments, a therapeutic agent modulates expression and/or activity of RPSAP9. In some embodiments, a therapeutic agent modulates expression and/or activity of FAM231A. In some embodiments, a therapeutic agent modulates expression and/or activity of LINC00700. In some embodiments, a therapeutic agent modulates expression and/or activity of ANKRD20A3. In some embodiments, a therapeutic agent modulates expression and/or activity of FAM138D. In some embodiments, a therapeutic agent modulates expression and/or activity of KRT20. In some embodiments, a therapeutic agent modulates expression and/or activity of UBTFL1. In some embodiments, a therapeutic agent modulates expression and/or activity of GAS7. In some embodiments, a therapeutic agent modulates expression and/or activity of GPNMB. In some embodiments, a therapeutic agent modulates expression and/or activity of TCF4. In some embodiments, a therapeutic agent modulates expression and/or activity of LINC00348. In some embodiments, a therapeutic agent modulates expression and/or activity of SRC. In some embodiments, a therapeutic agent modulates expression and/or activity of HSPB6. In some embodiments, a therapeutic agent modulates expression and/or activity of LOC100507006. In some embodiments, a therapeutic agent modulates expression and/or activity of TCF21. In some embodiments, a therapeutic agent modulates expression and/or activity of TMEM45B. In some embodiments, a therapeutic agent modulates expression and/or activity of LOC101927905. In some embodiments, a therapeutic agent modulates expression and/or activity of CXCL13. In some embodiments, a therapeutic agent modulates expression and/or activity of AQP7P3. In some embodiments, a therapeutic agent modulates expression and/or activity of PMP22. In some embodiments, a therapeutic agent modulates expression and/or activity of LOC101928163. In some embodiments, a therapeutic agent modulates expression and/or activity of REG3A. In some embodiments, a therapeutic agent modulates expression and/or activity of MMP19. In some embodiments, a therapeutic agent modulates expression and/or activity of PHLDB1. In some embodiments, a therapeutic agent modulates expression and/or activity of LOC100508046. In some embodiments, a therapeutic agent modulates expression and/or activity of SPINK4. In some embodiments, a therapeutic agent modulates expression and/or activity of HES4. In some embodiments, a therapeutic agent modulates expression and/or activity of TREM1. In some embodiments, a therapeutic agent modulates expression and/or activity of TNFRSF12A. In some embodiments, a therapeutic agent modulates expression and/or activity of PRKX-AS1. In some embodiments, a therapeutic agent modulates expression and/or activity of PLGLB1. In some embodiments, a therapeutic agent modulates expression and/or activity of SNAI1. In some embodiments, a therapeutic agent modulates expression and/or activity of NUCB1-AS1. In some embodiments, a therapeutic agent modulates expression and/or activity of BASP1. In some embodiments, a therapeutic agent modulates expression and/or activity of MGP. In some embodiments, a therapeutic agent modulates expression and/or activity of ANPEP. In some embodiments, a therapeutic agent modulates expression and/or activity of PHACTR1. In some embodiments, a therapeutic agent modulates expression and/or activity of ADM. In some embodiments, a therapeutic agent modulates expression and/or activity of DEFA6. In some embodiments, a therapeutic agent modulates expression and/or activity of VEGFA. In some embodiments, a therapeutic agent modulates expression and/or activity of EGR2. In some embodiments, a therapeutic agent modulates expression and/or activity of DEFA5. In some embodiments, a therapeutic agent modulates expression and/or activity of CXCL3. In some embodiments, a therapeutic agent modulates expression and/or activity of SDC4. In some embodiments, a therapeutic agent modulates expression and/or activity of TPSAB 1. In some embodiments, a therapeutic agent modulates expression and/or activity of CD68. In some embodiments, a therapeutic agent modulates expression and/or activity of EPAS1. In some embodiments, a therapeutic agent modulates expression and/or activity of MARCKS. In some embodiments, a therapeutic agent modulates expression and/or activity of TNFAIP2. In some embodiments, a therapeutic agent modulates expression and/or activity of MIR663B. In some embodiments, a therapeutic agent modulates expression and/or activity of TMEM114. In some embodiments, a therapeutic agent modulates expression and/or activity of SIRPA. In some embodiments, a therapeutic agent modulates expression and/or activity of GAS6. In some embodiments, a therapeutic agent modulates expression and/or activity of IGFBP7. In some embodiments, a therapeutic agent modulates expression and/or activity of ASB2. In some embodiments, a therapeutic agent modulates expression and/or activity of HES1. In some embodiments, a therapeutic agent modulates expression and/or activity of LOC284801. In some embodiments, a therapeutic agent modulates expression and/or activity of TNFRSF13B. In some embodiments, a therapeutic agent modulates expression and/or activity of MIR548I1. In some embodiments, a therapeutic agent modulates expression and/or activity of DERL3. In some embodiments, a therapeutic agent modulates expression and/or activity of SPARC. In some embodiments, a therapeutic agent modulates expression and/or activity of EMP1. In some embodiments, a therapeutic agent modulates expression and/or activity of LOC100240735. In some embodiments, a therapeutic agent modulates expression and/or activity of LOC101927817. In some embodiments, a therapeutic agent modulates expression and/or activity of STAB 1. In some embodiments, a therapeutic agent modulates expression and/or activity of UPK3B. In some embodiments, a therapeutic agent modulates expression and/or activity of RAB20. In some embodiments, a therapeutic agent modulates expression and/or activity of MMP9. In some embodiments, a therapeutic agent modulates expression and/or activity of MT1G. In some embodiments, a therapeutic agent modulates expression and/or activity of POC1B-GALNT4. In some embodiments, a therapeutic agent modulates expression and/or activity of CSF2RB. In some embodiments, a therapeutic agent modulates expression and/or activity of IL1RN. In some embodiments, a therapeutic agent modulates expression and/or activity of PLEKHA4. In some embodiments, a therapeutic agent modulates expression and/or activity of LOC644172. In some embodiments, a therapeutic agent modulates expression and/or activity of MAFF. In some embodiments, a therapeutic agent modulates expression and/or activity of FDCSP. In some embodiments, a therapeutic agent modulates expression and/or activity of DNASE1L3. In some embodiments, a therapeutic agent modulates expression and/or activity of PTGS2. In some embodiments, a therapeutic agent modulates expression and/or activity of TUBB6. In some embodiments, a therapeutic agent modulates expression and/or activity of LINC01194. In some embodiments, a therapeutic agent modulates expression and/or activity of CTAGE8. In some embodiments, a therapeutic agent modulates expression and/or activity of REG1A. In some embodiments, a therapeutic agent modulates expression and/or activity of ATP5J2-PTCD1. In some embodiments, a therapeutic agent modulates expression and/or activity of DOK3. In some embodiments, a therapeutic agent modulates expression and/or activity of EGR3. In some embodiments, a therapeutic agent modulates expression and/or activity of AOAH-IT1. In some embodiments, a therapeutic agent modulates expression and/or activity of RNASE1. In some embodiments, a therapeutic agent modulates expression and/or activity of CCL11. In some embodiments, a therapeutic agent modulates expression and/or activity of OR4F21. In some embodiments, a therapeutic agent modulates expression and/or activity of FAM157B. In some embodiments, a therapeutic agent modulates expression and/or activity of GATA2. In some embodiments, a therapeutic agent modulates expression and/or activity of CTGF. In some embodiments, a therapeutic agent modulates expression and/or activity of CXCL1. In some embodiments, a therapeutic agent modulates expression and/or activity of GPX3. In some embodiments, a therapeutic agent modulates expression and/or activity of FAM138A. In some embodiments, a therapeutic agent modulates expression and/or activity of FAM138F. In some embodiments, a therapeutic agent modulates expression and/or activity of FOSL1. In some embodiments, a therapeutic agent modulates expression and/or activity of FSCN1. In some embodiments, a therapeutic agent modulates expression and/or activity of FTH1P3. In some embodiments, a therapeutic agent modulates expression and/or activity of SPHK1. In some embodiments, a therapeutic agent modulates expression and/or activity of LOC441242. In some embodiments, a therapeutic agent modulates expression and/or activity of UGT2B10. In some embodiments, a therapeutic agent modulates expression and/or activity of MCTP1. In some embodiments, a therapeutic agent modulates expression and/or activity of IL21R-AS1. In some embodiments, a therapeutic agent modulates expression and/or activity of LOC285740. In some embodiments, a therapeutic agent modulates expression and/or activity of HLA-L. In some embodiments, a therapeutic agent modulates expression and/or activity of NPIPB9. In some embodiments, a therapeutic agent modulates expression and/or activity of SEPT10. In some embodiments, a therapeutic agent modulates expression and/or activity of DNAPK. In some embodiments, a therapeutic agent modulates expression and/or activity of CDK4. In some embodiments, a therapeutic agent modulates expression and/or activity of ERK1. In some embodiments, a therapeutic agent modulates expression and/or activity of HIPK2. In some embodiments, a therapeutic agent modulates expression and/or activity of CDC2. In some embodiments, a therapeutic agent modulates expression and/or activity of MAPK1. In some embodiments, a therapeutic agent modulates expression and/or activity of MAPK3. In some embodiments, a therapeutic agent modulates expression and/or activity of ERK2. In some embodiments, a therapeutic agent modulates expression and/or activity of CSNK2A1. In some embodiments, a therapeutic agent modulates expression and/or activity of CK2ALPHA. In some embodiments, a therapeutic agent modulates expression and/or activity of JNK1. In some embodiments, a therapeutic agent modulates expression and/or activity of CDK1. In some embodiments, a therapeutic agent modulates expression and/or activity of MAPK14. In some embodiments, a therapeutic agent modulates expression and/or activity of PKR. In some embodiments, a therapeutic agent modulates expression and/or activity of CDK2.

In some embodiments, a therapeutic agent modulates expression and/or activity of a biomolecule in a pathway comprising ADAMTS1. In some embodiments, a therapeutic agent modulates expression and/or activity of a biomolecule in a pathway comprising LCN2. In some embodiments, a therapeutic agent modulates expression and/or activity of a biomolecule in a pathway comprising ADAM28. In some embodiments, a therapeutic agent modulates expression and/or activity of a biomolecule in a pathway comprising TPSB2. In some embodiments, a therapeutic agent modulates expression and/or activity of a biomolecule in a pathway comprising PPIAP30. In some embodiments, a therapeutic agent modulates expression and/or activity of a biomolecule in a pathway comprising GFPT2. In some embodiments, a therapeutic agent modulates expression and/or activity of a biomolecule in a pathway comprising KIT. In some embodiments, a therapeutic agent modulates expression and/or activity of a biomolecule in a pathway comprising PLTP. In some embodiments, a therapeutic agent modulates expression and/or activity of a biomolecule in a pathway comprising MFSD2A. In some embodiments, a therapeutic agent modulates expression and/or activity of a biomolecule in a pathway comprising IL22. In some embodiments, a therapeutic agent modulates expression and/or activity of a biomolecule in a pathway comprising LMCD1. In some embodiments, a therapeutic agent modulates expression and/or activity of a biomolecule in a pathway comprising IL6. In some embodiments, a therapeutic agent modulates expression and/or activity of a biomolecule in a pathway comprising TBC1D9. In some embodiments, a therapeutic agent modulates expression and/or activity of a biomolecule in a pathway comprising CHAC1. In some embodiments, a therapeutic agent modulates expression and/or activity of a biomolecule in a pathway comprising SEPP1. In some embodiments, a therapeutic agent modulates expression and/or activity of a biomolecule in a pathway comprising SOD3. In some embodiments, a therapeutic agent modulates expression and/or activity of a biomolecule in a pathway comprising RAB13. In some embodiments, a therapeutic agent modulates expression and/or activity of a biomolecule in a pathway comprising LYZ. In some embodiments, a therapeutic agent modulates expression and/or activity of a biomolecule in a pathway comprising CPA3. In some embodiments, a therapeutic agent modulates expression and/or activity of a biomolecule in a pathway comprising SDS. In some embodiments, a therapeutic agent modulates expression and/or activity of a biomolecule in a pathway comprising DYRK3. In some embodiments, a therapeutic agent modulates expression and/or activity of a biomolecule in a pathway comprising DAB2. In some embodiments, a therapeutic agent modulates expression and/or activity of a biomolecule in a pathway comprising TBC1D8. In some embodiments, a therapeutic agent modulates expression and/or activity of a biomolecule in a pathway comprising CRYAB. In some embodiments, a therapeutic agent modulates expression and/or activity of a biomolecule in a pathway comprising TBC1D3. In some embodiments, a therapeutic agent modulates expression and/or activity of a biomolecule in a pathway comprising LRRC32. In some embodiments, a therapeutic agent modulates expression and/or activity of a biomolecule in a pathway comprising SERPING1. In some embodiments, a therapeutic agent modulates expression and/or activity of a biomolecule in a pathway comprising UBD. In some embodiments, a therapeutic agent modulates expression and/or activity of a biomolecule in a pathway comprising FABP1. In some embodiments, a therapeutic agent modulates expression and/or activity of a biomolecule in a pathway comprising SYK. In some embodiments, a therapeutic agent modulates expression and/or activity of a biomolecule in a pathway comprising ALDOB. In some embodiments, a therapeutic agent modulates expression and/or activity of a biomolecule in a pathway comprising SEMA6B. In some embodiments, a therapeutic agent modulates expression and/or activity of a biomolecule in a pathway comprising NANOGNB. In some embodiments, a therapeutic agent modulates expression and/or activity of a biomolecule in a pathway comprising DSE. In some embodiments, a therapeutic agent modulates expression and/or activity of a biomolecule in a pathway comprising FPR3. In some embodiments, a therapeutic agent modulates expression and/or activity of a biomolecule in a pathway comprising TNXB. In some embodiments, a therapeutic agent modulates expression and/or activity of a biomolecule in a pathway comprising OR4A5. In some embodiments, a therapeutic agent modulates expression and/or activity of a biomolecule in a pathway comprising DCN. In some embodiments, a therapeutic agent modulates expression and/or activity of a biomolecule in a pathway comprising CHST15. In some embodiments, a therapeutic agent modulates expression and/or activity of a biomolecule in a pathway comprising ADAMDEC1. In some embodiments, a therapeutic agent modulates expression and/or activity of a biomolecule in a pathway comprising HDC. In some embodiments, a therapeutic agent modulates expression and/or activity of a biomolecule in a pathway comprising RRAD. In some embodiments, a therapeutic agent modulates expression and/or activity of a biomolecule in a pathway comprising C1S. In some embodiments, a therapeutic agent modulates expression and/or activity of a biomolecule in a pathway comprising PLA2G2A. In some embodiments, a therapeutic agent modulates expression and/or activity of a biomolecule in a pathway comprising CYCSP52. In some embodiments, a therapeutic agent modulates expression and/or activity of a biomolecule in a pathway comprising C11orf96. In some embodiments, a therapeutic agent modulates expression and/or activity of a biomolecule in a pathway comprising SEPSECS-AS1. In some embodiments, a therapeutic agent modulates expression and/or activity of a biomolecule in a pathway comprising C1QC. In some embodiments, a therapeutic agent modulates expression and/or activity of a biomolecule in a pathway comprising SLC9B1. In some embodiments, a therapeutic agent modulates expression and/or activity of a biomolecule in a pathway comprising MLLT10P1. In some embodiments, a therapeutic agent modulates expression and/or activity of a biomolecule in a pathway comprising LOC102724034. In some embodiments, a therapeutic agent modulates expression and/or activity of a biomolecule in a pathway comprising SMOX. In some embodiments, a therapeutic agent modulates expression and/or activity of a biomolecule in a pathway comprising CKB. In some embodiments, a therapeutic agent modulates expression and/or activity of a biomolecule in a pathway comprising NCOR1P1. In some embodiments, a therapeutic agent modulates expression and/or activity of a biomolecule in a pathway comprising LOC646736. In some embodiments, a therapeutic agent modulates expression and/or activity of a biomolecule in a pathway comprising CLEC3B. In some embodiments, a therapeutic agent modulates expression and/or activity of a biomolecule in a pathway comprising SLCO4A1. In some embodiments, a therapeutic agent modulates expression and/or activity of a biomolecule in a pathway comprising APOC1P1. In some embodiments, a therapeutic agent modulates expression and/or activity of a biomolecule in a pathway comprising KGFLP2. In some embodiments, a therapeutic agent modulates expression and/or activity of a biomolecule in a pathway comprising ABI3BP. In some embodiments, a therapeutic agent modulates expression and/or activity of a biomolecule in a pathway comprising LINC01189. In some embodiments, a therapeutic agent modulates expression and/or activity of a biomolecule in a pathway comprising SEPT14. In some embodiments, a therapeutic agent modulates expression and/or activity of a biomolecule in a pathway comprising FSTL1. In some embodiments, a therapeutic agent modulates expression and/or activity of a biomolecule in a pathway comprising GEM. In some embodiments, a therapeutic agent modulates expression and/or activity of a biomolecule in a pathway comprising FAM27A. In some embodiments, a therapeutic agent modulates expression and/or activity of a biomolecule in a pathway comprising PTENP1-AS. In some embodiments, a therapeutic agent modulates expression and/or activity of a biomolecule in a pathway comprising LIMS3L. In some embodiments, a therapeutic agent modulates expression and/or activity of a biomolecule in a pathway comprising ST13P4. In some embodiments, a therapeutic agent modulates expression and/or activity of a biomolecule in a pathway comprising C1QB. In some embodiments, a therapeutic agent modulates expression and/or activity of a biomolecule in a pathway comprising HNRNPA1P33. In some embodiments, a therapeutic agent modulates expression and/or activity of a biomolecule in a pathway comprising MIR663A. In some embodiments, a therapeutic agent modulates expression and/or activity of a biomolecule in a pathway comprising LOC101927123. In some embodiments, a therapeutic agent modulates expression and/or activity of a biomolecule in a pathway comprising C2orf27A. In some embodiments, a therapeutic agent modulates expression and/or activity of a biomolecule in a pathway comprising LOC645166. In some embodiments, a therapeutic agent modulates expression and/or activity of a biomolecule in a pathway comprising ZNF582-AS1. In some embodiments, a therapeutic agent modulates expression and/or activity of a biomolecule in a pathway comprising HSPA2. In some embodiments, a therapeutic agent modulates expression and/or activity of a biomolecule in a pathway comprising COL1A1. In some embodiments, a therapeutic agent modulates expression and/or activity of a biomolecule in a pathway comprising COL5A1. In some embodiments, a therapeutic agent modulates expression and/or activity of a biomolecule in a pathway comprising GOLGA6L5P. In some embodiments, a therapeutic agent modulates expression and/or activity of a biomolecule in a pathway comprising PGM5-AS1. In some embodiments, a therapeutic agent modulates expression and/or activity of a biomolecule in a pathway comprising CLDN10. In some embodiments, a therapeutic agent modulates expression and/or activity of a biomolecule in a pathway comprising UBE2Q2L. In some embodiments, a therapeutic agent modulates expression and/or activity of a biomolecule in a pathway comprising LOC100129138. In some embodiments, a therapeutic agent modulates expression and/or activity of a biomolecule in a pathway comprising COL1A2. In some embodiments, a therapeutic agent modulates expression and/or activity of a biomolecule in a pathway comprising SPARCL1. In some embodiments, a therapeutic agent modulates expression and/or activity of a biomolecule in a pathway comprising FAM222A. In some embodiments, a therapeutic agent modulates expression and/or activity of a biomolecule in a pathway comprising LINC00857. In some embodiments, a therapeutic agent modulates expression and/or activity of a biomolecule in a pathway comprising CLIC4. In some embodiments, a therapeutic agent modulates expression and/or activity of a biomolecule in a pathway comprising FAM182B. In some embodiments, a therapeutic agent modulates expression and/or activity of a biomolecule in a pathway comprising LOC642426. In some embodiments, a therapeutic agent modulates expression and/or activity of a biomolecule in a pathway comprising GYPE. In some embodiments, a therapeutic agent modulates expression and/or activity of a biomolecule in a pathway comprising C8orf4. In some embodiments, a therapeutic agent modulates expression and/or activity of a biomolecule in a pathway comprising RPSAP9. In some embodiments, a therapeutic agent modulates expression and/or activity of a biomolecule in a pathway comprising FAM231A. In some embodiments, a therapeutic agent modulates expression and/or activity of a biomolecule in a pathway comprising LINC00700. In some embodiments, a therapeutic agent modulates expression and/or activity of a biomolecule in a pathway comprising ANKRD20A3. In some embodiments, a therapeutic agent modulates expression and/or activity of a biomolecule in a pathway comprising FAM138D. In some embodiments, a therapeutic agent modulates expression and/or activity of a biomolecule in a pathway comprising KRT20. In some embodiments, a therapeutic agent modulates expression and/or activity of a biomolecule in a pathway comprising UBTFL1. In some embodiments, a therapeutic agent modulates expression and/or activity of a biomolecule in a pathway comprising GAS7. In some embodiments, a therapeutic agent modulates expression and/or activity of a biomolecule in a pathway comprising GPNMB. In some embodiments, a therapeutic agent modulates expression and/or activity of a biomolecule in a pathway comprising TCF4. In some embodiments, a therapeutic agent modulates expression and/or activity of a biomolecule in a pathway comprising LINC00348. In some embodiments, a therapeutic agent modulates expression and/or activity of a biomolecule in a pathway comprising SRC. In some embodiments, a therapeutic agent modulates expression and/or activity of a biomolecule in a pathway comprising HSPB6. In some embodiments, a therapeutic agent modulates expression and/or activity of a biomolecule in a pathway comprising LOC100507006. In some embodiments, a therapeutic agent modulates expression and/or activity of a biomolecule in a pathway comprising TCF21. In some embodiments, a therapeutic agent modulates expression and/or activity of a biomolecule in a pathway comprising TMEM45B. In some embodiments, a therapeutic agent modulates expression and/or activity of a biomolecule in a pathway comprising LOC101927905. In some embodiments, a therapeutic agent modulates expression and/or activity of a biomolecule in a pathway comprising CXCL13. In some embodiments, a therapeutic agent modulates expression and/or activity of a biomolecule in a pathway comprising AQP7P3. In some embodiments, a therapeutic agent modulates expression and/or activity of a biomolecule in a pathway comprising PMP22. In some embodiments, a therapeutic agent modulates expression and/or activity of a biomolecule in a pathway comprising LOC101928163. In some embodiments, a therapeutic agent modulates expression and/or activity of a biomolecule in a pathway comprising REG3A. In some embodiments, a therapeutic agent modulates expression and/or activity of a biomolecule in a pathway comprising MMP19. In some embodiments, a therapeutic agent modulates expression and/or activity of a biomolecule in a pathway comprising PHLDB1. In some embodiments, a therapeutic agent modulates expression and/or activity of a biomolecule in a pathway comprising LOC100508046. In some embodiments, a therapeutic agent modulates expression and/or activity of a biomolecule in a pathway comprising SPINK4. In some embodiments, a therapeutic agent modulates expression and/or activity of a biomolecule in a pathway comprising HES4. In some embodiments, a therapeutic agent modulates expression and/or activity of a biomolecule in a pathway comprising TREM1. In some embodiments, a therapeutic agent modulates expression and/or activity of a biomolecule in a pathway comprising TNFRSF12A. In some embodiments, a therapeutic agent modulates expression and/or activity of a biomolecule in a pathway comprising PRKX-AS1. In some embodiments, a therapeutic agent modulates expression and/or activity of a biomolecule in a pathway comprising PLGLB1. In some embodiments, a therapeutic agent modulates expression and/or activity of a biomolecule in a pathway comprising SNAI1. In some embodiments, a therapeutic agent modulates expression and/or activity of a biomolecule in a pathway comprising NUCB1-AS1. In some embodiments, a therapeutic agent modulates expression and/or activity of a biomolecule in a pathway comprising BASP1. In some embodiments, a therapeutic agent modulates expression and/or activity of a biomolecule in a pathway comprising MGP. In some embodiments, a therapeutic agent modulates expression and/or activity of a biomolecule in a pathway comprising ANPEP. In some embodiments, a therapeutic agent modulates expression and/or activity of a biomolecule in a pathway comprising PHACTR1. In some embodiments, a therapeutic agent modulates expression and/or activity of a biomolecule in a pathway comprising ADM. In some embodiments, a therapeutic agent modulates expression and/or activity of a biomolecule in a pathway comprising DEFA6. In some embodiments, a therapeutic agent modulates expression and/or activity of a biomolecule in a pathway comprising VEGFA. In some embodiments, a therapeutic agent modulates expression and/or activity of a biomolecule in a pathway comprising EGR2. In some embodiments, a therapeutic agent modulates expression and/or activity of a biomolecule in a pathway comprising DEFA5. In some embodiments, a therapeutic agent modulates expression and/or activity of a biomolecule in a pathway comprising CXCL3. In some embodiments, a therapeutic agent modulates expression and/or activity of a biomolecule in a pathway comprising SDC4. In some embodiments, a therapeutic agent modulates expression and/or activity of a biomolecule in a pathway comprising TPSAB 1. In some embodiments, a therapeutic agent modulates expression and/or activity of a biomolecule in a pathway comprising CD68. In some embodiments, a therapeutic agent modulates expression and/or activity of a biomolecule in a pathway comprising EPAS1. In some embodiments, a therapeutic agent modulates expression and/or activity of a biomolecule in a pathway comprising MARCKS. In some embodiments, a therapeutic agent modulates expression and/or activity of a biomolecule in a pathway comprising TNFAIP2. In some embodiments, a therapeutic agent modulates expression and/or activity of a biomolecule in a pathway comprising MIR663B. In some embodiments, a therapeutic agent modulates expression and/or activity of a biomolecule in a pathway comprising TMEM114. In some embodiments, a therapeutic agent modulates expression and/or activity of a biomolecule in a pathway comprising SIRPA. In some embodiments, a therapeutic agent modulates expression and/or activity of a biomolecule in a pathway comprising GAS6. In some embodiments, a therapeutic agent modulates expression and/or activity of a biomolecule in a pathway comprising IGFBP7. In some embodiments, a therapeutic agent modulates expression and/or activity of a biomolecule in a pathway comprising ASB2. In some embodiments, a therapeutic agent modulates expression and/or activity of a biomolecule in a pathway comprising HES1. In some embodiments, a therapeutic agent modulates expression and/or activity of a biomolecule in a pathway comprising LOC284801. In some embodiments, a therapeutic agent modulates expression and/or activity of a biomolecule in a pathway comprising TNFRSF13B. In some embodiments, a therapeutic agent modulates expression and/or activity of a biomolecule in a pathway comprising MIR548I1. In some embodiments, a therapeutic agent modulates expression and/or activity of a biomolecule in a pathway comprising DERL3. In some embodiments, a therapeutic agent modulates expression and/or activity of a biomolecule in a pathway comprising SPARC. In some embodiments, a therapeutic agent modulates expression and/or activity of a biomolecule in a pathway comprising EMP1. In some embodiments, a therapeutic agent modulates expression and/or activity of a biomolecule in a pathway comprising LOC100240735. In some embodiments, a therapeutic agent modulates expression and/or activity of a biomolecule in a pathway comprising LOC101927817. In some embodiments, a therapeutic agent modulates expression and/or activity of a biomolecule in a pathway comprising STAB 1. In some embodiments, a therapeutic agent modulates expression and/or activity of a biomolecule in a pathway comprising UPK3B. In some embodiments, a therapeutic agent modulates expression and/or activity of a biomolecule in a pathway comprising RAB20. In some embodiments, a therapeutic agent modulates expression and/or activity of a biomolecule in a pathway comprising MMP9. In some embodiments, a therapeutic agent modulates expression and/or activity of a biomolecule in a pathway comprising MT1G. In some embodiments, a therapeutic agent modulates expression and/or activity of a biomolecule in a pathway comprising POC1B-GALNT4. In some embodiments, a therapeutic agent modulates expression and/or activity of a biomolecule in a pathway comprising CSF2RB. In some embodiments, a therapeutic agent modulates expression and/or activity of a biomolecule in a pathway comprising IL1RN. In some embodiments, a therapeutic agent modulates expression and/or activity of a biomolecule in a pathway comprising PLEKHA4. In some embodiments, a therapeutic agent modulates expression and/or activity of a biomolecule in a pathway comprising LOC644172. In some embodiments, a therapeutic agent modulates expression and/or activity of a biomolecule in a pathway comprising MAFF. In some embodiments, a therapeutic agent modulates expression and/or activity of a biomolecule in a pathway comprising FDCSP. In some embodiments, a therapeutic agent modulates expression and/or activity of a biomolecule in a pathway comprising DNASE1L3. In some embodiments, a therapeutic agent modulates expression and/or activity of a biomolecule in a pathway comprising PTGS2. In some embodiments, a therapeutic agent modulates expression and/or activity of a biomolecule in a pathway comprising TUBB6. In some embodiments, a therapeutic agent modulates expression and/or activity of a biomolecule in a pathway comprising LINC01194. In some embodiments, a therapeutic agent modulates expression and/or activity of a biomolecule in a pathway comprising CTAGE8. In some embodiments, a therapeutic agent modulates expression and/or activity of a biomolecule in a pathway comprising REG1A. In some embodiments, a therapeutic agent modulates expression and/or activity of a biomolecule in a pathway comprising ATP5J2-PTCD1. In some embodiments, a therapeutic agent modulates expression and/or activity of a biomolecule in a pathway comprising DOK3. In some embodiments, a therapeutic agent modulates expression and/or activity of a biomolecule in a pathway comprising EGR3. In some embodiments, a therapeutic agent modulates expression and/or activity of a biomolecule in a pathway comprising AOAH-IT1. In some embodiments, a therapeutic agent modulates expression and/or activity of a biomolecule in a pathway comprising RNASE1. In some embodiments, a therapeutic agent modulates expression and/or activity of a biomolecule in a pathway comprising CCL11. In some embodiments, a therapeutic agent modulates expression and/or activity of a biomolecule in a pathway comprising OR4F21. In some embodiments, a therapeutic agent modulates expression and/or activity of a biomolecule in a pathway comprising FAM157B. In some embodiments, a therapeutic agent modulates expression and/or activity of a biomolecule in a pathway comprising GATA2. In some embodiments, a therapeutic agent modulates expression and/or activity of a biomolecule in a pathway comprising CTGF. In some embodiments, a therapeutic agent modulates expression and/or activity of a biomolecule in a pathway comprising CXCL1. In some embodiments, a therapeutic agent modulates expression and/or activity of a biomolecule in a pathway comprising GPX3. In some embodiments, a therapeutic agent modulates expression and/or activity of a biomolecule in a pathway comprising FAM138A. In some embodiments, a therapeutic agent modulates expression and/or activity of a biomolecule in a pathway comprising FAM138F. In some embodiments, a therapeutic agent modulates expression and/or activity of a biomolecule in a pathway comprising FOSL1. In some embodiments, a therapeutic agent modulates expression and/or activity of a biomolecule in a pathway comprising FSCN1. In some embodiments, a therapeutic agent modulates expression and/or activity of a biomolecule in a pathway comprising FTH1P3. In some embodiments, a therapeutic agent modulates expression and/or activity of a biomolecule in a pathway comprising SPHK1. In some embodiments, a therapeutic agent modulates expression and/or activity of a biomolecule in a pathway comprising LOC441242. In some embodiments, a therapeutic agent modulates expression and/or activity of a biomolecule in a pathway comprising UGT2B10. In some embodiments, a therapeutic agent modulates expression and/or activity of a biomolecule in a pathway comprising MCTP1. In some embodiments, a therapeutic agent modulates expression and/or activity of a biomolecule in a pathway comprising IL21R-AS1. In some embodiments, a therapeutic agent modulates expression and/or activity of a biomolecule in a pathway comprising LOC285740. In some embodiments, a therapeutic agent modulates expression and/or activity of a biomolecule in a pathway comprising HLA-L. In some embodiments, a therapeutic agent modulates expression and/or activity of a biomolecule in a pathway comprising NPIPB9. In some embodiments, a therapeutic agent modulates expression and/or activity of a biomolecule in a pathway comprising SEPT10.

Adenylate Cyclase 7 (ADCY7) Modulators

Adenylate Cyclase 7 (ADCY7) (UniProtKB: P51828), and nucleic acids encoding ADCY7 (Entrez ID 113), is a membrane-bound adenylate cyclase that catalyzes the formation of adenosine monophosphate (cAMP) from adenosine triphosphate (ATP). ADCY7 functions in signaling cascades activated namely by thrombin and sphingosine 1-phosphate and mediates regulation of cAMP synthesis through synergistic action of the stimulatory G alpha protein with G Protein Subunit Alpha 13 (GNA13). Also, during inflammation, mediates zymosan-induced increase intracellular cAMP, leading to protein kinase A pathway activation in order to modulate innate immune responses through heterotrimeric GNA13/G Protein Subunit Alpha 12 (GNA12). ADCY7 also functions in signaling cascades activated namely by dopamine and C5 alpha chain and mediates regulation of cAMP synthesis through synergistic action of the stimulatory G protein with G beta:gamma complex. Through cAMP response regulation, ADCY7 keeps inflammation under control during bacterial infection by sensing the presence of serum factors, such as the bioactive lysophospholipid (LPA) that regulate LPS-induced TNF-alpha production.

Disclosed herein, in some embodiments, are therapeutic agents comprising modulators of Adenylate Cyclase 7 (ADCY7) useful for the treatment of a disease or condition, or symptom of the disease or condition, disclosed herein. In some embodiments, the therapeutic agent comprises a modulator, agonist, and/or antagonist of ADCY7. Disclosed herein, in some embodiments are methods of treating a disease or condition in a subject by administering a therapeutically effective amount of an agonist of ADCY7 to the subject, thereby increasing ADCY7 expression or activity. The agonist of ADCY7 expression or activity may be a direct agonist or indirect agonist. In some embodiments, the agonist of ADCY7 expression or activity comprises a complete agonist or a partial agonist. Non-limiting examples of an agonist of ADCY7 expression include RNA to protein ADCY7 translation agonists, antisense oligonucleotides targeting the ADCY7, or homolog thereof, mRNA (such as miRNAs, or siRNA), epigenetic editing (such as post-translational modifications of histone tails and/or DNA molecules). Non-limiting examples of an agonist of ADCY7 activity include antagonists to the ADCY7 antigen, and antagonists to gene expression products involved in ADCY7 mediated disease. Agonists as disclosed herein, may include, but are not limited to, an ADCY7 antibody, an ADCY7-binding antibody fragment, recombinant polypeptide, or a small molecule. The small molecule may be a small molecule that binds to ADCY7 or binding partners to ADCY7. The ADCY7 antibody may be monoclonal or polyclonal. The ADCY7 antibody may be humanized or chimeric. The ADCY7 antibody may be a fusion protein. The ADCY7 antibody may be a blocking ADCY7 antibody. A blocking antibody blocks binding between two proteins, e.g., a ligand and its receptor. In a non-limiting example, the ADCY7 blocking antibody binds to a binding partner of ADCY7. In some cases, the ADCY7 antibody is an ADCY7 antibody that specifically binds to ADCY7. In some cases, the ADCY7 is naturally occurring. In some embodiments, the ADCY7 agonists comprise one or more small molecule compounds that are pan-activators of adenylyl cyclases (ACs). Non-limiting examples of ADCY7 agonists that are pan-activators of ACs include forskolin, colforsin daropate, and analogs thereof.

Disclosed herein, in some embodiments are methods of treating a disease or condition in a subject by administering a therapeutically effective amount of an antagonist of ADCY7 to the subject, thereby decreasing ADCY7 expression or activity. The antagonist of ADCY7 expression or activity may be a direct antagonist or indirect antagonist. In some embodiments, the antagonist of ADCY7 expression or activity comprises a complete antagonist or a partial antagonist. Non-limiting examples of an antagonist of ADCY7 expression include RNA to protein ADCY7 translation antagonists, antisense oligonucleotides targeting the ADCY7C, or homolog thereof, mRNA (such as miRNAs, or siRNA), epigenetic editing (such as post-translational modifications of histone tails and/or DNA molecules). Non-limiting examples of an antagonist of ADCY7 activity include antagonists to the ADCY7 antigen, and antagonists to gene expression products involved in ADCY7 mediated disease. Antagonists as disclosed herein, may include, but are not limited to, an ADCY7 antibody, an ADCY7-binding antibody fragment, recombinant polypeptide, or a small molecule. The small molecule may be a small molecule that binds to ADCY7 or binding partners to ADCY7. The ADCY7 antibody may be monoclonal or polyclonal. The ADCY7 antibody may be humanized or chimeric. The ADCY7 antibody may be a fusion protein. The ADCY7 antibody may be a blocking ADCY7 antibody. A blocking antibody blocks binding between two proteins, e.g., a ligand and its receptor. In a non-limiting example, the ADCY7 blocking antibody binds to a binding partner of ADCY7. In some cases, the ADCY7 antibody is an ADCY7 antibody that specifically binds to ADCY7. In some cases, the ADCY7 is naturally occurring. In some embodiments, the ADCY7 antagonists comprise one or more small molecule compounds. In some embodiments, the small molecule comprises antagonist that are inverse agonists.

Disclosed herein, in some embodiments are methods of treating a disease or condition in a subject by administering a therapeutically effective amount of an allosteric modulator of ADCY7 activity or expression to the subject, thereby decreasing or increasing ADCY7 expression or activity. In some embodiments, the allosteric modulator of ADCY7 is a positive allosteric modulator (PAM) effective to enhance or potentiate a ligand of ADCY7. In some embodiments, the allosteric modulator of ADCY7 is a negative allosteric modulator (NAM) effective to reduce the effect of a primary ligand of ADCY7. In some embodiments, the allosteric modulator binds to a non-orthosteric binding site of ADCY7. In some embodiments, the modulator of ADCY7 affects a conformation of the orthosteric binding site of ADCY7 effective decrease or increase activity of ADCY7. In some embodiments, the modulator of ADCY7 is effective to increase or decrease a rate of catalysis of cyclic adenosine monophosphate (cAMP) from adenosine triphosphate (ATP) by ADCY7. In some embodiments, the modulator of ADCY7 is effective to reduce or enhance the inhibition of ADCY7 activity by calcium. Non-limiting examples of ligands that activate ADCY7 include G protein alpha subunit, G protein beta and gamma subunit complex, G Protein Subunit Alpha 13 (GNA13), G Protein Subunit Alpha 12 (GNA12), and ethanol. A non-limiting example of a ligand that inhibits ADCY7 includes lithium.

G Protein-Coupled Receptor 65 (GPR65) Modulators

G Protein-Coupled Receptor 65 (GPR65) (Entrez Gene: 8477) encodes Psychosine receptor (UniProtKB: Q81YL9) which is a receptor for the glycosphingolipid psychosine (PSY) and several related glycosphingolipids. GPR65 plays a role in immune response by maintaining lysosome function and supporting phagocytosis-mediated intracellular bacteria clearance.

In some embodiments, modulator of GPR65 is an agonist of GPR56 activity or expression. In some cases, the GPR65 agonist increases GPR65 expression or activity. The agonist of GPR65 expression or activity may be a direct agonist or indirect agonist. In some embodiments, the agonist of GPR65 expression or activity comprises a complete agonist or a partial agonist. Non-limiting examples of an agonist of GPR65 expression include RNA to protein GPR65 translation agonists, antisense oligonucleotides targeting the GPR65, or homolog thereof, mRNA (such as miRNAs, or siRNA), epigenetic editing (such as post-translational modifications of histone tails and/or DNA molecules). Non-limiting examples of an agonist of GPR65 activity include antagonists to the GPR65 antigen, and antagonists to gene expression products involved in GPR65 mediated disease. Agonists as disclosed herein, may include, but are not limited to, a GPR65 antibody, a GPR65-binding antibody fragment, recombinant polypeptide, or a small molecule. The small molecule may be a small molecule that binds to GPR65 or binding partners to GPR65. The GPR65 antibody may be monoclonal or polyclonal. The GPR65 antibody may be humanized or chimeric. The GPR65 antibody may be a fusion protein. The GPR65 antibody may be a blocking GPR65 antibody. A blocking antibody blocks binding between two proteins, e.g., a ligand and its receptor. In a non-limiting example, the GPR65 blocking antibody binds to a binding partner of GPR65. In some cases, the GPR65 antibody is a GPR65 antibody that specifically binds to GPR65. In some cases, the GPR65 is naturally occurring. In some embodiments, the agonist of GPR65 comprises a polypeptide. In some instances, the polypeptide is recombinant. In some instances, the antagonist of GPR65 is an allosteric agonist. In some instances the agonist of GPR65 comprises BTB09089 (3-[(2,4-dichlorophenyl)methylsulfanyl]-1,6-dimethylpyridazino[4,5-e][1,3,4]thiadiazin-5-one).

In some embodiments, the GPR65 modulator is a antagonist of GPR65 activity or expression. The antagonist of GPR65 expression or activity may be a direct antagonist or indirect antagonist. In some embodiments, the antagonist of GPR65 expression or activity comprises a complete antagonist or a partial antagonist. Non-limiting examples of an antagonist of GPR65 expression include RNA to protein GPR65 translation antagonists, antisense oligonucleotides targeting the GPR65, or homolog thereof, mRNA (such as miRNAs, or siRNA), epigenetic editing (such as post-translational modifications of histone tails and/or DNA molecules). Non-limiting examples of an antagonist of GPR65 activity include antagonists to the GPR65 antigen, and antagonists to gene expression products involved in GPR65 mediated disease. Antagonists as disclosed herein, may include, but are not limited to, a GPR65 antibody, a GPR65-binding antibody fragment, recombinant polypeptide, or a small molecule. The small molecule may be a small molecule that binds to GPR65 or binding partners to GPR65. The GPR65 antibody may be monoclonal or polyclonal. The GPR65 antibody may be humanized or chimeric. The GPR65 antibody may be a fusion protein. The GPR65 antibody may be a blocking GPR65 antibody. A blocking antibody blocks binding between two proteins, e.g., a ligand and its receptor. In a non-limiting example, the GPR65 blocking antibody binds to a binding partner of GPR65. In some cases, the GPR65 antibody is a GPR65 antibody that specifically binds to GPR65. In some cases the GPR65 is naturally occurring. In some embodiments, the GPR65 antagonists comprise one or more small molecule compounds. In some embodiments, the small molecule comprises antagonist that are inverse agonists. In some instances, the antagonist of GPR65 comprises a negative allosteric modulator (NAM). In some instances, the antagonist of GPR65 comprises ZINC62678696 ([(S)-phenyl(pyridin-4-yl)methyl]4-methyl-2-pyrimidin-2-yl-1,3-thiazole-5-carboxylate).

In some instances, the GPR56 modulator is an allosteric modulator acts as an antagonist, an agonist of GPR65. In some embodiments, the allosteric modulator of GPR65 is a positive allosteric modulator (PAM) effective to enhance or potentiate a ligand of GPR65. In some instances, the GPR65 PAM comprises BTB09089. In some embodiments, the allosteric modulator of GPR65 is a negative allosteric modulator (NAM) effective to reduce the effect of a primary ligand of GPR65. In some instances, the GPR65 NAM comprises ZINC62678696. In some embodiments, the allosteric modulator binds to a non-orthosteric binding site of GPR65. In some embodiments, the modulator of GPR65 affects a conformation of the orthosteric binding site of GPR65 effective decrease or increase activity of GPR65. In some embodiments, the modulator of GPR65 is effective to increase or decrease a pH sensing activity of GPR65.

In some embodiments, the antagonist or agonist of GPR65 comprises a GPR65 polypeptide. In some embodiments, the GPR65 polypeptide comprises a human GPR65 protein (huGPR65), or a homolog thereof. In some embodiments, the GPR65 polypeptide comprises a recombinant GPR65 polypeptide. In some embodiments, the recombinant human Psychosine receptor protein (encoded by the gene GPR65) comprises SEQ ID NO: 1)(MNSTCIEEQHDLDHYLFPIVYIFVIIVSIPANIGSLCVSFLQAKKESELGIYLFSLSLSDLLYALTLPL WIDYTWNKDNWTFSPALCKGSAFLMYMNFYSSTAFLTCIAVDRYLAVVYPLKFFFLRTRRFALM VSLSIWILETIFNAVMLWEDETVVEYCDAEKSNFTLCYDKYPLEKWQINLNLFRTCTGYAIPLVTI LICNRKVYQAVRHNKATENKEKKRIIKLLVSITVTFVLCFTPFHVMLLIRCILEHAVNFEDHSNSG KRTYTMYRITVALTSLNCVADPILYCFVTETGRYDMWNILKFCTGRCNTSQRQRKRILSVSTKDT MELEVLE), which is the amino acid sequence of human GPR65 (NCBI Reference Sequence No. NP_095256-1). In some embodiments, the huGPR65 comprises an amino acid sequence about 99%, 98%, 97%, 96%, 95%, 94%, 93%, 92%, 91%, or 90% homologous to SEQ ID NO: 1.

In some instances, the GPR65 polypeptide is truncated. In some instances, the truncation is an N-terminal deletion. In other instances, the truncation is a C-terminal deletion. In additional instances, the truncation comprises both N-terminal and C-terminal deletions. For example, the truncation can be a deletion of at least or about 1, 2, 3, 4, 5, 6, 7, 8, 9, 10, 11, 12, 13, 14, 15, 20, or more residues from either the N-terminus or the C-terminus, or both termini. In some cases, the GPR65 polypeptide comprises an N-terminal deletion of at least or about 1, 2, 3, 4, 5, 6, 7, 8, 9, 10, 11, 12, 13, 14, 15, 20, or more residues. In some cases, the GPR65 polypeptide comprises an N-terminal deletion of at least or about 1, 2, 3, 4, 5, 6, 7, 8, 9, or 10 residues. In some cases, the GPR65 polypeptide comprises an N-terminal deletion of at least or about 2 residues. In some cases, the GPR65 polypeptide comprises an N-terminal deletion of at least or about 3 residues. In some cases, the GPR65 polypeptide comprises an N-terminal deletion of at least or about 4 residues. In some cases, the GPR65 polypeptide comprises an N-terminal deletion of at least or about 5 residues. In some cases, the GPR65 polypeptide comprises an N-terminal deletion of at least or about 6 residues. In some cases, the GPR65 polypeptide comprises an N-terminal deletion of at least or about 7 residues. In some cases, the GPR65 polypeptide comprises an N-terminal deletion of at least or about 8 residues. In some cases, the GPR65 polypeptide comprises an N-terminal deletion of at least or about 9 residues. In some cases, the GPR65 polypeptide comprises an N-terminal deletion of at least or about 10 residues.

In some embodiments, the GPR65 polypeptide has an enhanced plasma half-life. In some instances, the plasma half-life comprises at least 30 minutes, 45 minutes, 60 minutes, 75 minutes, or 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer than the plasma half-life of the wild-type GPR65 protein.

In some embodiments, the GPR65 polypeptide is a conjugate. In some embodiments, the GPR65 conjugate comprises a GPR65 polypeptide comprising at least one amino acid and a conjugating moiety bound to the at least one 1 amino acid. In some embodiments, the at least one amino acid is located proximal to the N-terminus (e.g., proximal to the N-terminal residue). For example, the at least one amino acid is located optionally within the first 10, 20, 30, 40, or 50 residues from the N-terminus. In some cases, the at least one amino acid is located at the N-terminus (i.e., the at least one amino acid is the N-terminal residue of the GPR65 polypeptide). In other embodiments, the at least one amino acid is located proximal to the C-terminus (e.g., proximal to the C-terminal residue). For example, the at least one amino acid is located optionally within the first 10, 20, 30, 40, or 50 residues from the C-terminus. In some cases, the at least one amino acid is located at the C-terminus (i.e., the at least one amino acid is the C-terminal residue of the GPR65 polypeptide). In some instances, the GPR65 conjugate has an enhanced plasma half-life, such as the half-lives described herein. In some embodiments, the GPR65 conjugate is functionally active (e.g., retains activity). In some embodiments, the GPR65 conjugate is not functionally active (e.g., devoid of activity). In some embodiments, the conjugating moiety comprises a polymer comprising Polyethylene glycol (PEG).

In some embodiments, the GPR65 polypeptide is fused with a second polypeptide. In some embodiments, the second polypeptide comprises a polypeptide with a long plasma half-life relative to the plasma half-life of the GPR65 polypeptide. In some embodiments, the second polypeptide comprises an antibody or antibody fragment. In some embodiments, the antibody or antibody fragment comprises an IgG1, IgG2, IgG4, IgG3, or IgE. In some embodiments, the IgG is an Fc. In some embodiments, the IgG Fc is human. In some instances, the long plasma half-life polypeptide comprises HSA, transferrin, IgA monomer, Retinol-binding protein, Factor H, Factor XIII, C-reactive protein, Factor IX, Fibrinogen, IFN-alpha, Pentameric IgM, IL-2, or Thyroglobulin.

Intercellular Adhesion Molecule 3 (ICAM3)

Intercellular adhesion molecule 3 (ICAM3) (Entrez Gene: 3385) encodes an intercellular adhesion molecule family protein, which is a type I transmembrane glycoprotein that binds to the leukocyte adhesion LFA-1 protein and initiates the immune response.

Disclosed herein, in some embodiments, are therapeutic agents comprising modulators of ICAM3 useful for the treatment of a disease or condition, or symptom of the disease or condition, disclosed herein. In some embodiments, the therapeutic agents comprise a modulator of ICAM3. In some cases, the modulator of ICAM3 is an antagonist, partial antagonist, agonist, or partial agonist. In some cases, the ICAM3 is an allosteric modular of ICAM3.

In some embodiments, modulator of ICAM3 is an agonist of ICAM3 activity or expression. In some cases, the ICAM3 agonist increases ICAM3 expression or activity. The agonist of ICAM3 expression or activity may be a direct agonist or indirect agonist. In some embodiments, the agonist of ICAM3 expression or activity comprises a complete agonist or a partial agonist. Non-limiting examples of an agonist of ICAM3 expression include RNA to protein ICAM3 translation agonists, antisense oligonucleotides targeting the ICAM3, or homolog thereof, mRNA (such as miRNAs, or siRNA), epigenetic editing (such as post-translational modifications of histone tails and/or DNA molecules). Non-limiting examples of an agonist of ICAM3 activity include antagonists to the ICAM3 antigen, and antagonists to gene expression products involved in ICAM3 mediated disease. Agonists as disclosed herein, may include, but are not limited to, a ICAM3 antibody, a ICAM3-binding antibody fragment, recombinant polypeptide, or a small molecule. The small molecule may be a small molecule that binds to ICAM3 or binding partners to ICAM3. The ICAM3 antibody may be monoclonal or polyclonal. The ICAM3 antibody may be humanized or chimeric. The ICAM3 antibody may be a fusion protein. The ICAM3 antibody may be a blocking ICAM3 antibody. A blocking antibody blocks binding between two proteins, e.g., a ligand and its receptor. In a non-limiting example, the ICAM3 blocking antibody binds to a binding partner of ICAM3. In some cases, the ICAM3 antibody is an ICAM3 antibody that specifically binds to ICAM3. In some cases, the ICAM3 is naturally occurring. In some embodiments, the agonist of ICAM3 comprises a polypeptide. In some instances, the polypeptide is recombinant.

In some embodiments, the ICAM3 modulator is an antagonist of ICAM3 activity or expression. The antagonist of ICAM3 expression or activity may be a direct antagonist or indirect antagonist. In some embodiments, the antagonist of ICAM3 expression or activity comprises a complete antagonist or a partial antagonist. Non-limiting examples of an antagonist of ICAM3 expression include RNA to protein ICAM3 translation antagonists, antisense oligonucleotides targeting the ICAM3, or homolog thereof, mRNA (such as miRNAs, or siRNA), epigenetic editing (such as post-translational modifications of histone tails and/or DNA molecules). Non-limiting examples of an antagonist of ICAM3 activity include antagonists to the ICAM3 antigen, and antagonists to gene expression products involved in ICAM3 mediated disease. Antagonists as disclosed herein, may include, but are not limited to, an ICAM3 antibody, an ICAM3-binding antibody fragment, recombinant polypeptide, or a small molecule. The small molecule may be a small molecule that binds to ICAM3 or binding partners to ICAM3. The ICAM3 antibody may be monoclonal or polyclonal. The ICAM3 antibody may be humanized or chimeric. The ICAM3 antibody may be a fusion protein. The ICAM3 antibody may be a blocking ICAM3 antibody. A blocking antibody blocks binding between two proteins, e.g., a ligand and its receptor. In a non-limiting example, the ICAM3 blocking antibody binds to a binding partner of ICAM3. In some cases, the ICAM3 antibody is a ICAM3 antibody that specifically binds to ICAM3. In some cases, the ICAM3 is naturally occurring. In some embodiments, the ICAM3 antagonists comprise one or more small molecule compounds. In some embodiments, the small molecule comprises antagonist that are inverse agonists. In some instances, the antagonist of ICAM3 comprises a negative allosteric modulator (NAM).

In some instances, the GPR56 modulator is an allosteric modulator acts as an antagonist, an agonist of ICAM3. In some embodiments, the allosteric modulator of ICAM3 is a positive allosteric modulator (PAM) effective to enhance or potentiate a ligand of ICAM3. In some embodiments, the allosteric modulator of ICAM3 is a negative allosteric modulator (NAM) effective to reduce the effect of a primary ligand of ICAM3. In some embodiments, the allosteric modulator binds to a non-orthosteric binding site of ICAM3. In some embodiments, the modulator of ICAM3 affects a conformation of the orthosteric binding site of ICAM3 effective decrease or increase activity of ICAM3. In some embodiments, the modulator of ICAM3 is effective to increase or decrease a pH sensing activity of ICAM3.

In some embodiments, the antagonist or agonist of ICAM3 comprises a ICAM3 polypeptide. In some embodiments, the ICAM3 polypeptide comprises a human ICAM3 protein (huICAM3), or a homolog thereof. In some embodiments, the ICAM3 polypeptide comprises a recombinant ICAM3 polypeptide. In some embodiments, the recombinant Intercellular adhesion molecule 3 (encoded by the gene ICAM3) comprises SEQ ID NO: 2 (MGWAAFNLSNVTGNSRILCSVYCNGSQITGSSNITVYRLPERVELAPLPPWQPVGQNFTLRCQV EDGSPRTSLTVVLLRWEEELSRQPAVEEPAEVTATVLASRDDHGAPFSCRTELDMQPQGLGLFVN TSAPRQLRTFVLPVTPPRLVAPRFLEVETSWPVDCTLDGLFPASEAQVYLALGDQMLNATVMNH GDTLTATATATARADQEGAREIVCNVTLGGERREARENLTVFSFLGPIVNLSEPTAHEGSTVTVS CMAGARVQVTLDGVPAAAPGQPAQLQLNATESDDGRSFFCSATLEVDGEFLHRNSSVQLRVLY GPKIDRATCPQHLKWKDKTRHVLQCQARGNPYPELRCLKEGSSREVPVGIPFFVNVTHNGTYQC QASSSRGKYTLVVVMDIEAGSSHFVPVFVAVLLTLGVVTIVLALMYVFREHQRSGSYHVREEST YLPLTSMQPTEAMGEEPSRAE), which is the amino acid sequence of human ICAM3 (NCBI Reference Sequence No. NP_001307535.1). In some embodiments, the huICAM3 comprises an amino acid sequence about 99%, 98%, 97%, 96%, 95%, 94%, 93%, 92%, 91%, or 90% homologous to SEQ ID NO: 2.

In some instances, the ICAM3 polypeptide is truncated. In some instances, the truncation is an N-terminal deletion. In other instances, the truncation is a C-terminal deletion. In additional instances, the truncation comprises both N-terminal and C-terminal deletions. For example, the truncation can be a deletion of at least or about 1, 2, 3, 4, 5, 6, 7, 8, 9, 10, 11, 12, 13, 14, 15, 20, or more residues from either the N-terminus or the C-terminus, or both termini. In some cases, the ICAM3 polypeptide comprises an N-terminal deletion of at least or about 1, 2, 3, 4, 5, 6, 7, 8, 9, 10, 11, 12, 13, 14, 15, 20, or more residues. In some cases, the ICAM3 polypeptide comprises an N-terminal deletion of at least or about 1, 2, 3, 4, 5, 6, 7, 8, 9, or 10 residues. In some cases, the ICAM3 polypeptide comprises an N-terminal deletion of at least or about 2 residues. In some cases, the ICAM3 polypeptide comprises an N-terminal deletion of at least or about 3 residues. In some cases, the ICAM3 polypeptide comprises an N-terminal deletion of at least or about 4 residues. In some cases, the ICAM3 polypeptide comprises an N-terminal deletion of at least or about 5 residues. In some cases, the ICAM3 polypeptide comprises an N-terminal deletion of at least or about 6 residues. In some cases, the ICAM3 polypeptide comprises an N-terminal deletion of at least or about 7 residues. In some cases, the ICAM3 polypeptide comprises an N-terminal deletion of at least or about 8 residues. In some cases, the ICAM3 polypeptide comprises an N-terminal deletion of at least or about 9 residues. In some cases, the ICAM3 polypeptide comprises an N-terminal deletion of at least or about 10 residues.

In some embodiments, the ICAM3 polypeptide has an enhanced plasma half-life. In some instances, the plasma half-life comprises at least 30 minutes, 45 minutes, 60 minutes, 75 minutes, or 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer than the plasma half-life of the wild-type ICAM3 protein.

In some embodiments, the ICAM3 polypeptide is a conjugate. In some embodiments, the ICAM3 conjugate comprises an ICAM3 polypeptide comprising at least one amino acid and a conjugating moiety bound to the at least one 1 amino acid. In some embodiments, the at least one amino acid is located proximal to the N-terminus (e.g., proximal to the N-terminal residue). For example, the at least one amino acid is located optionally within the first 10, 20, 30, 40, or 50 residues from the N-terminus. In some cases, the at least one amino acid is located at the N-terminus (i.e., the at least one amino acid is the N-terminal residue of the ICAM3 polypeptide). In other embodiments, the at least one amino acid is located proximal to the C-terminus (e.g., proximal to the C-terminal residue). For example, the at least one amino acid is located optionally within the first 10, 20, 30, 40, or 50 residues from the C-terminus. In some cases, the at least one amino acid is located at the C-terminus (i.e., the at least one amino acid is the C-terminal residue of the ICAM3 polypeptide). In some instances, the ICAM3 conjugate has an enhanced plasma half-life, such as the half-lives described herein. In some embodiments, the ICAM3 conjugate is functionally active (e.g., retains activity). In some embodiments, the ICAM3 conjugate is not functionally active (e.g., devoid of activity). In some embodiments, the conjugating moiety comprises a polymer comprising Polyethylene glycol (PEG). In some embodiments, the ICAM3 polypeptide is fused with a second polypeptide. In some embodiments, the second polypeptide comprises a polypeptide with a long plasma half-life relative to the plasma half-life of the ICAM3 polypeptide. In some embodiments, the second polypeptide comprises an antibody or antibody fragment. In some embodiments, the antibody or antibody fragment comprises an IgG1, IgG2, IgG4, IgG3, or IgE. In some embodiments, the IgG is an Fc. In some embodiments, the IgG Fc is human. In some instances, the long plasma half-life polypeptide comprises HSA, transferrin, IgA monomer, Retinol-binding protein, Factor H, Factor XIII, C-reactive protein, Factor IX, Fibrinogen, IFN-alpha, Pentameric IgM, IL-2, or Thyroglobulin.

Mitogen-Activated Protein Kinase Kinase Kinase Kinase 4 (MAP4K4) Modulators

Mitogen-Activated Protein Kinase Kinase Kinase Kinase 4 (MAP4K4) refers to a peptide associated with one or more of the following reference sequences: HGNC: 6866, Entrez Gene: 9448, Ensembl: ENSG00000071054, OMIM: 604666, and UniProtKB: 095819.

Disclosed herein, in some embodiments, are therapeutic agents comprising modulators of MAP4K4 useful for the treatment of a disease or condition, or symptom of a disease or condition, disclosed herein. In some embodiments, the MAP4K4 modulator is a small molecule. In some embodiments, a small molecule has a molecular weight less than or equal to about 1000 Daltons. In some embodiments, a small molecule has a molecular weight less than or equal to about 900 Da. In some embodiments, a small molecule has a molecular weight less than or equal to about 800 Da. In some embodiments, a small molecule has a molecular weight less than or equal to about 700 Da. In some embodiments, a small molecule has a molecular weight less than or equal to about 600 Da. In some embodiments, a small molecule has a molecular weight less than or equal to about 500 Da. In some embodiments, the MAP4K4 modulator is an antagonist or inhibitor of MAP4K4.

Disclosed herein, in some embodiments, are therapeutic agents useful for the treatment of a disease or condition, or symptom of the disease or condition, disclosed herein. In some embodiments, the therapeutic agent comprises a modulator, agonist, and/or antagonist of MAP4K4.

In some embodiments, the MAP4K4 modulator is a compound of Formula (I):

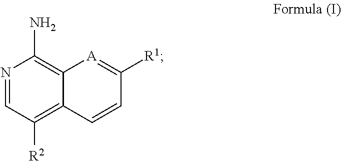

or a stereoisomers geometric isomer, tautomer, or a pharmaceutically acceptable salt thereof, wherein:
A is CH or N;
R₁ and R₂ are independently selected from:
   CN;
   C₁-C₁₂-alkyl which is unsubstituted or substituted by one or more substituents selected from the group consisting of cyano, halo, hydroxy, C₁-C₁₂-alkoxy, C₃-C₁₂-cycloalkyl, —NH₂, —NH—C₁-C₁₂-alkyl, —NH—C₂-C₁₂-heteroaryl, —NHC(O)—C₁-C₁₂-alkyl, —NHC(O)—C₃-C₁₂-cycloalkyl, —NHC(O)—C₆-C₂O-aryl, —NHC(O)—C₂-C₁₂-heteroaryl, —NHC(O)NH—C₁-C₁₂-alkyl, —NHC(O)NH—C₂-C₁₂-heteroaryl, —NHS(O)₂—C₁-C₁₂-alkyl, and —HS(O)₂—C₃-C₁₂-cycloalkyl;
   C₁-C₁₂-alkoxy which is unsubstituted or substituted by halo;
   C₃-C₁₂-cycloalkyl which is unsubstituted or substituted by one or more substituents selected from the group consisting of cyano, halo, hydroxy, —NH₂, C₁-C₁₂-alkyl, C₁-C₁₂-alkoxy, C₃-C₁₂-cycloalkyl, C₂-C₁₂-heterocyclyl, C₆-C₂₀-aryl, and C₂-C₁₂-heteroaryl, wherein said cycloalkyl, heterocyclyl, aryl, and heteroaryl can be unsubstituted or substituted by one or more substituents selected from the group consisting of: halo, OH, CN, NH₂, —NH(C₁-C₁₂-alkyl), —N(C₁-C₁₂-alkyl)₂, C₁-C₁₂-alkyl, C₁-C₁₂-alkoxy, C₁-C₁₂-haloalkyl, C₁-C₁₂-hydroxyalkyl, —C(O)C₁-C₁₂-alkyl, and —C(O)NHC₁-C₁₂-alkyl;
   —NHR^{a}, wherein W is selected from the group consisting of:
      C₃-C₁₂-cycloalkyl, which is unsubstituted or substituted by one or more C₁-C₁₂-alkyl;
      —C₂-C₁₂-heterocyclyl, which is unsubstituted or substituted by one or more substituents,
      C₁-C₁₂-alkylenyl-C₆-C₂O-aryl, wherein the aryl is unsubstituted or substituted by one or more substituents selected from the group consisting of: halo, C₁-C₁₂-alkyl and C₂-C₁₂-heterocyclyl;
      —C₁-C₁₂-alkylenyl-C₁-C₁₂-alkoxy-C₆-C₂₀-aryl, wherein the aryl is unsubstituted or substituted by halo; and —C₁-C₁₂-alkylenyl-C₂-C₁₂-heteroaryl, wherein the heteroaryl is unsubstituted or substituted by C₁-C₁₂-alkyl;
   —C(O)—C₂-C₁₂-heterocyclyl, which is unsubstituted or substituted by one or more substituents selected from the group consisting of: C₁-C₁₂-alkyl, —C₁-C₁₂-hydroxyalkyl, —C(O)—NH₂, —C(O)—N(H)(C₁-C₁₂-alkyl), —C(O)—N(C₁-C₁₂-alkyl)₂, —C₁-C₁₂-alkylenyl-C(O)—N(C₁-C₁₂-alkyl)₂, —NH—C(O)—C₁-C₁₂-alkyl, —NH—C(O)—C₃-C₁₂-cycloalkyl, and —N(C(O)—C₃-C₁₂-cycloalkyl)₂;
   —C(O)OH;
   —C(O)—C₁-C₁₂-alkoxy;
   —C(O)NR^{b}R^{c}, wherein R^{b} and R^{c} are independently selected from the group consisting of:
      H; —C₁-C₁₂-alkyl, which is unsubstituted or substituted by one or more substituent selected from the group consisting of:
         —OH, —CN, —NH₂, —C₃-C₁₂-cycloalkyl, —C(O)—NH₂, —C(O)—C₂-C₁₂-heterocyclyl, —N(H)(C(O)—C₁-C₁₂-alkyl), —N(H)(C₁-C₁₂-alkyl), —N(C₁-C₁₂-alkyl)₂, —NHC(O)—NH(C₃-C₁₂-cycloalkyl), —NHC(O)—NH(C₁-C₁₂-alkyl);
         C₂-C₁₂-heterocyclyl, wherein the heterocyclyl is unsubstituted or substituted by one or more substituents selected from the group consisting of oxo, hydroxy and C₁-C₁₂-alkyl;
         —C(O)—C₂-C₁₂-heterocyclyl, wherein the heterocyclyl is unsubstituted or substituted by one or more substituents selected from the group consisting of oxo, hydroxy and C₁-C₁₂-alkyl;
         —SO₂—C₂-C₁₂-heterocyclyl;
      aryl, which is unsubstituted or substituted by one or more substituents selected from the group consisting of C₁-C₁₂-alkyl, C₁-C₁₂-alkoxy, —C₁-C₁₂-hydroxyalkyl, C₁-C₁₂-alkylenyl-C₁-C₁₂-alkoxy, C₁-C₁₂-alkylenyl-NH₂, and —O—C₂-C₁₂-heterocyclyl, which heterocyclyl is unsubstituted or substituted by C₁-C₁₂-alkyl; and
      C₂-C₁₂-heteroaryl, which is unsubstituted or substituted by one or more substituents selected from the group consisting of C₁-C₁₂-alkyl, C₁-C₁₂-alkoxy, —C(O)—NH₂, —C(O)—N(H)(C₁-C₁₂-alkyl), —C(O)—N(C₁-C₁₂-alkyl)₂, and —C₁-C₁₂-alkylenyl-C(O)—N(C₁-C₁₂-alkyl)₂;
   —C₃-C₁₂-cycloalkyl, which is unsubstituted or substituted by one ore more hydroxy;
   —C₂-C₁₂-heterocyclyl, which is unsubstituted or substituted by one or more substituents selected from the group consisting of:
      C₁-C₁₂-alkyl, —SO₂—C₁-C₁₂-alkyl, —C(O)—C₁-C₁₂-alkyl, —C(O)—NH₂, —C(O)—N(H)(C₁-C₁₂-alkyl), —C(O)—N(C₁-C₁₂-alkyl)₂, —C(O)—C₁-C₁₂-alkylenyl-C₁-C₁₂-alkoxy, —C₁-C₁₂-alkylenyl-C(O)—N(C₁-C₁₂-alkyl)₂, —C(O)—C₃-C₁₂-cycloalkyl, and —C(O)NH—C₃-C₁₂-cycloalkyl;
      —C₆-C₂O-aryl, which is unsubstituted or substituted by one or more substituents selected from the group consisting of C₁-C₁₂-alkyl, C₁-C₁₂-alkoxy, C1-C₁₂-hydroxyalkyl, C₁-C₁₂-alkylenyl-C₁-C₁₂-alkoxy, C₁-C₁₂-alkylenyl-NH₂, and —O—C₂-C₁₂-heterocyclyl, which heterocyclyl is unsubstituted or substituted by C₁-C₁₂-alkyl; and
      C₆-C₂O-heteroaryl, which is unsubstituted or substituted by one or more substituents selected from the group consisting of C₁-C₁₂-alkyl, C₁-C₁₂-hydroxyalkyl, and —O—C₂-C₁₂-heterocyclyl which heterocyclyl is unsubstituted or substituted by C₁-C₁₂-alkyl;
   —O—C₃-C₁₂-cycloalkyl, —O—C₂-C₁₂-heterocyclyl, —O—C₆-C₂₀-aryl and —O—C₆-C₂O-heteroaryl, wherein the cycloalkyl, heterocyclyl, aryl and heteroaryl is unsubstituted or substituted by one or more substituent(s) selected from the group consisting of: halo, cyano, C₁-C₁₂-alkyl, C₁-C₁₂-alkoxy and —C(O)NH₂;
   C₂-C₁₂-heterocyclyl, which is unsubstituted or substituted by one or more substituents selected from the group consisting of:
      halo, oxo, —NH₂;
      C₁-C₁₂-alkyl, which is unsubstituted or substituted by one or more substituents selected from the group consisting of: OH, halo, C₁-C₁₂-alkoxy, —C(O)—NH—C₁-C₁₂-alkyl, and C₂-C₁₂-heteroaryl, which heteroaryl is unsubstituted or substituted by one or more substituents selected from the group consisting of: C₁-C₁₂-alkyl, C₁-C₁₂-alkylenyl-C₁-C₁₂-alkoxy and —N(C₁-C₁₂-alkyl)₂;
      C₁-C₁₂-alkoxy;
      —C(O)—R^{d}, wherein R^{d} is selected from the group consisting of C₁-C₁₂-alkyl, —C₂-C₁₂-heterocyclyl, —NH₂, —NH—C₃-C₁₂-cycloalkyl, and —O—C₁-C₁₂-alkyl;
      —N(C₁-C₁₂-alkyl)₂;
      —N(C₁-C₁₂-alkyl)C(O)—C₁-C₁₂-alkyl;
      —NH(CO)—C₁-C₁₂-alkylenyl-N(C₁-C₁₂-alkyl)₂;
      —NH(CO)—C₁-C₁₂-alkylenyl-C₁-C₁₂-alkoxy;
      —NH(CO)O—C₁-C₁₂-alkyl;
      —NH(CO)—C₁-C₁₂-alkylenyl-N(C₁-C₁₂-alkyl)-C(O)—C₁-C₁₂-alkyl;
      —NH(CO)—C₂-C₁₂-heterocyclyl, wherein the heterocyclyl is unsubstituted or substituted by one or more substituents selected from the group consisting of: oxo, C₁-C₁₂-alkyl, —C(O)—C₁-C₁₂-alkyl and —S(O)₂—C₁-C₁₂-alkyl;
      —NH(CO)—C₁-C₁₂-alkylenyl-C₂-C₁₂-heterocyclyl, wherein the heterocyclyl is unsubstituted or substituted by one or more substituents selected from the group consisting of oxo, C₁-C₁₂-alkyl and —C(O)—C₁-C₁₂-alkyl;
      —NH(CO)—C₁-C₁₂-alkylenyl-C₂-C₁₂-heteroaryl, wherein the heteroaryl is unsubstituted or substituted by C₁-C₁₂-alkyl; and
      C₃-C₁₂-cycloalkyl, C₂-C₁₂-heterocyclyl, C₆-C₂₀-aryl or C₂-C₁₂-heteroaryl wherein the cycloalkyl, heterocycloalkyl, aryl or heteroaryl is unsubstituted or substituted by one or more substituents selected from the group consisting of: OH, C₁-C₁₂-alkyl, C₁-C₁₂-haloalkyl, C₁-C₁₂-hydroxyalkyl, C₁-C₁₂-alkylenyl —C₁-C₁₂-alkoxy, —NH—C₁-C₁₂-alkyl, —N(C₁-C₁₂-alkyl)₂, —C(O)NH₂, —C(O)NH—C₁-C₁₂-alkyl, —C(O)N(C₁-C₁₂-alkyl)₂ and —S(O)₂—C₁-C₁₂-alkyl;
   C₆-C₂O-aryl which is unsubstituted or substituted by one or more substituents selected from the group consisting of: halo; CN; OH; —NH₂; C₁-C₁₂-alkyl, which is unsubstituted or substituted by one or more substituents selected from the group consisting of:
      —NH(C₁-C₁₂-alkyl), wherein the alkyl is unsubstituted or substituted by OH or —N(C₁-C₁₂-alkyl)₂, —C(O)N(C₁-C₁₂-alkyl)₂, (C₃-C₁₂-cycloalkyl, C₂-C₁₂-heterocyclyl, which cycloalkyl or heterocyclyl is unsubstituted or substituted by C₁-C₁₂-alkyl;
      —NH(C₂-C₁₂-heterocyclyl) which heterocyclyl is unsubstituted or substituted by one or more substituents selected from the group consisting of: oxo and C₁-C₁₂-alkyl;
      —N(C₁-C₁₂-alkyl)(C₂-C₁₂-heterocyclyl) which heterocyclyl is unsubstituted or substituted by one or more substituents selected from the group consisting of: oxo and C₁-C₁₂-alkyl;
      —N(C₁-C₁₂-alkyl)(C₁-C₁₂-alkylenyl-C₁-C₁₂-alkoxy);
      —N(C₁-C₁₂-alkyl)-C(O)—C₂-C₁₂-heterocyclyl;
      —NH(C₃-C₁₂-cycloalkyl), wherein the cycloalkyl is unsubstituted or substituted by halo, C₁-C₁₂-hydroxyalkyl;
      C₂-C₁₂-heterocyclyl which is unsubstituted or substituted by one or more substituents selected from the group consisting of: halo, oxo, OH, C₁-C₁₂-alkyl, C₁-C₁₂-hydroxyalkyl, alkoxy, C₁-C₁₂-alkylenlyl-C(O)—C₁-C₁₂-alkyl, C₁-C₁₂-alkylenyl-C(O)N(C₁-C₁₂-alkyl)₂, —N(C₁-C₁₂-alkyl)₂, —N(C₁-C₁₂-alkyl)-C(O)—C₁-C₁₂-alkyl, —C(O)NH₂, —C(O)NH(C₁-C₁₂-alkyl), —C(O)NH(C₁-C₁₂-haloalkyl), —C(O)NH(C₁-C₁₂-hydroxyalkyl), —S(O)₂—C₁-C₁₂-alkyl, —S(O)₂—N(C₁-C₁₂-alkyl)₂, —C(O)NH(C₂-C₁₂-heterocyclyl), —C(O)N(C₁-C₁₂-alkyl)₂, and —C(O)—C₂-C₁₂-heterocyclyl, which heterocyclyl is unsubstituted or substituted by C₁-C₁₂-alkyl, —C(O)OH, —S(O)₂—C₁-C₁₂-alkyl, —S(O)₂—N(C₁-C₁₂-alkyl)₂, or C₂-C₁₂-heterocyclyl; and —C₁-C₁₂-alkoxy which is unsubstituted or substituted by one or more substituents selected from the group consisting of: halo and C₂-C₁₂-heterocyclyl;
   C₁-C₁₂-alkoxy which is unsubstituted or substituted by C₂-C₁₂-heterocyclyl;
   C₁-C₁₂-haloalkoxy;
   —NH—C₁-C₁₂-alkylenyl-N(C₁-C₁₂-alkyl)₂;
   —NH—C₁-C₁₂-alkylenyl-C₂-C₁₂-heterocyclyl;
   —NH—C₁-C₁₂-alkylenyl-C₆-C₂₀-aryl, wherein the C₆-C₂O-aryl is unsubstituted or substituted by halo;
   —NH—C₂-C₁₂-heterocyclyl-C₁-C₁₂-alkylenyl-C₂-C₁₂-heterocyclyl;
   —NHC(O)—C₁-C₁₂-alkyl, wherein the C₁-C₁₂-alkyl group is unsubstituted or substituted by one or more substituents selected from the group consisting of: halo, C₁-C₁₂-alkoxy, —N(C₁-C₁₂-alkyl)₂, —C₃-C₁₂-cycloalkyl, and —C₂-C₁₂-heterocyclyl, wherein the heterocyclyl is unsubstituted or substituted by oxo;
   —NHC(O)—C₁-C₁₂-alkoxy;
   —NHC(O)—C₃-C₁₂-cycloalkyl, wherein the cycloalkyl is unsubstituted or substituted by halo;
   —NHC(O)—C₂-C₁₂-heterocyclyl, wherein the heterocyclyl is unsubstituted or substituted by one or more substituents selected from the group consisting of: C₁-C₁₂-alkyl and —C(O)—C₁-C₁₂-alkyl;
   —NHC(O)NH—C₃-C₁₂-cycloalkyl, wherein the cycloalkyl is unsubstituted or substituted by OH;
   —NH(SO₂)—C₁-C₁₂-alkylenyl-C₂-C₁₂-heterocyclyl, wherein the heterocyclyl is unsubstituted or substituted by C₁-C₁₂-alkyl;
   —NH(SO₂)—C₂-C₁₂-heterocyclyl;
   —C(O)NH₂;
   —C(O)NH—C₁-C₁₂-alkyl;
   —C(O)NH—C₁-C₁₂-alkylenyl-N(C₁-C₁₂-alkyl)₂;
   —C(O)NH—C₁-C₁₂-alkylenyl-C₂-C₁₂-heterocyclyl, wherein the heterocyclyl is unsubstituted or substituted by one or more substituents selected from the group consisting of C₁-C₁₂-alkyl, —C(O)NH—C₁-C₁₂-hydroxyalkyl, and —C(O)NH—C₃-C₁₂-cycloalkyl; and
   C₃-C₁₂-cycloalkyl, C₂-C₁₂-heterocyclyl, C₆-C₂O-aryl; C₂-C₁₂-heteroaryl; and —O—C₂-C₁₂-heteroaryl wherein said cycloalkyl, heterocyclyl, aryl, and heteroaryl can be unsubstituted or substituted by one or more substituents selected from the group consisting of: halo, oxo, OH, CN, NH₂, —NH(C₁-C₁₂-alkyl), —N(C₁-C₁₂-alkyl)₂, C₁-C₁₂-alkoxy, C₁-C₁₂-haloalkyl, C₁-C₁₂-hydroxyalkyl, —C(O)C₁-C₁₂-alkyl, and —C(O)NHC₁-C₁₂-alkyl; and
—C₂-C₁₂-heteroaryl which is unsubstituted or substituted by one or more substituents selected from the group consisting of: halo, oxo, CN, —NH₂—NH—C₁-C₁₂-alkylenyl-N(C₁-C₁₂-alkylenyl)₂, —NH—C₁-C₁₂-alkylenyl-C₂-C₁₂-heterocyclyl;
   C₁-C₁₂-alkyl, which is unsubstituted or substituted by one or more substituents selected from the group consisting of halo, —C(O)—N(C₁-C₁₂-alkyl)₂, and —C₂-C₁₂-heterocyclyl, wherein the C₂-C₁₂-heterocyclyl is unsubstituted or substituted by C₁-C₁₂-alkyl;
   —C(O)—NH₂,
   —C(O)—N(H)(C₁-C₁₂-alkyl),
   —C(O)—N(C₁-C₁₂-alkyl)₂, and
   C₃-C₁₂-cycloalkyl, C₂-C₁₂-heterocyclyl, C₆-C₂O-aryl, C₂-C₁₂-heteroaryl wherein said cycloalkyl, heterocyclyl, aryl, and heteroaryl can be unsubstituted or substituted by one or more substituents selected from the group consisting of: halo, OH, CN, NH₂, —NH(C₁-C₁₂-alkyl), —N(C₁-C₁₂-alkyl)₂, C₁-C₁₂-alkyl, C₁-C₁₂-alkoxy, C₁-C₁₂-haloalkyl, C₁-C₁₂-hydroxyalkyl, —C(O)C₁-C₁₂-alkyl, and —C(O)NHC₁-C₁₂-alkyl;
      with the proviso that when A is CH, R¹ is selected from phenyl that is unsubstituted or substituted by halo.

In some embodiments of the compound of Formula (I), A is CH or N;
R¹ is C₆-C₂O-aryl, which is unsubstituted or substituted by one or more halo, CN, C₁-C₁₂-haloalkoxy, —C(O)NH—C₁-C₁₂-alkyl, which is unsubstituted or substituted by one or more halo; or
   C₂-C₁₂-heteroaryl which is unsubstituted or substituted by one or more substituents selected from the group consisting of halo and C₁-C₁₂-alkyl;
R² is: CN, C₁-C₁₂-alkyl which is unsubstituted or substituted by one or more substituents selected from the group consisting of cyano, halo, hydroxy, C₁-C₁₂-alkoxy, C₃-C₁₂-cycloalkyl, —NH₂, —NH—C₁-C₁₂-alkyl, —NH—C₂-C₁₂-heteroaryl, —NHC(O)—C₁-C₁₂-alkyl, —NHC(O)—C₃-C₁₂-cycloalkyl, —NHC(O)—C₆-C₂O-aryl, —NHC(O)—C₂-C₁₂-heteroaryl, —NHC(O)NH—C₁-C₁₂-alkyl, —NHC(O)NH—C₂-C₁₂-heteroaryl, —NHS(O)₂—C₁-C₁₂-alkyl, and —NHS(O)₂—C₃-C₁₂-cycloalkyl;
   —C(O)—C₂-C₁₂-heterocyclyl, which is unsubstituted or substituted by one or more substituents selected from the group consisting of: C₁-C₁₂-alkyl, C₁-C₁₂-hydroxyalkyl, —C(O)—NH₂, —C(O)—N(H)(C₁-C₁₂-alkyl), —C(O)—N(C₁-C₁₂-alkyl)₂, —C₁-C₁₂-alkylenyl-C(O)—N(C₁-C₁₂-alkyl)₂, —NH—C(O)—C₁-C₁₂-alkyl, —NH—C(O)—C₃-C₁₂-cycloalkyl, and —N(C(O)—C₃-C₁₂-cycloalkyl)₂;
   —C(O)OH;
   —C(O)—C₁-C₁₂-alkoxy;
   —C(O)NR^{b}R^{c}, wherein R^{b} and R^{c} are independently selected from the group consisting of: H; —C₁-C₁₂-alkyl, which is unsubstituted or substituted by one or more substituent selected from the group consisting of:
      OH, CN, NH₂, —C₃-C₁₂-cycloalkyl, —C(O)—NH₂, —C(O)—C₂-C₁₂-heterocyclyl, —N(H)(C(O)—C₁-C₁₂-alkyl), —N(H)(C₁-C₁₂-alkyl), —N(C₁-C₁₂-alkyl)₂, —NHC(O)—NH(C₃-C₁₂-cycloalkyl), —NHC(O)—NH(C₁-C₁₂-alkyl);
      C₂-C₁₂-heterocyclyl, wherein the heterocyclyl is unsubstituted or substituted by one or more substituents selected from the group consisting of oxo, hydroxy and C₁-C₁₂-alkyl;
      —C(O)—C₂-C₁₂-heterocyclyl, wherein the heterocyclyl is unsubstituted or substituted by one or more substituents selected from the group consisting of oxo, hydroxy and C₁-C₁₂-alkyl;
      —SO₂—C₂-C₁₂-heterocyclyl;
      C₆-C₂₀-aryl, which is unsubstituted or substituted by one or more substituents selected from the group consisting of C₁-C₁₂-alkyl, C₁-C₁₂-alkoxy, —C₁-C₁₂-hydroxyalkyl, C₁-C₁₂-alkylenyl-C₁-C₁₂-alkoxy; C₁-C₁₂-alkylenyl-NH₂, and —O—C₂-C₁₂-heterocyclyl, which heterocyclyl is unsubstituted or substituted by C₁-C₁₂-alkyl; and
      C₂-C₁₂-heteroaryl, which is unsubstituted or substituted by one or more substituents selected from the group consisting of C₁-C₁₂-alkyl, C₁-C₁₂-alkoxy, —C(O)—NH₂, —C(O)—N(H)(C₁-C₁₂-alkyl), —C(O)—N(C₁-C₁₂-alkyl)₂, and —C₁-C₁₂-alkylenyl-C(O)—N(C₁-C₁₂-alkyl)₂;
      —C₃-C₁₂-cycloalkyl, which is unsubstituted or substituted by one or more hydroxy; —C₂-C₁₂-heterocyclyl, which is unsubstituted or substituted by one or more substituents selected from the group consisting of: C₁-C₁₂-alkyl, —SO₂—C₁-C₁₂-alkyl, —C(O)—C₁-C₁₂-alkyl, —C(O)—NH₂, —C(O)—N(H)(C₁-C₁₂-alkyl), —C(O)—N(C₁-C₁₂-alkyl)₂, —C(O)—C₁-C₁₂-alkylenyl-C₁-C₁₂-alkoxy, —C₁-C₁₂-alkylenyl-C(O)—N(C₁-C₁₂-alkyl)₂, —C(O)—C₃-C₁₂-cycloalkyl, and —C(O)NH—C₃-C₁₂-cycloalkyl;
      C₆-C₂O-aryl, which is unsubstituted or substituted by one or more substituents selected from the group consisting of C₁-C₁₂-alkyl, C₁-C₁₂-alkoxy, —C₁-C₁₂-hydroxyalkyl, C₁-C₁₂-alkylenyl-C₁-C₁₂-alkoxy; C₁-C₁₂-alkylenyl-NH₂, and —O—C₂-C₁₂-heterocyclyl, which heterocyclyl is unsubstituted or substituted by C₁-C₁₂-alkyl; and
      C₆-C₂₀-heteroaryl, which is unsubstituted or substituted by one or more substituents selected from the group consisting of C₁-C₁₂-alkyl, C₁-C₁₂-hydroxyalkyl, and —O—C₂-C₁₂-heterocyclyl which heterocyclyl is unsubstituted or substituted by C₁-C₁₂-alkyl; and
      C₂-C₁₂-heteroaryl which is unsubstituted or substituted by C₁-C₁₂-alkyl, which alkyl is unsubstituted or substituted by one or more substituents selected from the group consisting of halo, —C(O)—N(C₁-C₁₂-alkyl)₂, and —C₂-C₁₂-heterocyclyl, wherein the C₂-C₁₂-heterocyclyl is unsubstituted or substituted by C₁-C₁₂-alkyl;
         with the proviso that when A is CH, R¹ is selected from phenyl that is unsubstituted or substituted by halo.

In some embodiments of the compound of Formula (I), A is CH or N;
R¹ is C₆-C₂O-aryl substituted by one two or three halo;
R² is CN, C₁-C₁₂-alkyl which is unsubstituted or substituted by one or more substituents selected from the group consisting of —NH₂, —NH—C₁-C₁₂-alkyl, —NHC(O)—C₃-C₁₂-cycloalkyl, —NHC(O)—C₆-C₂O-aryl, and —NHS(O)₂—C₃-C₁₂-cycloalkyl;
   —C(O)—C₂-C₁₂-heterocyclyl, which is unsubstituted or substituted by one or more substituents selected from the group consisting of: —C₁-C₁₂-hydroxyalkyl, —C₁-C₁₂-alkylenyl-C(O)—N(C₁-C₁₂-alkyl)₂, —NH—C(O)—C₁-C₁₂-alkyl, —NH—C(O)—C₃-C₁₂-cycloalkyl, and —N(C(O)—C₃-C₁₂-cycloalkyl)₂;
   —C(O)OH;
   —C(O)—C₁-C₁₂-alkoxy;
   —C(O)NR^{b}R^{c}, wherein R^{b} and R^{c} are independently selected from the group consisting of: H; alkyl, which is unsubstituted or substituted by one or more substituent selected from the group consisting of: OH, CN, NH₂, —C₃-C₁₂-cycloalkyl, —C(O)—NH₂, —C(O)—C₂-C₁₂-heterocyclyl, —N(H)(C(O)—C₁-C₁₂-alkyl), —N(H)(C₁-C₁₂alkyl), —N(C₁-C₁₂-alkyl)₂, —NHC(O)—NH(C₃-C₁₂-cycloalkyl), —NHC(O)—NH(C₁-C₁₂-alkyl);
      C₂-C₁₂-heterocyclyl, wherein the heterocyclyl is unsubstituted or substituted by one or more substituents selected from the group consisting of oxo, hydroxy and C₁-C₁₂-alkyl; —C(O)—C₂-C₁₂-heterocyclyl;
      —SO₂—C₂-C₁₂-heterocyclyl;
         C₆-C₂O-aryl, which is unsubstituted or substituted by C₁-C₁₂alkoxy; and
         C₂-C₁₂-heteroaryl, which is unsubstituted or substituted by one or more substituents selected from the group consisting of C₁-C₁₂-alkyl, —C(O)—NH₂, —C(O)—N(H)(C₁-C₁₂-alkyl), and —C(O)—N(C₁-C₁₂-alkyl)₂;
   —C₃-C₁₂-cycloalkyl, which is unsubstituted or substituted by one or more hydroxy; —C₂-C₁₂-heterocyclyl, which is unsubstituted or substituted by one or more substituents selected from the group consisting of: C₁-C₁₂-alkyl, —SO₂—C₁-C₁₂-alkyl, —C(O)—C₁-C₁₂-alkyl, —C(O)—NH₂, —C(O)—N(H)(C₁-C₁₂-alkyl), —C(O)—N(C₁-C₁₂-alkyl)₂, —C(O)—C₁-C₁₂-alkylenyl-C₁-C₁₂-alkoxy, —C₁-C₁₂-alkylenyl-C(O)—N(C₁-C₁₂-alkyl)₂, —C(O)—C₃-C₁₂-cycloalkyl and —C(O)NH—C₃-C₁₂-cycloalkyl;
   C₆-C₂₀-aryl, which is unsubstituted or substituted by one or more substituents selected from the group consisting of C₁-C₁₂-alkyl, C₁-C₁₂-alkoxy, —C₁-C₁₂-hydroxyalkyl, C₁-C₁₂-alkylenyl-C₁-C₁₂-alkoxy; C₁-C₁₂-alkylenyl-NH₂, and —O—C₂-C₁₂-heterocyclyl, which heterocyclyl is unsubstituted or substituted by C₁-C₁₂-alkyl; and
   C₆-C₂₀-heteroaryl, which is unsubstituted or substituted by one or more substituents selected from the group consisting of C₁-C₁₂-alkyl, which is unsubstituted or substituted by C₂-C₁₂-heterocyclyl, C₁-C₁₂-hydroxyalkyl, and —O—C₂-C₁₂-heterocyclyl which is unsubstituted or substituted by C alkyl; and C₂-C₁₂-heteroaryl which is unsubstituted or substituted by C₁-C₁₂-alkyl, which alkyl is unsubstituted or substituted by one or more substituents selected from the group consisting of halo, —C(O)—N(C₁-C₁₂-alkyl)₂, and —C₂-C₁₂-heterocyclyl, wherein the heterocyclyl is unsubstituted or substituted by C₁-C₁₂-alkyl;
      with the proviso that when A is CH, R¹ is selected from phenyl that is unsubstituted or substituted by halo.

In some embodiments, the MAP4K4 is a compound selected from:

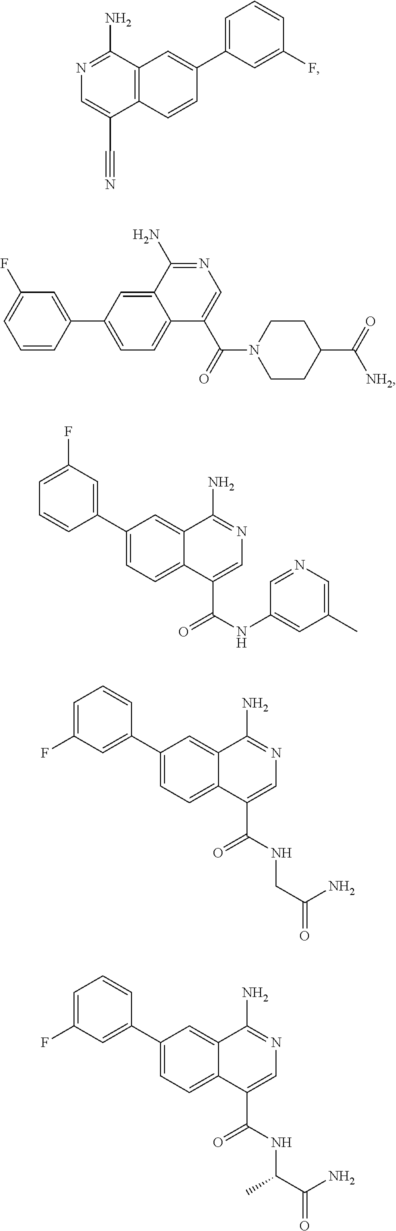 [SMILES: N#Cc1cnc(N)c2cc(-c3cccc(F)c3)ccc12 ; NC(=O)C1CCN(C(=O)c2cnc(N)c3cc(-c4cccc(F)c4)ccc23)CC1 ; Cc1cncc(NC(=O)c2cnc(N)c3cc(-c4cccc(F)c4)ccc23)c1 ; NC(=O)CNC(=O)c1cnc(N)c2cc(-c3cccc(F)c3)ccc12 ; C[C@H](NC(=O)c1cnc(N)c2cc(-c3cccc(F)c3)ccc12)C(N)=O]

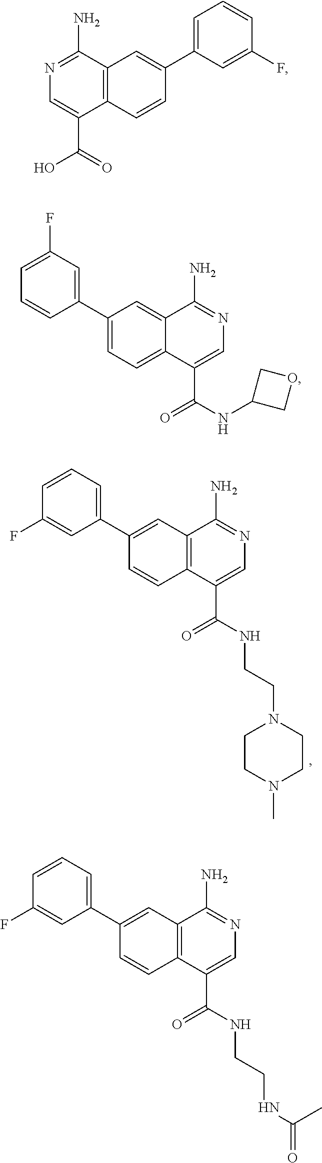 [SMILES: Nc1ncc(C(=O)O)c2ccc(-c3cccc(F)c3)cc12 ; Nc1ncc(C(=O)NC2COC2)c2ccc(-c3cccc(F)c3)cc12 ; CN1CCN(CCNC(=O)c2cnc(N)c3cc(-c4cccc(F)c4)ccc23)CC1 ; CC(=O)NCCNC(=O)c1cnc(N)c2cc(-c3cccc(F)c3)ccc12]

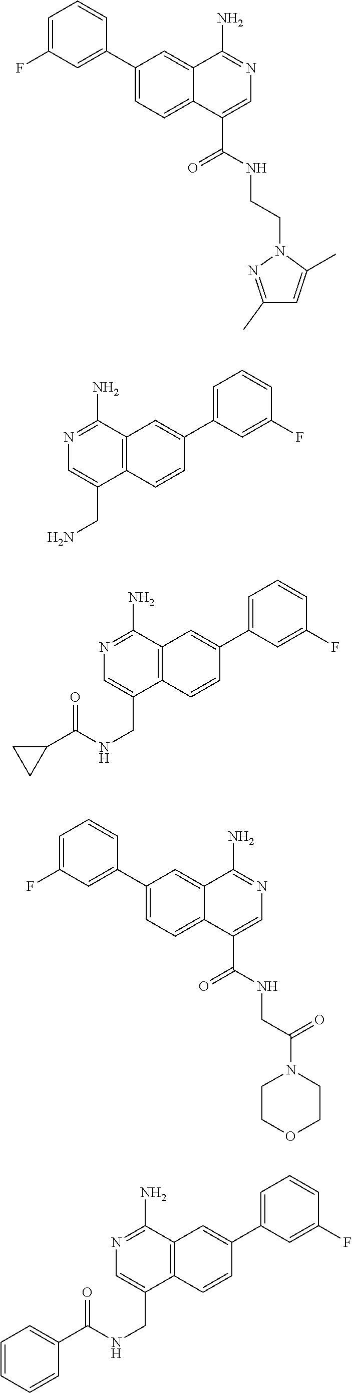

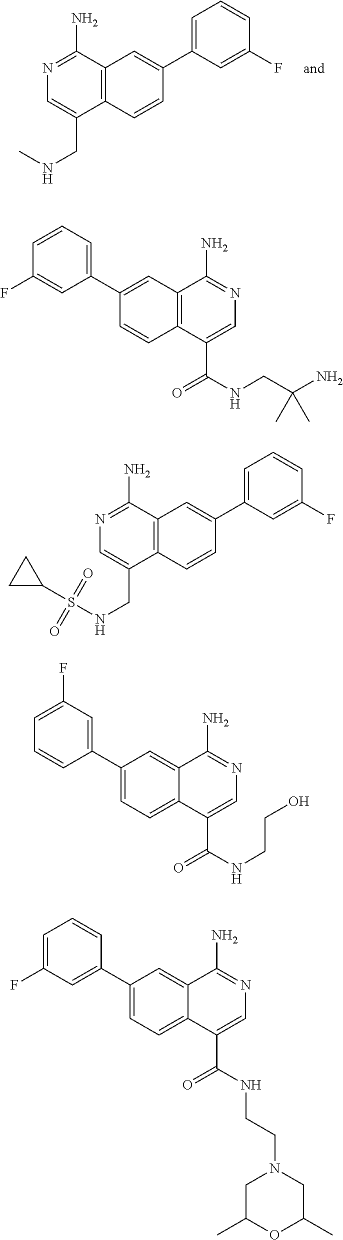

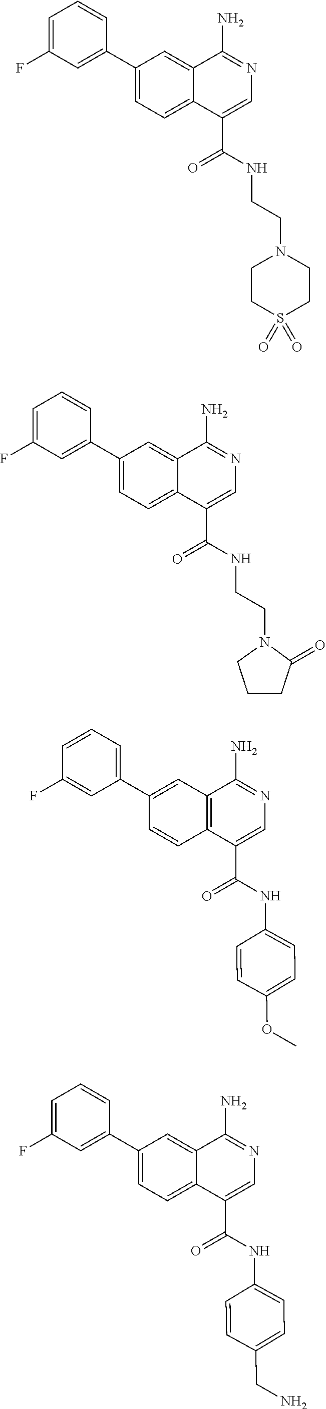 [SMILES: Nc1ncc(C(=O)NCCN2CCS(=O)(=O)CC2)c2ccc(-c3cccc(F)c3)cc12 ; Nc1ncc(C(=O)NCCN2CCCC2=O)c2ccc(-c3cccc(F)c3)cc12 ; COc1ccc(NC(=O)c2cnc(N)c3cc(-c4cccc(F)c4)ccc23)cc1 ; NCc1ccc(NC(=O)c2cnc(N)c3cc(-c4cccc(F)c4)ccc23)cc1]

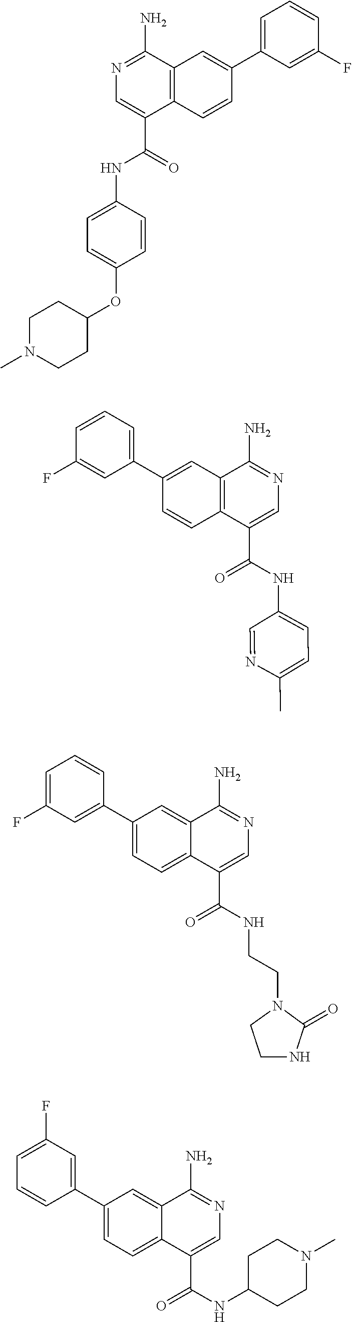 [SMILES: CN1CCC(Oc2ccc(NC(=O)c3cnc(N)c4cc(-c5cccc(F)c5)ccc34)cc2)CC1 ; Cc1ccc(NC(=O)c2cnc(N)c3cc(-c4cccc(F)c4)ccc23)cn1 ; Nc1ncc(C(=O)NCCN2CCNC2=O)c2ccc(-c3cccc(F)c3)cc12 ; CN1CCC(NC(=O)c2cnc(N)c3cc(-c4cccc(F)c4)ccc23)CC1]

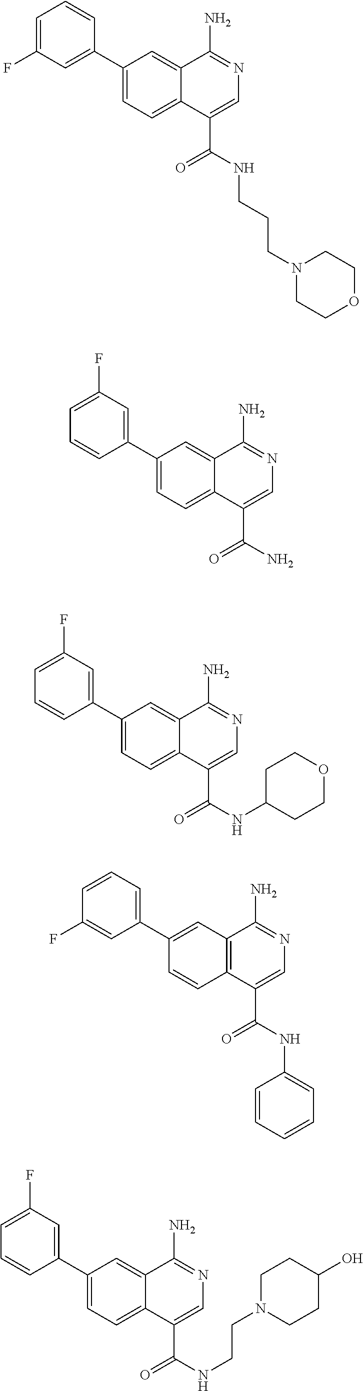 [SMILES: Nc1ncc(C(=O)NCCCN2CCOCC2)c2ccc(-c3cccc(F)c3)cc12 ; NC(=O)c1cnc(N)c2cc(-c3cccc(F)c3)ccc12 ; Nc1ncc(C(=O)NC2CCOCC2)c2ccc(-c3cccc(F)c3)cc12 ; Nc1ncc(C(=O)Nc2ccccc2)c2ccc(-c3cccc(F)c3)cc12 ; Nc1ncc(C(=O)NCCN2CCC(O)CC2)c2ccc(-c3cccc(F)c3)cc12]

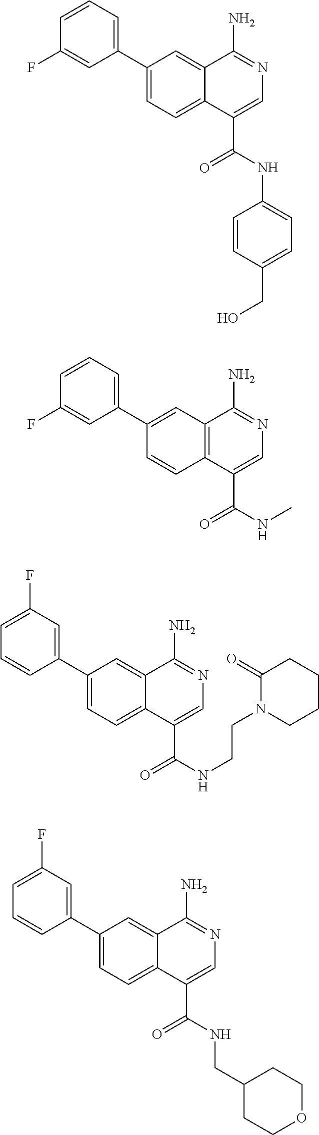 [SMILES: Nc1ncc(C(=O)Nc2ccc(CO)cc2)c2ccc(-c3cccc(F)c3)cc12 ; CNC(=O)c1cnc(N)c2cc(-c3cccc(F)c3)ccc12 ; Nc1ncc(C(=O)NCCN2CCCCC2=O)c2ccc(-c3cccc(F)c3)cc12 ; Nc1ncc(C(=O)NCC2CCOCC2)c2ccc(-c3cccc(F)c3)cc12]

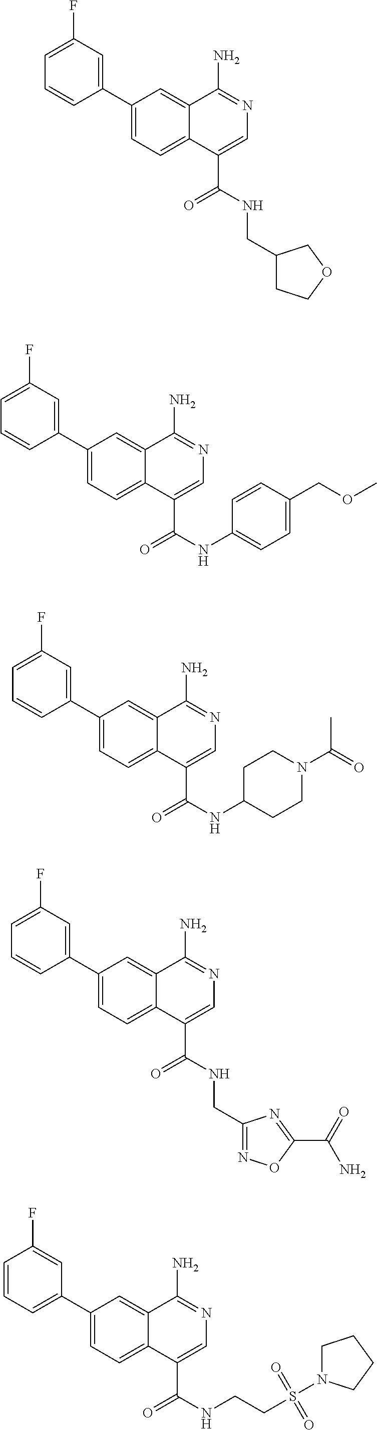 [SMILES: Nc1ncc(C(=O)NCC2CCOC2)c2ccc(-c3cccc(F)c3)cc12 ; COCc1ccc(NC(=O)c2cnc(N)c3cc(-c4cccc(F)c4)ccc23)cc1 ; CC(=O)N1CCC(NC(=O)c2cnc(N)c3cc(-c4cccc(F)c4)ccc23)CC1 ; NC(=O)c1nc(CNC(=O)c2cnc(N)c3cc(-c4cccc(F)c4)ccc23)no1 ; Nc1ncc(C(=O)NCCS(=O)(=O)N2CCCC2)c2ccc(-c3cccc(F)c3)cc12]

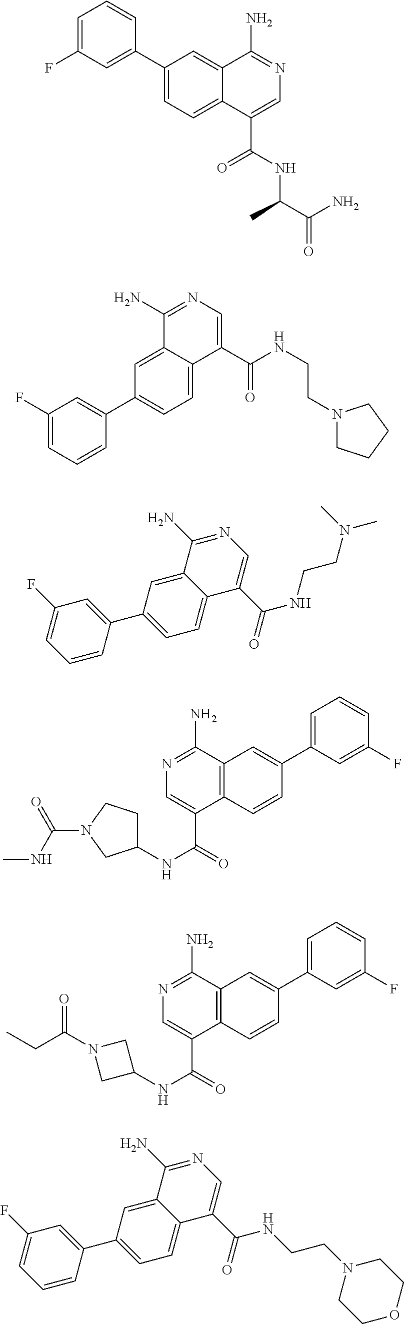

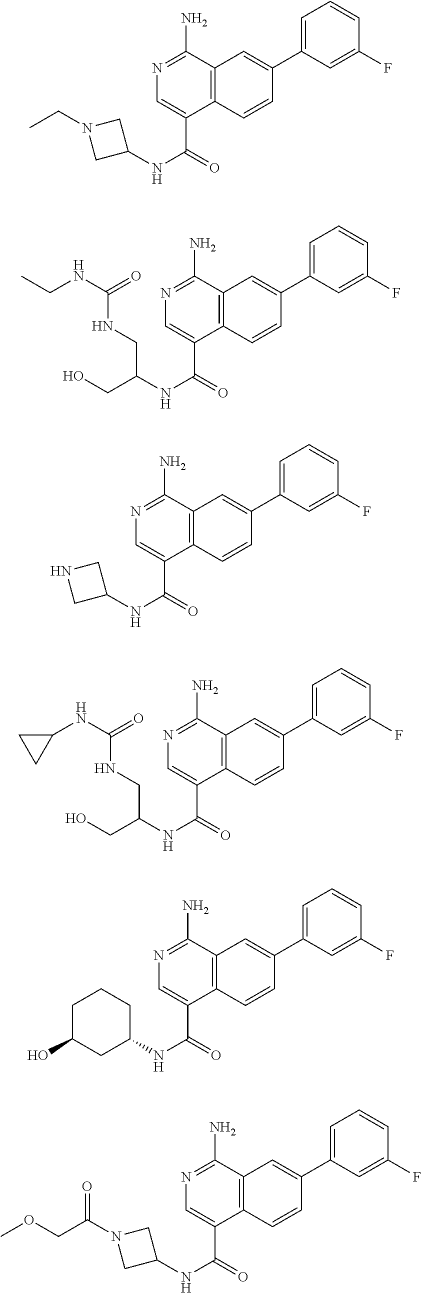 [SMILES: CCN1CC(NC(=O)c2cnc(N)c3cc(-c4cccc(F)c4)ccc23)C1 ; CCNC(=O)NCC(CO)NC(=O)c1cnc(N)c2cc(-c3cccc(F)c3)ccc12 ; Nc1ncc(C(=O)NC2CNC2)c2ccc(-c3cccc(F)c3)cc12 ; Nc1ncc(C(=O)NC(CO)CNC(=O)NC2CC2)c2ccc(-c3cccc(F)c3)cc12 ; Nc1ncc(C(=O)N[C@H]2CCC[C@H](O)C2)c2ccc(-c3cccc(F)c3)cc12 ; COCC(=O)N1CC(NC(=O)c2cnc(N)c3cc(-c4cccc(F)c4)ccc23)C1]

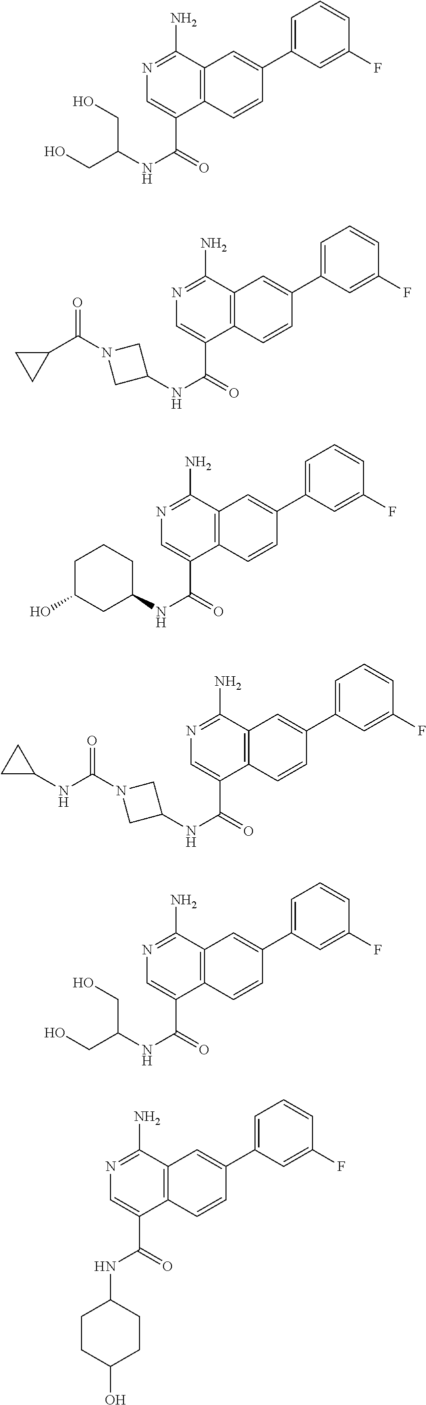 [SMILES: Nc1ncc(C(=O)NC(CO)CO)c2ccc(-c3cccc(F)c3)cc12 ; Nc1ncc(C(=O)NC2CN(C(=O)C3CC3)C2)c2ccc(-c3cccc(F)c3)cc12 ; Nc1ncc(C(=O)N[C@@H]2CCC[C@@H](O)C2)c2ccc(-c3cccc(F)c3)cc12 ; Nc1ncc(C(=O)NC2CN(C(=O)NC3CC3)C2)c2ccc(-c3cccc(F)c3)cc12 ; Nc1ncc(C(=O)NC(CO)CO)c2ccc(-c3cccc(F)c3)cc12 ; Nc1ncc(C(=O)NC2CCC(O)CC2)c2ccc(-c3cccc(F)c3)cc12]

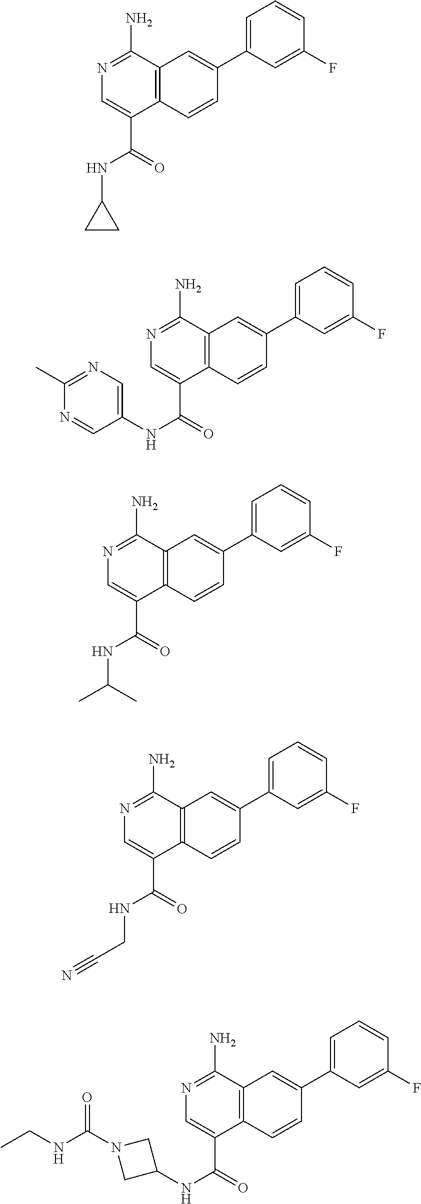 [SMILES: Nc1ncc(C(=O)NC2CC2)c2ccc(-c3cccc(F)c3)cc12 ; Cc1ncc(NC(=O)c2cnc(N)c3cc(-c4cccc(F)c4)ccc23)cn1 ; CC(C)NC(=O)c1cnc(N)c2cc(-c3cccc(F)c3)ccc12 ; N#CCNC(=O)c1cnc(N)c2cc(-c3cccc(F)c3)ccc12 ; CCNC(=O)N1CC(NC(=O)c2cnc(N)c3cc(-c4cccc(F)c4)ccc23)C1]

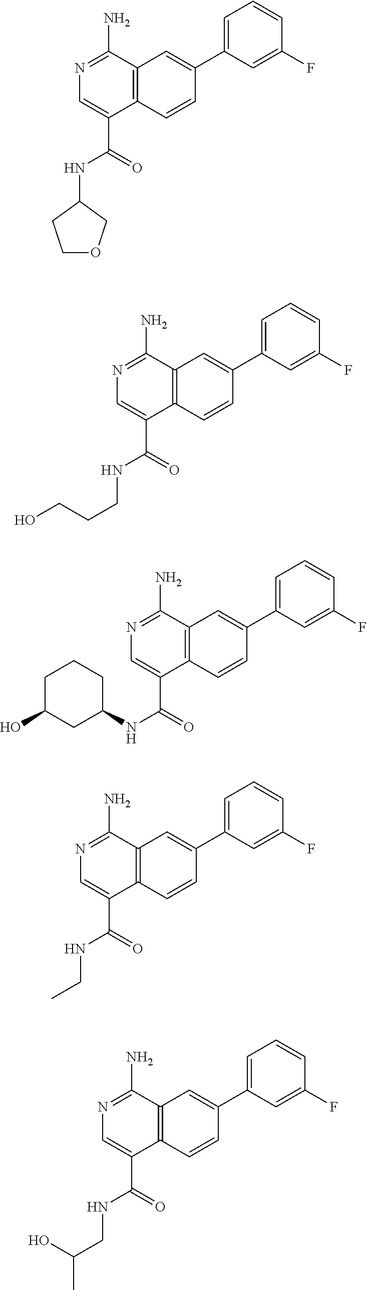 [SMILES: Nc1ncc(C(=O)NC2CCOC2)c2ccc(-c3cccc(F)c3)cc12 ; Nc1ncc(C(=O)NCCCO)c2ccc(-c3cccc(F)c3)cc12 ; Nc1ncc(C(=O)N[C@@H]2CCC[C@H](O)C2)c2ccc(-c3cccc(F)c3)cc12 ; CCNC(=O)c1cnc(N)c2cc(-c3cccc(F)c3)ccc12 ; CC(O)CNC(=O)c1cnc(N)c2cc(-c3cccc(F)c3)ccc12]

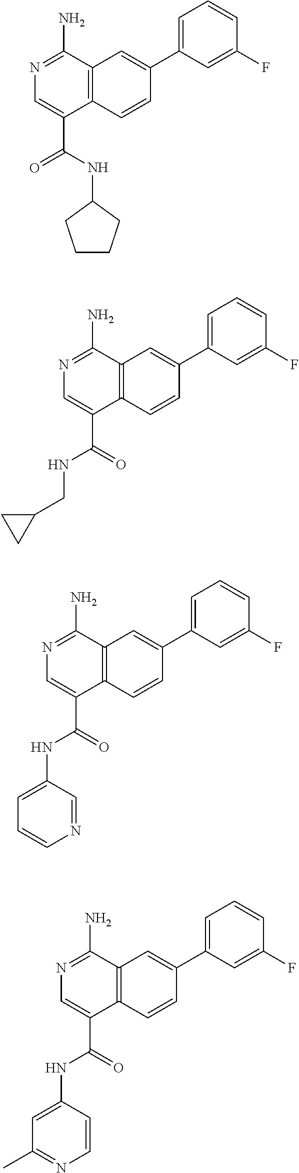 [SMILES: Nc1ncc(C(=O)NC2CCCC2)c2ccc(-c3cccc(F)c3)cc12 ; Nc1ncc(C(=O)NCC2CC2)c2ccc(-c3cccc(F)c3)cc12 ; Nc1ncc(C(=O)Nc2cccnc2)c2ccc(-c3cccc(F)c3)cc12 ; Cc1cc(NC(=O)c2cnc(N)c3cc(-c4cccc(F)c4)ccc23)ccn1]

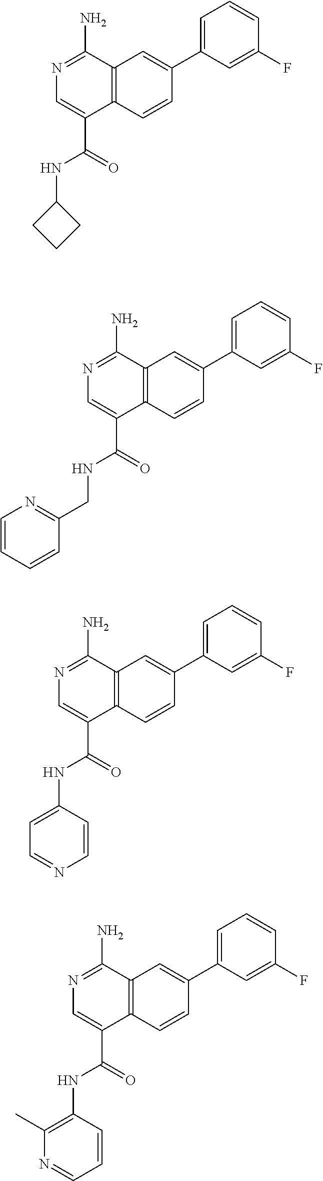 [SMILES: Nc1ncc(C(=O)NC2CCC2)c2ccc(-c3cccc(F)c3)cc12 ; Nc1ncc(C(=O)NCc2ccccn2)c2ccc(-c3cccc(F)c3)cc12 ; Nc1ncc(C(=O)Nc2ccncc2)c2ccc(-c3cccc(F)c3)cc12 ; Cc1ncccc1NC(=O)c1cnc(N)c2cc(-c3cccc(F)c3)ccc12]

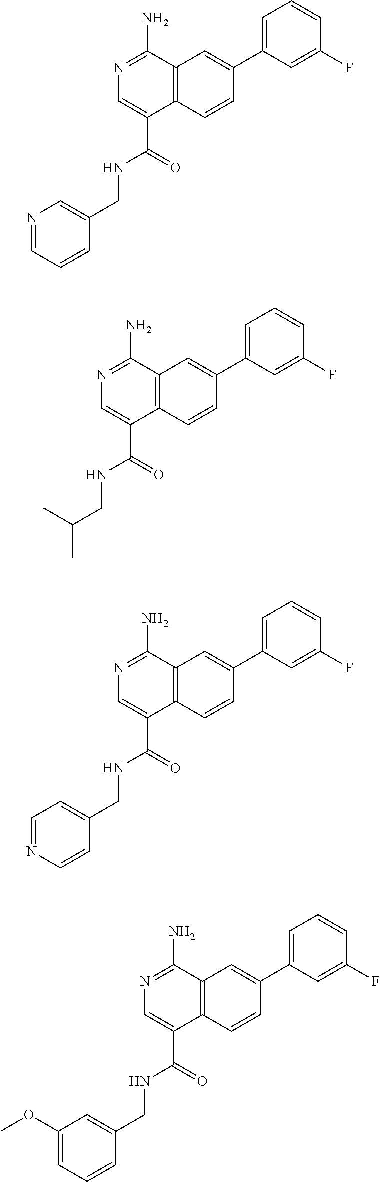 [SMILES: Nc1ncc(C(=O)NCc2cccnc2)c2ccc(-c3cccc(F)c3)cc12 ; CC(C)CNC(=O)c1cnc(N)c2cc(-c3cccc(F)c3)ccc12 ; Nc1ncc(C(=O)NCc2ccncc2)c2ccc(-c3cccc(F)c3)cc12 ; COc1cccc(CNC(=O)c2cnc(N)c3cc(-c4cccc(F)c4)ccc23)c1]

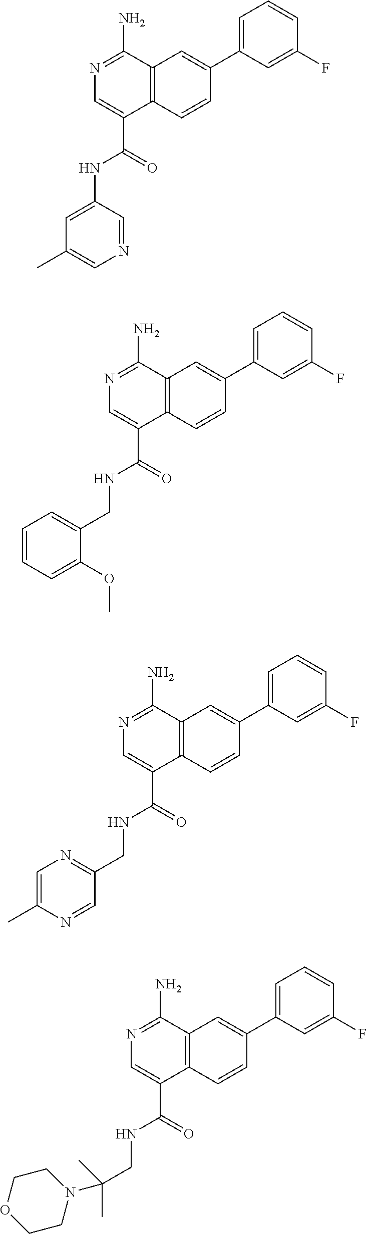 [SMILES: Cc1cncc(NC(=O)c2cnc(N)c3cc(-c4cccc(F)c4)ccc23)c1 ; COc1ccccc1CNC(=O)c1cnc(N)c2cc(-c3cccc(F)c3)ccc12 ; Cc1cnc(CNC(=O)c2cnc(N)c3cc(-c4cccc(F)c4)ccc23)cn1 ; CC(C)(CNC(=O)c1cnc(N)c2cc(-c3cccc(F)c3)ccc12)N1CCOCC1]

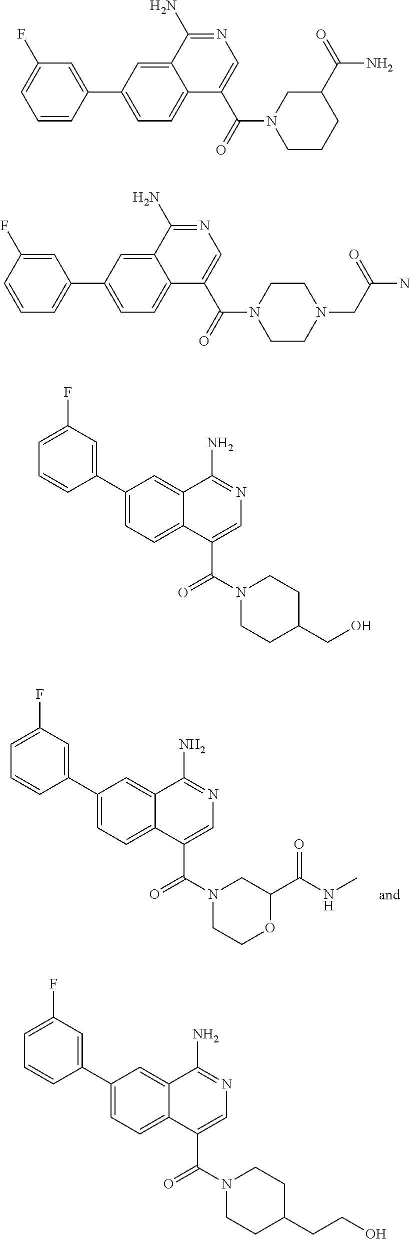 [SMILES: NC(=O)C1CCCN(C(=O)c2cnc(N)c3cc(-c4cccc(F)c4)ccc23)C1 ; NC(=O)CN1CCN(C(=O)c2cnc(N)c3cc(-c4cccc(F)c4)ccc23)CC1 ; Nc1ncc(C(=O)N2CCC(CO)CC2)c2ccc(-c3cccc(F)c3)cc12 ; CNC(=O)C1CN(C(=O)c2cnc(N)c3cc(-c4cccc(F)c4)ccc23)CCO1 ; Nc1ncc(C(=O)N2CCC(CCO)CC2)c2ccc(-c3cccc(F)c3)cc12]

In some embodiments, the MAP4K4 modulator is a compound of Formula (II):

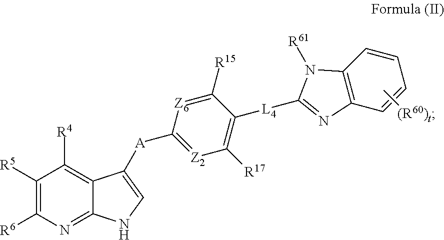

or a pharmaceutically acceptable salt, tautomer, or isomers thereof, wherein:
t is 0, 1, 2, or 3;
Z₂ is N or CR¹²;
Z₆ is N or CR¹⁶;
L₄ is selected from the group consisting of —(CR¹⁰R¹¹)ₚ—NR²⁵—(CR¹⁰R¹¹)_{q}—, —(CR¹⁰R¹¹)ₚ—X—(CR¹⁰R¹¹)_{q}—, —(CR¹⁰R¹¹)ₚ—C(X)—(CR¹⁰R¹¹)_{q}—, —(CR¹⁰R¹¹)ₚ—S(O)—(CR¹⁰R¹¹)_{q}—, —(CR¹⁰R¹¹)ₚS(O)₂—(CR¹⁰R¹¹)_{q}—, —(CR¹⁰R¹¹)ₚ—C(X)NR²⁵—(CR¹⁰R¹¹)_{q}—, —(CR¹⁰R¹¹)ₚ—S(O)₂NR²⁵—(CR¹⁰R¹¹)ₚ—, —(CR¹⁰R¹¹)ₚ—NR²⁵C(X)—(CR¹⁰R¹¹)_{q}—, —(CR¹⁰R¹¹)ₚ—NR²⁵S(O)₂—(CR¹⁰R¹¹)_{q}—, —(CR¹⁰R¹¹)ₚ—NR²⁵C(X)NR²⁵—(CR¹⁰R¹¹)_{q}—, and —(CR¹⁰R¹¹)ₚ—NR²⁵S(O)₂NR²⁵—(CR¹⁰R¹¹)_{q}—;
p and q are independently 0, 1, or 2 provided, however, that at least one of p and q is 0;
R⁶⁰ is selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —CN, —NO₂, —CR^{a}R^{b}R²⁶, and -LR²⁶;
R⁶¹ is hydrogen, lower alkyl, or fluoro substituted lower alkyl;
A is selected from the group consisting of —O—, —S—, —CR^{a}R^{b}—, —NR¹—, —C(O)—, —C(S)—, —S(O)—, and —S(O)₂—;
R^{a} and R^{b} at each occurrence are independently selected from the group consisting of hydrogen, fluoro, —OH, —NH₂, lower alkyl, lower alkoxy, lower alkylthio, mono-alkylamino, di-alkylamino, and —NR⁸R⁹, wherein the alkyl chain(s) of lower alkyl, lower alkoxy, lower alkylthio, mono-alkylamino, or di-alkylamino are optionally substituted with one or more substituents selected from the group consisting of fluoro, —OH, —NH₂, lower alkoxy, fluoro substituted lower alkoxy, lower alkylthio, fluoro substituted lower alkylthio, mono-alkylamino, di-alkylamino, and cycloalkylamino, provided, however, that any substitution of the alkyl chain carbon bound to O of alkoxy, S of thioalkyl or N of mono- or di-alkylamino is fluoro; or
R^{a} and R^{b} combine to form a 3-7 membered monocyclic cycloalkyl or 5-7 membered monocyclic heterocycloalkyl, wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
R¹ is selected from the group consisting of hydrogen, lower alkyl, cycloalkyl, heterocycloalkyl, aryl, heteroaryl, —C(O)R⁷, —C(S)R⁷, —S(O)₂R⁷, —C(O)NHR⁷, —C(S)NHR⁷, and —S(O)₂NHR⁷, wherein lower alkyl is optionally substituted with one or more substituents selected from the group consisting of fluoro, —OH, —NH₂, lower alkoxy, lower alkylthio, mono-alkylamino, di-alkylamino, and —NR⁸R⁹, wherein the alkyl chain(s) of lower alkoxy, lower alkylthio, mono-alkylamino, or di-alkylamino are optionally substituted with one or more substituents selected from the group consisting of fluoro, —OH, —NH₂, lower alkoxy, fluoro substituted lower alkoxy, lower alkylthio, fluoro substituted lower alkylthio, mono-alkylamino, di-alkylamino, and cycloalkylamino, provided, however, that any substitution of the alkyl chain carbon bound to O of alkoxy, S of thioalkyl or N of mono- or di-alkylamino is fluoro, further provided that when R¹ is lower alkyl, any substitution on the lower alkyl carbon bound to the N of —NR¹— is fluoro, and wherein cycloalkyl, heterocycloalkyl, aryl or heteroaryl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
R⁷ is selected from the group consisting of lower alkyl, cycloalkyl, heterocycloalkyl, aryl, and heteroaryl, wherein lower alkyl is optionally substituted with one or more substituents selected from the group consisting of fluoro, —OH, —NH₂, lower alkoxy, lower alkylthio, mono-alkylamino, di-alkylamino, and —NR⁸R⁹, provided, however, that any substitution of the alkyl carbon bound to the N of —C(O)NHR⁷, —C(S)NHR⁷ or —S(O)₂NHR⁷ is fluoro, wherein the alkyl chain(s) of lower alkoxy, lower alkylthio, mono-alkylamino, or di-alkylamino are optionally substituted with one or more substituents selected from the group consisting of fluoro, —OH, —NH₂, lower alkoxy, fluoro substituted lower alkoxy, lower alkylthio, fluoro substituted lower alkylthio, mono-alkylamino, di-alkylamino, and cycloalkylamino, provided, however, that any substitution of the alkyl chain carbon bound to O of alkoxy, S of thioalkyl or N of mono- or di-alkylamino is fluoro, and wherein cycloalkyl, heterocycloalkyl, aryl and heteroaryl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
each of R⁴, R⁵, R⁶, R¹², R¹⁵, and R¹⁶, are independently selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —CN, —NO₂, —CR^{a}R^{b}R²⁶, and -LR²⁶; L at each occurrence is independently selected from the group consisting of -(alk)ₐ-X-(alk)_{b}-, -(alk)ₐ-NR²⁵-(alk)_{b}-, -(alk)ₐ-C(X)-(alk)_{b}-, -(alk)ₐ-S(O)-(alk)_{b}-, -(alk)ₐ-S(O)₂-(alk)_{b}-, -(alk)ₐ-OC(X)-(alk)_{b}-, -(alk)ₐ-C(X)O-(alk)_{b}-, -(alk)ₐ-C(X)NR²⁵-(alk)_{b}-, -(alk)ₐ-S(O)₂NR²⁵-(alk)_{b}-, -(alk)ₐ-NR²⁵C(X)-(alk)_{b}-, -(alk)ₐ-NR²⁵S(O)₂-(alk)_{b}-, -(alk)ₐ-NR²⁵C(X)O-(alk)_{b}-, -(alk)ₐ-OC(X)NR²⁵-(alk)_{b}-, -(alk)ₐ-NR²⁵C(X)NR²⁵-(alk)_{b}-, and -(alk)ₐ-NR²⁵S(O)₂NR²⁵-(alk)_{b}-;
a and b are independently 0 or 1;
alk at each occurrence is independently C₁₋₃ alkylene or C₁₋₃ alkylene substituted with one or more substituents selected from the group consisting of fluoro, —OH, —NH₂, lower alkyl, lower alkoxy, lower alkylthio, mono-alkylamino, di-alkylamino, and —NR⁸R⁹, wherein lower alkyl or the alkyl chain(s) of lower alkoxy, lower alkylthio, mono-alkylamino or di-alkylamino are optionally substituted with one or more substituents selected from the group consisting of fluoro, —OH, —NH₂, lower alkoxy, fluoro substituted lower alkoxy, lower alkylthio, fluoro substituted lower alkylthio, mono-alkylamino, di-alkylamino and cycloalkylamino, provided, however, that any substitution of the alkyl chain carbon bound to O of alkoxy, S of thioalkyl or N of mono- or di-alkylamino is fluoro;
X at each occurrence is independently O or S;
R²⁵ at each occurrence is independently selected from the group consisting of hydrogen, optionally substituted lower alkyl, optionally substituted cycloalkyl, optionally substituted heterocycloalkyl, optionally substituted aryl, and optionally substituted heteroaryl;
R²⁶ at each occurrence is independently selected from the group consisting of hydrogen, provided, however, that hydrogen is not bound to any of S(O), S(O)₂, C(O) or C(S) of L, optionally substituted lower alkyl, optionally substituted lower alkenyl, provided, however, that when R²⁶ is optionally substituted lower alkenyl, no alkene carbon thereof is bound to N, S, O, S(O), S(O)₂, C(O) or C(S) of L, optionally substituted lower alkynyl, provided, however, that when R²⁶ is optionally substituted lower alkynyl, no alkyne carbon thereof is bound to N, S, O, S(O), S(O)₂, C(O) or C(S) of L, optionally substituted cycloalkyl, optionally substituted heterocycloalkyl, optionally substituted aryl, and optionally substituted heteroaryl;
R¹⁰ and R¹¹ at each occurrence are independently selected from the group consisting of hydrogen, fluoro, lower alkyl, and lower alkyl optionally substituted with one or more substituents selected from the group consisting of fluoro, —OH, —NH₂, lower alkoxy, fluoro substituted lower alkoxy, lower alkylthio, fluoro substituted lower alkylthio, mono-alkylamino, di-alkylamino, and cycloalkylamino; or any two of R¹⁰ and R¹¹ on the same or adjacent carbon atoms combine to form a 3-7 membered monocyclic cycloalkyl or 5-7 membered monocyclic heterocycloalkyl, and any others of R¹⁰ and R¹¹ are independently selected from the group consisting of hydrogen, fluoro, lower alkyl, and lower alkyl optionally substituted with one or more substituents selected from the group consisting of fluoro, —OH, —NH₂, lower alkoxy, fluoro substituted lower alkoxy, lower alkylthio, fluoro substituted lower alkylthio, mono-alkylamino, di-alkylamino, and cycloalkylamino, and wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
R⁸ and R⁹ combine with the nitrogen to which they are attached to form a 5-7 membered heterocycloalkyl optionally substituted with one or more substituents selected from the group consisting of fluoro, —OH, —NH₂, lower alkyl, fluoro substituted lower alkyl, lower alkoxy, fluoro substituted lower alkoxy, lower alkylthio, and fluoro substituted lower alkylthio;
R¹⁷ is selected from the group consisting of hydrogen, halogen, optionally substituted lower alkyl and —OR¹⁸; and
R¹⁸ is hydrogen or optionally substituted lower alkyl.

In some embodiments, the MAP4K4 modulator is the compound selected from: from 2-[5-Chloro-4-(5-chloro-1H-pyrrolo[2,3-b]pyridin-3-ylmethyl)-2-fluoro-phenoxymethyl]-1H-benzoimidazole, 2-[4-(5-Chloro-1H-pyrrolo[2,3-b]pyridin-3-ylmethyl)-2,5-difluoro-phenoxymethyl]-1H-benzoimidazole, 2-[2,5-Difluoro-4-(5-methoxy-1H-pyrrolo[2,3-b]pyridin-3-ylmethyl)-phenoxymethyl]-1H-benzoimidazole, 2-[3,5-Difluoro-4-(1H-pyrrolo[2,3-b]pyridin-3-ylmethyl)-phenoxymethyl]-1H-benzoimidazole,2-[5-Chloro-2-methoxy-4-(1H-pyrrolo[2,3-b]pyridin-3-ylmethyl)-phenoxymethyl]-1H-benzoimidazole, 2-[5-Chloro-4-(5-chloro-1H-pyrrolo[2,3-b]pyridin-3-ylmethyl)-2-methoxy-phenoxymethyl]-1H-benzoimidazole,2-[4-(5-Chloro-1H-pyrrolo[2,3-b]pyridin-3-ylmethyl)-3,5-difluoro-phenoxymethyl]-1H-benzoimidazole,2-[4-(5-Chloro-1H-pyrrolo[2,3-b]pyridin-3-ylmethyl)-5-fluoro-2-methoxy-phenoxymethyl]-1-methyl-1H-benzoimidazole,2-[4-(5-Bromo-1H-pyrrolo[2,3-b]pyridin-3-ylmethyl)-2,5-difluoro-phenoxymethyl]-1H-benzoimidazole,2-{2,5-Difluoro-4-[5-(1-methyl-1H-pyrazol-4-yl)-1H-pyrrolo[2,3-b]pyridin-3-ylmethyl]-phenoxymethyl}-1H-benzoimidazole,2-{5-Chloro-2-fluoro-4-[5-(1-methyl-1H-pyrazol-4-yl)-1H-pyrrolo[2,3-b]pyridin-3-ylmethyl]-phenoxymethyl}-1H-benzoimidazole,2-{1-[4-(5-Chloro-1H-pyrrolo[2,3-b]pyridin-3-ylmethyl)-5-fluoro-2-methoxy-phenoxyl]-ethyl}-1H-benzoimidazole,6-Chloro-2-[4-(5-chloro-1H-pyrrolo[2,3-b]pyridin-3-ylmethyl)-5-fluoro-2-methoxy-phenoxymethyl]-1H-benzoimidazole,6-Chloro-2-[5-fluoro-2-methoxy-4-(1H-pyrrolo[2,3-b]pyridin-3-ylmethyl)-phenoxymethyl]-1H-benzoimidazole,2-[5-Fluoro-2-methoxy-4-(1H-pyrrolo[2,3-b]pyridin-3-ylmethyl)-phenoxymethyl]-6-methoxy-1H-benzoimidazole,2-[5-Chloro-2-fluoro-4-(5-methoxy-1H-pyrrolo[2,3-b]pyridin-3-ylmethyl)-phenoxymethyl]-1H-benzoimidazole,2-[5-Fluoro-4-(5-fluoro-1H-pyrrolo[2,3-b]pyridin-3-ylmethyl)-2-methoxy-phenoxymethyl]-1H-benzoimidazole,2-[2-Chloro-5-fluoro-4-(5-methoxy-1H-pyrrolo[2,3-b]pyridin-3-ylmethyl)-phenoxymethyl]-1H-benzoimidazole,2-{2-Chloro-5-fluoro-4-[5-(1-methyl-1H-pyrazol-4-yl)-1H-pyrrolo[2,3-b]pyridin-3-ylmethyl]-phenoxymethyl}-1H-benzoimidazole,2-{4-[(5-Chloro-1H-pyrrolo[2,3-b]pyridin-3-yl)-methoxy-methyl]-5-fluoro-2-methoxy-phenoxymethyl}-1H-benzoimidazole,[4-(1H-Benzoimidazol-2-ylmethoxy)-2-fluoro-5-methoxy-phenyl]-(5-chloro-1H-pyrrolo[2,3-b]pyridin-3-yl)-methanone,2-[2,5-Difluoro-4-(5-methanesulfonyl-1H-pyrrolo[2,3-b]pyridin-3-ylmethyl)-phenoxymethyl]-1H-benzoimidazole,3-[4-(1H-Benzoimidazol-2-ylmethoxy)-2-fluoro-5-methoxy-benzyl]-1H-pyrrolo[2,3-b]pyridine-5-carbonitrile,5,6-Dichloro-2-[4-(5-chloro-1H-pyrrolo[2,3-b]pyridin-3-ylmethyl)-5-fluoro-2-methoxy-phenoxymethyl]-1H-benzoimidazole,2-[4-(5-Chloro-1H-pyrrolo[2,3-b]pyridin-3-ylmethyl)-5-fluoro-2-methoxy-phenoxymethyl]-1H-benzoimidazole-5-sulfonic acid dimethylamide,3-[4-(1H-Benzoimidazol-2-ylmethoxy)-2-fluoro-5-methoxy-benzyl]-1H-pyrrolo[2,3-b]pyridine-5-carboxylic acid methyl ester,3-[4-(1H-Benzoimidazol-2-ylmethoxy)-2-fluoro-5-methoxy-benzyl]-1H-pyrrolo[2,3-b]pyridine-5-carboxylic acid,2-{2,5-Difluoro-4-[5-(2-methoxy-ethoxy)-1H-pyrrolo[2,3-b]pyridin-3-ylmethyl]-phenoxymethyl}-1H-benzoimidazole,2-[4-(5-Chloro-1H-pyrrolo[2,3-b]pyridin-3-ylmethyl)-5-fluoro-2-methoxy-phenoxymethyl]-1-ethyl-1H-benzoimidazole,2-[4-(5-Chloro-1H-pyrrolo[2,3-b]pyridin-3-ylmethyl)-5-fluoro-2-methoxy-phenoxymethyl]-5-trifluoromethyl-1H-benzoimidazole,2-[4-(5-Chloro-1H-pyrrolo[2,3-b]pyridin-3-ylmethyl)-5-fluoro-2-methoxy-phenoxymethyl]-5-fluoro-1H-benzoimidazole,2-{2-[4-(5-Chloro-1H-pyrrolo[2,3-b]pyridin-3-ylmethyl)-5-fluoro-2-methoxy-phenoxy]-ethyl}-1H-benzoimidazole,2-[4-(5-Bromo-1H-pyrrolo[2,3-b]pyridin-3-ylmethyl)-5-fluoro-2-methoxy-phenoxymethyl]-1H-benzoimidazole,2-[4-(5-Chloro-1H-pyrrolo[2,3-b]pyridin-3-ylmethyl)-5-fluoro-2-methoxy-phenoxymethyl]-5-methoxy-1H-benzoimidazole,5-Chloro-2-[5-fluoro-2-methoxy-4-(5-methyl-1H-pyrrolo[2,3-b]pyridin-3-ylmethyl)-phenoxymethyl]-1-benzoimidazole,3-[4-(1H-Benzoimidazol-2-ylmethoxy)-2,5-difluoro-benzyl]-1H-pyrrolo[2,3-b]pyridine-5-carbonitrile, and 2-[5-Fluoro-4-(5-methanesulfonyl-1H-pyrrolo[2,3-b]pyridin-3-ylmethyl)-2-methoxy-phenoxymethyl]-1H-benzoimidazole, and all pharmaceutically acceptable salts, tautomers, and isomers thereof.

### Prostaglandin E Receptor 4 (PTGER4) Modulators

Disclosed herein, in some embodiments, are therapeutic agents comprising Prostaglandin E Receptor 4 (PTGER4) Modulators useful for the treatment of a disease or condition, or symptom of the disease or condition, disclosed herein. In some embodiments, the therapeutic agent comprises a modulator, agonist, and/or antagonist of PTGER4 (e.g., a PTGER4 having a sequence set forth in SEQ ID NO: 3). In some embodiments, PTGER4 refers to a peptide associated with one or more of the following reference sequences: HGNC: 9596 Entrez Gene: 5734 Ensembl: ENSG00000171522 OMIM: 601586 UniProtKB: P35408.

In some instances, the therapeutic agent comprises a modulator of PTGER4 activity or expression. In some instances, the modulator of PTGER4 activity or expression comprises an agonist or a partial agonist of PTGER4. In some instances, the modulator of PTGER4 activity or expression comprises an antagonist or a partial antagonist of PTGER4. In some instances, the agonist or partial agonist comprises an inverse antagonist. In some instances, the agonist or partial agonist comprises a positive allosteric modulator (PAM). In some instances, the agonist or partial agonist comprises a negative allosteric modulator (NAM).

In some instances, the PTGER4 modulator is effective to increase PTGER4 expression or activity in the subject (e.g., an agonist of PTGER4). The agonist of PTGER4 expression or activity may be a direct agonist or indirect agonist. In some embodiments, the agonist of PTGER4 expression or activity comprises a complete agonist or a partial agonist. Non-limiting examples of an agonist of PTGER4 expression include RNA to protein PTGER4 translation agonists, antisense oligonucleotides targeting the PTGER4, or homolog thereof, mRNA (such as miRNAs, or siRNA), epigenetic editing (such as post-translational modifications of histone tails and/or DNA molecules). Non-limiting examples of an agonist of PTGER4 activity include antagonists to the PTGER4 antigen, and antagonists to gene expression products involved in PTGER4 mediated disease.

In some instances, the PTGER4 modulator is effective to decrease PTGER4 expression or activity in the subject (e.g., an antagonist of PTGER4). The antagonist of PTGER4 expression or activity may be a direct antagonist or indirect antagonist. In some embodiments, the antagonist of PTGER4 expression or activity comprises a complete antagonist or a partial antagonist. Non-limiting examples of an antagonist of PTGER4 expression include RNA to protein PTGER4 translation antagonists, antisense oligonucleotides targeting the PTGER4, or homolog thereof, mRNA (such as miRNAs, or siRNA), epigenetic editing (such as post-translational modifications of histone tails and/or DNA molecules). Non-limiting examples of an antagonist of PTGER4 activity include antagonists to the PTGER4 antigen, and antagonists to gene expression products involved in PTGER4 mediated disease.

In some instances, the modulator of PTGER4 activity or expression (e.g., PTGER antagonist or agonists described herein) comprises an antibody or antigen-binding fragment, peptide, or small molecule. In some embodiments, the PTGER4 modulator is a small molecule. In some instances, the small molecule may be a small molecule that binds to PTGER4 or binding partners to PTGER4. In some embodiments, a small molecule has a molecular weight less than or equal to about 1000 Daltons. In some embodiments, a small molecule has a molecular weight less than or equal to about 900 Da. In some embodiments, a small molecule has a molecular weight less than or equal to about 800 Da. In some embodiments, a small molecule has a molecular weight less than or equal to about 700 Da. In some embodiments, a small molecule has a molecular weight less than or equal to about 600 Da. In some embodiments, a small molecule has a molecular weight less than or equal to about 500 Da. In some embodiments, the modulator of PTGER4 activity or expression comprises a compound selected from grapiprant (CJ-023,423), ONO-AE3-208, GW627368X, AH23848, ONO-AE2-227, ONO-AE1-734, AGN205203, rivenprost (ONO-4819), CJ-023,423, and BGC20-1531.

In some instances, the PTGER4 modulator (e.g., PTGER antagonist or agonists described herein) is an antibody. In some instances, the PTGER4 antibody may be monoclonal or polyclonal. In some instances, the PTGER4 antibody may be humanized or chimeric. The PTGER4 antibody may be a fusion protein. The PTGER4 antibody may be a blocking PTGER4 antibody. A blocking antibody blocks binding between two proteins, e.g., a ligand and its receptor. In a non-limiting example, the PTGER4 blocking antibody binds to a binding partner of PTGER4. In some cases, the PTGER4 antibody is an PTGER4 antibody that specifically binds to PTGER4.

### Ribonuclease T2 (RNASET2) Modulators

In some embodiments, the therapeutic agent comprises a modulator, agonist, or partial agonist of Ribonuclease T2 (RNASET2). In some embodiments, the agonist of RNASET2 comprises an RNASET2 polypeptide. In some embodiments, the RNASET2 polypeptide comprises a human RNASET2 protein (huRNASET2). In some embodiments, the RNASET2 polypeptide comprises a recombinant RNASET2 polypeptide. In some embodiments, the recombinant huRNASET2 protein comprises SEQ ID NO: 4 (MRPAALRGALLGCLCLALLCLGGADKRLRDNHEWKKLIMVQHWPETVCEKIQNDCRDPPDYW TIHGLWPDKSEGCNRSWPFNLEEIKDLLPEMRAYWPDVIHSFPNRSRFWKHEWEKHGTCAAQVD ALNSQKKYFGRSLELYRELDLNSVLLKLGIKPSINYYQVADFKDALARVYGVIPKIQCLPPSQDEE VQTIGQIELCLTKQDQQLQNCTEPGEQPSPKQEVWLANGAAESRGLRVCEDGPVFYPPPKKTKH), which is the amino acid sequence of human RNASET2 (NCBI Reference Sequence No. NP_003721.2). In some embodiments, the huRNASET2 comprises an amino acid sequence about 99%, 98%, 97%, 96%, 95%, 94%, 93%, 92%, 91%, or 90% homologous to SEQ ID NO: 4.

In some instances, the RNASET2 polypeptide is truncated. In some instances, the truncation is an N-terminal deletion. In other instances, the truncation is a C-terminal deletion. In additional instances, the truncation comprises both N-terminal and C-terminal deletions. For example, the truncation can be a deletion of at least or about 1, 2, 3, 4, 5, 6, 7, 8, 9, 10, 11, 12, 13, 14, 15, 20, or more residues from either the N-terminus or the C-terminus, or both termini. In some cases, the RNASET2 polypeptide comprises an N-terminal deletion of at least or about 1, 2, 3, 4, 5, 6, 7, 8, 9, 10, 11, 12, 13, 14, 15, 20, or more residues. In some cases, the RNASET2 polypeptide comprises an N-terminal deletion of at least or about 1, 2, 3, 4, 5, 6, 7, 8, 9, or 10 residues. In some cases, the RNASET2 polypeptide comprises an N-terminal deletion of at least or about 2 residues. In some cases, the RNASET2 polypeptide comprises an N-terminal deletion of at least or about 3 residues. In some cases, the RNASET2 polypeptide comprises an N-terminal deletion of at least or about 4 residues. In some cases, the RNASET2 polypeptide comprises an N-terminal deletion of at least or about 5 residues. In some cases, the RNASET2 polypeptide comprises an N-terminal deletion of at least or about 6 residues. In some cases, the RNASET2 polypeptide comprises an N-terminal deletion of at least or about 7 residues. In some cases, the RNASET2 polypeptide comprises an N-terminal deletion of at least or about 8 residues. In some cases, the RNASET2 polypeptide comprises an N-terminal deletion of at least or about 9 residues. In some cases, the RNASET2 polypeptide comprises an N-terminal deletion of at least or about 10 residues. In some embodiments, the truncated RNASET2 has reduced or ameliorated ribonucleolytic activity. Non-limiting examples of truncated RNASET2 polypeptides include hrtrRNASE-70 (MRAYWPDVIH SFPNRSRFWK HEWEKHGTCA AQVDALNSQK KYFGRSLELYRELDLNSVLL KLGIKPSINY YQVADFKDAL ARVYGVIPKI QCLPPSQDEEVQTIGQIELC LTKQDQQLQN CTEPGEQPSP KQEVWLANGA AESRGLRVCE DGPVFYPPPK KTKH; SEQ ID NO: 6), and hrtrRNASE-50 (EGCNRSWPFN LEEIKDLLPE MRAYWPDVIH SFPNRSRFWK HEWEKHGTCAAQVDALNSQK KYFGRSLELY RELDLNSVLL KLGIKPSINY YQVADFKDALARVYGVIPKI QCLPPSQDEE VQTIGQIELC LTKQDQQLQN CTEPGEQPSPKQEVWLANGA AESRGLRVCE DGPVFYPPPK KTKH; SEQ ID NO: 5). In some instances, the truncated RNASET2 has functionally active ribonucleolytic activity. In some instances, the RNASET2 polypeptide has an internal deletion or substitution.

In some embodiments, the RNASET2 polypeptide has an enhanced plasma half-life. In some instances, the plasma half-life comprises at least 30 minutes, 45 minutes, 60 minutes, 75 minutes, or 90 minutes, 2 hours, 3 hours, 4 hours, 5 hours, 6 hours, 7 hours, 8 hours, 9 hours, 10 hours, 11 hours, 12 hours, 18 hours, 24 hours, 36 hours, 48 hours, 3 days, 4 days, 5 days, 6 days, 7 days, 10 days, 12 days, 14 days, 21 days, 28 days, 30 days, or longer than the plasma half-life of the wild-type RNASET2 protein.

In some embodiments, the RNASET2 polypeptide is a conjugate. In some embodiments, the RNASET2 conjugate comprises an RNASET2 polypeptide comprising at least one amino acid and a conjugating moiety bound to the at least one 1 amino acid. In some embodiments, the at least one amino acid is located proximal to the N-terminus (e.g., proximal to the N-terminal residue). For example, the at least one amino acid is located optionally within the first 10, 20, 30, 40, or 50 residues from the N-terminus. In some cases, the at least one amino acid is located at the N-terminus (i.e., the at least one amino acid is the N-terminal residue of the RNASET2 polypeptide). In other embodiments, the at least one amino acid is located proximal to the C-terminus (e.g., proximal to the C-terminal residue). For example, the at least one amino acid is located optionally within the first 10, 20, 30, 40, or 50 residues from the C-terminus. In some cases, the at least one amino acid is located at the C-terminus (i.e., the at least one amino acid is the C-terminal residue of the RNASET2 polypeptide). In some instances, the RNASET2 conjugate has an enhanced plasma half-life, such as the half-lifes described herein. In some embodiments, the RNASET2 conjugate is functionally active (e.g., retains ribonucleolytic activity). In some embodiments, the RNASET2 conjugate is not functionally active (e.g., devoid of ribonucleolytic activity). In some embodiments, the conjugating moiety comprises a polymer comprising Polyethylene glycol (PEG).

In some embodiments, the RNASET2 polypeptide is fused with a second polypeptide. In some embodiments, the second polypeptide comprises a polypeptide with a long plasma half-life relative to the plasma half-life of the RNASET2 polypeptide. In some embodiments, the second polypeptide comprises an antibody or antibody fragment. In some embodiments, the antibody or antibody fragment comprises an IgG1, IgG2, IgG4, IgG3, or IgE. In some embodiments, the IgG is an Fc. In some embodiments, the IgG Fc is human. In some instances, the long plasma half-life polypeptide comprises HSA, transferrin, IgA monomer, Retinol-binding protein, Factor H, Factor XIII, C-reactive protein, Factor IX, Fibrinogen, IFN-alpha, Pentameric IgM, IL-2, or Thyroglobulin. In some instances, the RNASET2-Fc comprises RSLV-132.

### TNF Superfamily Member 15 (TL1A) TL1A Modulators

In some embodiments, the therapeutic agent comprises a modulator and/or antagonist of TNF Superfamily Member 15 (TL1A), or the gene encoding TL1A (TNFSF15). In some embodiments, the modulator of TL1A is an antagonist of TL1A. In some embodiments the therapeutic agent or the additional therapeutic agent comprises an inhibitor of TL1A expression or activity. In some embodiments the therapeutic agent comprises an inhibitor of TL1A expression or activity. In some cases, the inhibitor of TL1A expression or activity is effective to inhibit TL1A-DR3 binding. In some embodiments, the inhibitor of TL1A expression or activity comprises an allosteric modulator of TL1A. An allosteric modulator of TL1A may indirectly influence the effects TL1A on DR3, or TR6/DcR3 on TL1A or DR3. The inhibitor of TL1A expression or activity may be a direct inhibitor or indirect inhibitor. Non-limiting examples of an inhibitor of TL1A expression include RNA to protein TL1A translation inhibitors, antisense oligonucleotides targeting the TNFSF15 mRNA (such as miRNAs, or siRNA), epigenetic editing (such as targeting the DNA-binding domain of TNFSF15, or post-translational modifications of histone tails and/or DNA molecules). Non-limiting examples of an inhibitor of TL1A activity include antagonists to the TL1A receptors, (DR3 and TR6/DcR3), antagonists to TL1A antigen, and antagonists to gene expression products involved in TL1A mediated disease. Antagonists as disclosed herein, may include, but are not limited to, an anti-TL1A antibody, an anti-TL1A-binding antibody fragment, or a small molecule. The small molecule may be a small molecule that binds to TL1A or DR3. The anti-TL1A antibody may be monoclonal or polyclonal. The anti-TL1A antibody may be humanized or chimeric. The anti-TL1A antibody may be a fusion protein. The anti-TL1A antibody may be a blocking anti-TL1A antibody. A blocking antibody blocks binding between two proteins, e.g., a ligand and its receptor. Therefore, a TL1A blocking antibody includes an antibody that prevents binding of TL1A to DR3 or TR6/DcR3 receptors. In a non-limiting example, the TL1A blocking antibody binds to DR3. In another example, the TL1A blocking antibody binds to DcR3. In some cases, the anti-TL1A antibody is an anti-TL1A antibody that specifically binds to TL1A.

The anti-TL1A antibody may comprise one or more of the antibody sequences of Table 8. The anti-DR3 antibody may comprise an amino acid sequence that is at least 85% identical to any one of SEQ ID NOS: 358-370 and an amino acid sequence that is at least 85% identical to any one of SEQ ID NOS: 371-375. The anti-DR3 antibody may comprise an amino acid sequence comprising the HCDR1, HCDR2, HCDR3 domains of any one of SEQ ID NOS: 358-370 and the LCDR1, LCDR2, and LCDR3 domains of any one of SEQ ID NOS: 371-375.

In some embodiments, an anti-TL1A antibody comprises a heavy chain comprising three complementarity-determining regions: HCDR1, HCDR2, and HCDR3; and a light chain comprising three complementarity-determining regions: LCDR1, LCDR2, and LCDR3. In some embodiments, the anti-TL1A antibody comprises a HCDR1 comprising SEQ ID NO: 209, a HCDR2 comprising SEQ ID NO: 210, a HCDR3 comprising SEQ ID NO: 211, a LCDR1 comprising SEQ ID NO: 212, a LCDR2 comprising SEQ ID NO: 213, and a LCDR3 comprising SEQ ID NO: 214. In some cases, the anti-TL1A antibody comprises a heavy chain (HC) variable domain comprising SEQ ID NO: 215 and a light chain (LC) variable domain comprising SEQ ID NO: 216.

In some embodiments, the anti-TL1A antibody comprises a HCDR1 comprising SEQ ID NO: 217, a HCDR2 comprising SEQ ID NO: 218, a HCDR3 comprising SEQ ID NO: 219, a LCDR1 comprising SEQ ID NO: 220, a LCDR2 comprising SEQ ID NO: 221, and a LCDR3 comprising SEQ ID NO: 222. In some cases, the anti-TL1A antibody comprises a heavy chain (HC) variable domain comprising SEQ ID NO: 223 and a light chain (LC) variable domain comprising SEQ ID NO: 224.

In some embodiments, the anti-TL1A antibody comprises a HCDR1 comprising SEQ ID NO: 225, a HCDR2 comprising SEQ ID NO: 226, a HCDR3 comprising SEQ ID NO: 227, a LCDR1 comprising SEQ ID NO: 228, a LCDR2 comprising SEQ ID NO: 229, and a LCDR3 comprising SEQ ID NO: 230. In some cases, the anti-TL1A antibody comprises a heavy chain (HC) variable domain comprising SEQ ID NO: 231 and a light chain (LC) variable domain comprising SEQ ID NO: 232.

In some embodiments, the anti-TL1A antibody comprises a HCDR1 comprising SEQ ID NO: 233, a HCDR2 comprising SEQ ID NO: 234, a HCDR3 comprising SEQ ID NO: 235, a LCDR1 comprising SEQ ID NO: 239, a LCDR2 comprising SEQ ID NO: 240, and a LCDR3 comprising SEQ ID NO: 241. In some cases, the anti-TL1A antibody comprises a HCDR1 comprising SEQ ID NO: 236, a HCDR2 comprising SEQ ID NO: 237, a HCDR3 comprising SEQ ID NO: 238, a LCDR1 comprising SEQ ID NO: 239, a LCDR2 comprising SEQ ID NO: 240, and a LCDR3 comprising SEQ ID NO: 241. In some cases, the anti-TL1A antibody comprises a heavy chain (HC) variable domain comprising SEQ ID NO: 242 and a light chain (LC) variable domain comprising SEQ ID NO: 243. In some cases, the anti-TL1A antibody comprises a heavy chain comprising SEQ ID NO: 244. In some cases, the anti-TL1A antibody comprises a light chain comprising SEQ ID NO: 245.

In some embodiments, the anti-TL1A antibody comprises a HCDR1 comprising SEQ ID NO: 246, a HCDR2 comprising SEQ ID NO: 247, a HCDR3 comprising SEQ ID NO: 248, a LCDR1 comprising SEQ ID NO: 249, a LCDR2 comprising SEQ ID NO: 250, and a LCDR3 comprising SEQ ID NO: 251. In some cases, the anti-TL1A antibody comprises a heavy chain (HC) variable domain comprising SEQ ID NO: 252 and a light chain (LC) variable domain comprising SEQ ID NO: 253.

In some embodiments, the anti-TL1A antibody comprises a HCDR1 comprising SEQ ID NO: 254, a HCDR2 comprising SEQ ID NO: 255, a HCDR3 comprising SEQ ID NO: 256, a LCDR1 comprising SEQ ID NO: 257, a LCDR2 comprising SEQ ID NO: 258, and a LCDR3 comprising SEQ ID NO: 259. In some cases, the anti-TL1A antibody comprises a heavy chain (HC) variable domain comprising SEQ ID NO: 260 and a light chain (LC) variable domain comprising SEQ ID NO: 261.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 267, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 271 and a light chain (LC) variable domain comprising SEQ ID NO: 275. In some cases, the anti-TL1A antibody comprises a heavy chain (HC) variable domain comprising SEQ ID NO: 271 and a light chain (LC) variable domain comprising SEQ ID NO: 276. In some cases, the anti-TL1A antibody comprises a heavy chain (HC) variable domain comprising SEQ ID NO: 271 and a light chain (LC) variable domain comprising SEQ ID NO: 277. In some cases, the anti-TL1A antibody comprises a heavy chain (HC) variable domain comprising SEQ ID NO: 271 and a light chain (LC) variable domain comprising SEQ ID NO: 278.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 268, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 271 and a light chain (LC) variable domain comprising SEQ ID NO: 279. In some cases, the anti-TL1A antibody comprises a heavy chain (HC) variable domain comprising SEQ ID NO: 271 and a light chain (LC) variable domain comprising SEQ ID NO: 280. In some cases, the anti-TL1A antibody comprises a heavy chain (HC) variable domain comprising SEQ ID NO: 271 and a light chain (LC) variable domain comprising SEQ ID NO: 281. In some cases, the anti-TL1A antibody comprises a heavy chain (HC) variable domain comprising SEQ ID NO: 271 and a light chain (LC) variable domain comprising SEQ ID NO: 282.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 267, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 272 and a light chain (LC) variable domain comprising SEQ ID NO: 275. In some cases, the anti-TL1A antibody comprises a heavy chain (HC) variable domain comprising SEQ ID NO: 272 and a light chain (LC) variable domain comprising SEQ ID NO: 276. In some cases, the anti-TL1A antibody comprises a heavy chain (HC) variable domain comprising SEQ ID NO: 272 and a light chain (LC) variable domain comprising SEQ ID NO: 277. In some cases, the anti-TL1A antibody comprises a heavy chain (HC) variable domain comprising SEQ ID NO: 272 and a light chain (LC) variable domain comprising SEQ ID NO: 278.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 268, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 272 and a light chain (LC) variable domain comprising SEQ ID NO: 279. In some cases, the anti-TL1A antibody comprises a heavy chain (HC) variable domain comprising SEQ ID NO: 272 and a light chain (LC) variable domain comprising SEQ ID NO: 280. In some cases, the anti-TL1A antibody comprises a heavy chain (HC) variable domain comprising SEQ ID NO: 272 and a light chain (LC) variable domain comprising SEQ ID NO: 281. In some cases, the anti-TL1A antibody comprises a heavy chain (HC) variable domain comprising SEQ ID NO: 272 and a light chain (LC) variable domain comprising SEQ ID NO: 282.

In some embodiments, the anti-TL1A antibody comprises a HCDR1 comprising SEQ ID NO: 263, a HCDR2 comprising SEQ ID NO: 264, a HCDR3 comprising SEQ ID NO: 266, a LCDR1 comprising SEQ ID NO: 267, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 273 and a light chain (LC) variable domain comprising SEQ ID NO: 275. In some cases, the anti-TL1A antibody comprises a heavy chain (HC) variable domain comprising SEQ ID NO: 273 and a light chain (LC) variable domain comprising SEQ ID NO: 276. In some cases, the anti-TL1A antibody comprises a heavy chain (HC) variable domain comprising SEQ ID NO: 273 and a light chain (LC) variable domain comprising SEQ ID NO: 277. In some cases, the anti-TL1A antibody comprises a heavy chain (HC) variable domain comprising SEQ ID NO: 273 and a light chain (LC) variable domain comprising SEQ ID NO: 278. In some cases, the anti-TL1A antibody comprises a heavy chain (HC) variable domain comprising SEQ ID NO: 273 and a light chain (LC) variable domain comprising SEQ ID NO: 279. In some cases, the anti-TL1A antibody comprises a heavy chain (HC) variable domain comprising SEQ ID NO: 273 and a light chain (LC) variable domain comprising SEQ ID NO: 280. In some cases, the anti-TL1A antibody comprises a heavy chain (HC) variable domain comprising SEQ ID NO: 273 and a light chain (LC) variable domain comprising SEQ ID NO: 281. In some cases, the anti-TL1A antibody comprises a heavy chain (HC) variable domain comprising SEQ ID NO: 273 and a light chain (LC) variable domain comprising SEQ ID NO: 282.

In some embodiments, the anti-TL1A antibody comprises a HCDR1 comprising SEQ ID NO: 263, a HCDR2 comprising SEQ ID NO: 264, a HCDR3 comprising SEQ ID NO: 266, a LCDR1 comprising SEQ ID NO: 268, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 274 and a light chain (LC) variable domain comprising SEQ ID NO: 279. In some cases, the anti-TL1A antibody comprises a heavy chain (HC) variable domain comprising SEQ ID NO: 274 and a light chain (LC) variable domain comprising SEQ ID NO: 280. In some cases, the anti-TL1A antibody comprises a heavy chain (HC) variable domain comprising SEQ ID NO: 274 and a light chain (LC) variable domain comprising SEQ ID NO: 281. In some cases, the anti-TL1A antibody comprises a heavy chain (HC) variable domain comprising SEQ ID NO: 274 and a light chain (LC) variable domain comprising SEQ ID NO: 282. In some cases, the anti-TL1A antibody comprises a heavy chain (HC) variable domain comprising SEQ ID NO: 274 and a light chain (LC) variable domain comprising SEQ ID NO: 275. In some cases, the anti-TL1A antibody comprises a heavy chain (HC) variable domain comprising SEQ ID NO: 274 and a light chain (LC) variable domain comprising SEQ ID NO: 276. In some cases, the anti-TL1A antibody comprises a heavy chain (HC) variable domain comprising SEQ ID NO: 274 and a light chain (LC) variable domain comprising SEQ ID NO: 277. In some cases, the anti-TL1A antibody comprises a heavy chain (HC) variable domain comprising SEQ ID NO: 274 and a light chain (LC) variable domain comprising SEQ ID NO: 278.

In some embodiments, the anti-TL1A antibody comprises a HCDR1 comprising SEQ ID NO: 283, a HCDR2 comprising SEQ ID NO: 284, a HCDR3 comprising SEQ ID NO: 285, a LCDR1 comprising SEQ ID NO: 286, a LCDR2 comprising SEQ ID NO: 287, and a LCDR3 comprising SEQ ID NO: 288. In some cases, the anti-TL1A antibody comprises a heavy chain (HC) variable domain comprising SEQ ID NO: 289 and a light chain (LC) variable domain comprising SEQ ID NO: 294. In some cases, the anti-TL1A antibody comprises a heavy chain (HC) variable domain comprising SEQ ID NO: 289 and a light chain (LC) variable domain comprising SEQ ID NO: 295. In some cases, the anti-TL1A antibody comprises a heavy chain (HC) variable domain comprising SEQ ID NO: 289 and a light chain (LC) variable domain comprising SEQ ID NO: 296. In some cases, the anti-TL1A antibody comprises a heavy chain (HC) variable domain comprising SEQ ID NO: 289 and a light chain (LC) variable domain comprising SEQ ID NO: 297. In some cases, the anti-TL1A antibody comprises a heavy chain (HC) variable domain comprising SEQ ID NO: 290 and a light chain (LC) variable domain comprising SEQ ID NO: 294. In some cases, the anti-TL1A antibody comprises a heavy chain (HC) variable domain comprising SEQ ID NO: 290 and a light chain (LC) variable domain comprising SEQ ID NO: 295. In some cases, the anti-TL1A antibody comprises a heavy chain (HC) variable domain comprising SEQ ID NO: 290 and a light chain (LC) variable domain comprising SEQ ID NO: 296. In some cases, the anti-TL1A antibody comprises a heavy chain (HC) variable domain comprising SEQ ID NO: 290 and a light chain (LC) variable domain comprising SEQ ID NO: 297. In some cases, the anti-TL1A antibody comprises a heavy chain (HC) variable domain comprising SEQ ID NO: 291 and a light chain (LC) variable domain comprising SEQ ID NO: 294. In some cases, the anti-TL1A antibody comprises a heavy chain (HC) variable domain comprising SEQ ID NO: 291 and a light chain (LC) variable domain comprising SEQ ID NO: 295. In some cases, the anti-TL1A antibody comprises a heavy chain (HC) variable domain comprising SEQ ID NO: 291 and a light chain (LC) variable domain comprising SEQ ID NO: 296. In some cases, the anti-TL1A antibody comprises a heavy chain (HC) variable domain comprising SEQ ID NO: 291 and a light chain (LC) variable domain comprising SEQ ID NO: 297. In some cases, the anti-TL1A antibody comprises a heavy chain (HC) variable domain comprising SEQ ID NO: 292 and a light chain (LC) variable domain comprising SEQ ID NO: 294. In some cases, the anti-TL1A antibody comprises a heavy chain (HC) variable domain comprising SEQ ID NO: 292 and a light chain (LC) variable domain comprising SEQ ID NO: 295. In some cases, the anti-TL1A antibody comprises a heavy chain (HC) variable domain comprising SEQ ID NO: 292 and a light chain (LC) variable domain comprising SEQ ID NO: 296. In some cases, the anti-TL1A antibody comprises a heavy chain (HC) variable domain comprising SEQ ID NO: 292 and a light chain (LC) variable domain comprising SEQ ID NO: 297. In some cases, the anti-TL1A antibody comprises a heavy chain (HC) variable domain comprising SEQ ID NO: 293 and a light chain (LC) variable domain comprising SEQ ID NO: 294. In some cases, the anti-TL1A antibody comprises a heavy chain (HC) variable domain comprising SEQ ID NO: 293 and a light chain (LC) variable domain comprising SEQ ID NO: 295. In some cases, the anti-TL1A antibody comprises a heavy chain (HC) variable domain comprising SEQ ID NO: 293 and a light chain (LC) variable domain comprising SEQ ID NO: 296. In some cases, the anti-TL1A antibody comprises a heavy chain (HC) variable domain comprising SEQ ID NO: 293 and a light chain (LC) variable domain comprising SEQ ID NO: 297.

In some embodiments, the anti-TL1A antibody comprises a HCDR1 comprising SEQ ID NO: 298, a HCDR2 comprising SEQ ID NO: 299, a HCDR3 comprising SEQ ID NO: 300, a LCDR1 comprising SEQ ID NO: 301, a LCDR2 comprising SEQ ID NO: 302, and a LCDR3 comprising SEQ ID NO: 303. In some cases, the anti-TL1A antibody comprises a heavy chain (HC) variable domain comprising SEQ ID NO: 304 and a light chain (LC) variable domain comprising SEQ ID NO: 305. In some cases, the anti-TL1A antibody comprises a heavy chain (HC) variable domain comprising SEQ ID NO: 306 and a light chain (LC) variable domain comprising SEQ ID NO: 307. In some cases, the anti-TL1A antibody comprises a heavy chain (HC) variable domain comprising SEQ ID NO: 308 and a light chain (LC) variable domain comprising SEQ ID NO: 309. In some cases, the anti-TL1A antibody comprises a heavy chain (HC) variable domain comprising SEQ ID NO: 310 and a light chain (LC) variable domain comprising SEQ ID NO: 311. In some cases, the anti-TL1A antibody comprises a heavy chain (HC) variable domain comprising SEQ ID NO: 312 and a light chain (LC) variable domain comprising SEQ ID NO: 313. In some cases, the anti-TL1A antibody comprises a heavy chain (HC) variable domain comprising SEQ ID NO: 314 and a light chain (LC) variable domain comprising SEQ ID NO: 315. In some cases, the anti-TL1A antibody comprises a heavy chain (HC) variable domain comprising SEQ ID NO: 316 and a light chain (LC) variable domain comprising SEQ ID NO: 317. In some cases, the anti-TL1A antibody comprises a heavy chain (HC) variable domain comprising SEQ ID NO: 318 and a light chain (LC) variable domain comprising SEQ ID NO: 319. In some cases, the anti-TL1A antibody comprises a heavy chain (HC) variable domain comprising SEQ ID NO: 320 and a light chain (LC) variable domain comprising SEQ ID NO: 321. In some cases, the anti-TL1A antibody comprises a heavy chain (HC) variable domain comprising SEQ ID NO: 322 and a light chain (LC) variable domain comprising SEQ ID NO: 323. In some cases, the anti-TL1A antibody comprises a heavy chain (HC) variable domain comprising SEQ ID NO: 324 and a light chain (LC) variable domain comprising SEQ ID NO: 325. In some cases, the anti-TL1A antibody comprises a heavy chain (HC) variable domain comprising SEQ ID NO: 326 and a light chain (LC) variable domain comprising SEQ ID NO: 327.

In some embodiments, the anti-TL1A antibody comprises a HCDR1 comprising SEQ ID NO: 328, a HCDR2 comprising SEQ ID NO: 329, a HCDR3 comprising SEQ ID NO: 330, a LCDR1 comprising SEQ ID NO: 331, a LCDR2 comprising SEQ ID NO: 332, and a LCDR3 comprising SEQ ID NO: 333. In some cases, the anti-TL1A antibody comprises a heavy chain (HC) variable domain comprising SEQ ID NO: 334 and a light chain (LC) variable domain comprising SEQ ID NO: 335.

In some embodiments, the anti-TL1A antibody comprises a HCDR1 comprising SEQ ID NO: 336, a HCDR2 comprising SEQ ID NO: 337, a HCDR3 comprising SEQ ID NO: 338, a LCDR1 comprising SEQ ID NO: 339, a LCDR2 comprising SEQ ID NO: 340, and a LCDR3 comprising SEQ ID NO: 341. In some cases, the anti-TL1A antibody comprises a heavy chain (HC) variable domain comprising SEQ ID NO: 342 and a light chain (LC) variable domain comprising SEQ ID NO: 343.

In some embodiments, the anti-TL1A antibody comprises a HCDR1 comprising SEQ ID NO: 346, a HCDR2 comprising SEQ ID NO: 347, a HCDR3 comprising SEQ ID NO: 348, a LCDR1 comprising SEQ ID NO: 349, a LCDR2 comprising SEQ ID NO: 350, and a LCDR3 comprising SEQ ID NO: 351. In some cases, the anti-TL1A antibody comprises a heavy chain (HC) variable domain comprising SEQ ID NO: 344 and a light chain (LC) variable domain comprising SEQ ID NO: 345. In some cases, the anti-TL1A antibody comprises a heavy chain (HC) variable domain comprising SEQ ID NO: 352 and a light chain (LC) variable domain comprising SEQ ID NO: 353. In some cases, the anti-TL1A antibody comprises a heavy chain (HC) variable domain comprising SEQ ID NO: 354 and a light chain (LC) variable domain comprising SEQ ID NO: 355. In some cases, the anti-TL1A antibody comprises a heavy chain (HC) variable domain comprising SEQ ID NO: 356 and a light chain (LC) variable domain comprising SEQ ID NO: 357.

In some embodiments, the anti-TL1A antibody comprises a HCDR1 comprising SEQ ID NO: 376, a HCDR2 comprising SEQ ID NO: 377, a HCDR3 comprising SEQ ID NO: 378, a LCDR1 comprising SEQ ID NO: 379, a LCDR2 comprising SEQ ID NO: 380, and a LCDR3 comprising SEQ ID NO: 381. In some cases, the anti-TL1A antibody comprises a heavy chain (HC) variable domain comprising SEQ ID NO: 382 and a light chain (LC) variable domain comprising SEQ ID NO: 383.

In some embodiments, the anti-TL1A antibody comprises a HCDR1 comprising SEQ ID NO: 384, a HCDR2 comprising SEQ ID NO: 385, a HCDR3 comprising SEQ ID NO: 386, a LCDR1 comprising SEQ ID NO: 387, a LCDR2 comprising SEQ ID NO: 388, and a LCDR3 comprising SEQ ID NO: 399. In some cases, the anti-TL1A antibody comprises a heavy chain (HC) variable domain comprising SEQ ID NO: 390 and a light chain (LC) variable domain comprising SEQ ID NO: 391.

In some embodiments, the anti-TL1A antibody comprises one or more of A101-A177 of Table 2. In some embodiments, the anti-TL1A antibody is A100. In some embodiments, the anti-TL1A antibody is A101. In some embodiments, the anti-TL1A antibody is A102. In some embodiments, the anti-TL1A antibody is A103. In some embodiments, the anti-TL1A antibody is A104. In some embodiments, the anti-TL1A antibody is A105. In some embodiments, the anti-TL1A antibody is A106. In some embodiments, the anti-TL1A antibody is A107. In some embodiments, the anti-TL1A antibody is A108. In some embodiments, the anti-TL1A antibody is A109. In some embodiments, the anti-TL1A antibody is A110. In some embodiments, the anti-TL1A antibody is A111. In some embodiments, the anti-TL1A antibody is A112. In some embodiments, the anti-TL1A antibody is A113. In some embodiments, the anti-TL1A antibody is A114. In some embodiments, the anti-TL1A antibody is A115. In some embodiments, the anti-TL1A antibody is A116. In some embodiments, the anti-TL1A antibody is A117. In some embodiments, the anti-TL1A antibody is A118. In some embodiments, the anti-TL1A antibody is A119. In some embodiments, the anti-TL1A antibody is A120. In some embodiments, the anti-TL1A antibody is A121. In some embodiments, the anti-TL1A antibody is A122. In some embodiments, the anti-TL1A antibody is A123. In some embodiments, the anti-TL1A antibody is A124. In some embodiments, the anti-TL1A antibody is A125. In some embodiments, the anti-TL1A antibody is A126. In some embodiments, the anti-TL1A antibody is A127. In some embodiments, the anti-TL1A antibody is A128. In some embodiments, the anti-TL1A antibody is A129. In some embodiments, the anti-TL1A antibody is A130. In some embodiments, the anti-TL1A antibody is A131. In some embodiments, the anti-TL1A antibody is A132. In some embodiments, the anti-TL1A antibody is A133. In some embodiments, the anti-TL1A antibody is A134. In some embodiments, the anti-TL1A antibody is A135. In some embodiments, the anti-TL1A antibody is A136. In some embodiments, the anti-TL1A antibody is A137. In some embodiments, the anti-TL1A antibody is A138. In some embodiments, the anti-TL1A antibody is A139. In some embodiments, the anti-TL1A antibody is A140. In some embodiments, the anti-TL1A antibody is A141. In some embodiments, the anti-TL1A antibody is A142. In some embodiments, the anti-TL1A antibody is A143. In some embodiments, the anti-TL1A antibody is A144. In some embodiments, the anti-TL1A antibody is A145. In some embodiments, the anti-TL1A antibody is A146. In some embodiments, the anti-TL1A antibody is A147. In some embodiments, the anti-TL1A antibody is A148. In some embodiments, the anti-TL1A antibody is A149. In some embodiments, the anti-TL1A antibody is A150. In some embodiments, the anti-TL1A antibody is A151. In some embodiments, the anti-TL1A antibody is A152. In some embodiments, the anti-TL1A antibody is A153. In some embodiments, the anti-TL1A antibody is A154. In some embodiments, the anti-TL1A antibody is A155. In some embodiments, the anti-TL1A antibody is A156. In some embodiments, the anti-TL1A antibody is A157. In some embodiments, the anti-TL1A antibody is A158. In some embodiments, the anti-TL1A antibody is A159. In some embodiments, the anti-TL1A antibody is A160. In some embodiments, the anti-TL1A antibody is A161. In some embodiments, the anti-TL1A antibody is A162. In some embodiments, the anti-TL1A antibody is A163. In some embodiments, the anti-TL1A antibody is A164. In some embodiments, the anti-TL1A antibody is A165. In some embodiments, the anti-TL1A antibody is A166. In some embodiments, the anti-TL1A antibody is A167. In some embodiments, the anti-TL1A antibody is A168. In some embodiments, the anti-TL1A antibody is A169. In some embodiments, the anti-TL1A antibody is A170. In some embodiments, the anti-TL1A antibody is A171. In some embodiments, the anti-TL1A antibody is A172. In some embodiments, the anti-TL1A antibody is A173. In some embodiments, the anti-TL1A antibody is A174. In some embodiments, the anti-TL1A antibody is A175. In some embodiments, the anti-TL1A antibody is A176. In some embodiments, the anti-TL1A antibody is A177.

In some embodiments, the anti-DR3 is A178. In some embodiments, the anti-DR3 is A179. In some embodiments, the anti-DR3 is A180. In some embodiments, the anti-DR3 is A181. In some embodiments, the anti-DR3 is A182. In some embodiments, the anti-DR3 is A183. In some embodiments, the anti-DR3 is A184. In some embodiments, the anti-DR3 is A185. In some embodiments, the anti-DR3 is A186. In some embodiments, the anti-DR3 is A187. In some embodiments, the anti-DR3 is A188. In some embodiments, the anti-DR3 is A189. In some embodiments, the anti-DR3 is A190. In some embodiments, the anti-DR3 is A191. In some embodiments, the anti-DR3 is A192. In some embodiments, the anti-DR3 is A193. In some embodiments, the anti-DR3 is A194. In some embodiments, the anti-DR3 is A195. In some embodiments, the anti-DR3 is A196. In some embodiments, the anti-DR3 is A197. In some embodiments, the anti-DR3 is A198. In some embodiments, the anti-DR3 is A199. In some embodiments, the anti-DR3 is A200. In some embodiments, the anti-DR3 is A201. In some embodiments, the anti-DR3 is A202. In some embodiments, the anti-DR3 is A203. In some embodiments, the anti-DR3 is A204. In some embodiments, the anti-DR3 is A205. In some embodiments, the anti-DR3 is A206. In some embodiments, the anti-DR3 is A207. In some embodiments, the anti-DR3 is A208. In some embodiments, the anti-DR3 is A209. In some embodiments, the anti-DR3 is A210. In some embodiments, the anti-DR3 is A211. In some embodiments, the anti-DR3 is A212. In some embodiments, the anti-DR3 is A213. In some embodiments, the anti-DR3 is A214. In some embodiments, the anti-DR3 is A215. In some embodiments, the anti-DR3 is A216. In some embodiments, the anti-DR3 is A217. In some embodiments, the anti-DR3 is A218. In some embodiments, the anti-DR3 is A219. In some embodiments, the anti-DR3 is A220. In some embodiments, the anti-DR3 is A221. In some embodiments, the anti-DR3 is A222. In some embodiments, the anti-DR3 is A223. In some embodiments, the anti-DR3 is A224. In some embodiments, the anti-DR3 is A225. In some embodiments, the anti-DR3 is A226. In some embodiments, the anti-DR3 is A227. In some embodiments, the anti-DR3 is A228. In some embodiments, the anti-DR3 is A229. In some embodiments, the anti-DR3 is A230. In some embodiments, the anti-DR3 is A231. In some embodiments, the anti-DR3 is A232. In some embodiments, the anti-DR3 is A233. In some embodiments, the anti-DR3 is A234. In some embodiments, the anti-DR3 is A235. In some embodiments, the anti-DR3 is A236. In some embodiments, the anti-DR3 is A237. In some embodiments, the anti-DR3 is A238. In some embodiments, the anti-DR3 is A239. In some embodiments, the anti-DR3 is A240. In some embodiments, the anti-DR3 is A241. In some embodiments, the anti-DR3 is A242.

TABLE 2
Non-Limiting Examples of anti-TL1A and anti-DR3 Antibodies

| Antibody | HC Variable Domain | LC Variable Domain |
|---|---|---|
| Name | (SEQ ID NO) | (SEQ ID NO) |
| A100 | 215 | 216 |
| A101 | 223 | 224 |
| A102 | 231 | 232 |
| A103 | 242 | 243 |
| A104 | 252 | 253 |
| A105 | 260 | 261 |
| A106 | 271 | 275 |
| A107 | 271 | 276 |
| A108 | 271 | 277 |
| A109 | 271 | 278 |
| A110 | 271 | 279 |
| A111 | 271 | 280 |
| A112 | 271 | 281 |
| A113 | 271 | 282 |
| A114 | 272 | 275 |
| A115 | 272 | 276 |
| A116 | 272 | 277 |
| A117 | 272 | 278 |
| A118 | 272 | 279 |
| A119 | 272 | 280 |
| A120 | 272 | 281 |
| A121 | 272 | 282 |
| A122 | 273 | 275 |
| A123 | 273 | 276 |
| A124 | 273 | 277 |

A therapeutic agent may be used alone or in combination with an additional therapeutic agent. In some cases, an “additional therapeutic agent” as used herein is administered alone. The therapeutic agents may be administered together or sequentially. The combination therapies may be administered within the same day, or may be administered one or more days, weeks, months, or years apart. In some cases, a therapeutic agent provided herein is administered if the subject is determined to be non-responsive to a first line of therapy, e.g., such as TNF inhibitor. Such determination may be made by treatment with the first line therapy and monitoring of disease state and/or diagnostic determination that the subject would be non-responsive to the first line therapy.

In some embodiments, the additional therapeutic agent comprises an anti-TNF therapy, e.g., an anti-TNFα therapy. In some embodiments, the additional therapeutic agent comprises a second-line treatment to an anti-TNF therapy. In some embodiments, the additional therapeutic agent comprises an immunosuppressant, or a class of drugs that suppress, or reduce, the strength of the immune system. In some embodiments, the immunosuppressant is an antibody. Non-limiting examples of immunosuppressant therapeutic agents include STELARA® (ustekinumab) azathioprine (AZA), 6-mercaptopurine (6-MP), methotrexate, cyclosporin A. (CsA).

In some embodiments, the additional therapeutic agent comprises a selective anti-inflammatory drug, or a class of drugs that specifically target pro-inflammatory molecules in the body. In some embodiments, the anti-inflammatory drug comprises an antibody. In some embodiments, the anti-inflammatory drug comprises a small molecule. Non-limiting examples of anti-inflammatory drugs include ENTYVIO (vedolizumab), corticosteroids, aminosalicylates, mesalamine, balsalazide (Colazal) and olsalazine (Dipentum).

In some embodiments, the additional therapeutic agent comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject. In some embodiments, the therapeutic agent comprises Cx601/Alofisel® (darvadstrocel).

In some embodiments, the additional therapeutic agent comprises a small molecule. The small molecule may be used to treat inflammatory diseases or conditions, or fibrostenonic or fibrotic disease. Non-limiting examples of small molecules include Otezla® (apremilast), alicaforsen, or ozanimod (RPC-1063).

In some embodiments, the additional therapeutic agent comprises an agonist or antagonist Janus Kinase 1 (JAK1). Non-limiting examples of JAK1 inhibitors include Ruxolitinib (INCB018424), S-Ruxolitinib (INCB018424), Baricitinib (LY3009104, INCB028050), Filgotinib (GLPG0634), Momelotinib (CYT387), Cerdulatinib (PRT062070, PRT2070), LY2784544, NVP-BSK805, 2HC1, Tofacitinib (CP-690550,Tasocitinib), XL019, Pacritinib (SB1518), or ZM 39923 HCl.

Pharmaceutical Compositions, Formulations, and Methods of Administration

In one aspect, methods of treating a subject, e.g., a subject having a CD-PBmu subtype, monocyte 2 subtype, monocyte 1 subtype, or any combination thereof, involve administration of a pharmaceutical composition comprising a therapeutic agent described herein, e.g., a modulatory of expression and/or activity of a biomarker in Table 4 or Table 7B, or of a biomolecule in a pathway of a biomarker in Table 4 or Table 7B, in therapeutically effective amounts to said subject. In some embodiments, a therapeutic agent described herein is used in the preparation of medicaments for treating an inflammatory disease, such as Crohn's Disease.

In certain embodiments, the compositions containing the therapeutic agent described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial. In some cases, a therapeutic agent is administered to a patient suffering from an inflammatory disease such as CD, and optionally comprises a CD-PBmu subtype and/or monocyte 1 or 2 subtype.

In prophylactic applications, compositions containing a therapeutic agent described herein are administered to a patient susceptible to or otherwise at risk of a particular disease, disorder or condition, e.g., an inflammatory disease. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition.

In certain embodiments wherein a patient's status does improve, the dose of therapeutic agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given therapeutic agent that corresponds to such an amount varies depending upon factors such as the particular therapeutic agent, disease condition and its severity, the identity (e.g., weight, sex, age) of the subject in need of treatment, but can nevertheless be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day.

In some embodiments, as a patient is started on a regimen of a therapeutic agent, the patient is also weaned off (e.g., step-wise decrease in dose) a second treatment regimen.

In one embodiment, the daily dosages appropriate for a therapeutic agent herein are from about 0.01 to about 10 mg/kg per body weight. In specific embodiments, an indicated daily dosage in a large mammal, including, but not limited to, humans, is in the range from about 0.5 mg to about 1000 mg, conveniently administered in divided doses, including, but not limited to, up to four times a day. In some embodiments, the daily dosage is administered in extended release form. In certain embodiments, suitable unit dosage forms for oral administration comprise from about 1 to 500 mg active ingredient. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the therapeutic agent described herein lies within a range of circulating concentrations that include the ED₅₀ with minimal toxicity. In certain embodiments, the daily dosage range and/or the unit dosage amount varies within this range depending upon the dosage form employed and the route of administration utilized.

Disclosed herein are therapeutic agents formulated into pharmaceutical compositions. Pharmaceutical compositions are formulated in a conventional manner using one or more pharmaceutically acceptable inactive ingredients that facilitate processing of the active therapeutic agent into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

Provided herein are pharmaceutical compositions that include a therapeutic agent described herein, and at least one pharmaceutically acceptable inactive ingredient. In some embodiments, the therapeutic agents described herein are administered as pharmaceutical compositions in which the therapeutic agents are mixed with other active ingredients, as in combination therapy. In some embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In some embodiments, the pharmaceutical compositions include other therapeutically valuable substances.

A pharmaceutical composition, as used herein, refers to a mixture of a therapeutic agent, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more therapeutic agent as discussed herein. In practicing the methods of treatment or use provided herein, therapeutically effective amounts of therapeutic agents described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the therapeutic agent used and other factors. The therapeutic agents can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a therapeutic agent are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a therapeutic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In some embodiments, a therapeutic agent exists as a tautomer. All tautomers are included within the scope of the agents presented herein. As such, it is to be understood that a therapeutic agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound.

In some embodiments, a therapeutic agent exists as an enantiomer, diastereomer, or other steroisomeric form. The agents disclosed herein include all enantiomeric, diastereomeric, and epimeric forms as well as mixtures thereof.

In some embodiments, therapeutic agents described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a therapeutic agent described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the therapeutic agent. In certain embodiments, a prodrug is enzymatically metabolized by one or more steps or processes to the biologically, pharmaceutically or therapeutically active form of the therapeutic agent.

Prodrug forms of the therapeutic agents, wherein the prodrug is metabolized in vivo to produce an agent as set forth herein are included within the scope of the claims. Prodrug forms of the herein described therapeutic agents, wherein the prodrug is metabolized in vivo to produce an agent as set forth herein are included within the scope of the claims. In some cases, some of the therapeutic agents described herein may be a prodrug for another derivative or active compound. In some embodiments described herein, hydrazones are metabolized in vivo to produce a therapeutic agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, a therapeutic agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch.

Representative intranasal formulations are described in, for example, U.S. Pat. Nos. 4,476,116, 5,116,817 and 6,391,452. Formulations that include a therapeutic agent are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the therapeutic agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, pharmaceutical formulations of a therapeutic agent are in the form of a capsules, including push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration. In one aspect, solid oral dosage forms are prepared by mixing a therapeutic agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of a therapeutic agent from the formulation. In other embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a therapeutic agent are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a therapeutic agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™ PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a therapeutic agents and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a therapeutic agent is formulated to include one or more pharmaceutical excipients and flavors. Such a powder is prepared, for example, by mixing the therapeutic agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical dosage forms are formulated to provide a controlled release of a therapeutic agent. Controlled release refers to the release of the therapeutic agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a therapeutic agent that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following: Shellac—this coating dissolves in media of pH>7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)—PVAP dissolves in pH>5, and it is much less permeable to water vapor and gastric fluids. Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of a therapeutic agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a therapeutic agent and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a therapeutic agent, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, Glycyrrhiza (licorice) syrup, monoammonium glyrrhizinate (MagnaSweet®), malitol, mannitol, menthol, neohesperidine DC, neotame, Prosweet® Powder, saccharin, sorbitol, Stevia, sucralose, sucrose, sodium saccharin, saccharin, aspartame, acesulfame potassium, mannitol, sucralose, tagatose, thaumatin, vanilla, xylitol, or any combination thereof.

In some embodiments, a therapeutic agent is prepared as transdermal dosage form. In some embodiments, the transdermal formulations described herein include at least three components: (1) a therapeutic agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of a therapeutic agent described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the therapeutic agents described herein can be accomplished by means of iontophoretic patches and the like. In one aspect, transdermal patches provide controlled delivery of a therapeutic agent. In one aspect, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical therapeutic agents may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a therapeutic agent described herein may be administered topically and can be formulated into a variety of topically administrable compositions, such as solutions, suspensions, lotions, gels, pastes, medicated sticks, balms, creams or ointments. Such pharmaceutical therapeutic agents can contain solubilizers, stabilizers, tonicity enhancing agents, buffers and preservatives.

### Kits

The disclosure also provides kits for detecting expression of one or more genes in Table 1A or Table 7A. Exemplary kits include nucleic acids configured for specific hybridization to one or more genes in Table 1A or Table 7A. In some cases a kit comprises a plurality of such nucleic acids immobilized on a substrate, such as a microarray, welled plate, chip, or other material suitable for microfluidic processing.

In some embodiments, the kit includes nucleic acid and/or polypeptide isolation reagents. In some embodiments, the kit includes one or more detection reagents, for example probes and/or primers for amplification of, or hybridization to, a gene in Table 1A or Table 7A. In some embodiments, the kit includes primers and probes for control genes, such as housekeeping genes. In some embodiments, the primers and probes for control genes are used, for example, in AG calculations. In some embodiments, the probes or primers are labeled with an enzymatic, florescent, or radionuclide label.

In some instances, a kit comprises a nucleic acid polymer (e.g., primer and/or probe) comprising at least about 10 contiguous nucleobases having at least about 70%, 80%, 85%, 90%, 91%, 92%, 93%, 94%, 95%, 96%, 97%, 98% or 99% sequence identity or homology to a biomarker of Table 1A or Table 7A.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

In some embodiments, a kit includes one or more additional containers, each with one or more of various materials (such as reagents, optionally in concentrated form, and/or devices) desirable from a commercial and user standpoint for use of described herein. Non-limiting examples of such materials include, but not limited to, buffers, primers, enzymes, diluents, filters, carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

### Systems

Disclosed herein, in some embodiments, is a system for detecting a particular subtype of IBD or CD in a subject. In some embodiments, the subtype is CD-PBmu. In some embodiments, the subtype is CD PBT. In some embodiments, the subtype is monocyte 2 subtype. In some embodiments, the subtype is monocyte 1 subtype. The system is configured to implement the methods described in this disclosure, including, but not limited to, detecting the presence of a particular CD subtype to determine whether the subject is suitable for treatment with a particular therapy.

In some embodiments, disclosed herein is a system for detecting a IBD subtype in a subject, comprising: (a) a computer processing device, optionally connected to a computer network; and (b) a software module executed by the computer processing device to analyze a target nucleic acid sequence of a transcriptomic profile in a sample from a subject. In some instances, the system comprises a central processing unit (CPU), memory (e.g., random access memory, flash memory), electronic storage unit, computer program, communication interface to communicate with one or more other systems, and any combination thereof. In some instances, the system is coupled to a computer network, for example, the Internet, intranet, and/or extranet that is in communication with the Internet, a telecommunication, or data network. In some embodiments, the system comprises a storage unit to store data and information regarding any aspect of the methods described in this disclosure. Various aspects of the system are a product or article or manufacture.

One feature of a computer program includes a sequence of instructions, executable in the digital processing device's CPU, written to perform a specified task. In some embodiments, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions are combined or distributed as desired in various environments. In some instances, a computer program comprises one sequence of instructions or a plurality of sequences of instructions. A computer program may be provided from one location. A computer program may be provided from a plurality of locations. In some embodiment, a computer program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof

### Web Application

In some embodiments, a computer program includes a web application. In light of the disclosure provided herein, those of skill in the art will recognize that a web application may utilize one or more software frameworks and one or more database systems. A web application, for example, is created upon a software framework such as Microsoft®.NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

Standalone Application

In some embodiments, a computer program includes a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB NET, or combinations thereof. Compilation may be often performed, at least in part, to create an executable program. In some instances, a computer program includes one or more executable complied applications.

Web Browser Plug-in

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

Databases

The medium, method, and system disclosed herein comprise one or more databases, or use of the same. In view of the disclosure provided herein, those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

Data Transmission

The subject matter described herein, including methods for detecting a particular CD subtype, are configured to be performed in one or more facilities at one or more locations. Facility locations are not limited by country and include any country or territory. In some instances, one or more steps are performed in a different country than another step of the method. In some instances, one or more steps for obtaining a sample are performed in a different country than one or more steps for detecting the presence or absence of a particular CD subtype from a sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy, or the like, in the same or different location or country.

Business Methods Utilizing a Computer

The gene expression profiling methods may utilize one or more computers. The computer may be used for managing customer and sample information such as sample or customer tracking, database management, analyzing molecular profiling data, analyzing cytological data, storing data, billing, marketing, reporting results, storing results, or a combination thereof. The computer may include a monitor or other graphical interface for displaying data, results, billing information, marketing information (e.g. demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a gene expression profile may enter sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### EXAMPLES

While preferred embodiments have been shown and described herein, it will be obvious to those skilled in the art that such embodiments are provided by way of example only. Numerous variations, changes, and substitutions will now occur to those skilled in the art without departing from the embodiments provided. It should be understood that various alternatives to the embodiments described herein may be employed.

### Example 1: Blood Based Pre-Surgical Transcriptomic Signature

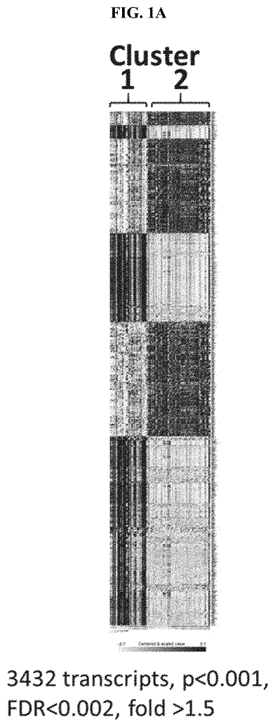

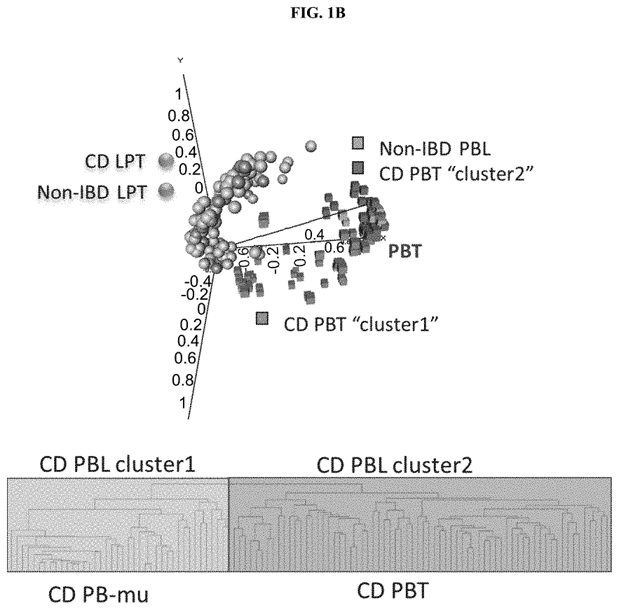

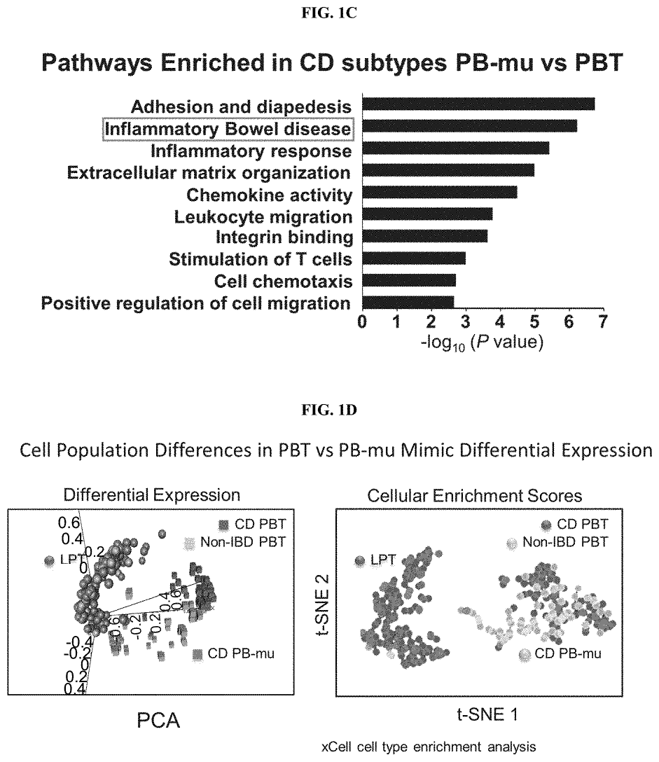

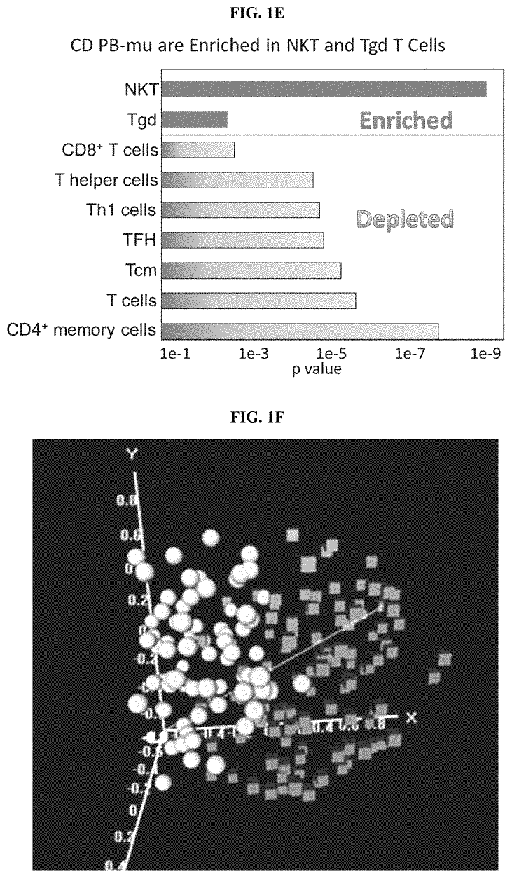

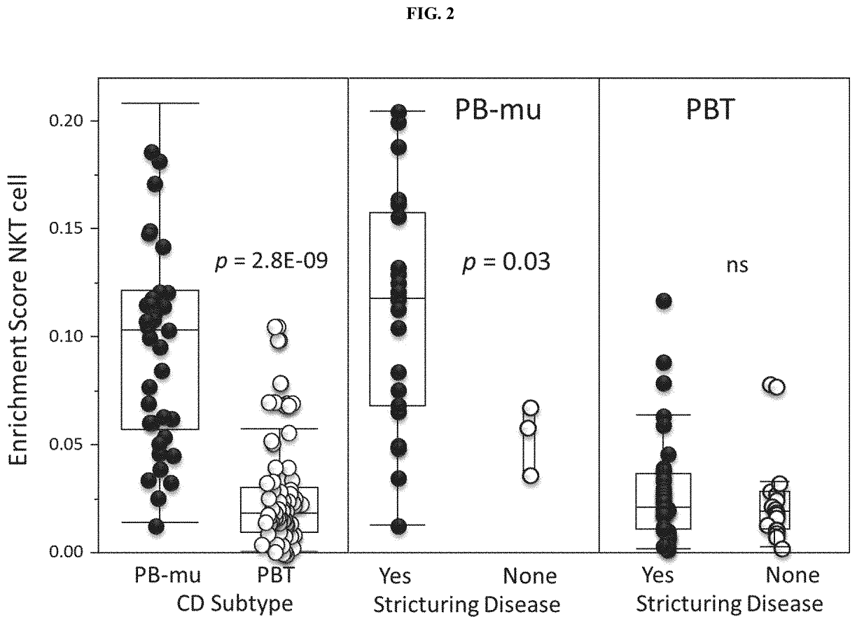

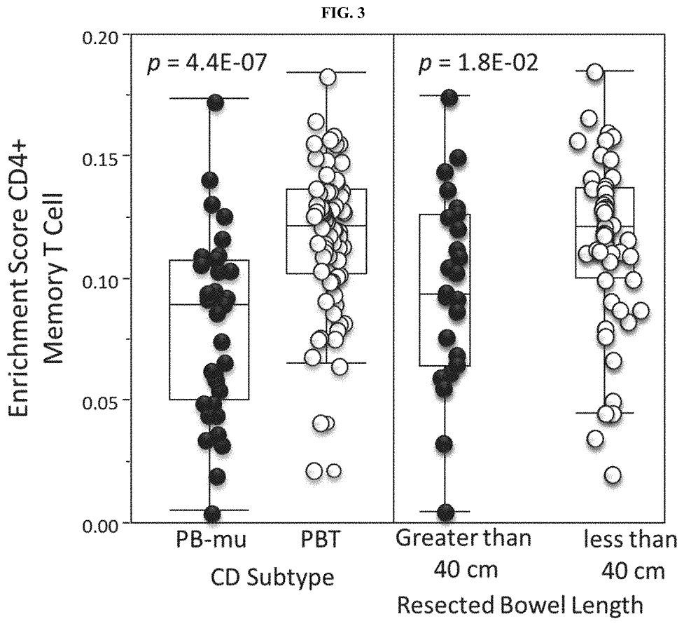

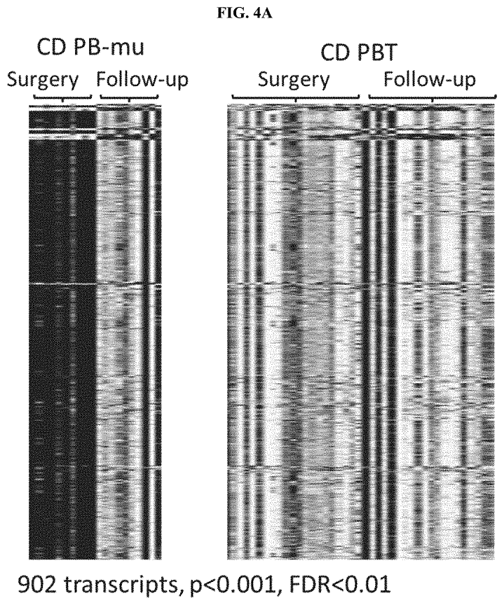

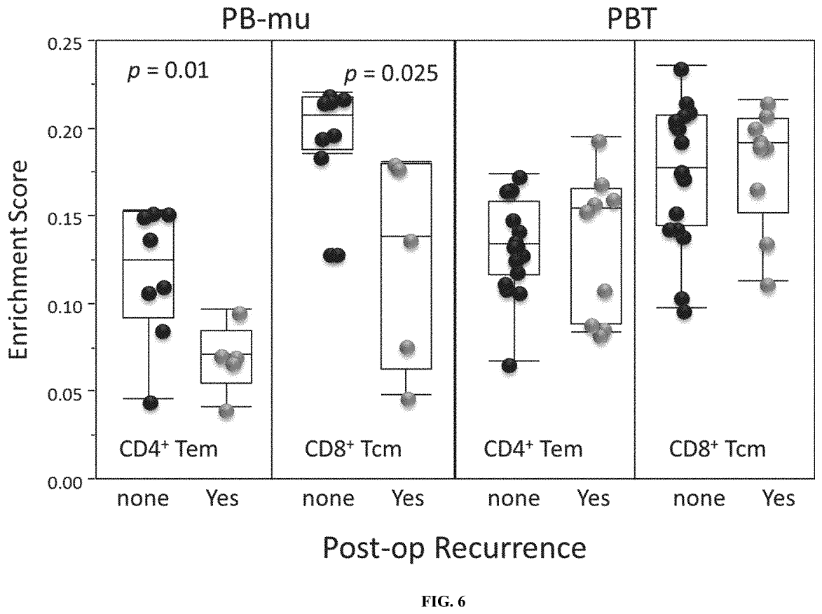

RNA expression analysis was performed on purified CD3⁺ T-cells isolated from paired blood and mucosal tissue from 101 CD patients with severe disease who required surgical intervention for disease management and 17 individuals without IBD. Transcriptional profiles were analyzed by RNA-seq and T-cell subset composition using xCell bioinformatic tool. Unsupervised clustering of peripheral T cells (PBT) revealed 2 distinct transcriptomic signatures: 1) gene expression from a classified CD-PBT (63%) clustered tightly with non-IBD subjects, and 2) gene expression drifted from a peripheral toward a mucosal profile, classified as CD-PBmu(cosal) (37%) (FIG. 1A, 1B). Pathways enriched in the CD-PBmu subtype as compared to CD-PBT subtype include pathways related to inflammatory bowel disease, leukocyte migration, cell chemotaxis, and positive regulation of cell migration, as shown in FIG. 1C. CD-PBmu phenotype was defined by a distinctive peripheral T-cell subset composition (FIG. 1D, FIG. 1E). The PBmu compared to PBT subtype demonstrated inferred enrichment for NKT cells and depletion of TH1 and CD4+ and CD8+ memory and naïve cell subsets (FIG. 1E and Table 3). The impact of altered T cell subset composition and clinical characteristics of disease activity was assessed. In the CD PBmu, but not CD PBT sub-type, NKT cell enrichment scores were associated with structuring disease (p=0.3) and ASCA levels (p=0.03) (FIG. 2 and Table 3). Conversely, depletion of CD4+ memory T cells observed in the CD PBmu vs PBT subtype was associated with (Table 3: increased length of intestinal resection (p=0.02) (FIG. 3 and Table 3), post-operative endoscopic recurrence of disease (p=0.01) (FIG. 6 and Table 3), increased serological quartile sum scores (QSS) (p=0.02), and QSS are associated with increased length of bowel resection (p=0.036).

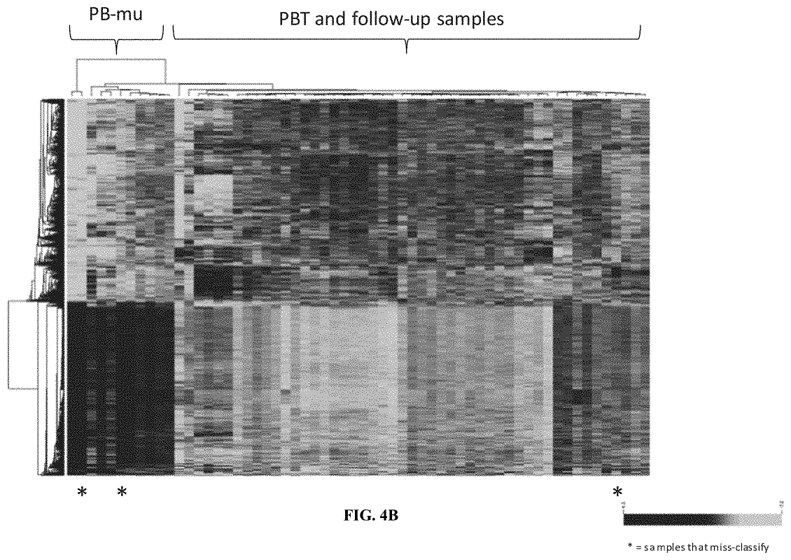

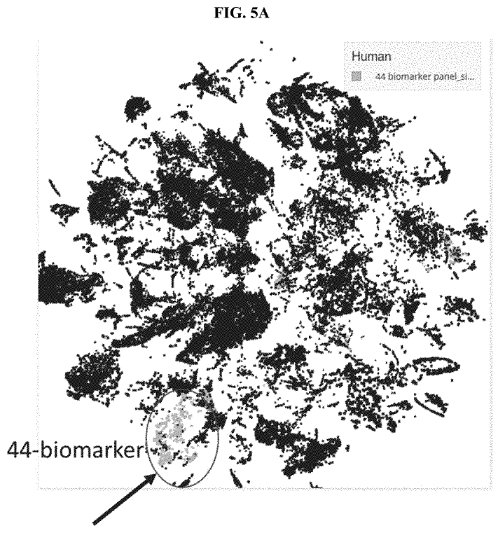

A defining hallmark for the CD-PBmu subtype, validated in an additional patient cohort (n=19), was profoundly downregulated expression of pro-inflammatory cytokines, chemokines and adhesion molecules following surgery (FIGS. 4A-4B, Table 4). As seen in FIG. 4B following surgery gene expression of the PBmu-subtype reverts to that observed for the PBT and non-IBD subjects at time of surgery. No post-surgery alteration in gene expression was detected in the CD-PBT subtype (FIG. 4A).

TABLE 3
Unique CD-PBmu vs CD-PBT signature attributes

| | |
|---|---|
| Differential Expression | |
| Differential Gene Expression of CD-PBmu vs CD-PBT | 1566 transcripts, p = 9.91E−04, |
| | FDR < 0.002, fold > 2 |
| Enriched in pathways mediating inflammatory response, leukocyte | p = 9.9E−03-5.1E−07 |
| adhesion, migration and integrin binding | |
| T Cell Subset composition and clinical associations | |
| NKT cell enrichment in CD-PBmu vs CD-PBT | p = 2.8E−09 |
| NKT cell enrichment in CD-PBmu, but not CD-PBT, is associated | p = 2.5E−04 |
| with stricturing disease | |
| NKT cell enrichment in CD-PBmu, but not CD-PBT, correlated with | p = 3.3E−02 |
| ASCA serological response levels | |
| Decreased CD4+/CD8+ T cells in CD-PBmu vs CD-PBT | p = 6.1E−03-1.7E−07 |
| Decreased CD4+ memory T cell is associated with increased length of | p = 1.8E−02 |
| bowel resection | |
| Serological quartile sum scores in CD-PBmu, but not CD-PBT, are | p = 2.9E−02 |
| associated with increased length of bowel resection | |
| Attenuated gene expression in CD-PBmu, but not in CD-PBT, | 900 transcripts, p = 9.9E−04, |
| following surgery | FDR < 0.01, fold > 1.5 |

It was also found that the PBL (peripheral blood lymphocytes) cell population changes in CD-PBmu patients following surgery: There is a decrease in the elevated pre-surgery NKT (p=0.026) and enhancement in the levels of depleted pre-surgery Th1 cells (p=5e-03).

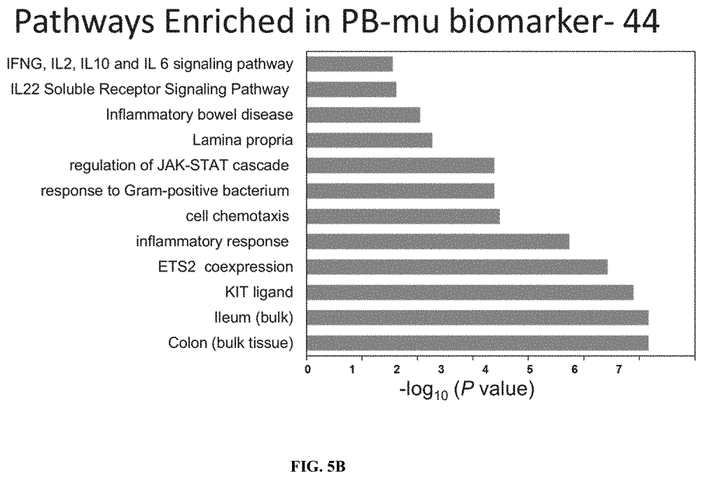

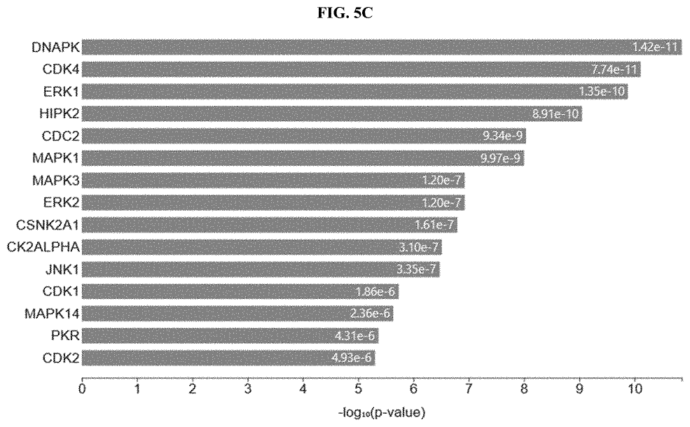

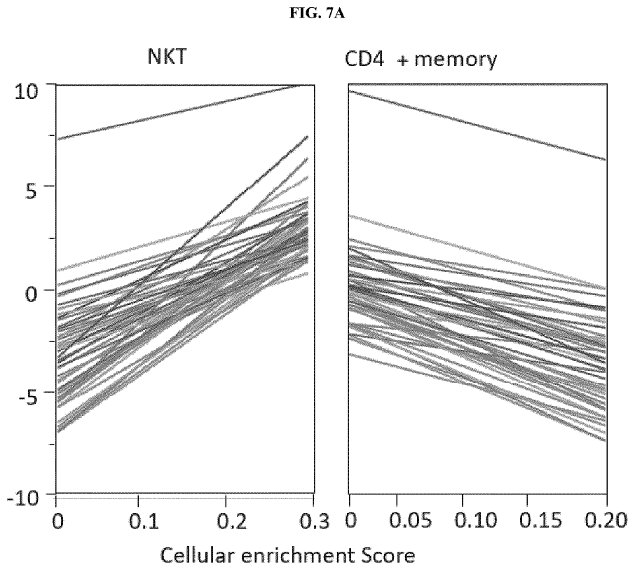

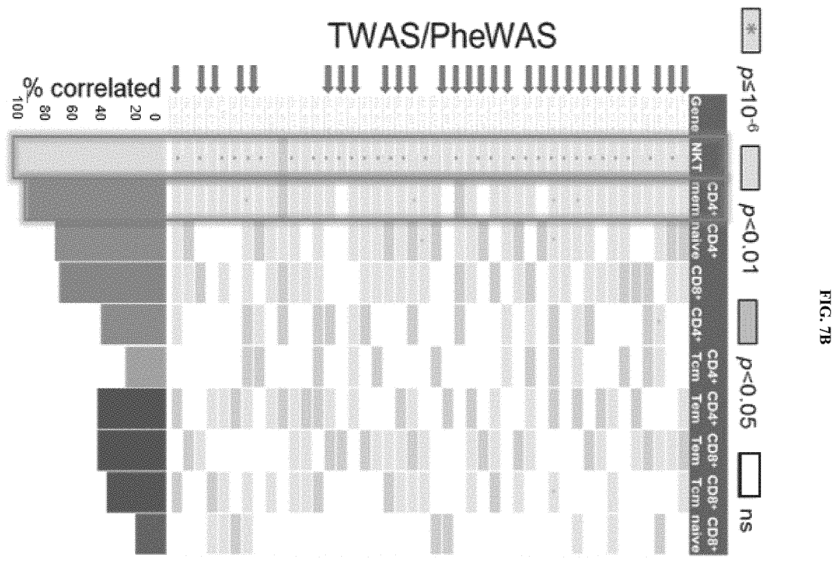

Findings were validated in independent datasets derived from either whole blood (isolated from CD patients who had failed anti-TNF therapy, n=204, (FIG. 1F), ileal tissue (from pediatric CD patients, 3 studies n>600, GSE62207, GSE57945, GSE93624), or IBD patients (3 studies, n>300, GSE83687, GSE81266, GSE72819) and confirmed the CD-PBmu signature and peripheral mucosal-like T-cell expression profile. These findings were refined into a 200 (Table 1A), 117 (Genes 1-117 of Table 1A), and then a 44-gene panel (Table 1A, FIG. 5A) to facilitate clinical application. The 44-gene biomarker panel accurately identified the CD-PBmu signature in peripheral T-cells and overlapped with transcriptome-wide (TWAS) signals predicted for associations with IBD (>50% of biomarker panel) (FIG. 7B). Pathways enriched in the 44-gene panel are shown in FIG. 5B.

The 44-gene panel correlated with T cell subsets: NKT, CD4+ memory, CD4+ native, CD8+, CD4+, CD4+ Tcm, CD4+ Tem, CD8+ Tem, CD8+ Tcm, and CD8+ naive, as shown in FIGS. 7A-7B. The NKT and CD4+ memory cells display >90% correlation. FIG. 7A-7B show that 44 Biomarker expression gene panel correlates PB-mu enriched NKT and depleted CD4+ memory T cell subsets. FIG. 7A shows enrichment of NKT cells and negative enrichment of CD4+ T cells by cellular enrichment score. FIG. 7B shows enrichment of gene markers of NKT cells and negative enrichment of gene markers of CD4+ T cells.

The 44-gene biomarker panel includes A disintegrin and metalloproteinase with thrombospondin motifs 1 (ADAMTS1), Neutrophil gelatinase-associated lipocalin (LCN2), Disintegrin and metalloproteinase domain-containing protein 28 (ADAM28), Tryptase beta-2 (TPSB2), peptidylprolyl isomerase A pseudogene 30 (PPIAP30), glutamine-fructose-6-phosphate transaminase 2 (GFPT2), KIT proto-oncogene, receptor tyrosine kinase (KIT), phospholipid transfer protein (PLTP), major facilitator superfamily domain containing 2A (MFSD2A), interleukin 22 (IL22), LIM and cysteine rich domains 1 (LMCD1), interleukin 6 (IL6), TBC1 domain family member 9 (TBC1D9), ChaC glutathione specific gamma-glutamylcyclotransferase 1 (CHAC1), selenoprotein P (SEPP1), superoxide dismutase 3 (SOD3), RAB13, member RAS oncogene family (RAB13), lysozyme (LYZ), carboxypeptidase A3 (CPA3), serine dehydratase (SDS), dual specificity tyrosine phosphorylation regulated kinase 3 (DYRK3), DAB adaptor protein 2 (DAB2), TBC1 domain family member 8 (TBC1D8), crystallin alpha B (CRYAB), TBC1 domain family member 3 (TBC1D3), leucine rich repeat containing 32 (LRRC32), serpin family G member 1 (SERPING1), ubiquitin D (UBD), fatty acid binding protein 1 (FABP1), spleen associated tyrosine kinase (SYK), aldolase, fructose-bisphosphate B (ALDOB), semaphorin 6B (SEMA6B), NANOG neighbor homeobox (NANOGNB), dermatan sulfate epimerase (DSE), formyl peptide receptor 3 (FPR3), tenascin XB (TNXB), olfactory receptor family 4 subfamily A member 5 (OR4A5), decorin (DCN), carbohydrate sulfotransferase 15 (CHST15), ADAM like decysin 1 (ADAMDEC1), histidine decarboxylase (HDC), RRAD, Ras related glycolysis inhibitor and calcium channel regulator (RRAD), complement C1s (C1S), or phospholipase A2 group IIA (PLA2G2A).

CD patients with severe disease can be stratified into 2 sub-populations based on transcriptomic profiling of their peripheral T-cells. A mucosal-like expression profile defined the CD-PBmu subtype which was associated with an altered composition of T-cell subsets, clinical disease severity markers and decreased pro-inflammatory gene expression following surgery. These findings hold potential to identify targets for patient-subtype specific therapeutic development. Moreover, the 44-gene biomarker panel confirmed the CD-PBmu gene signature in multiple independent pediatric CD datasets, suggesting this may provide a unique tool to improve accuracy in predicting clinical progression and facilitate treatment stratification early in the disease process. In some cases, the

In some cases, the 44-gene biomarker panel can be narrowed to a 27-gene biomarker panel with similar predictive capability as the 44-gene biomarker panel. The 27-gene biomarker panel, in some cases is ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, and UBD.

TABLE 4
Selected Cytokines, Chemokines and Adhesion Molecules
Decreased in PB-mu Patient Subtype Following Surgery

| Molecule | P value |
|---|---|
| IL10 | 1.7E−03 |
| IL11 | 4.0E−04 |
| IL15 | 1.8E−03 |
| IL18 | 1.9E−02 |
| IL22 | 8.5E−03 |
| IL6 | 1.0E−03 |
| IL12RB1 | 4.0E−04 |
| IL12RB2 | 1.1E−02 |
| IL17RD | 5.0E−04 |
| IL1R1 | 2.2E−03 |
| IL1RL1 | 7.9E−03 |
| IL31RA | 1.4E−03 |
| TNFRSF9 | 7.0E−04 |
| TNFSF14 | 3.3E−02 |
| TNFSF15 | 5.7E−03 |
| TNFAIP8L1 | 1.0E−03 |
| TNFAIP8L3 | 4.7E−03 |
| TNFRSF10A | 4.6E−02 |
| TNFRSF10B | 6.2E−03 |
| TNFRSF13B | 2.9E−02 |
| CCL11 | 1.1E−02 |
| CCL16 | 2.2E−03 |
| CCL21 | 2.7E−02 |
| CCL22 | 7.0E−04 |
| CCL28 | 5.5E−03 |
| CCL5 | 2.0E−04 |
| CCR6 | 7.6E−03 |
| CCR9 | 4.0E−03 |
| CXCL1 | 2.3E−02 |
| CXCL12 | 1.9E−02 |
| CXCL13 | 8.2E−03 |
| CXCL14 | 8.0E−04 |
| CXCL16 | 2.3E−02 |
| CXCL3 | 3.4E−02 |
| CXCL9 | 1.0E−04 |
| CLDN10 | 3.4E−02 |
| CLDN16 | 1.0E−03 |
| CLDN19 | 2.0E−04 |
| CLDN3 | 1.2E−03 |
| ICAM4 | 4.0E−03 |
| ITGAX | 2.2E−02 |

### Example 2. Transcriptomic Profiling

Expression levels of each of genes 1-44 in Table 1A are determined in a CD patient using RNA sequencing. The patient's expression levels are compared to reference expression levels from subjects who have a PBT subtype. All of the 44-genes from the patient have expression levels at least 2-fold higher than the PBT reference. The patient is characterized as having a CD-PBmu subtype.

### Example 3. Identification of Therapeutic Agents

A library of compounds is screened for a subpopulation of compounds that modulate the activity and/or expression of one or more biomarkers of Table 5, or of a biomolecule in a pathway of the one or more biomarkers of Table 5. The subpopulation of compounds is screened for efficacy in an in vitro PBmu patient model to identify candidate therapeutic agents.

### Example 4. Monocyte Signature

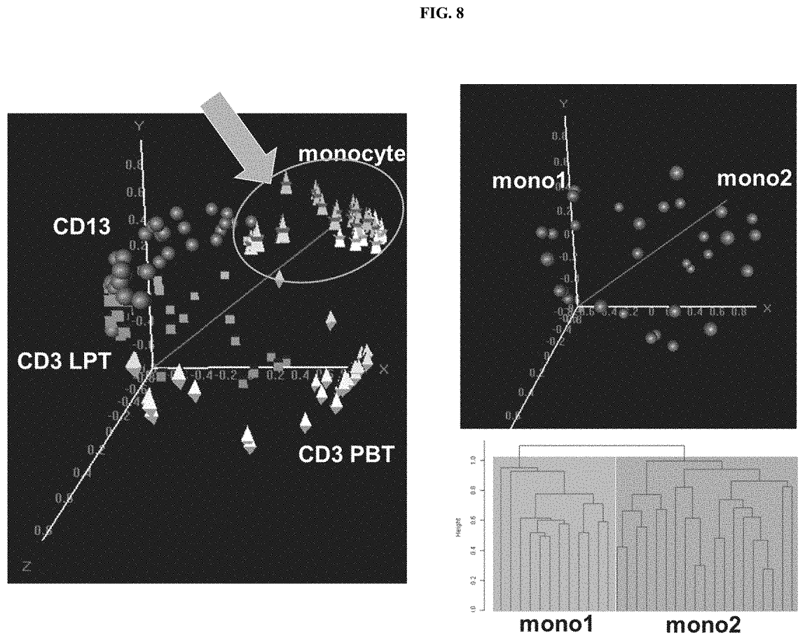

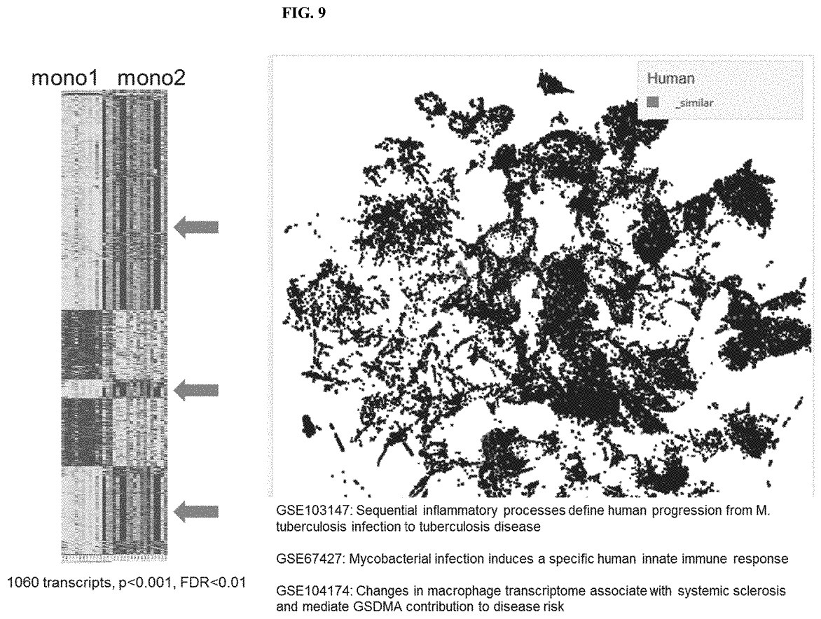

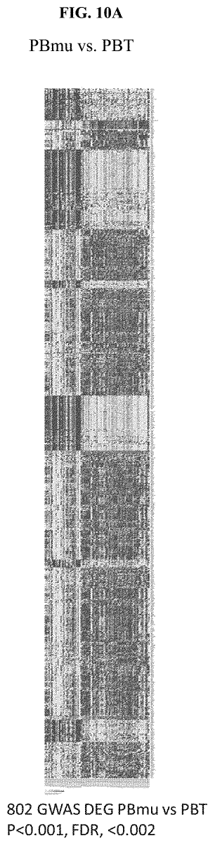

Peripheral and mucosal cells were obtained from untreated freshly isolated cells from 30 Crohn's disease (CD) subjects and 10 non-IBD subjects. RNA expression analysis was performed on peripheral CD3+ and monocyte cells, and mucosal CD3+ and CD13+ cells. Unsupervised clustering of CD monocytes revealed two signatures: monocyte 1 subtype (mono1) and monocyte 2 (mono2) subtype (FIG. 8). Differential gene expression in mono1 versus mono2 subtypes is shown in FIG. 9.

The CD mono2 subset was found to be associated with clinical and genetic parameters: ATG16L1 rs10210302 risk allele carriage (z score 2.2, p value 0.014), family history (z score 2.2, p value 0.014), IgG ASCA positive (z score 3, p value 0.0013), Serologic Quartile sum score (avg 11.4) (p value 0.049), failure on anti-TNF therapy (z score 1.8, p value 0.03), failure on 6-mercaptopurine/methotrexate (z score 3.4, p value 0.0004), and PBmu subjects (z score 1.4, p value 0.07).

### Example 5. Identifying Therapeutic Agents of Particular Relevance to PBmu CD Subtype

A two-tailed test was performed, which measured the statistical significance of an association of the differential gene expression of a target of interstest in the PBmu patient subset. Table 5 provides a list of putative therapeutic targets, the differential expression of which, are statistically associated with the PBmu subtype.

TABLE 5
Therapeutic Targets for PBmu Subtype

| Gene | Pbmu | PBT | Prob > \|t\| |
|---|---|---|---|
| ADCY7 | 19.91897 | 24.43544 | 2.86E−03 |
| GPR65 | 32.85385 | 18.49456 | 4.62E−05 |
| GSDMB | 8.521538 | 5.792059 | 2.07E−04 |
| ICAM3 | 64.45026 | 84.52338 | 3.61E−06 |
| MAP4K4 | 24.32692 | 27.24235 | 4.35E−02 |
| PRKCQ | 23.15692 | 28.36426 | 2.42E−04 |
| PTGER4 | 23.70487 | 34.84235 | 7.49E−04 |
| RNASET2 | 60.94795 | 77.84529 | 6.13E−04 |
| TNFSF15 | 3.208718 | 1.245882 | 1.46E−03 |

The 44-biomarker panel is associated with kinases provided in FIG. 5C. Without being bound by any particular theory, CD-PBmu patients would likely benefit from a targeted therapy to the kinases provided in FIG. 5C.

### Example 6. Monocyte Profiling

The expression level of one or more genes from Table 5 is determined in a CD patient using RNA sequencing. The patient's expression levels are compared to reference expression levels from subjects who have a mono1 or mono2 subtype. If the patient's expression levels are comparable to reference subjects having a mono2 subtype, the patient is characterized as having the mono2 CD subtype.

### Example 7. Treatment of Crohn's Disease Patient with PBmu Profile

The patient having the PBmu phenotype of Example 1 is treated with a candidate therapeutic agent of Example 3 or a therapeutic agent comprising a modulator of one or more of TL1A, ADCY7, GPR65, ICAM3, MAP4K4, PTGER4, RNASET2, TNFSF15.

### Example 8. Treatment of Crohn's Disease Patient with Monocyte 2 Profile

The patient having the monocyte 2 subtype of Example 6 is treated with a candidate therapeutic agent targeting a kinase selected from: DNAPK, CDK4, ERK1, HIPK2, CDC2, MAPK1, MAPK3, ERK2, CSNK2A1, CK2ALPHA, JNK1, CDK1, MAPK14, PKR, and CDK2.

### Example 9. Pathways Enriched that Overlap with GWAS DEG in CD-PBmu Subtype

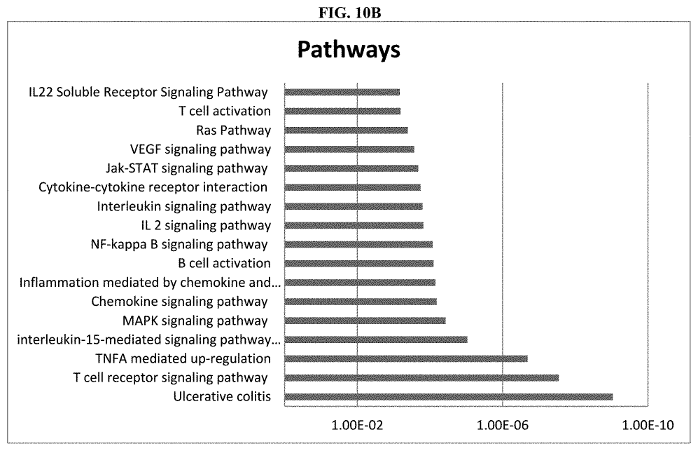

2616 genes potentially associated with IBD GWAS risk variant loci were identified. Of these genes, 1177 were not expressed in T cell data, 1429 were expressed in the T cell data, and 802 were differentially expressed between CD-PBmu and PBT subtypes (FIG. 10A). FIG. 10B shows pathways enriched that overlap with GWAS DEG CD-PBmu: IL22 soluble receptor signaling pathway, T cell activation, Ras pathway, VEGF signaling pathway, Jak-STAT signaling pathway, Cytokine-cytokine receptor interaction, interleukin signaling pathway, IL-2 signaling pathway, NF-kappa B signaling pathway, B cell activation, inflammation mediated by chemokine and cytokine signaling pathway, chemokine signaling pathway, MAPK signaling pathway, interleukin-15-mediated signaling pathway, TNF alpha mediated up-regulation, T cell receptor signaling pathway, and ulcerative colitis. In some examples, treatment of a patient having a CD-PBmu subtype comprises a molecule in one or more of the pathways shown in FIG. 10B.

While preferred embodiments have been shown and described herein, it will be obvious to those skilled in the art that such embodiments are provided by way of example only. Numerous variations, changes, and substitutions will occur to those skilled in the art without departing from the scope of this application. Various alternatives to the embodiments described herein may be employed in practicing the scope of this application.

TABLE 6
Genes Associated with Transcriptomic Signature.

| | | fold | | | | | |
|---|---|---|---|---|---|---|---|
| | predictor | Pbmu/ | | | | | |
| | fold | post | | | | | |
| | Pbmu/ | surgery | | Entrez | | | |
| Gene | PBT | follow up | Name | ID | Accession | UGCluster | Ensembl |
| AADACL2-AS1 | 6.09 | 6.44 | AADACL2 Antisense RNA 1 | 101928142 | | | ENSG00000242908 |
| AARS2 | 2.34 | 2.05 | alanyl-tRNA synthetase 2, | 57505 | NM_020745 | Hs.158381 | ENSG00000124608 |
| | | | mitochondrial | | | | |
| AASS | 3.55 | 2.96 | aminoadipate-semialdehyde synthase | 10157 | NM_005763 | Hs.156738 | ENSG00000008311 |
| ABCB5 | 4.08 | 2.97 | ATP-binding cassette, sub-family B | 340273 | NM_001163941 | Hs.404102 | ENSG00000004846 |
| | | | (MDR/TAP), member 5 | | | | |
| ABCC9 | 4.77 | 3.61 | ATP-binding cassette, sub-family C | 10060 | NM_005691 | Hs.732701 | ENSG00000069431 |
| | | | (CFTR/MRP), member 9 | | | | |
| ABHD11 | 2.6 | 2.24 | abhydrolase domain containing 11 | 83451 | NM_001145363 | Hs.647045 | ENSG00000106077 |
| ACADSB | 2.65 | 2.32 | acyl-CoA dehydrogenase, | 36 | NM_001609 | Hs.81934 | ENSG00000196177 |
| | | | short/branched chain | | | | |
| ACBD4 | 2.62 | 2.72 | acyl-CoA binding domain containing 4 | 79777 | NM_001135704 | Hs.110298 | ENSG00000181513 |
| ACBD7 | 4.58 | 3.42 | acyl-CoA binding domain containing 7 | 414149 | NM_001039844 | Hs.644598 | ENSG00000176244 |
| ADAMTS4 | 3.64 | 3.21 | ADAM metallopeptidase with | 9507 | NM_005099 | Hs.211604 | ENSG00000158859 |
| | | | thrombospondin type 1 motif, 4 | | | | |
| ADAT1 | 2.23 | 2 | adenosine deaminase, tRNA-specific 1 | 23536 | NM_012091 | Hs.729312 | ENSG00000065457 |
| ADRA1A | 3.91 | 3.36 | adrenoceptor alpha 1A | 148 | NM_000680 | Hs.709175 | ENSG00000120907 |
| AFMID | 3.83 | 3.03 | arylformamidase | 125061 | NM_001010982 | Hs.558614 | ENSG00000183077 |
| AICDA | 4.43 | 3.46 | activation-induced cytidine deaminase | 57379 | NM_020661 | Hs.149342 | ENSG00000111732 |
| AIPL1 | 4.1 | 3.6 | aryl hydrocarbon receptor | 23746 | NM_001033054 | Hs.279887 | ENSG00000129221 |
| | | | interacting protein-like 1 | | | | |
| AK3 | 2.27 | 1.98 | adenylate kinase 3 | 50808 | NM_001199852 | Hs.732022 | ENSG00000147853 |
| AKAP5 | 3.11 | 2.76 | A kinase (PRKA) anchor protein 5 | 9495 | NM_004857 | Hs.656683 | ENSG00000179841 |
| AKIP1 | 3.7 | 2.86 | A kinase (PRKA) interacting protein 1 | 56672 | NM_001206645 | Hs.131180 | ENSG00000166452 |
| ALDH6A1 | 3.37 | 2.79 | aldehyde dehydrogenase 6 | 4329 | NM_001278593 | Hs.293970 | ENSG00000119711 |
| | | | family, member A1 | | | | |
| ALG1 | 2.74 | 2.19 | ALG1, chitobiosyldiphosphodolichol | 56052 | NM_019109 | Hs.592086 | ENSG00000033011 |
| | | | beta-mannosyltransferase | | | | |
| ALG1L | 3.44 | 3.36 | ALG1, chitobiosyldiphosphodolichol | 200810 | NM_001015050 | Hs.591299 | ENSG00000189366 |
| | | | beta-mannosyltransferase-like | | | | |
| ALG1L9P | 3.65 | 3.19 | asparagine-linked glycosylation 1-like | 285407 | NR_073386 | Hs.546711 | ENSG00000248671 |
| | | | 9, pseudogene | | | | |
| ANKLE1 | 4.38 | 3.25 | ankyrin repeat and LEM | 126549 | NM_001278443 | Hs.721610 | ENSG00000160117 |
| | | | domain containing 1 | | | | |
| ANKRD20A9P | 4.63 | 3.49 | ankyrin repeat domain 20 family, | 284232 | NR_027995 | Hs.679496 | |
| | | | member A9, pseudogene | | | | |
| ANP32A-IT1 | 3.07 | 2.55 | ANP32A intronic transcript 1 | 80035 | NM_001040150 | Hs.662150 | |
| AP1S3 | 3.85 | 3.15 | adaptor-related protein | 130340 | NM_001039569 | Hs.632555 | ENSG00000152056 |
| | | | complex 1, sigma 3 subunit | | | | |
| AP4B1-AS1 | 3.41 | 2.86 | AP4B1 antisense RNA 1 | 100287722 | NR_037864 | Hs.664669 | ENSG00000226167 |
| AP4S1 | 2.79 | 2.43 | adaptor-related protein | 11154 | NM_001128126 | Hs.293411 | ENSG00000100478 |
| | | | complex 4, sigma 1 subunit | | | | |
| APOBEC3A | 4.49 | 3.41 | apolipoprotein B mRNA editing | 200315 | NM_001270406 | Hs.226307 | ENSG00000128383 |
| | | | enzyme, catalytic polypeptide-like 3A | | | | |
| APOBEC3B-AS1 | 4.84 | 3.26 | APOBEC3B antisense RNA 1 | 100874530 | NR_104187 | Hs.626951 | ENSG00000249310 |
| APOL1 | 2.69 | 2.24 | apolipoprotein L, 1 | 8542 | NM_001136540 | Hs.114309 | ENSG00000100342 |
| APOL4 | 4.11 | 3.23 | apolipoprotein L, 4 | 80832 | NM_030643 | Hs.115099 | ENSG00000100336 |
| AQP6 | 4.2 | 3.48 | aquaporin 6, kidney specific | 363 | NM_001652 | Hs.54505 | ENSG00000086159 |
| ARGFX | 3.85 | 2.97 | arginine-fifty homeobox | 503582 | NM_001012659 | Hs.224976 | ENSG00000186103 |
| ARHGEF26-AS1 | 4.74 | 3.55 | ARHGEF26 antisense RNA 1 | 100507524 | NR_037901 | Hs.370221 | ENSG00000243069 |
| ARIH2OS | 2.44 | 2.17 | ariadne homolog 2 opposite strand | 646450 | NM_001123040 | Hs.720727 | ENSG00000221883 |
| ARRDC3-AS1 | 3.78 | 2.91 | ARRDC3 antisense RNA 1 | 100129716 | NR_027435 | Hs.116364 | ENSG00000281357 |
| ARSA | 2.71 | 2.3 | arylsulfatase A | 410 | NM_000487 | Hs.88251 | ENSG00000100299 |
| ASTN2 | 4.05 | 3.07 | astrotactin 2 | 23245 | NM_001184734 | Hs.601562 | ENSG00000148219 |
| ATAD3C | 3.66 | 3.08 | ATPase family, AAA | 219293 | NM_001039211 | Hs.724767 | ENSG00000215915 |
| | | | domain containing 3C | | | | |
| ATCAY | 4.24 | 3.42 | ataxia, cerebellar, Cayman type | 85300 | NM_033064 | Hs.418055 | ENSG00000167654 |
| B3GNT6 | 4.52 | 3.67 | UDP-GlcNAc:betaGal beta-1,3-N- | 192134 | NM_138706 | Hs.352622 | ENSG00000198488 |
| | | | acetylglucosaminyltransferase 6 | | | | |
| BAIAP2-AS1 | 3.08 | 2.86 | BAIAP2 antisense RNA 1 | 440465 | NM_001004336 | Hs.448889 | ENSG00000226137 |
| | | | (head to head) | | | | |
| BBS5 | 4.12 | 3.56 | Bardet-Biedl syndrome 5 | 129880 | NM_152384 | Hs.233398 | ENSG00000163093 |
| BCDIN3D | 2.27 | 1.92 | BCDIN3 domain containing | 144233 | NM_181708 | Hs.142736 | ENSG00000186666 |
| BHMT2 | 3.9 | 3.18 | betaine--homocysteine S- | 23743 | NM_001178005 | Hs.114172 | ENSG00000132840 |
| | | | methyltransferase 2 | | | | |
| BIN3-IT1 | 2.99 | 2.57 | BIN3 intronic transcript 1 | 80094 | NM_025026 | Hs.675917 | |
| BMP7 | 4.73 | 3.55 | bone morphogenetic protein 7 | 655 | NM_001719 | Hs.473163 | ENSG00000101144 |
| BMS1P4 | 2.39 | 2.3 | BMS1 ribosome biogenesis | 729096 | NR_026592 | Hs.709171 | ENSG00000271816 |
| | | | factor pseudogene 4 | | | | |
| BMS1P5 | 2.6 | 2.44 | BMS1 ribosome biogenesis | 399761 | NM_001040053 | Hs.711898 | ENSG00000204177 |
| | | | factor pseudogene 5 | | | | |
| BMS1P6 | 2.03 | 2.31 | BMS1 ribosome biogenesis | 642826 | NR_024495 | Hs.463017 | |
| | | | factor pseudogene 6 | | | | |
| BNIPL | 4.17 | 3.13 | BCL2/adenovirus E1B 19 kD | 149428 | NM_001159642 | Hs.591473 | ENSG00000163141 |
| | | | interacting protein like | | | | |
| BPNT1 | 2.68 | 2.19 | 3′(2′), 5′-bisphosphate | 10380 | NM_001286149 | Hs.406134 | ENSG00000162813 |
| | | | nucleotidase 1 | | | | |
| BREA2 | 3.07 | 2.25 | breast cancer estrogen- | 286076 | NM_001024610 | Hs.178095 | |
| | | | induced apoptosis 2 | | | | |
| BRIP1 | 4.26 | 3.38 | BRCA1 interacting protein | 83990 | NM_032043 | Hs.128903 | ENSG00000136492 |
| | | | C-terminal helicase 1 | | | | |
| BSN-AS2 | 4.42 | 3.38 | BSN antisense RNA 2 (head to head) | 100132677 | NR_038866 | Hs.435651 | ENSG00000226913 |
| C12orf65 | 3.15 | 2.61 | chromosome 12 open reading frame 65 | 91574 | NM_001143905 | Hs.319128 | ENSG00000130921 |
| C12orf77 | 4.03 | 3.21 | chromosome 12 open reading frame 77 | 196415 | NM_001101339 | Hs.434453 | |
| C14orf105 | 3.8 | 3.53 | chromosome 14 open reading frame 105 | 55195 | NM_001283056 | Hs.659706 | ENSG00000100557 |
| C14orf178 | 3.54 | 3.36 | chromosome 14 open reading frame 178 | 283579 | NM_001173978 | Hs.375834 | ENSG00000197734 |
| C17orf75 | 3.56 | 2.87 | chromosome 17 open reading frame 75 | 64149 | NM_022344 | Hs.655257 | ENSG00000108666 |
| C19orf35 | 5.46 | 5.13 | chromosome 19 open reading frame 35 | 374872 | NM_198532 | Hs.511803 | ENSG00000188305 |
| C1orf174 | 2.88 | 2.45 | chromosome 1 open reading frame 174 | 339448 | NM_207356 | Hs.103939 | ENSG00000198912 |
| C1orf210 | 4.09 | 3.15 | chromosome 1 open reading frame 210 | 149466 | NM_001164829 | Hs.158963 | ENSG00000253313 |
| C1orf229 | 5.51 | 3.73 | chromosome 1 open reading frame 229 | 388759 | NM_207401 | Hs.456511 | |
| C1QTNF6 | 2.67 | 2.39 | C1q and tumor necrosis | 114904 | NM_031910 | Hs.22011 | ENSG00000133466 |
| | | | factor related protein 6 | | | | |
| C21orf62 | 4.33 | 3.47 | chromosome 21 open reading frame 62 | 56245 | NM_001162495 | Hs.517235 | ENSG00000205929 |
| C2orf91 | 5.18 | 4.02 | chromosome 2 open reading frame 91 | 400950 | NM_001242815 | Hs.738713 | ENSG00000205086 |
| C3orf33 | 3 | 2.97 | chromosome 3 open reading frame 33 | 285315 | NM_001308229 | Hs.350846 | ENSG00000174928 |
| C4orf19 | 3.63 | 3.26 | chromosome 4 open reading frame 19 | 55286 | NM_001104629 | Hs.107527 | ENSG00000154274 |
| C4orf26 | 4.14 | 3.6 | chromosome 4 open reading frame 26 | 152816 | NM_001206981 | Hs.24510 | ENSG00000174792 |
| C6orf25 | 2.81 | 2.48 | chromosome 6 open reading frame 25 | 80739 | NM_025260 | Hs.247879 | ENSG00000204420 |
| C7orf55 | 3.94 | 3.41 | chromosome 7 open reading frame 55 | 154791 | NM_197964 | Hs.718441 | ENSG00000164898 |
| C8orf44 | 3.44 | 3.16 | chromosome 8 open reading frame 44 | 56260 | NM_019607 | Hs.661238 | ENSG00000213865 |
| C9orf3 | 2.69 | 2.13 | chromosome 9 open reading frame 3 | 84909 | NM_001193329 | Hs.434253 | ENSG00000148120 |
| CABP4 | 3.24 | 3.29 | calcium binding protein 4 | 57010 | NM_001300895 | Hs.143036 | ENSG00000175544 |
| CASC9 | 4.66 | 3.98 | cancer susceptibility candidate 9 | 101805492 | NR_103848 | Hs.571424 | |
| | | | (non-protein coding) | | | | |
| CC2D2A | 3.76 | 3.39 | coiled-coil and C2 domain | 57545 | NM_001080522 | Hs.590928 | ENSG00000048342 |
| | | | containing 2A | | | | |
| CCDC122 | 3.01 | 2.62 | coiled-coil domain containing 122 | 160857 | NM_144974 | Hs.170849 | ENSG00000151773 |
| CCDC142 | 3.75 | 2.99 | coiled-coil domain containing 142 | 84865 | NM_032779 | Hs.430199 | ENSG00000135637 |
| CCDC148 | 5.74 | 3.89 | coiled-coil domain containing 148 | 130940 | NM_001171637 | Hs.668597 | ENSG00000153237 |
| CCDC30 | 3.52 | 2.99 | coiled-coil domain containing 30 | 728621 | NM_001080850 | Hs.729640 | ENSG00000186409 |
| CCL22 | 3.73 | 3.09 | chemokine (C-C motif) ligand 22 | 6367 | NM_002990 | Hs.534347 | ENSG00000102962 |
| CCL5 | 2.14 | 1.84 | chemokine (C-C motif) ligand 5 | 6352 | NM_001278736 | Hs.514821 | ENSG00000271503 |
| CD24 | 4.3 | 2.67 | CD24 molecule | 100133941 | NM_001291737 | Hs.644105 | ENSG00000272398 |
| CD300LG | 4.84 | 3.95 | CD300 molecule-like family member g | 146894 | NM_001168322 | Hs.147313 | ENSG00000161649 |
| CD3EAP | 3.67 | 2.87 | CD3e molecule, epsilon | 10849 | NM_001297590 | Hs.710495 | ENSG00000117877 |
| | | | associated protein | | | | |
| CD82 | 3.21 | 2.56 | CD82 molecule | 3732 | NM_001024844 | Hs.527778 | ENSG00000085117 |
| CDH23 | 3.32 | 2.79 | cadherin-related 23 | 64072 | NM_001171930 | Hs.656032 | ENSG00000107736 |
| CDKN2B-AS1 | 3.49 | 2.95 | CDKN2B antisense RNA 1 | 100048912 | NR_003529 | Hs.493614 | ENSG00000240498 |
| CEACAM22P | 4.41 | 3.57 | carcinoembryonic antigen-related cell | 388550 | NR_027754 | Hs.446909 | ENSG00000230666 |
| | | | adhesion molecule 22, pseudogene | | | | |
| CEACAM8 | 4.28 | 3.04 | carcinoembryonic antigen-related cell | 1088 | NM_001816 | Hs.41 | ENSG00000124469 |
| | | | adhesion molecule 8 | | | | |
| CENPBD1P1 | 2.78 | 2.54 | CENPB DNA-binding domains | 65996 | NM_023939 | Hs.541177 | ENSG00000213753 |
| | | | containing 1 pseudogene 1 | | | | |
| CENPN | 2.66 | 2.18 | centromere protein N | 55839 | NM_001100624 | Hs.726537 | ENSG00000166451 |
| CEP41 | 3.09 | 2.44 | centrosomal protein 41 kDa | 95681 | NM_001257158 | Hs.368315 | ENSG00000106477 |
| CES3 | 5.51 | 4.61 | carboxylesterase 3 | 23491 | NM_001185176 | Hs.268700 | ENSG00000172828 |
| CFLAR | 2.01 | 1.87 | CASP8 and FADD-like | 8837 | NM_001127183 | Hs.390736 | ENSG00000003402 |
| | | | apoptosis regulator | | | | |
| CHP1 | 2.48 | 2.17 | calcineurin-like EF-hand protein 1 | 11261 | NM_007236 | Hs.406234 | ENSG00000187446 |
| CHP2 | 3.66 | 2.96 | calcineurin-like EF-hand protein 2 | 63928 | NM_022097 | Hs.178589 | ENSG00000166869 |
| CHRM3 | 4.65 | 3.87 | cholinergic receptor, muscarinic 3 | 1131 | NM_000740 | Hs.7138 | ENSG00000133019 |
| CHRNB1 | 2.9 | 2.34 | cholinergic receptor, | 1140 | NM_000747 | Hs.330386 | ENSG00000170175 |
| | | | nicotinic, beta 1 (muscle) | | | | |
| CHST6 | 3.77 | 3.11 | carbohydrate (N-acetylglucosamine | 4166 | NM_021615 | Hs.655622 | ENSG00000183196 |
| | | | 6-O) sulfotransferase 6 | | | | |
| CIITA | 2.59 | 2.11 | class II, major histocompatibility | 4261 | NM_000246 | Hs.701991 | ENSG00000179583 |
| | | | complex, transactivator | | | | |
| CKMT2-AS1 | 2.71 | 2.28 | CKMT2 antisense RNA 1 | 100131067 | NR_034121 | Hs.655855 | ENSG00000247572 |
| CMBL | 4.29 | 3.43 | carboxymethylenebutenolidase | 134147 | NM_138809 | Hs.192586 | ENSG00000164237 |
| | | | homolog (Pseudomonas) | | | | |
| COA1 | 2.15 | 1.89 | cytochrome c oxidase | 55744 | NM_018224 | Hs.654779 | ENSG00000106603 |
| | | | assembly factor 1 homolog | | | | |
| COA7 | 3.1 | 2.43 | cytochrome c oxidase | 65260 | NM_023077 | Hs.349905 | ENSG00000162377 |
| | | | assembly factor 7 (putative) | | | | |
| COMMD2 | 2.34 | 1.97 | COMM domain containing 2 | 51122 | NM_016094 | Hs.432729 | ENSG00000114744 |
| COX10-AS1 | 2.4 | 2.23 | COX10 antisense RNA 1 | 100874058 | NR_049718 | Hs.720411 | ENSG00000236088 |
| COX18 | 2.38 | 2.1 | COX18 cytochrome c | 285521 | NM_001033760 | Hs.356697 | ENSG00000163626 |
| | | | oxidase assembly factor | | | | |
| COX6B2 | 4.89 | 4.27 | cytochrome c oxidase subunit VIb | 125965 | NM_144613 | Hs.550544 | ENSG00000160471 |
| | | | polypeptide 2 (testis) | | | | |
| CPB2-AS1 | 3.85 | 3.34 | CPB2 antisense RNA 1 | 100509894 | NR_046226 | Hs.626139 | ENSG00000235903 |
| CPM | 3.42 | 2.83 | carboxypeptidase M | 1368 | NM_001005502 | Hs.654387 | ENSG00000135678 |
| CPPED1 | 2.76 | 2.26 | calcineurin-like phosphoesterase | 55313 | NM_001099455 | Hs.460002 | ENSG00000103381 |
| | | | domain containing 1 | | | | |
| CRHR1-IT1 | 2.19 | 2.35 | CRHR1 intronic transcript 1 | 147081 | NM_152466 | Hs.128813 | ENSG00000204650 |
| CRLF2 | 4.72 | 4.02 | cytokine receptor-like factor 2 | 64109 | NM_001012288 | Hs.287729 | ENSG00000205755 |
| CRX | 4.76 | 3.8 | cone-rod homeobox | 1406 | NM_000554 | Hs.617342 | ENSG00000105392 |
| CRYBB2P1 | 3.5 | 2.54 | crystallin, beta B2 pseudogene 1 | 1416 | NR_033733 | Hs.571835 | ENSG00000100058 |
| CRYM-AS1 | 4.41 | 3.55 | CRYM antisense RNA 1 | 400508 | NM_001101368 | Hs.578949 | |
| CSAD | 3.09 | 2.47 | cysteine sulfinic acid decarboxylase | 51380 | NM_001244705 | Hs.279815 | ENSG00000139631 |
| CSTF3-AS1 | 4.09 | 3.38 | CSTF3 antisense RNA 1 | 338739 | NR_034027 | Hs.423476 | ENSG00000247151 |
| | | | (head to head) | | | | |
| CTBP2 | 2.56 | 2.38 | C-terminal binding protein 2 | 1488 | NM_001083914 | Hs.501345 | ENSG00000175029 |
| CTCFL | 4.52 | 3.12 | CCCTC-binding factor (zinc | 140690 | NM_001269040 | Hs.131543 | ENSG00000124092 |
| | | | finger protein)-like | | | | |
| CXorf36 | 4.4 | 3.54 | chromosome X open reading frame 36 | 79742 | NM_024689 | Hs.98321 | ENSG00000147113 |
| CXorf56 | 3.27 | 2.74 | chromosome X open reading frame 56 | 63932 | NM_001170569 | Hs.248572 | ENSG00000018610 |
| CYB5D2 | 2.48 | 2.3 | cytochrome b5 domain containing 2 | 124936 | NM_001254755 | Hs.513871 | ENSG00000167740 |
| CYP20A1 | 2.1 | 1.91 | cytochrome P450, family 20, | 57404 | NM_020674 | Hs.446065 | ENSG00000119004 |
| | | | subfamily A, polypeptide 1 | | | | |
| CYP4V2 | 2.25 | 2.04 | cytochrome P450, family 4, | 285440 | NM_207352 | Hs.587231 | ENSG00000145476 |
| | | | subfamily V, polypeptide 2 | | | | |
| CYP51A1 | 2.45 | 2.32 | cytochrome P450, family 51, | 1595 | NM_000786 | Hs.417077 | ENSG00000001630 |
| | | | subfamily A, polypeptide 1 | | | | |
| DAND5 | 4.28 | 3.4 | DAN domain family member | 199699 | NM_152654 | Hs.331981 | ENSG00000179284 |
| | | | 5, BMP antagonist | | | | |
| DAPP1 | 2.61 | 1.93 | dual adaptor of phosphotyrosine and | 27071 | NM_001306151 | Hs.436271 | ENSG00000070190 |
| | | | 3-phosphoinositides | | | | |
| DCUN1D2 | 4.15 | 3.39 | DCN1, defective in cullin neddylation | 55208 | NM_001014283 | Hs.682987 | ENSG00000150401 |
| | | | 1, domain containing 2 | | | | |
| DDX51 | 2.06 | 2.08 | DEAD (Asp-Glu-Ala-Asp) | 317781 | NM_175066 | Hs.445168 | ENSG00000185163 |
| | | | box polypeptide 51 | | | | |
| DESI1 | 2.29 | 2.1 | desumoylating isopeptidase 1 | 27351 | NM_015704 | Hs.570455 | ENSG00000100418 |
| DFFA | 2.4 | 2.14 | DNA fragmentation factor, | 1676 | NM_004401 | Hs.484782 | ENSG00000160049 |
| | | | 45 kDa, alpha polypeptide | | | | |
| DFFB | 2.59 | 2.21 | DNA fragmentation factor, | 1677 | NM_001004285 | Hs.133089 | ENSG00000169598 |
| | | | 40 kDa, beta polypeptide | | | | |
| | | | (caspase-activated DNase) | | | | |
| DHODH | 2.44 | 2.15 | dihydroorotate | 1723 | NM_001025193 | Hs.654427 | ENSG00000102967 |
| | | | dehydrogenase (quinone) | | | | |
| DLEU2 | 3.35 | 2.69 | deleted in lymphocytic leukemia 2 | 8847 | NR_002612 | Hs.547964 | |
| | | | (non-protein coding) | | | | |
| DLGAP1-AS2 | 4.82 | 3.67 | DLGAP1 antisense RNA 2 | 84777 | NM_032691 | Hs.659053 | |
| DLK2 | 3.07 | 2.84 | delta-like 2 homolog (Drosophila) | 65989 | NM_001286655 | Hs.337251 | ENSG00000171462 |
| DMC1 | 4 | 3.5 | DNA meiotic recombinase 1 | 11144 | NM_001278208 | Hs.339396 | ENSG00000100206 |
| DNAH17-AS1 | 3.55 | 2.95 | DNAH17 antisense RNA 1 | 100996295 | NR_102401 | Hs.615304 | |
| DNAJC22 | 4.1 | 3.66 | DnaJ (Hsp40) homolog, | 79962 | NM_001304944 | Hs.659300 | ENSG00000178401 |
| | | | subfamily C, member 22 | | | | |
| DNAJC27-AS1 | 3.67 | 3.24 | DNAJC27 antisense RNA 1 | 729723 | NR_034113 | Hs.436366 | ENSG00000224165 |
| DNAJC9-AS1 | 3.6 | 2.87 | DNAJC9 antisense RNA 1 | 414245 | NR_038373 | Hs.661857 | ENSG00000236756 |
| DNAL1 | 2.89 | 2.54 | dynein, axonemal, light chain 1 | 83544 | NM_001201366 | Hs.271270 | ENSG00000119661 |
| DNASE1 | 2.67 | 2.36 | deoxyribonuclease I | 1773 | NM_005223 | Hs.629638 | ENSG00000213918 |
| DNM1P46 | 2.57 | 2.24 | dynamin 1 pseudogene 46 | 196968 | NM_194295 | Hs.567763 | ENSG00000182397 |
| DPH3P1 | 3.77 | 3.43 | diphthamide biosynthesis 3 | 100132911 | NM_080750 | | |
| | | | pseudogene 1 | | | | |
| DPY19L1P1 | 2.75 | 3 | DPY19L1 pseudogene 1 | 100129460 | NR_036680 | Hs.633705 | |
| DPY19L2P2 | 3.48 | 3.02 | DPY19L2 pseudogene 2 | 349152 | NM_182634 | Hs.732579 | ENSG00000170629 |
| DSG2 | 4.49 | 3.32 | desmoglein 2 | 1829 | NM_001943 | Hs.412597 | ENSG00000046604 |
| DSG3 | 3.99 | 3.27 | desmoglein 3 | 1830 | NM_001944 | Hs.1925 | ENSG00000134757 |
| DTD2 | 2.58 | 2.22 | D-tyrosyl-tRNA deacylase 2 (putative) | 112487 | NM_080664 | Hs.116014 | ENSG00000129480 |
| DUXA | 4.59 | 3.88 | double homeobox A | 503835 | NM_001012729 | Hs.585857 | ENSG00000258873 |
| DYDC1 | 3.82 | 3.41 | DPY30 domain containing 1 | 143241 | NM_001269053 | Hs.407751 | ENSG00000170788 |
| DYNAP | 5.01 | 3.36 | dynactin associated protein | 284254 | NM_001307955 | Hs.376146 | ENSG00000178690 |
| ECT2 | 3.62 | 2.68 | epithelial cell transforming 2 | 1894 | NM_001258315 | Hs.518299 | ENSG00000114346 |
| EEF2K | 2.06 | 1.87 | eukaryotic elongation factor 2 kinase | 29904 | NM_013302 | Hs.498892 | ENSG00000103319 |
| EFCAB11 | 3.61 | 3.02 | EF-hand calcium binding domain 11 | 90141 | NM_001284266 | Hs.123232 | ENSG00000140025 |
| EGFEM1P | 4.33 | 3.45 | EGF-like and EMI domain | 93556 | NR_021485 | Hs.478158 | |
| | | | containing 1, pseudogene | | | | |
| EID2B | 2.85 | 2.44 | EP300 interacting inhibitor | 126272 | NM_152361 | Hs.135181 | ENSG00000176401 |
| | | | of differentiation 2B | | | | |
| ELMOD1 | 4.61 | 3.72 | ELMO/CED-12 domain | 55531 | NM_001130037 | Hs.495779 | ENSG00000110675 |
| | | | containing 1 | | | | |
| EMP2 | 3.97 | 3.43 | epithelial membrane protein 2 | 2013 | NM_001424 | Hs.531561 | ENSG00000213853 |
| EMX2OS | 4.29 | 3.68 | EMX2 opposite strand/antisense RNA | 196047 | NR_002791 | Hs.312592 | ENSG00000229847 |
| ENTPD1 | 3.56 | 2.8 | ectonucleoside triphosphate | 953 | NM_001098175 | Hs.576612 | ENSG00000138185 |
| | | | diphosphohydrolase 1 | | | | |
| ENTPD1-AS1 | 3.75 | 3.18 | ENTPD1 antisense RNA 1 | 728558 | NR_038444 | Hs.538374 | ENSG00000226688 |
| EP300-AS1 | 5.34 | 3.95 | EP300 antisense RNA 1 | 101927279 | NR_110514 | Hs.517517 | |
| EPGN | 4.64 | 3.69 | epithelial mitogen | 255324 | NM_001013442 | Hs.401237 | ENSG00000182585 |
| EPHA10 | 4.57 | 3.81 | EPH receptor A10 | 284656 | NM_001004338 | Hs.129435 | ENSG00000183317 |
| EPPIN | 4.35 | 3.47 | epididymal peptidase inhibitor | 57119 | NM_001302861 | Hs.121084 | ENSG00000101448 |
| ERVK13-1 | 2.07 | 1.94 | endogenous retrovirus group | 100507321 | NM_001012731 | Hs.406976 | |
| | | | K13, member 1 | | | | |
| ERVV-1 | 4.55 | 4.02 | endogenous retrovirus group | 147664 | NM_152473 | Hs.44329 | ENSG00000269526 |
| | | | V, member 1 | | | | |
| ESRG | 5.79 | 4.25 | embryonic stem cell related | 790952 | NR_027122 | Hs.720658 | ENSG00000265992 |
| | | | (non-protein coding) | | | | |
| EXD1 | 4.36 | 3.54 | exonuclease 3′-5′ domain | 161829 | NM_001286441 | Hs.307999 | ENSG00000178997 |
| | | | containing 1 | | | | |
| EXOC3L2 | 3.78 | 2.96 | exocyst complex component 3-like 2 | 90332 | NM_138568 | Hs.337557 | ENSG00000130201 |
| EXPH5 | 3.01 | 2.73 | exophilin 5 | 23086 | NM_001144763 | Hs.28540 | ENSG00000110723 |
| F5 | 2.55 | 1.87 | coagulation factor V | 2153 | NM_000130 | Hs.30054 | ENSG00000198734 |
| | | | (proaccelerin, labile factor) | | | | |
| FAIM | 3.04 | 2.51 | Fas apoptotic inhibitory molecule | 55179 | NM_001033030 | Hs.173438 | ENSG00000158234 |
| FAM106A | 4.01 | 3.81 | family with sequence | 80039 | NM_024974 | Hs.674403 | ENSG00000213077 |
| | | | similarity 106, member A | | | | |
| FAM114A1 | 4.04 | 2.87 | family with sequence | 92689 | NM_138389 | Hs.476517 | ENSG00000197712 |
| | | | similarity 114, member A1 | | | | |
| FAM122C | 3.02 | 2.67 | family with sequence similarity 122C | 159091 | NM_001170779 | Hs.269127 | ENSG00000156500 |
| FAM153C | 3.38 | 3.2 | family with sequence similarity 153, | 653316 | NM_001079527 | Hs.652193 | ENSG00000204677 |
| | | | member C, pseudogene | | | | |
| FAM231A | 2.11 | 2.64 | family with sequence similarity 231, | 729574 | NM_001282321 | | ENSG00000237847 |
| | | | member A | | | | |
| FAM71F2 | 3.98 | 3.49 | family with sequence similarity 71, | 346653 | NM_001012454 | Hs.445236 | ENSG00000205085 |
| | | | member F2 | | | | |
| FAM73A | 2.49 | 2.21 | family with sequence similarity 73, | 374986 | NM_001270384 | Hs.437755 | ENSG00000180488 |
| | | | member A | | | | |
| FAM74A3 | 4.26 | 3.66 | family with sequence similarity 74, | 728495 | NM_001098718 | Hs.723007 | |
| | | | member A3 | | | | |
| FAM83H-AS1 | 4.13 | 3.28 | FAM83H antisense RNA 1 | 100128338 | NR_033849 | Hs.493171 | ENSG00000203499 |
| | | | (head to head) | | | | |
| FBLIM1 | 4.42 | 3.64 | filamin binding LIM protein 1 | 54751 | NM_001024215 | Hs.530101 | ENSG00000162458 |
| FBLN1 | 5.56 | 4.26 | fibulin 1 | 2192 | NM_001996 | Hs.24601 | ENSG00000077942 |
| FBXL18 | 3.27 | 2.48 | F-box and leucine-rich repeat | 80028 | NM_024963 | Hs.623974 | ENSG00000155034 |
| | | | protein 18 | | | | |
| FBXO17 | 4.71 | 3.73 | F-box protein 17 | 115290 | NM_024907 | Hs.531770 | ENSG00000269190 |
| FBXO27 | 4.21 | 3.63 | F-box protein 27 | 126433 | NM_178820 | Hs.187461 | ENSG00000161243 |
| FBXO45 | 2.56 | 2.08 | F-box protein 45 | 200933 | NM_001105573 | Hs.169815 | ENSG00000174013 |
| FBXO6 | 2.95 | 2.66 | F-box protein 6 | 26270 | NM_018438 | Hs.464419 | ENSG00000116663 |
| FCAR | 4.27 | 3.38 | Fc fragment of IgA receptor | 2204 | NM_002000 | Hs.659872 | ENSG00000275136 |
| FDPSP2 | 4.04 | 3.16 | farnesyl diphosphate | 619190 | NR_003262 | Hs.609978 | ENSG00000233980 |
| | | | synthase pseudogene 2 | | | | |
| FEZ1 | 3.19 | 3.63 | fasciculation and elongation | 9638 | NM_005103 | Hs.224008 | ENSG00000149557 |
| | | | protein zeta 1 (zygin I) | | | | |
| FGD5P1 | 4.12 | 3.22 | FYVE, RhoGEF and PH domain | 100132526 | NR_036481 | Hs.637770 | ENSG00000275340 |
| | | | containing 5 pseudogene 1 | | | | |
| FGF5 | 3.97 | 3.16 | fibroblast growth factor 5 | 2250 | NM_001291812 | Hs.37055 | ENSG00000138675 |
| FGFR1OP | 2.9 | 2.36 | FGFR1 oncogene partner | 11116 | NM_001278690 | Hs.487175 | ENSG00000213066 |
| FILIP1 | 5.4 | 4 | filamin A interacting protein 1 | 27145 | NM_001289987 | Hs.696158 | ENSG00000118407 |
| FKBP14 | 3.47 | 2.87 | FK506 binding protein 14, 22 kDa | 55033 | NM_017946 | Hs.390838 | ENSG00000106080 |
| FLCN | 2.43 | 2.18 | folliculin | 201163 | NM_144606 | Hs.31652 | ENSG00000154803 |
| FLJ31104 | 3.77 | 3.06 | uncharacterized LOC441072 | 441072 | NR_102755 | Hs.482141 | ENSG00000227908 |
| FLJ31356 | 4.5 | 3.72 | uncharacterized protein FLJ31356 | 403150 | NR_103831 | Hs.562970 | ENSG00000229951 |
| FLJ31662 | 5.11 | 4.05 | uncharacterized LOC440594 | 440594 | NR_033966 | Hs.514123 | ENSG00000233907 |
| FLJ42102 | 4.48 | 3.47 | uncharacterized LOC399923 | 399923 | NM_001001680 | Hs.128191 | ENSG00000172900 |
| FRMD6-S1 | 53.64 | 71.46 | FRMD6 antisense RNA 1 | 145438 | NR_037676 | Hs.645410 | ENSG00000273888 |
| FRRS1 | 5.18 | 4.33 | ferric-chelate reductase 1 | 391059 | NM_001013660 | Hs.454779 | ENSG00000156869 |
| FRY-AS1 | 4.5 | 3.46 | FRY antisense RNA 1 | 100507099 | NR_103839 | Hs.536364 | |
| FTX | 2.53 | 2.33 | FTX transcript, XIST regulator | 100302692 | NR_028379 | Hs.349570 | ENSG00000230590 |
| | | | (non-protein coding) | | | | |
| FUT1 | 4.07 | 3.23 | fucosyltransferase 1 (galactoside 2- | 2523 | NM_000148 | Hs.69747 | ENSG00000174951 |
| | | | alpha-L-fucosyltransferase, H blood | | | | |
| | | | group) | | | | |
| FUT2 | 4.29 | 3.4 | fucosyltransferase 2 (secretor | 2524 | NM_000511 | Hs.579928 | ENSG00000176920 |
| | | | status included) | | | | |
| FUT6 | 3.68 | 3.11 | fucosyltransferase 6 (alpha | 2528 | NM_000150 | Hs.631846 | ENSG00000156413 |
| | | | (1,3) fucosyltransferase) | | | | |
| GAL3ST4 | 2.8 | 2.17 | galactose-3-O-sulfotransferase 4 | 79690 | NM_024637 | Hs.44856 | ENSG00000197093 |
| GALNT15 | 3.4 | 3.03 | polypeptide N- | 117248 | NM_054110 | Hs.411308 | ENSG00000131386 |
| | | | acetylgalactosaminyltransferase 15 | | | | |
| GAS6-AS2 | 4.23 | 3.72 | GAS6 antisense RNA 2 | 100506394 | NR_044993 | Hs.132168 | ENSG00000272695 |
| | | | (head to head) | | | | |
| GATAD1 | 2.19 | 2.09 | GATA zinc finger domain | 57798 | NM_021167 | Hs.21145 | ENSG00000157259 |
| | | | containing 1 | | | | |
| GDPD1 | 3.86 | 3.45 | glycerophosphodiester | 284161 | NM_001165993 | Hs.631744 | ENSG00000153982 |
| | | | phosphodiesterase domain | | | | |
| | | | containing 1 | | | | |
| GEMIN8 | 3.59 | 2.91 | gem (nuclear organelle) | 54960 | NM_001042479 | Hs.592237 | ENSG00000046647 |
| | | | associated protein 8 | | | | |
| GFOD2 | 3.21 | 2.57 | glucose-fructose oxidoreductase | 81577 | NM_001243650 | Hs.307084 | ENSG00000141098 |
| | | | domain containing 2 | | | | |
| GGT6 | 4.3 | 3.55 | gamma-glutamyltransferase 6 | 124975 | NM_001122890 | Hs.130749 | ENSG00000167741 |
| GGT8P | 4.96 | 3.97 | gamma-glutamyltransferase 8 | 645367 | NR_003503 | Hs.650223 | |
| | | | pseudogene | | | | |
| GK5 | 2.43 | 2.21 | glycerol kinase 5 (putative) | 256356 | NM_001039547 | Hs.135904 | ENSG00000175066 |
| GLIPR1L2 | 5.27 | 3.7 | GLI pathogenesis-related 1 like 2 | 144321 | NM_001270396 | Hs.406728 | ENSG00000180481 |
| GNB4 | 3.49 | 2.72 | guanine nucleotide binding protein | 59345 | NM_021629 | Hs.173030 | ENSG00000114450 |
| | | | (G protein), beta polypeptide 4 | | | | |
| GNE | 3.01 | 2.47 | glucosamine (UDP-N- | 10020 | NM_001128227 | Hs.5920 | ENSG00000159921 |
| | | | acetyl)-2-epimerase/N- | | | | |
| | | | acetylmannosamine kinase | | | | |
| GNRHR2 | 4.67 | 3.52 | gonadotropin-releasing hormone | 114814 | NM_057163 | Hs.356873 | ENSG00000211451 |
| | | | (type 2) receptor 2, pseudogene | | | | |
| GOLGA2 | 2.53 | 2.02 | golgin A2 | 2801 | NM_004486 | Hs.155827 | ENSG00000167110 |
| GOLGA6L22 | 5.03 | 3.86 | golgin A6 family-like 22 | 440243 | NM_001271664 | | ENSG00000274404 |
| GOLGA6L6 | 4.6 | 3.28 | golgin A6 family-like 6 | 727832 | NM_001145004 | Hs.569472 | ENSG00000277322 |
| GOSR1 | 3.08 | 2.61 | golgi SNAP receptor | 9527 | NM_001007024 | Hs.462680 | ENSG00000108587 |
| | | | complex member 1 | | | | |
| GPR1-AS | 4.67 | 3.49 | GPR1 antisense RNA | 101669764 | NR_104359 | Hs.574781 | ENSG00000279220 |
| GPR37L1 | 4.34 | 3.11 | G protein-coupled receptor 37 like 1 | 9283 | NM_004767 | Hs.132049 | ENSG00000170075 |
| GPR82 | 3.34 | 3.21 | G protein-coupled receptor 82 | 27197 | NM_080817 | Hs.567457 | ENSG00000171657 |
| GREB1 | 4.84 | 3.69 | growth regulation by | 9687 | NM_014668 | Hs.467733 | ENSG00000196208 |
| | | | estrogen in breast cancer 1 | | | | |
| GRTP1 | 3.91 | 3.25 | growth hormone regulated | 79774 | NM_001286732 | Hs.745043 | ENSG00000139835 |
| | | | TBC protein 1 | | | | |
| GSDMA | 3.59 | 3.02 | gasdermin A | 284110 | NM_178171 | Hs.448873 | ENSG00000167914 |
| GSG1 | 4.38 | 3.34 | germ cell associated 1 | 83445 | NM_001080554 | Hs.240053 | ENSG00000111305 |
| GSTM3 | 3.45 | 2.8 | glutathione S-transferase mu 3 (brain) | 2947 | NM_000849 | Hs.2006 | ENSG00000134202 |
| GTF2E1 | 3.49 | 2.48 | general transcription factor IIE, | 2960 | NM_005513 | Hs.445272 | ENSG00000153767 |
| | | | polypeptide 1, alpha 56 kDa | | | | |
| GTF2H2 | 2.46 | 2.1 | general transcription factor | 2966 | NM_001515 | Hs.191356 | ENSG00000145736 |
| | | | IIH, polypeptide 2, 44 kDa | | | | |
| GUCA1B | 4.5 | 3.03 | guanylate cyclase activator 1B (retina) | 2979 | NM_002098 | Hs.446529 | ENSG00000112599 |
| GUSBP3 | 2.42 | 2.31 | glucuronidase, beta pseudogene 3 | 653188 | NR_027386 | Hs.631974 | ENSG00000253203 |
| H1FX-AS1 | 2.98 | 2.44 | H1FX antisense RNA 1 | 339942 | NM_001025468 | Hs.450096 | |
| HCAR1 | 3.99 | 3.68 | hydroxycarboxylic acid receptor 1 | 27198 | NM_032554 | Hs.610873 | ENSG00000196917 |
| HEATR5A | 2.38 | 2.18 | HEAT repeat containing 5A | 25938 | NM_015473 | Hs.744979 | ENSG00000129493 |
| HES2 | 4.11 | 3.44 | hes family bHLH transcription factor 2 | 54626 | NM_019089 | Hs.118727 | ENSG00000069812 |
| HHLA2 | 2.46 | 2.41 | HERV-H LTR-associating 2 | 11148 | NM_001282556 | Hs.225968 | ENSG00000114455 |
| HILPDA | 3.18 | 2.83 | hypoxia inducible lipid | 29923 | NM_001098786 | Hs.706124 | ENSG00000135245 |
| | | | droplet-associated | | | | |
| HIPK1-AS1 | 2.57 | 2.41 | HIPK1 antisense RNA 1 | 101928846 | NR_110725 | Hs.232534 | ENSG00000235527 |
| HMGB3P1 | 4.77 | 4.13 | high mobility group box 3 | 128872 | NR_002165 | Hs.558624 | |
| | | | pseudogene 1 | | | | |
| HNF1A-AS1 | 4.5 | 3.36 | HNF1A antisense RNA 1 | 283460 | NR_024345 | Hs.612351 | ENSG00000241388 |
| HOGA1 | 4.32 | 3.68 | 4-hydroxy-2-oxoglutarate aldolase 1 | 112817 | NM_001134670 | Hs.180346 | ENSG00000241935 |
| HP09025 | 3.99 | 3.31 | uncharacterized LOC100652929 | 100652929 | NR_109783 | Hs.559249 | ENSG00000267719 |
| HPSE | 2.05 | 1.79 | heparanase | 10855 | NM_001098540 | Hs.44227 | ENSG00000173083 |
| HSD17B13 | 4.35 | 3.67 | hydroxysteroid (17-beta) | 345275 | NM_001136230 | Hs.284414 | ENSG00000170509 |
| | | | dehydrogenase 13 | | | | |
| HSP90AB4P | 4.23 | 3.1 | heat shock protein 90 kDa | 664618 | NR_002927 | Hs.670224 | |
| | | | alpha (cytosolic), class B | | | | |
| | | | member 4, pseudogene | | | | |
| HTRA4 | 4.59 | 3.31 | HtrA serine peptidase 4 | 203100 | NM_153692 | Hs.661014 | ENSG00000169495 |
| IAPP | 3.94 | 3.28 | islet amyloid polypeptide | 3375 | NM_000415 | Hs.46835 | ENSG00000121351 |
| IBA57 | 2.85 | 2.58 | IBA57 homolog, iron-sulfur | 200205 | NM_001010867 | Hs.237017 | ENSG00000181873 |
| | | | cluster assembly | | | | |
| ICA1L | 3.12 | 2.91 | islet cell autoantigen 1.69 kDa-like | 130026 | NM_001288622 | Hs.516629 | ENSG00000163596 |
| IDO1 | 5.13 | 4.37 | indoleamine 2,3-dioxygenase 1 | 3620 | NM_002164 | Hs.840 | ENSG00000131203 |
| IFNLR1 | 4.24 | 3.59 | interferon, lambda receptor 1 | 163702 | NM_170743 | Hs.221375 | ENSG00000185436 |
| IFT22 | 3.1 | 2.69 | intraflagellar transport 22 | 64792 | NM_001130820 | Hs.389104 | ENSG00000128581 |
| IL10 | 4.12 | 3.83 | interleukin 10 | 3586 | NM_000572 | Hs.193717 | ENSG00000136634 |
| IL15 | 3.25 | 2.56 | interleukin 15 | 3600 | NM_000585 | Hs.168132 | ENSG00000164136 |
| IL17RD | 3.95 | 3.23 | interleukin 17 receptor D | 54756 | NM_017563 | Hs.150725 | ENSG00000144730 |
| INE1 | 2.81 | 2.75 | inactivation escape 1 (non- | 8552 | NM_003669 | Hs.657350 | ENSG00000224975 |
| | | | protein coding) | | | | |
| INGX | 4.56 | 3.7 | inhibitor of growth family, | 27160 | NR_002226 | Hs.721806 | ENSG00000243468 |
| | | | X-linked, pseudogene | | | | |
| INIP | 2.46 | 1.99 | INTS3 and NABP interacting protein | 58493 | NM_021218 | Hs.658575 | ENSG00000148153 |
| INMT | 4.25 | 3.37 | indolethylamine N-methyltransferase | 11185 | NM_001199219 | Hs.632629 | ENSG00000241644 |
| IPO5P1 | 2.4 | 2.1 | importin 5 pseudogene 1 | 100132815 | NR_103741 | Hs.629249 | |
| IRGQ | 3.27 | 2.78 | immunity-related GTPase family, Q | 126298 | NM_001007561 | Hs.6217 | ENSG00000167378 |
| ITIH5 | 4.34 | 3.6 | inter-alpha-trypsin inhibitor heavy | 80760 | NM_001001851 | Hs.498586 | ENSG00000123243 |
| | | | chain family, member 5 | | | | |
| JPX | 3.59 | 3.15 | JPX transcript, XIST activator | 554203 | NR_024582 | Hs.648316 | ENSG00000225470 |
| | | | (non-protein coding) | | | | |
| KANTR | 2.83 | 2.58 | KDM5C adjacent non- | 102723508 | NR_110456 | Hs.633244 | |
| | | | coding transcript | | | | |
| KBTBD12 | 4.43 | 3.83 | kelch repeat and BTB (POZ) | 166348 | NM_207335 | Hs.132087 | ENSG00000187715 |
| | | | domain containing 12 | | | | |
| KBTBD6 | 2.46 | 2.05 | kelch repeat and BTB (POZ) | 89890 | NM_152903 | Hs.534040 | ENSG00000165572 |
| | | | domain containing 6 | | | | |
| KCNA7 | 4.88 | 3.92 | potassium channel, voltage gated | 3743 | NM_031886 | Hs.306973 | ENSG00000104848 |
| | | | shaker related subfamily A, member 7 | | | | |
| KCNJ11 | 3.9 | 3.43 | potassium channel, inwardly | 3767 | NM_000525 | Hs.248141 | ENSG00000187486 |
| | | | rectifying subfamily J, member 11 | | | | |
| KCNJ5 | 4.19 | 3.57 | potassium channel, inwardly | 3762 | NM_000890 | Hs.444595 | ENSG00000120457 |
| | | | rectifying subfamily J, member 5 | | | | |
| KCNQ1OT1 | 4.08 | 3.18 | KCNQ1 opposite strand/antisense | 10984 | NR_002728 | Hs.604823 | ENSG00000269821 |
| | | | transcript 1 (non-protein coding) | | | | |
| KDELC2 | 2.63 | 2.33 | KDEL (Lys-Asp-Glu-Leu) | 143888 | NM_153705 | Hs.83286 | ENSG00000178202 |
| | | | containing 2 | | | | |
| KDM4A-S1 | 4.3 | 3.3 | KDM4A antisense RNA 1 | 100132774 | NR_033827 | Hs.655569 | |
| KIAA0101 | 4.61 | 2.88 | KIAA0101 | 9768 | NM_001029989 | Hs.81892 | ENSG00000166803 |
| KIAA1161 | 4.87 | 3.65 | KIAA1161 | 57462 | NM_020702 | Hs.522083 | ENSG00000164976 |
| KIAA1324 | 2.42 | 2.31 | KIAA1324 | 57535 | NM_001267048 | Hs.708190 | ENSG00000116299 |
| KIAA1456 | 4.71 | 3.43 | KIAA1456 | 57604 | NM_001099677 | Hs.202521 | ENSG00000250305 |
| KIAA1614 | 3.66 | 2.84 | KIAA1614 | 57710 | NM_020950 | Hs.734816 | ENSG00000135835 |
| KIAA1919 | 3.49 | 2.61 | KIAA1919 | 91749 | NM_153369 | Hs.400572 | ENSG00000173214 |
| KIF18B | 4.05 | 3.41 | kinesin family member 18B | 146909 | NM_001080443 | Hs.135094 | ENSG00000186185 |
| KIF1B | 2.07 | 1.75 | kinesin family member 1B | 23095 | NM_015074 | Hs.97858 | ENSG00000054523 |
| KIF3A | 2.18 | 1.94 | kinesin family member 3A | 11127 | NM_001300791 | Hs.43670 | ENSG00000131437 |
| KIR3DX1 | 3.74 | 3.28 | killer cell immunoglobulin-like | 90011 | NM_001047605 | Hs.288520 | ENSG00000104970 |
| | | | receptor, three domains, X1 | | | | |
| KLF3-AS1 | 2.34 | 2.48 | KLF3 antisense RNA 1 | 79667 | NM_024614 | Hs.29725 | ENSG00000231160 |
| KLRD1 | 2.65 | 2.36 | killer cell lectin-like receptor | 3824 | NM_001114396 | Hs.562457 | ENSG00000134539 |
| | | | subfamily D, member 1 | | | | |
| KREMEN1 | 4.81 | 3.4 | kringle containing | 83999 | NM_001039570 | Hs.229335 | ENSG00000183762 |
| | | | transmembrane protein 1 | | | | |
| KRT18 | 2.8 | 2.75 | keratin 18, type I | 3875 | NM_000224 | Hs.406013 | ENSG00000111057 |
| KRT8 | 3.45 | 2.95 | keratin 8, type II | 3856 | NM_001256282 | Hs.533782 | ENSG00000170421 |
| L1TD1 | 4.64 | 3.67 | LINE-1 type transposase | 54596 | NM_001164835 | Hs.685462 | ENSG00000240563 |
| | | | domain containing 1 | | | | |
| L2HGDH | 4.91 | 3.54 | L-2-hydroxyglutarate dehydrogenase | 79944 | NM_024884 | Hs.256034 | ENSG00000087299 |
| LAIR1 | 2.87 | 2.24 | leukocyte-associated immunoglobulin- | 3903 | NM_001289023 | Hs.572535 | ENSG00000167613 |
| | | | like receptor 1 | | | | |
| LARS2-AS1 | 3.66 | 2.87 | LARS2 antisense RNA 1 | 100885795 | NR_048543 | Hs.641094 | |
| LDLR | 2.41 | 2.26 | low density lipoprotein receptor | 3949 | NM_000527 | Hs.213289 | ENSG00000130164 |
| LGMN | 3.31 | 2.67 | legumain | 5641 | NM_001008530 | Hs.18069 | ENSG00000100600 |
| LIFR-AS1 | 5.36 | 3.4 | LIFR antisense RNA 1 | 100506495 | NR_103553 | Hs.657602 | ENSG00000244968 |
| LINC00092 | 2.59 | 2.33 | long intergenic non-protein | 100188953 | NR_024129 | Hs.434310 | ENSG00000225194 |
| | | | coding RNA 92 | | | | |
| LINC00260 | 2.91 | 2.68 | long intergenic non-protein | 84719 | NM_032633 | Hs.661178 | |
| | | | coding RNA 260 | | | | |
| LINC00294 | 3.93 | 3.15 | long intergenic non-protein | 283267 | NR_015451 | Hs.533701 | ENSG00000280798 |
| | | | coding RNA 294 | | | | |
| LINC00311 | 3.85 | 3.74 | long intergenic non-protein | 197196 | NM_153238 | Hs.679002 | ENSG00000179219 |
| | | | coding RNA 311 | | | | |
| LINC00346 | 5.19 | 3.87 | long intergenic non-protein | 283487 | NM_178514 | Hs.245390 | ENSG00000255874 |
| | | | coding RNA 346 | | | | |
| LINC00371 | 6.06 | 4.54 | long intergenic non-protein | 647166 | NR_102431 | Hs.195052 | |
| | | | coding RNA 371 | | | | |
| LINC00381 | 4.68 | 3.4 | long intergenic non-protein | 100874151 | NR_047005 | Hs.564552 | ENSG00000226240 |
| | | | coding RNA 381 | | | | |
| LINC00458 | 5.94 | 4.75 | long intergenic non-protein | 100507428 | NR_108062 | Hs.351262 | ENSG00000234787 |
| | | | coding RNA 458 | | | | |
| LINC00470 | 3.85 | 2.95 | long intergenic non-protein | 56651 | NM_031416 | Hs.541165 | |
| | | | coding RNA 470 | | | | |
| LINC00483 | 3.52 | 3.19 | long intergenic non-protein | 55018 | NM_017928 | Hs.389460 | ENSG00000167117 |
| | | | coding RNA 483 | | | | |
| LINC00485 | 7.11 | 5.02 | long intergenic non-protein | 283432 | NR_033855 | Hs.382110 | ENSG00000258169 |
| | | | coding RNA 485 | | | | |
| LINC00501 | 4.51 | 4.36 | long intergenic non-protein | 100820709 | NR_047465 | Hs.518409 | ENSG00000203645 |
| | | | coding RNA 501 | | | | |
| LINC00506 | 4.36 | 3.32 | long intergenic non-protein | 100846978 | NR_047469 | Hs.570649 | ENSG00000281392 |
| | | | coding RNA 506 | | | | |
| LINC00507 | 5.42 | 4.12 | long intergenic non-protein | 100862680 | NR_046392 | Hs.385496 | ENSG00000256193 |
| | | | coding RNA 507 | | | | |
| LINC00547 | 5.74 | 4.09 | long intergenic non-protein | 400121 | NR_040244 | Hs.558894 | ENSG00000275226 |
| | | | coding RNA 547 | | | | |
| LINC00578 | 4.72 | 3.66 | long intergenic non-protein | 100505566 | NR_047568 | Hs.581170 | |
| | | | coding RNA 578 | | | | |
| LINC00620 | 5.09 | 3.5 | long intergenic non-protein | 285375 | NR_027103 | Hs.319969 | ENSG00000224514 |
| | | | coding RNA 620 | | | | |
| LINC00649 | 2.97 | 2.45 | long intergenic non-protein | 400863 | NM_001288961 | Hs.729814 | ENSG00000237945 |
| | | | coding RNA 649 | | | | |
| LINC00652 | 4.41 | 3.52 | long intergenic non-protein | 29075 | NM_014162 | Hs.584899 | |
| | | | coding RNA 652 | | | | |
| LINC00663 | 4.14 | 3.4 | long intergenic non-protein | 284440 | NR_026956 | Hs.665307 | |
| | | | coding RNA 663 | | | | |
| LINC00665 | 3.09 | 2.84 | long intergenic non-protein | 100506930 | NR_038278 | Hs.595153 | ENSG00000232677 |
| | | | coding RNA 665 | | | | |
| LINC00670 | 4.75 | 3.48 | long intergenic non-protein | 284034 | NR_034144 | Hs.376614 | ENSG00000179136 |
| | | | coding RNA 670 | | | | |
| LINC00672 | 3.85 | 3 | long intergenic non-protein | 100505576 | NR_038847 | Hs.634043 | ENSG00000263874 |
| | | | coding RNA 672 | | | | |
| LINC00678 | 5.57 | 3.89 | long intergenic non-protein | 101410541 | NR_102708 | Hs.471439 | ENSG00000254934 |
| | | | coding RNA 678 | | | | |
| LINC00889 | 5.99 | 4.42 | long intergenic non-protein | 158696 | NR_026935 | Hs.558664 | |
| | | | coding RNA 889 | | | | |
| LINC00907 | 3.95 | 3.12 | long intergenic non-protein | 284260 | NR_046174 | Hs.652819 | ENSG00000267586 |
| | | | coding RNA 907 | | | | |
| LINC00910 | 3 | 2.48 | long intergenic non-protein | 100130581 | NR_027412 | Hs.546897 | ENSG00000188825 |
| | | | coding RNA 910 | | | | |
| LINC00923 | 2.84 | 2.56 | long intergenic non-protein | 91948 | NR_024172 | Hs.130423 | ENSG00000251209 |
| | | | coding RNA 923 | | | | |
| LINC00924 | 4.4 | 3.05 | long intergenic non-protein | 145820 | NR_027132 | Hs.652702 | ENSG00000259134 |
| | | | coding RNA 924 | | | | |
| LINC00941 | 3.79 | 3.38 | long intergenic non-protein | 100287314 | NR_040245 | Hs.355210 | ENSG00000235884 |
| | | | coding RNA 941 | | | | |
| LINC00958 | 4.91 | 3.92 | long intergenic non-protein | 100506305 | NR_038904 | Hs.153408 | ENSG00000251381 |
| | | | coding RNA 958 | | | | |
| LINC00963 | 2.8 | 2.51 | long intergenic non-protein | 100506190 | NR_038955 | Hs.529860 | |
| | | | coding RNA 963 | | | | |
| LINC00965 | 4.43 | 3.51 | long intergenic non-protein | 349196 | NM_001025473 | Hs.559040 | |
| | | | coding RNA 965 | | | | |
| LINC00970 | 4.24 | 3.32 | long intergenic non-protein | 101978719 | NR_104091 | Hs.517849 | ENSG00000203601 |
| | | | coding RNA 970 | | | | |
| LINC01012 | 3.13 | 2.69 | long intergenic non-protein | 100507173 | NR_038292 | Hs.635987 | ENSG00000281706 |
| | | | coding RNA 1012 | | | | |
| LINC01021 | 6.93 | 4.93 | long intergenic non-protein | 643401 | NR_038848 | Hs.533212 | ENSG00000250337 |
| | | | coding RNA 1021 | | | | |
| LINC01057 | 5.48 | 3.49 | long intergenic non-protein | 101928079 | NR_104131 | Hs.596857 | ENSG00000224081 |
| | | | coding RNA 1057 | | | | |
| LINC01087 | 5.01 | 3.44 | long intergenic non-protein | 101927994 | NR_108087 | Hs.635757 | ENSG00000224559 |
| | | | coding RNA 1087 | | | | |
| LINC01099 | 4.33 | 3.58 | long intergenic non-protein | 101928656 | NR_108092 | Hs.508131 | ENSG00000251504 |
| | | | coding RNA 1099 | | | | |
| LINC01160 | 4.12 | 3.22 | long intergenic non-protein | 100129269 | NR_034126 | Hs.689728 | |
| | | | coding RNA 1160 | | | | |
| LINC01204 | 4.02 | 3.25 | long intergenic non-protein | 101927528 | NR_104644 | Hs.550772 | ENSG00000229563 |
| | | | coding RNA 1204 | | | | |
| LINC01205 | 4.55 | 3.45 | long intergenic non-protein | 401082 | NM_001145553 | Hs.477089 | ENSG00000228980 |
| | | | coding RNA 1205 | | | | |
| LINC01207 | 4.05 | 3.59 | long intergenic non-protein | 100505989 | NR_038834 | Hs.328236 | ENSG00000248771 |
| | | | coding RNA 1207 | | | | |
| LINC01209 | 4.74 | 3.4 | long intergenic non-protein | 101928684 | NR_110819 | Hs.639352 | ENSG00000228308 |
| | | | coding RNA 1209 | | | | |
| LINC01212 | 3.81 | 3.27 | long intergenic non-protein | 101927152 | NR_110000 | Hs.382046 | ENSG00000240405 |
| | | | coding RNA 1212 | | | | |
| LINC01226 | 4.31 | 3.34 | long intergenic non-protein | 284551 | NR_027085 | Hs.658659 | ENSG00000223907 |
| | | | coding RNA 1226 | | | | |
| LINC01247 | 4.93 | 3.53 | long intergenic non-protein | 101929390 | NR_110251 | Hs.434407 | ENSG00000227007 |
| | | | coding RNA 1247 | | | | |
| LINC01252 | 4.11 | 2.85 | long intergenic non-protein | 338817 | NR_033890 | Hs.733066 | ENSG00000247157 |
| | | | coding RNA 1252 | | | | |
| LINC01299 | 3.88 | 3.19 | long intergenic non-protein | 286186 | NR_033893 | Hs.449427 | ENSG00000254081 |
| | | | coding RNA 1299 | | | | |
| LINC01356 | 4.31 | 3.22 | long intergenic non-protein | 100996702 | NR_103746 | Hs.632431 | ENSG00000215866 |
| | | | coding RNA 1356 | | | | |
| LOC100128233 | 4.52 | 3.88 | uncharacterized LOC100128233 | 100128233 | NR_103769 | Hs.497323 | ENSG00000255002 |
| LOC100128288 | 4.28 | 3.38 | uncharacterized LOC100128288 | 100128288 | NR_024447 | Hs.549913 | |
| LOC100128398 | 3.21 | 2.39 | uncharacterized LOC100128398 | 100128398 | NR_036508 | Hs.655081 | ENSG00000176593 |
| LOC100128531 | 3.85 | 2.9 | uncharacterized LOC100128531 | 100128531 | NR_038941 | Hs.662126 | ENSG00000203280 |
| LOC100128573 | 2.46 | 2.68 | uncharacterized LOC100128573 | 100128573 | NR_024491 | Hs.465761 | |
| LOC100129940 | 3.73 | 3.44 | uncharacterized LOC100129940 | 100129940 | NM_001292023 | Hs.685856 | ENSG00000197301 |
| LOC100130451 | 4.59 | 3.55 | uncharacterized LOC100130451 | 100130451 | NM_001242575 | | |
| LOC100131257 | 4.35 | 3.29 | zinc finger protein 655 pseudogene | 100131257 | NR_034022 | Hs.551110 | |
| LOC100131564 | 2.81 | 2.26 | uncharacterized LOC100131564 | 100131564 | NR_034089 | Hs.732666 | |
| LOC100131626 | 4.21 | 3.02 | uncharacterized LOC100131626 | 100131626 | NR_046369 | Hs.721614 | |
| LOC100132077 | 3.76 | 3.1 | uncharacterized LOC100132077 | 100132077 | NR_033937 | Hs.679111 | ENSG00000232063 |
| LOC100190986 | 2.12 | 2.25 | uncharacterized LOC100190986 | 100190986 | NR_024456 | Hs.648439 | |
| LOC100268168 | 4 | 3.55 | uncharacterized LOC100268168 | 100268168 | NR_026682 | Hs.519766 | ENSG00000204758 |
| LOC100287015 | 3.01 | 2.93 | uncharacterized LOC100287015 | 100287015 | NR_040040 | Hs.156928 | ENSG00000246089 |
| LOC100287042 | 2.11 | 1.98 | uncharacterized LOC100287042 | 100287042 | NR_036520 | Hs.514470 | ENSG00000263843 |
| LOC100287792 | 3.43 | 3.04 | uncharacterized LOC100287792 | 100287792 | NM_001001690 | Hs.517026 | ENSG00000204117 |
| LOC100287846 | 4.08 | 2.69 | patched 1 pseudogene | 100287846 | NR_037168 | Hs.21550 | |
| LOC100335030 | 4.83 | 3.91 | FGFR1 oncogene partner 2 | 100335030 | NR_033267 | Hs.687044 | |
| | | | pseudogene | | | | |
| LOC100420587 | 5.27 | 3.7 | SHC SH2-domain binding | 100420587 | NR_110759 | Hs.569956 | ENSG00000267243 |
| | | | protein 1 pseudogene | | | | |
| LOC100506023 | 3.79 | 2.76 | uncharacterized LOC100506023 | 100506023 | NR_037845 | Hs.731284 | |
| LOC100506083 | 3.67 | 3.08 | uncharacterized LOC100506083 | 100506083 | NR_039997 | Hs.635008 | ENSG00000261777 |
| LOC100506127 | 3.73 | 3.1 | putative uncharacterized | 100506127 | NM_001013634 | Hs.503319 | ENSG00000179240 |
| | | | protein FLJ37770-like | | | | |
| LOC100506472 | 3.36 | 2.68 | uncharacterized LOC100506472 | 100506472 | NR_040535 | Hs.729080 | |
| LOC100506551 | 4.19 | 3.53 | uncharacterized LOC100506551 | 100506551 | NR_103809 | Hs.657861 | ENSG00000257279 |
| LOC100506688 | 4.09 | 3.23 | uncharacterized LOC100506688 | 100506688 | NM_001242737 | Hs.532063 | ENSG00000215246 |
| LOC100506746 | 3.32 | 2.75 | uncharacterized LOC100506746 | 100506746 | NR_038841 | Hs.657766 | ENSG00000163633 |
| LOC100506990 | 2.84 | 2.36 | uncharacterized LOC100506990 | 100506990 | NR_040091 | Hs.656893 | |
| LOC100996251 | 4 | 3.37 | uncharacterized LOC100996251 | 100996251 | NR_103777 | Hs.382067 | ENSG00000238198 |
| LOC101409256 | 3.94 | 3.49 | cell division cycle 42 pseudogene | 101409256 | NR_102424 | | |
| LOC101926889 | 4.24 | 3.31 | uncharacterized LOC101926889 | 101926889 | NR_109994 | Hs.585997 | |
| LOC101927181 | 2.82 | 2.67 | uncharacterized LOC101927181 | 101927181 | NR_108066 | Hs.288853 | ENSG00000136213 |
| LOC101927257 | 3.78 | 3.16 | uncharacterized LOC101927257 | 101927257 | NR_109965 | Hs.662725 | ENSG00000232564 |
| LOC101927274 | 4.46 | 3.67 | uncharacterized LOC101927274 | 101927274 | NR_110751 | Hs.591168 | ENSG00000249383 |
| LOC101927374 | 4.86 | 3.64 | uncharacterized LOC101927374 | 101927374 | NR_110133 | Hs.570644 | |
| LOC101927415 | 3.2 | 2.84 | uncharacterized LOC101927415 | 101927415 | NR_110049 | Hs.636524 | |
| LOC101927476 | 4.99 | 4.19 | uncharacterized LOC101927476 | 101927476 | NR_110386 | Hs.522607 | ENSG00000236393 |
| LOC101927575 | 4.56 | 3.2 | uncharacterized LOC101927575 | 101927575 | NR_110995 | Hs.459826 | ENSG00000227463 |
| LOC101927740 | 4.04 | 3.36 | uncharacterized LOC101927740 | 101927740 | NR_109890 | Hs.738721 | ENSG00000245812 |
| LOC101927797 | 3.21 | 2.79 | uncharacterized LOC101927797 | 101927797 | NR_109925 | Hs.551743 | |
| LOC101927884 | 5.21 | 3.69 | uncharacterized LOC101927884 | 101927884 | NR_110281 | Hs.671110 | ENSG00000231172 |
| LOC101928103 | 4.63 | 3.08 | uncharacterized LOC101928103 | 101928103 | NR_110292 | Hs.665619 | ENSG00000229267 |
| LOC101928137 | 4.58 | 3.44 | uncharacterized LOC101928137 | 101928137 | NR_110130 | Hs.694666 | ENSG00000258123 |
| LOC101928254 | 4.24 | 4.15 | uncharacterized LOC101928254 | 101928254 | NR_110182 | Hs.571236 | ENSG00000219445 |
| LOC101928303 | 4.56 | 3.27 | uncharacterized LOC101928303 | 101928303 | NR_110698 | Hs.375067 | ENSG00000236155 |
| LOC101928336 | 4.87 | 3.73 | uncharacterized LOC101928336 | 101928336 | NR_110396 | | ENSG00000230392 |
| LOC101928372 | 3.85 | 3.11 | uncharacterized LOC101928372 | 101928372 | NR_110695 | | ENSG00000198358 |
| LOC101928401 | 3.63 | 3.01 | uncharacterized LOC101928401 | 101928401 | NR_108099 | Hs.385614 | ENSG00000233288 |
| LOC101928495 | 5.19 | 3.89 | uncharacterized LOC101928495 | 101928495 | NR_110409 | Hs.545998 | ENSG00000237208 |
| LOC101928514 | 5.14 | 3.96 | uncharacterized LOC101928514 | 101928514 | NR_110837 | Hs.617206 | ENSG00000267065 |
| LOC101928567 | 4.39 | 3.45 | uncharacterized LOC101928567 | 101928567 | NR_110839 | Hs.569757 | ENSG00000237057 |
| LOC101928580 | 3.93 | 3.68 | uncharacterized LOC101928580 | 101928580 | NR_120556 | Hs.569025 | ENSG00000246211 |
| LOC101928597 | 4.26 | 3.35 | uncharacterized LOC101928597 | 101928597 | NR_110091 | Hs.638942 | ENSG00000246394 |
| LOC101928600 | 4.9 | 3.96 | uncharacterized LOC101928600 | 101928600 | NR_109904 | Hs.694699 | ENSG00000250127 |
| LOC101928738 | 3.84 | 3.53 | uncharacterized LOC101928738 | 101928738 | NR_110851 | Hs.399280 | ENSG00000262188 |
| LOC101928936 | 4.73 | 3.78 | uncharacterized LOC101928936 | 101928936 | NR_110867 | Hs.533080 | |
| LOC101929181 | 3.42 | 2.44 | uncharacterized LOC101929181 | 101929181 | NR_104624 | Hs.568616 | ENSG00000235643 |
| LOC101929224 | 4.44 | 3.84 | uncharacterized LOC101929224 | 101929224 | NR_110787 | Hs.639369 | ENSG00000260088 |
| LOC101929259 | 4.17 | 3.67 | uncharacterized LOC101929259 | 101929259 | NR_120424 | Hs.638490 | |
| LOC101929486 | 4.25 | 3.06 | uncharacterized LOC101929486 | 101929486 | NR_109868 | Hs.548761 | ENSG00000233048 |
| LOC101929567 | 4.72 | 3.61 | uncharacterized LOC101929567 | 101929567 | NR_110257 | Hs.634706 | ENSG00000236008 |
| LOC101929586 | 4.34 | 3.59 | uncharacterized LOC101929586 | 101929586 | NR_120363 | Hs.569426 | ENSG00000259175 |
| LOC101929698 | 3.64 | 2.61 | uncharacterized LOC101929698 | 101929698 | NR_110619 | Hs.638392 | ENSG00000277301 |
| LOC102467081 | 4.99 | 3.91 | uncharacterized LOC102467081 | 102467081 | NR_104662 | | |
| LOC102723769 | 4.8 | 3.53 | uncharacterized LOC102723769 | 102723769 | NR_110761 | Hs.652926 | |
| LOC102724927 | 4.39 | 3.7 | uncharacterized LOC102724927 | 102724927 | NR_120311 | Hs.364739 | ENSG00000262185 |
| LOC143666 | 2.94 | 2.59 | uncharacterized LOC143666 | 143666 | NR_026967 | Hs.337054 | |
| LOC150935 | 4.82 | 4.54 | uncharacterized LOC150935 | 150935 | NR_037808 | Hs.555582 | |
| LOC151475 | 3.63 | 3.2 | uncharacterized LOC151475 | 151475 | NR_040038 | Hs.528154 | ENSG00000226125 |
| LOC257396 | 3.45 | 2.42 | uncharacterized LOC257396 | 257396 | NR_034107 | Hs.12326 | ENSG00000247796 |
| LOC283683 | 4.2 | 4 | uncharacterized LOC283683 | 283683 | NR_040057 | Hs.534616 | ENSG00000274253 |
| LOC284023 | 3.54 | 2.88 | uncharacterized LOC284023 | 284023 | NR_024349 | Hs.744470 | ENSG00000179859 |
| LOC284379 | 4.31 | 3.51 | solute carrier family 7 (cationic amino | 284379 | NR_002938 | Hs.631571 | ENSG00000268864 |
| | | | acid transporter, y+ system), | | | | |
| | | | member 3 pseudogene | | | | |
| LOC284412 | 6.66 | 4.68 | uncharacterized LOC284412 | 284412 | NR_029390 | Hs.635932 | |
| LOC284454 | 4.32 | 3.54 | uncharacterized LOC284454 | 284454 | NR_036515 | Hs.436426 | ENSG00000267519 |
| LOC284581 | 4.12 | 3.17 | uncharacterized LOC284581 | 284581 | NR_046097 | | |
| LOC284865 | 4.37 | 3.67 | uncharacterized LOC284865 | 284865 | NR_038460 | Hs.638498 | ENSG00000249923 |
| LOC284950 | 4.2 | 3.63 | uncharacterized LOC284950 | 284950 | NR_038888 | Hs.570227 | |
| LOC285696 | 4.41 | 3.57 | uncharacterized LOC285696 | 285696 | NM_173669 | Hs.646924 | ENSG00000215196 |
| LOC286437 | 4.49 | 3.29 | uncharacterized LOC286437 | 286437 | NR_039980 | Hs.656786 | |
| LOC339166 | 3.75 | 2.65 | uncharacterized LOC339166 | 339166 | NR_040000 | Hs.736088 | ENSG00000179314 |
| LOC339803 | 3.45 | 2.76 | uncharacterized LOC339803 | 339803 | NR_036496 | Hs.252433 | ENSG00000212978 |
| LOC389641 | 3.53 | 2.91 | uncharacterized LOC389641 | 389641 | NR_033928 | Hs.591835 | ENSG00000246582 |
| LOC400958 | 4.62 | 3.57 | uncharacterized LOC400958 | 400958 | NR_036586 | Hs.591565 | ENSG00000237638 |
| LOC401052 | 4.04 | 3.52 | uncharacterized LOC401052 | 401052 | NM_001008737 | Hs.662766 | |
| LOC440173 | 5.21 | 3.95 | uncharacterized LOC440173 | 440173 | NR_027471 | Hs.127361 | ENSG00000269994 |
| LOC440300 | 3.9 | 3.42 | chondroitin sulfate | 440300 | NR_033738 | Hs.546565 | ENSG00000259295 |
| | | | proteoglycan 4 pseudogene | | | | |
| LOC441242 | 2.11 | 2.07 | uncharacterized LOC441242 | 441242 | NM_001013464 | Hs.373941 | ENSG00000272693 |
| LOC643406 | 4.43 | 3.27 | uncharacterized LOC643406 | 643406 | NM_175877 | Hs.431161 | |
| LOC644919 | 4.98 | 3.81 | uncharacterized LOC644919 | 644919 | NR_109757 | Hs.434414 | |
| LOC646214 | 4.3 | 3.38 | p21 protein (Cdc42/Rac)-activated | 646214 | NR_027053 | Hs.510697 | |
| | | | kinase 2 pseudogene | | | | |
| LOC650293 | 6.38 | 3.67 | seven transmembrane helix receptor | 650293 | NM_001040071 | Hs.535167 | |
| LOC727896 | 3.8 | 2.72 | cysteine and histidine-rich domain | 727896 | NR_026659 | Hs.673126 | |
| | | | (CHORD) containing 1 pseudogene | | | | |
| LOC728613 | 2.3 | 2.03 | programmed cell death 6 pseudogene | 728613 | NR_003713 | Hs.720393 | |
| LOC728752 | 4.03 | 3.31 | uncharacterized LOC728752 | 728752 | NR_036504 | Hs.729762 | ENSG00000267309 |
| LOC729603 | 4.36 | 3.16 | calcineurin-like EF-hand | 729603 | NR_003288 | Hs.674810 | ENSG00000213073 |
| | | | protein 1 pseudogene | | | | |
| LOC729732 | 3.63 | 2.93 | uncharacterized LOC729732 | 729732 | NR_047662 | Hs.322761 | |
| LOC729987 | 4.36 | 3.05 | uncharacterized LOC729987 | 729987 | NR_046088 | Hs.683961 | ENSG00000226053 |
| LOC731424 | 4.17 | 3.03 | uncharacterized LOC731424 | 731424 | NR_037867 | Hs.427740 | |
| LOH12CR2 | 4.49 | 3.39 | loss of heterozygosity, 12, | 503693 | NR_024061 | Hs.67553 | ENSG00000205791 |
| | | | chromosomal region 2 (non- | | | | |
| | | | protein coding) | | | | |
| LPAL2 | 3.58 | 2.94 | lipoprotein, Lp(a)-like 2, | 80350 | NM_024492 | Hs.654503 | ENSG00000213071 |
| | | | pseudogene | | | | |
| LPCAT2 | 3.36 | 2.61 | lysophosphatidylcholine | 54947 | NM_017839 | Hs.460857 | ENSG00000087253 |
| | | | acyltransferase 2 | | | | |
| LPP | 2.85 | 2.51 | LIM domain containing preferred | 4026 | NM_001167671 | Hs.720220 | ENSG00000145012 |
| | | | translocation partner in lipoma | | | | |
| LRPAP1 | 2.05 | 1.91 | low density lipoprotein receptor- | 4043 | NM_002337 | Hs.40966 | ENSG00000163956 |
| | | | related protein associated protein 1 | | | | |
| LRRC27 | 3.6 | 2.88 | leucine rich repeat containing 27 | 80313 | NM_001143757 | Hs.119897 | ENSG00000148814 |
| LRRC57 | 3.77 | 3.17 | leucine rich repeat containing 57 | 255252 | NM_153260 | Hs.234681 | ENSG00000180979 |
| LRRN4CL | 4.42 | 4.03 | LRRN4 C-terminal like | 221091 | NM_203422 | Hs.427449 | ENSG00000177363 |
| LRTOMT | 4.01 | 3.25 | leucine rich transmembrane and O- | 220074 | NM_001145307 | Hs.317243 | ENSG00000184154 |
| | | | methyltransferase domain containing | | | | |
| LUCAT1 | 5.28 | 4.95 | lung cancer associated transcript 1 | 100505994 | NR_103548 | Hs.628363 | ENSG00000248323 |
| | | | (non-protein coding) | | | | |
| LYRM7 | 2.35 | 2.03 | LYR motif containing 7 | 90624 | NM_001293735 | Hs.115467 | ENSG00000186687 |
| MAB21L3 | 4.04 | 3.19 | mab-21-like 3 (C. elegans) | 126868 | NM_152367 | Hs.376194 | ENSG00000173212 |
| MAGEA10 | 3.73 | 3.52 | melanoma antigen family A10 | 4109 | NM_001011543 | Hs.18048 | ENSG00000124260 |
| MAN1B1-AS1 | 2.93 | 2.7 | MAN1B1 antisense RNA 1 | 100289341 | NR_027447 | Hs.593896 | ENSG00000268996 |
| | | | (head to head) | | | | |
| MANEAL | 6.55 | 4.78 | mannosidase, endo-alpha-like | 149175 | NM_001031740 | Hs.534562 | ENSG00000185090 |
| MAP1LC3C | 5.17 | 3.96 | microtubule-associated protein 1 | 440738 | NM_001004343 | Hs.534971 | ENSG00000197769 |
| | | | light chain 3 gamma | | | | |
| MAP3K13 | 2.6 | 2.25 | mitogen-activated protein | 9175 | NM_001242314 | Hs.591306 | ENSG00000073803 |
| | | | kinase kinase kinase 13 | | | | |
| MAP7D3 | 2.78 | 2.32 | MAP7 domain containing 3 | 79649 | NM_001173516 | Hs.446275 | ENSG00000129680 |
| MARVELD3 | 4.25 | 3.45 | MARVEL domain containing 3 | 91862 | NM_001017967 | Hs.513706 | ENSG00000140832 |
| MBOAT1 | 4.45 | 3.23 | membrane bound O-acyltransferase | 154141 | NM_001080480 | Hs.377830 | ENSG00000172197 |
| | | | domain containing 1 | | | | |
| MBOAT2 | 4.33 | 2.81 | membrane bound O-acyltransferase | 129642 | NM_138799 | Hs.467634 | ENSG00000143797 |
| | | | domain containing 2 | | | | |
| MCFD2 | 3.28 | 2.63 | multiple coagulation factor | 90411 | NM_001171506 | Hs.662152 | ENSG00000180398 |
| | | | deficiency 2 | | | | |
| MCUR1 | 2.23 | 1.92 | mitochondrial calcium | 63933 | NM_001031713 | Hs.214043 | ENSG00000050393 |
| | | | uniporter regulator 1 | | | | |
| MED15P9 | 4.39 | 3.57 | mediator complex subunit 15 | 285103 | NR_033903 | Hs.570106 | ENSG00000223760 |
| | | | pseudogene 9 | | | | |
| MED18 | 3.5 | 2.65 | mediator complex subunit 18 | 54797 | NM_001127350 | Hs.479911 | ENSG00000130772 |
| MEFV | 4.22 | 3.28 | Mediterranean fever | 4210 | NM_000243 | Hs.632221 | ENSG00000103313 |
| METTL20 | 3.37 | 2.51 | methyltransferase like 20 | 254013 | NM_001135863 | Hs.740628 | ENSG00000139160 |
| METTL21A | 3.85 | 3.08 | methyltransferase like 21A | 151194 | NM_001127395 | Hs.664764 | ENSG00000144401 |
| METTL2A | 2.57 | 2.09 | methyltransferase like 2A | 339175 | NM_001005372 | Hs.381204 | ENSG00000087995 |
| METTL2B | 2.5 | 2.04 | methyltransferase like 2B | 55798 | NM_018396 | Hs.433213 | ENSG00000165055 |
| METTL8 | 3.18 | 2.57 | methyltransferase like 8 | 79828 | NM_024770 | Hs.135146 | ENSG00000123600 |
| MFAP5 | 4.32 | 4.07 | microfibrillar associated protein 5 | 8076 | NM_001297709 | Hs.512842 | ENSG00000197614 |
| MFSD11 | 2.35 | 2.09 | major facilitator superfamily | 79157 | NM_001242532 | Hs.73965 | ENSG00000092931 |
| | | | domain containing 11 | | | | |
| MGC27345 | 2.95 | 2.55 | uncharacterized protein MGC27345 | 157247 | NM_175880 | Hs.552129 | |
| MIRLET7BHG | 3.92 | 2.93 | MIRLET7B host gene | 400931 | NM_207477 | Hs.235838 | ENSG00000197182 |
| MLANA | 3.42 | 3.21 | melan-A | 2315 | NM_005511 | Hs.154069 | ENSG00000120215 |
| MMD2 | 4.97 | 4.03 | monocyte to macrophage | 221938 | NM_001100600 | Hs.558694 | ENSG00000136297 |
| | | | differentiation-associated 2 | | | | |
| MMS22L | 2.35 | 2.08 | MMS22-like, DNA repair protein | 253714 | NM_198468 | Hs.444292 | ENSG00000146263 |
| MOCS3 | 3.22 | 2.55 | molybdenum cofactor synthesis 3 | 27304 | NM_014484 | Hs.159410 | ENSG00000124217 |
| MOG | 4.36 | 3.37 | myelin oligodendrocyte glycoprotein | 4340 | NM_001008228 | Hs.141308 | ENSG00000204655 |
| MORN4 | 3.72 | 2.72 | MORN repeat containing 4 | 118812 | NM_001098831 | Hs.217409 | ENSG00000171160 |
| MPPE1 | 2.83 | 2.35 | metallophosphoesterase 1 | 65258 | NM_001242904 | Hs.712666 | ENSG00000154889 |
| MPV17L | 3.24 | 2.81 | MPV17 mitochondrial | 255027 | NM_001128423 | Hs.720673 | ENSG00000275543 |
| | | | membrane protein-like | | | | |
| MPZL3 | 2.69 | 2.15 | myelin protein zero-like 3 | 196264 | NM_001286152 | Hs.15396 | ENSG00000160588 |
| MREG | 3.18 | 2.62 | melanoregulin | 55686 | NM_018000 | Hs.620391 | ENSG0000011822 |
| MRGPRX3 | 4.79 | 3.53 | MAS-related GPR, member X3 | 117195 | NM_054031 | Hs.380177 | ENSG00000179826 |
| MS4A10 | 3.65 | 3 | membrane-spanning 4-domains, | 341116 | NM_206893 | Hs.591956 | ENSG00000172689 |
| | | | subfamily A, member 10 | | | | |
| MTFMT | 3.44 | 2.79 | mitochondrial methionyl- | 123263 | NM_139242 | Hs.531615 | ENSG00000103707 |
| | | | tRNA formyltransferase | | | | |
| MTG2 | 2.51 | 2.02 | mitochondrial ribosome- | 26164 | NM_015666 | Hs.340636 | ENSG00000101181 |
| | | | associated GTPase 2 | | | | |
| MTRNR2L5 | 6.94 | 5.47 | MT-RNR2-like 5 | 100463289 | NM_001190478 | Hs.727204 | ENSG00000249860 |
| MXRA7 | 2.44 | 2.12 | matrix-remodelling associated 7 | 439921 | NM_001008528 | Hs.250723 | ENSG00000182534 |
| MYEOV2 | 0.48 | 0.51 | myeloma overexpressed 2 | 150678 | NM_001163424 | Hs.293884 | ENSG00000172428 |
| MYLK3 | 3.77 | 3.22 | myosin light chain kinase 3 | 91807 | NM_001308301 | Hs.130465 | ENSG00000140795 |
| NANOG | 4.75 | 3.1 | Nanog homeobox | 79923 | NM_001297698 | Hs.635882 | ENSG00000111704 |
| NCRUPAR | 4.14 | 3.71 | non-protein coding RNA, | 100302746 | NR_028375 | | |
| | | | upstream of F2R/PAR1 | | | | |
| NEK2 | 4.2 | 3.18 | NIMA-related kinase 2 | 4751 | NM_001204182 | Hs.153704 | ENSG00000117650 |
| NEK8 | 2.71 | 2.3 | NIMA-related kinase 8 | 284086 | NM_178170 | Hs.448468 | ENSG00000160602 |
| NEXN-AS1 | 3.79 | 3.32 | NEXN antisense RNA 1 | 374987 | NM_001039463 | Hs.632414 | ENSG00000235927 |
| NLRP12 | 4.78 | 3.59 | NLR family, pyrin domain | 91662 | NM_001277126 | Hs.631573 | ENSG00000142405 |
| | | | containing 12 | | | | |
| NMNAT1 | 3.68 | 2.96 | nicotinamide nucleotide | 64802 | NM_001297778 | Hs.633762 | ENSG00000173614 |
| | | | adenylyltransferase 1 | | | | |
| NPFFR2 | 4.64 | 3.53 | neuropeptide FF receptor 2 | 10886 | NM_001144756 | Hs.99231 | ENSG00000056291 |
| NPHS1 | 3.6 | 3.16 | nephrosis 1, congenital, | 4868 | NM_004646 | Hs.122186 | ENSG00000161270 |
| | | | Finnish type (nephrin) | | | | |
| NQO1 | 3.2 | 2.27 | NAD(P)H dehydrogenase, quinone 1 | 1728 | NM_000903 | Hs.406515 | ENSG00000181019 |
| NRIP2 | 2.49 | 2.5 | nuclear receptor interacting protein 2 | 83714 | NM_031474 | Hs.530816 | ENSG00000053702 |
| NRIP3 | 3.99 | 2.93 | nuclear receptor interacting protein 3 | 56675 | NM_020645 | Hs.523467 | ENSG00000175352 |
| NT5DC3 | 3.57 | 2.85 | 5′-nucleotidase domain containing 3 | 51559 | NM_001031701 | Hs.48428 | ENSG00000111696 |
| NUBPL | 3.17 | 2.32 | nucleotide binding protein-like | 80224 | NM_001201573 | Hs.288981 | ENSG00000151413 |
| NUGGC | 2.57 | 2.48 | nuclear GTPase, germinal | 389643 | NM_001010906 | Hs.370129 | ENSG00000189233 |
| | | | center associated | | | | |
| NXN | 4.95 | 3.79 | nucleoredoxin | 64359 | NM_001205319 | Hs.527989 | ENSG00000167693 |
| NXNL2 | 4.52 | 3.62 | nucleoredoxin-like 2 | 158046 | NM_001161625 | Hs.734507 | ENSG00000130045 |
| NYAP2 | 3.86 | 3.11 | neuronal tyrosine-phosphorylated | 57624 | NM_020864 | Hs.224409 | ENSG00000144460 |
| | | | phosphoinositide-3-kinase adaptor 2 | | | | |
| OCLN | 2.79 | 2.4 | occludin | 100506658 | NM_001205254 | Hs.592605 | ENSG00000197822 |
| ODF2L | 4.02 | 3.1 | outer dense fiber of sperm tails 2-like | 57489 | NM_001007022 | Hs.149360 | ENSG00000122417 |
| OLAH | 4.85 | 3.6 | oleoyl-ACP hydrolase | 55301 | NM_001039702 | Hs.24309 | ENSG00000152463 |
| OPHN1 | 4.66 | 3.31 | oligophrenin 1 | 4983 | NM_002547 | Hs.128824 | ENSG00000079482 |
| OR11A1 | 4.75 | 3.53 | olfactory receptor, family 11, subfamily | 26531 | NM_013937 | Hs.676010 | ENSG00000204694 |
| | | | A, member 1 | | | | |
| OR7D2 | 3.8 | 3.11 | olfactory receptor, family 7, subfamily | 162998 | NM_175883 | Hs.531755 | ENSG00000188000 |
| | | | D, member 2 | | | | |
| OR7E91P | 6.26 | 4.84 | olfactory receptor, family 7, subfamily | 79315 | NR_002185 | Hs.327033 | ENSG00000205847 |
| | | | E, member 91 pseudogene | | | | |
| ORAI2 | 3.08 | 2.65 | ORAI calcium release- | 80228 | NM_001126340 | Hs.363308 | ENSG00000160991 |
| | | | activated calcium modulator 2 | | | | |
| ORC4 | 4.42 | 3.38 | origin recognition complex, subunit 4 | 5000 | NM_001190879 | Hs.558364 | ENSG00000115947 |
| ORC6 | 3.75 | 3.32 | origin recognition complex, subunit 6 | 23594 | NM_014321 | Hs.49760 | ENSG00000091651 |
| OSBPL2 | 2.32 | 1.98 | oxysterol binding protein-like 2 | 9885 | NM_001001691 | Hs.473254 | ENSG00000130703 |
| OSGEPL1-AS1 | 3.23 | 2.45 | OSGEPL1 antisense RNA 1 | 101409258 | NR_102429 | Hs.738558 | |
| OTUD6A | 5.09 | 4.14 | OTU deubiquitinase 6A | 139562 | NM_207320 | Hs.447381 | ENSG00000189401 |
| P2RX5- | 3.14 | 2.64 | P2RX5-TAX1BP3 readthrough | 100533970 | NR_037928 | Hs.731607 | ENSG00000257950 |
| TAX1BP3 | | | (NMD candidate) | | | | |
| PABPC1P2 | 3.85 | 2.98 | poly(A) binding protein, | 728773 | NR_026904 | Hs.334462 | |
| | | | cytoplasmic 1 pseudogene 2 | | | | |
| PACS2 | 2.18 | 2.2 | phosphofurin acidic cluster | 23241 | NM_001100913 | Hs.525626 | ENSG00000179364 |
| | | | sorting protein 2 | | | | |
| PAQR7 | 3.26 | 2.65 | progestin and adipoQ | 164091 | NM_178422 | Hs.523652 | ENSG00000182749 |
| | | | receptor family member VII | | | | |
| PARD6G | 4.04 | 3.5 | par-6 family cell polarity | 84552 | NM_032510 | Hs.654920 | ENSG00000178184 |
| | | | regulator gamma | | | | |
| PARK2 | 3.51 | 3 | parkin RBR E3 ubiquitin protein ligase | 5071 | NM_004562 | Hs.132954 | ENSG00000185345 |
| PART1 | 4.74 | 3.77 | prostate androgen-regulated transcript | 25859 | NM_001039499 | Hs.146312 | ENSG00000152931 |
| | | | 1 (non-protein coding) | | | | |
| PAXBP1-AS1 | 4.11 | 3.3 | PAXBP1 antisense RNA 1 | 100506215 | NR_038879 | Hs.657123 | ENSG00000238197 |
| PCAT18 | 4.34 | 3.61 | prostate cancer associated transcript 18 | 728606 | NR_024259 | Hs.170599 | ENSG00000265369 |
| | | | (non-protein coding) | | | | |
| PCBD2 | 2.76 | 2.39 | pterin-4 alpha-carbinolamine | 84105 | NM_032151 | Hs.710014 | ENSG00000132570 |
| | | | dehydratase/dimerization cofactor of | | | | |
| | | | hepatocyte nuclear factor 1 alpha | | | | |
| | | | (TCF1)2 | | | | |
| PCDH11X | 4.44 | 3.85 | protocadherin 11 X-linked | 27328 | NM_001168360 | Hs.655673 | ENSG00000102290 |
| PCDH11Y | 5.34 | 3.89 | protocadherin 11 Y-linked | 83259 | NM_001278619 | Hs.661308 | ENSG00000099715 |
| PCDHB9 | 4.21 | 3.51 | protocadherin beta 9 | 56127 | NM_019119 | Hs.662726 | ENSG00000177839 |
| PDDC1 | 3.44 | 2.9 | Parkinson disease 7 domain | 347862 | NM_182612 | Hs.218362 | ENSG00000177225 |
| | | | containing 1 | | | | |
| PDE4C | 4.64 | 3.82 | phosphodiesterase 4C, cAMP-specific | 5143 | NM_000923 | Hs.132584 | ENSG00000105650 |
| PDE6A | 4.29 | 3.55 | phosphodiesterase 6A, | 5145 | NM_000440 | Hs.567314 | ENSG00000132915 |
| | | | cGMP-specific, rod, alpha | | | | |
| PDLIM5 | 2.84 | 2.51 | PDZ and LIM domain 5 | 10611 | NM_001011513 | Hs.480311 | ENSG00000163110 |
| PDP2 | 3.15 | 2.47 | pyruvate dehyrogenase phosphatase | 57546 | NM_020786 | Hs.632214 | ENSG00000172840 |
| | | | catalytic subunit 2 | | | | |
| PEX13 | 2.2 | 1.9 | peroxisomal biogenesis factor 13 | 5194 | NM_002618 | Hs.161377 | ENSG00000162928 |
| PGAM5 | 2.87 | 2.34 | PGAM family member 5, | 192111 | NM_001170543 | Hs.102558 | ENSG00000247077 |
| | | | serine/threonine protein | | | | |
| | | | phosphatase, mitochondrial | | | | |
| PGM2L1 | 2.47 | 2.14 | phosphoglucomutase 2-like 1 | 283209 | NM_173582 | Hs.26612 | ENSG00000165434 |
| PGM5P2 | 4.87 | 3.76 | phosphoglucomutase 5 pseudogene 2 | 595135 | NR_002836 | Hs.571593 | ENSG00000277778 |
| PHACTR4 | 2.21 | 1.9 | phosphatase and actin regulator 4 | 65979 | NM_001048183 | Hs.225641 | ENSG00000204138 |
| PHAX | 2.1 | 1.93 | phosphorylated adaptor for | 51808 | NM_032177 | Hs.555731 | ENSG00000164902 |
| | | | RNA export | | | | |
| PHYHD1 | 4.22 | 3.21 | phytanoyl-CoA dioxygenase | 254295 | NM_001100876 | Hs.709447 | ENSG00000175287 |
| | | | domain containing 1 | | | | |
| PIGX | 2.62 | 2.3 | phosphatidylinositol glycan | 54965 | NM_001166304 | Hs.223296 | ENSG00000163964 |
| | | | anchor biosynthesis, class X | | | | |
| PIN4P1 | 3.8 | 3.03 | protein (peptidylprolyl | 728758 | NR_003571 | Hs.658099 | |
| | | | cis/trans isomerase) NIMA- | | | | |
| | | | interacting, 4 pseudogene 1 | | | | |
| PLCXD1 | 2.76 | 2.34 | phosphatidylinositol-specific | 55344 | NM_018390 | Hs.522568 | ENSG00000182378 |
| | | | phospholipase C, X domain | | | | |
| | | | containing 1 | | | | |
| PLEKHA5 | 3.35 | 2.56 | pleckstrin homology domain | 54477 | NM_001143821 | Hs.188614 | ENSG00000052126 |
| | | | containing, family A member 5 | | | | |
| PNMA2 | 3.78 | 2.97 | paraneoplastic Ma antigen 2 | 10687 | NM_007257 | Hs.591838 | ENSG00000240694 |
| PNPO | 3.15 | 2.5 | pyridoxamine 5′-phosphate oxidase | 55163 | NM_018129 | Hs.631742 | ENSG00000108439 |
| PNPT1 | 2.47 | 2.21 | polyribonucleotide | 87178 | NM_033109 | Hs.388733 | ENSG00000138035 |
| | | | nucleotidyltransferase 1 | | | | |
| POU2AF1 | 3.86 | 2.91 | POU class 2 associating factor 1 | 5450 | NM_006235 | Hs.654525 | ENSG00000110777 |
| POU5F1 | 4.39 | 3.66 | POU class 5 homeobox 1 | 5460 | NM_001173531 | Hs.249184 | ENSG00000204531 |
| PPARA | 2.01 | 1.91 | peroxisome proliferator- | 5465 | NM_001001928 | Hs.103110 | ENSG00000186951 |
| | | | activated receptor alpha | | | | |
| PPFIBP1 | 2.93 | 2.51 | PTPRF interacting protein, binding | 8496 | NM_001198915 | Hs.172445 | ENSG00000110841 |
| | | | protein 1 (liprin beta 1) | | | | |
| PPIEL | 3.32 | 2.92 | peptidylprolyl isomerase E- | 728448 | NR_003929 | Hs.472508 | |
| | | | like pseudogene | | | | |
| PPIL6 | 3.58 | 2.99 | peptidylprolyl isomerase | 285755 | NM_001111298 | Hs.32234 | ENSG00000185250 |
| | | | (cyclophilin)-like 6 | | | | |
| PPP1R3B | 3.14 | 2.43 | protein phosphatase 1, | 79660 | NM_001201329 | Hs.458513 | ENSG00000173281 |
| | | | regulatory subunit 3B | | | | |
| PQLC2 | 3.19 | 3.02 | PQ loop repeat containing 2 | 54896 | NM_001040125 | Hs.647620 | ENSG00000040487 |
| PRELID2 | 3.66 | 2.93 | PRELI domain containing 2 | 153768 | NM_138492 | Hs.314261 | ENSG00000186314 |
| PRICKLE2-AS3 | 5.03 | 3.97 | PRICKLE2 antisense RNA 3 | 100874243 | NR_046702 | Hs.670840 | ENSG00000226017 |
| PRKAR2A-AS1 | 3.81 | 3.22 | PRKAR2A antisense RNA 1 | 100506637 | NR_109996 | Hs.634259 | ENSG00000224424 |
| PRNCR1 | 3.97 | 3.27 | prostate cancer associated | 101867536 | NR_109833 | Hs.652970 | ENSG00000282961 |
| | | | non-coding RNA 1 | | | | |
| PRR11 | 3.89 | 3.18 | proline rich 11 | 55771 | NM_018304 | Hs.631750 | ENSG00000068489 |
| PRR7-AS1 | 2.95 | 2.56 | PRR7 antisense RNA 1 | 340037 | NR_038915 | Hs.570879 | |
| PSPH | 2.58 | 1.85 | phosphoserine phosphatase | 5723 | NM_004577 | Hs.512656 | ENSG00000146733 |
| PSTPIP2 | 3.42 | 2.89 | proline-serine-threonine phosphatase | 9050 | NM_024430 | Hs.567384 | ENSG00000152229 |
| | | | interacting protein 2 | | | | |
| PTCHD4 | 5.32 | 4.04 | patched domain containing 4 | 442213 | NM_001013732 | Hs.659409 | ENSG00000244694 |
| PTCSC3 | 4.39 | 3.47 | papillary thyroid carcinoma | 100886964 | NR_049735 | Hs.742592 | |
| | | | susceptibility candidate 3 | | | | |
| | | | (non-protein coding) | | | | |
| PTGER4P2- | 5.14 | 3.65 | PTGER4P2-CDK2AP2P2 | 442421 | NR_024496 | Hs.585349 | |
| CDK2AP2P2 | | | readthrough transcribed | | | | |
| | | | pseudogene | | | | |
| PTGES2-AS1 | 2.85 | 2.88 | PTGES2 antisense RNA 1 | 389791 | NM_001013652 | Hs.632678 | ENSG00000232850 |
| | | | (head to head) | | | | |
| PTK6 | 3.01 | 2.83 | protein tyrosine kinase 6 | 5753 | NM_001256358 | Hs.51133 | ENSG00000101213 |
| PTOV1-AS1 | 2.32 | 2.25 | PTOV1 antisense RNA 1 | 100506033 | NR_040037 | Hs.654814 | ENSG00000268006 |
| PTPRG-AS1 | 4.5 | 3.35 | PTPRG antisense RNA 1 | 100506994 | NR_038281 | Hs.656620 | ENSG00000241472 |
| PXMP4 | 3.14 | 2.44 | peroxisomal membrane | 11264 | NM_007238 | Hs.654857 | ENSG00000101417 |
| | | | protein 4.24 kDa | | | | |
| QPCTL | 3.58 | 3.17 | glutaminyl-peptide | 54814 | NM_001163377 | Hs.631556 | ENSG00000011478 |
| | | | cyclotransferase-like | | | | |
| QPRT | 2.91 | 2.92 | quinolinate phosphoribosyltransferase | 23475 | NM_014298 | Hs.513484 | ENSG00000103485 |
| RAB36 | 3.79 | 3.16 | RAB36, member RAS | 9609 | NM_004914 | Hs.369557 | ENSG00000100228 |
| | | | oncogene family | | | | |
| RAB42 | 4.45 | 3.72 | RAB42, member RAS | 115273 | NM_001193532 | Hs.652321 | ENSG00000188060 |
| | | | oncogene family | | | | |
| RAMP2-AS1 | 5.2 | 3.97 | RAMP2 antisense RNA 1 | 100190938 | NR_024461 | Hs.655265 | ENSG00000197291 |
| RASAL2-AS1 | 4.08 | 3.27 | RASAL2 antisense RNA 1 | 100302401 | NR_027982 | Hs.736117 | ENSG00000224687 |
| RBBP5 | 2.29 | 1.81 | retinoblastoma binding protein 5 | 5929 | NM_001193272 | Hs.519230 | ENSG00000117222 |
| RBBP9 | 2.52 | 2 | retinoblastoma binding protein 9 | 10741 | NM_006606 | Hs.69330 | ENSG00000089050 |
| RBM34 | 2.81 | 2.35 | RNA binding motif protein 34 | 23029 | NM_001161533 | Hs.535224 | ENSG00000188739 |
| RBMS2 | 3.36 | 2.82 | RNA binding motif, single | 5939 | NM_002898 | Hs.505729 | ENSG00000076067 |
| | | | stranded interacting protein 2 | | | | |
| RDH10 | 2.61 | 2.36 | retinol dehydrogenase 10 (all-trans) | 157506 | NM_172037 | Hs.244940 | ENSG00000121039 |
| RFT1 | 2.28 | 2.1 | RFT1 homolog | 91869 | NM_052859 | Hs.631910 | ENSG00000163933 |
| RHBG | 3.7 | 3.21 | Rh family, B glycoprotein | 57127 | NM_001256395 | Hs.131835 | ENSG00000132677 |
| | | | (gene/pseudogene) | | | | |
| RHD | 2.91 | 2.68 | Rh blood group, D antigen | 6007 | NM_001127691 | Hs.449968 | ENSG00000187010 |
| RIPPLY3 | 4.26 | 3.28 | ripply transcriptional repressor 3 | 53820 | NM_018962 | Hs.254560 | ENSG00000183145 |
| RNF144A-AS1 | 4.07 | 2.8 | RNF144A antisense RNA 1 | 386597 | NR_033997 | Hs.559010 | ENSG00000228203 |
| RNF207 | 3.76 | 2.95 | ring finger protein 207 | 388591 | NM_173795 | Hs.716549 | ENSG00000158286 |
| RNF222 | 3.81 | 3.32 | ring finger protein 222 | 643904 | NM_001146684 | Hs.526550 | ENSG00000189051 |
| ROR1-AS1 | 4.17 | 3.15 | ROR1 antisense RNA 1 | 101927034 | NR_110665 | Hs.680824 | ENSG00000223949 |
| RPL23AP53 | 3.22 | 2.57 | ribosomal protein L23a pseudogene 53 | 644128 | NR_003572 | Hs.652159 | |
| RUNDC1 | 3.11 | 2.63 | RUN domain containing 1 | 146923 | NM_173079 | Hs.632255 | ENSG00000198863 |
| S1PR2 | 3.38 | 2.86 | sphingosine-1-phosphate receptor 2 | 9294 | NM_004230 | Hs.655405 | ENSG00000267534 |
| SAA2 | 4.24 | 3.13 | serum amyloid A2 | 6289 | NM_001127380 | Hs.731376 | ENSG00000134339 |
| SCAI | 2.63 | 2.39 | suppressor of cancer cell invasion | 286205 | NM_001144877 | Hs.59504 | ENSG00000173611 |
| SCD5 | 4.02 | 3.09 | stearoyl-CoA desaturase 5 | 79966 | NM_001037582 | Hs.379191 | ENSG00000145284 |
| SCHLAP1 | 4.03 | 3.25 | SWI/SNF complex antagonist | 101669767 | NR_104319 | | ENSG00000281131 |
| | | | associated with prostate cancer 1 | | | | |
| | | | (non-protein coding) | | | | |
| SEC14L4 | 4.22 | 3.23 | SEC14-like lipid binding 4 | 284904 | NM_001161368 | Hs.517541 | ENSG00000133488 |
| SEC24B-AS1 | 3.14 | 2.68 | SEC24B antisense RNA 1 | 100533182 | NR_039978 | Hs.518927 | ENSG00000247950 |
| SEPSECS-AS1 | 2.6 | 4.21 | SEPSECS antisense RNA 1 | 285540 | NR_037934 | Hs.732278 | |
| | | | (head to head) | | | | |
| SFTPB | 3.95 | 3.17 | surfactant protein B | 6439 | NM_000542 | Hs.512690 | ENSG00000168878 |
| SGCB | 2.27 | 1.97 | sarcoglycan, beta (43 kDa dystrophin- | 6443 | NM_000232 | Hs.438953 | ENSG00000163069 |
| | | | associated glycoprotein) | | | | |
| SGOL1 | 3.16 | 2.77 | shugoshin-like 1 (S. pombe) | 151648 | NM_001012409 | Hs.105153 | ENSG00000129810 |
| SGSM1 | 3.88 | 3.15 | small G protein signaling modulator 1 | 129049 | NM_001039948 | Hs.474397 | ENSG00000167037 |
| SHANK2-AS3 | 4.12 | 3.3 | SHANK2 antisense RNA 3 | 220070 | NM_145308 | Hs.326766 | ENSG00000171671 |
| SHISA9 | 5.02 | 3.8 | shisa family member 9 | 729993 | NM_001145204 | Hs.130661 | ENSG00000237515 |
| SHOX | 2.82 | 2.39 | short stature homeobox | 6473 | NM_000451 | Hs.105932 | ENSG00000185960 |
| SHROOM1 | 4.92 | 3.67 | shroom family member 1 | 134549 | NM_001172700 | Hs.519574 | ENSG00000164403 |
| SIGLEC10 | 3.86 | 2.8 | sialic acid binding Ig-like lectin 10 | 89790 | NM_001171156 | Hs.284813 | ENSG00000142512 |
| SIRPB2 | 3.31 | 2.78 | signal-regulatory protein beta 2 | 284759 | NM_001122962 | Hs.721685 | ENSG00000196209 |
| SIX4 | 4.13 | 3.33 | SIX homeobox 4 | 51804 | NM_017420 | Hs.97849 | ENSG00000100625 |
| SKA1 | 4.3 | 3.38 | spindle and kinetochore | 220134 | NM_001039535 | Hs.134726 | ENSG00000154839 |
| | | | associated complex subunit 1 | | | | |
| SKP2 | 2.69 | 2.14 | S-phase kinase-associated protein 2, | 6502 | NM_001243120 | Hs.23348 | ENSG00000145604 |
| | | | E3 ubiquitin protein ligase | | | | |
| SLC14A2 | 4.34 | 3.33 | solute carrier family 14 (urea | 8170 | NM_001242692 | Hs.710927 | ENSG00000132874 |
| | | | transporter), member 2 | | | | |
| SLC15A1 | 3.52 | 2.91 | solute carrier family 15 (oligopeptide | 6564 | NM_005073 | Hs.436893 | ENSG00000088386 |
| | | | transporter), member 1 | | | | |
| SLC16A4 | 3.6 | 2.98 | solute carrier family 16, member 4 | 9122 | NM_001201546 | Hs.351306 | ENSG00000168679 |
| SLC25A15 | 3.84 | 3.09 | solute carrier family 25 | 10166 | NM_014252 | Hs.646645 | ENSG00000102743 |
| | | | (mitochondrial carrier; | | | | |
| | | | ornithine transporter) | | | | |
| | | | member 15 | | | | |
| SLC28A2 | 4.35 | 3.53 | solute carrier family 28 (concentrative | 9153 | NM_004212 | Hs.367833 | ENSG00000137860 |
| | | | nucleoside transporter), member 2 | | | | |
| SLC31A1 | 4.06 | 3.11 | solute carrier family 31 (copper | 1317 | NM_001859 | Hs.532315 | ENSG00000136868 |
| | | | transporter), member 1 | | | | |
| SLC35E3 | 2.91 | 2.43 | solute carrier family 35, member E3 | 55508 | NM_018656 | Hs.506011 | ENSG00000175782 |
| SLC36A2 | 4.11 | 3.19 | solute carrier family 36 (proton/amino | 153201 | NM_181776 | Hs.483877 | ENSG00000186335 |
| | | | acid symporter), member 2 | | | | |
| SLC37A2 | 4.9 | 3.85 | solute carrier family 37 (glucose-6- | 219855 | NM_001145290 | Hs.352661 | ENSG00000134955 |
| | | | phosphate transporter), member 2 | | | | |
| SLC44A4 | 5.05 | 3.52 | solute carrier family 44, member 4 | 80736 | NM_001178044 | Hs.335355 | ENSG00000204385 |
| SLC4A1 | 3.33 | 2.73 | solute carrier family 4 (anion | 6521 | NM_000342 | Hs.210751 | ENSG00000004939 |
| | | | exchanger), member 1 | | | | |
| | | | (Diego blood group) | | | | |
| SLC4A8 | 3.56 | 2.94 | solute carrier family 4, sodium | 9498 | NM_001039960 | Hs.4749 | ENSG00000050438 |
| | | | bicarbonate cotransporter, member 8 | | | | |
| SLC50A1 | 2.28 | 1.92 | solute carrier family 50 (sugar efflux | 55974 | NM_001122837 | Hs.292154 | ENSG00000169241 |
| | | | transporter), member 1 | | | | |
| SLC5A5 | 3.63 | 3.05 | solute carrier family 5 (sodium/iodide | 6528 | NM_000453 | Hs.584804 | ENSG00000105641 |
| | | | cotransporter), member 5 | | | | |
| SLC6A4 | 3.93 | 3.47 | solute carrier family 6 | 6532 | NM_001045 | Hs.29792 | ENSG00000108576 |
| | | | (neurotransmitter | | | | |
| | | | transporter), member 4 | | | | |
| SLC7A5P2 | 2.63 | 2.57 | solute carrier family 7 | 387254 | NR_002594 | Hs.448808 | |
| | | | (amino acid transporter light | | | | |
| | | | chain, L system), member 5 | | | | |
| | | | pseudogene 2 | | | | |
| SLC9A4 | 5.08 | 3.5 | solute carrier family 9, subfamily A | 389015 | NM_001011552 | Hs.447686 | ENSG00000180251 |
| | | | (NHE4, cation proton antiporter 4), | | | | |
| | | | member 4 | | | | |
| SLFNL1-AS1 | 3.57 | 2.93 | SLFNL1 antisense RNA 1 | 100507178 | NR_037868 | Hs.660056 | ENSG00000281207 |
| SMG1P7 | 3.5 | 3.15 | SMG1 pseudogene 7 | 100506060 | NR_033959 | Hs.655258 | ENSG00000261556 |
| SMIM14 | 3.45 | 2.75 | small integral membrane protein 14 | 201895 | NM_174921 | Hs.205952 | ENSG00000163683 |
| SMIM17 | 5.46 | 3.85 | small integral membrane protein 17 | 147670 | NM_001193628 | Hs.336588 | ENSG00000268182 |
| SNHG20 | 3.48 | 3.1 | small nucleolar RNA host gene 20 | 654434 | NR_027058 | Hs.720923 | ENSG00000234912 |
| SNHG4 | 4.17 | 3.63 | small nucleolar RNA host gene 4 | 724102 | NR_003141 | Hs.268939 | |
| SNX22 | 2.51 | 2.18 | sorting nexin 22 | 79856 | NM_024798 | Hs.744250 | ENSG00000157734 |
| SOX9-AS1 | 5.1 | 3.32 | SOX9 antisense RNA 1 | 400618 | NR_103737 | Hs.657374 | ENSG00000234899 |
| SPATS2 | 2.59 | 2.32 | spermatogenesis associated, | 65244 | NM_001293285 | Hs.654826 | ENSG00000123352 |
| | | | serine-rich 2 | | | | |
| SPATS2L | 2.78 | 2.29 | spermatogenesis associated, | 26010 | NM_001100422 | Hs.120323 | ENSG00000196141 |
| | | | serine-rich 2-like | | | | |
| SPC25 | 4.52 | 3.46 | SPC25, NDC80 kinetochore | 57405 | NM_020675 | Hs.421956 | ENSG00000152253 |
| | | | complex component | | | | |
| SPDYE8P | 2.11 | 1.98 | speedy/RINGO cell cycle regulator | 728524 | NM_001023562 | Hs.571275 | |
| | | | family member E8, pseudogene | | | | |
| SPIB | 3.67 | 2.75 | Spi-B transcription factor | 6689 | NM_001243998 | Hs.437905 | ENSG00000269404 |
| | | | (Spi-1/PU.1 related) | | | | |
| SPRED1 | 4.19 | 3.24 | sprouty-related, EVH1 | 161742 | NM_152594 | Hs.525781 | ENSG00000166068 |
| | | | domain containing 1 | | | | |
| SRRM2-AS1 | 3.86 | 3.24 | SRRM2 antisense RNA 1 | 100128788 | NR_027274 | Hs.311208 | ENSG00000205913 |
| SRSF12 | 3.63 | 3.27 | serine/arginine-rich splicing factor 12 | 135295 | NM_080743 | Hs.254414 | ENSG00000154548 |
| STAC2 | 4.17 | 3.11 | SH3 and cysteine rich domain 2 | 342667 | NM_198993 | Hs.145068 | ENSG00000141750 |
| STAP2 | 3.25 | 2.98 | signal transducing adaptor | 55620 | NM_001013841 | Hs.194385 | ENSG00000178078 |
| | | | family member 2 | | | | |
| STAR | 3.6 | 2.66 | steroidogenic acute regulatory protein | 6770 | NM_000349 | Hs.521535 | ENSG00000147465 |
| STAU2-AS1 | 4.02 | 4.08 | STAU2 antisense RNA 1 | 100128126 | NR_038406 | Hs.679921 | ENSG00000253302 |
| STRIP2 | 3.61 | 3.14 | striatin interacting protein 2 | 57464 | NM_001134336 | Hs.489988 | ENSG00000128578 |
| SWSAP1 | 2.79 | 2.31 | SWIM-type zinc finger 7 | 126074 | NM_175871 | Hs.631619 | ENSG00000173928 |
| | | | associated protein 1 | | | | |
| TAF8 | 2.86 | 2.4 | TAF8 RNA polymerase II, TATA | 129685 | NM_138572 | Hs.520122 | ENSG00000137413 |
| | | | box binding protein (TBP)-associated | | | | |
| | | | factor, 43 kDa | | | | |
| TANGO2 | 2.65 | 2.04 | transport and golgi | 128989 | NM_001283106 | Hs.474233 | ENSG00000183597 |
| | | | organization 2 homolog | | | | |
| TARS2 | 2.64 | 2.13 | threonyl-tRNA synthetase 2, | 80222 | NM_001271895 | Hs.288974 | ENSG00000143374 |
| | | | mitochondrial (putative) | | | | |
| TATDN3 | 3.33 | 2.85 | TatD DNase domain containing 3 | 128387 | NM_001042552 | Hs.530538 | ENSG00000203705 |
| TBC1D24 | 2.97 | 2.82 | TBC1 domain family, member 24 | 57465 | NM_001199107 | Hs.353087 | ENSG00000162065 |
| TBCCD1 | 2.64 | 2.29 | TBCC domain containing 1 | 55171 | NM_001134415 | Hs.518469 | ENSG00000113838 |
| TBXA2R | 3.83 | 3.16 | thromboxane A2 receptor | 6915 | NM_001060 | Hs.442530 | ENSG00000006638 |
| TEX101 | 3.65 | 3.17 | testis expressed 101 | 83639 | NM_001130011 | Hs.97978 | ENSG00000131126 |
| TFDP2 | 2.02 | 1.98 | transcription factor Dp-2 | 7029 | NM_001178138 | Hs.379018 | ENSG00000114126 |
| | | | (E2F dimerization partner 2) | | | | |
| THRIL | 3.15 | 2.63 | TNF and HNRNPL related | 102659353 | NR_110375 | Hs.596464 | ENSG00000280634 |
| | | | immunoregulatory long non- | | | | |
| | | | coding RNA | | | | |
| TIGD1 | 2.33 | 2.38 | rigger transposable element derived 1 | 200765 | NM_145702 | Hs.211823 | ENSG00000221944 |
| TINCR | 2.55 | 2.42 | tissue differentiation-inducing non- | 257000 | NM_153375 | Hs.515575 | ENSG00000223573 |
| | | | protein coding RNA | | | | |
| TLCD2 | 4.68 | 3.6 | TLC domain containing 2 | 727910 | NM_001164407 | Hs.531005 | ENSG00000185561 |
| TLR10 | 3.68 | 2.57 | toll-like receptor 10 | 81793 | NM_001017388 | Hs.120551 | ENSG00000174123 |
| TLR8-AS1 | 5.69 | 3.95 | TLR8 antisense RNA 1 | 349408 | NR_030727 | Hs.685035 | ENSG00000233338 |
| TMCC1-AS1 | 4.46 | 3.15 | TMCC1 antisense RNA 1 | 100507032 | NR_037893 | Hs.529562 | ENSG00000271270 |
| | | | (head to head) | | | | |
| TMEM106A | 3.32 | 2.82 | transmembrane protein 106A | 113277 | NM_001291586 | Hs.536474 | ENSG00000184988 |
| TMEM120B | 2.62 | 2.28 | transmembrane protein 120B | 144404 | NM_001080825 | Hs.644504 | ENSG00000188735 |
| TMEM168 | 2.41 | 2.01 | transmembrane protein 168 | 64418 | NM_001287497 | Hs.606345 | ENSG00000146802 |
| TMEM212 | 4.48 | 3.34 | transmembrane protein 212 | 389177 | NM_001164436 | Hs.642307 | ENSG00000186329 |
| TMEM213 | 3.63 | 3.05 | transmembrane protein 213 | 155006 | NM_001085429 | Hs.567729 | ENSG00000214128 |
| TMEM236 | 3.74 | 3.34 | transmembrane protein 236 | 653567 | NM_001013629 | Hs.564139 | ENSG00000148483 |
| TMEM254-AS1 | 3.57 | 2.82 | TMEM254 antisense RNA 1 | 219347 | NR_027428 | Hs.524453 | |
| TMEM38A | 3.65 | 3.13 | transmembrane protein 38A | 79041 | NM_024074 | Hs.436068 | ENSG00000072954 |
| TMEM41B | 2.92 | 2.24 | transmembrane protein 41B | 440026 | NM_001165030 | Hs.594563 | ENSG00000166471 |
| TMIGD2 | 2.08 | 1.98 | transmembrane and immunoglobulin | 126259 | NM_001169126 | Hs.263928 | ENSG00000167664 |
| | | | domain containing 2 | | | | |
| TNFAIP8L1 | 3.03 | 2.55 | tumor necrosis factor, alpha- | 126282 | NM_001167942 | Hs.465643 | ENSG00000185361 |
| | | | induced protein 8-like 1 | | | | |
| TNFAIP8L2- | 6.07 | 4.08 | TNFAIP8L2-SCNM1 readthrough | 100534012 | NM_001204848 | Hs.732060 | ENSG00000163156 |
| SCNM1 | | | | | | | |
| TONSL | 2.99 | 2.48 | tonsoku-like, DNA repair protein | 4796 | NM_013432 | Hs.675285 | ENSG00000160949 |
| TOR1AIP2 | 2.24 | 1.91 | torsin A interacting protein 2 | 163590 | NM_001199260 | Hs.571797 | ENSG00000169905 |
| TOR4A | 3.61 | 2.93 | torsin family 4, member A | 54863 | NM_017723 | Hs.495541 | ENSG00000198113 |
| TPMT | 2.9 | 2.63 | thiopurine S-methyltransferase | 7172 | NM_000367 | Hs.444319 | ENSG00000137364 |
| TPTEP1 | 3.95 | 2.81 | transmembrane phosphatase with | 387590 | NR_001591 | Hs.474116 | ENSG00000100181 |
| | | | tensin homology pseudogene 1 | | | | |
| TRAF3IP2 | 3.47 | 2.98 | TRAF3 interacting protein 2 | 10758 | NM_001164281 | Hs.561514 | ENSG00000056972 |
| TRAPPC2 | 2.11 | 2.01 | trafficking protein particle complex 2 | 6399 | NM_001011658 | Hs.592238 | ENSG00000196459 |
| TRIM16 | 2.7 | 2.5 | tripartite motif containing 16 | 10626 | NM_006470 | Hs.123534 | ENSG00000221926 |
| TRIM45 | 4.23 | 3.39 | tripartite motif containing 45 | 80263 | NM_001145635 | Hs.301526 | ENSG00000134253 |
| TRPV1 | 3.44 | 3.24 | transient receptor potential cation | 7442 | NM_018727 | Hs.579217 | ENSG00000196689 |
| | | | channel, subfamily V, member 1 | | | | |
| TSG1 | 4.82 | 3.93 | tumor suppressor TSG1 | 643432 | NR_015362 | Hs.509936 | |
| TSIX | 4.23 | 3.42 | TSIX transcript, XIST antisense RNA | 9383 | NR_003255 | Hs.529901 | ENSG00000270641 |
| TSTD3 | 3.37 | 3.14 | thiosulfate sulfurtransferase | 100130890 | NM_001195131 | Hs.634506 | ENSG00000228439 |
| | | | (rhodanese)-like domain containing 3 | | | | |
| TUBA3FP | 3.9 | 3.23 | tubulin, alpha 3f, pseudogene | 113691 | NR_003608 | Hs.585006 | ENSG00000161149 |
| TUFT1 | 3.19 | 2.99 | tuftelin 1 | 7286 | NM_001126337 | Hs.489922 | ENSG00000143367 |
| TVP23C | 2.66 | 2.56 | trans-golgi network vesicle protein 23 | 201158 | NM_001135036 | Hs.164595 | ENSG00000175106 |
| | | | homolog C (S. cerevisiae) | | | | |
| UBE2Q2P1 | 3.73 | 3.08 | ubiquitin-conjugating enzyme E2Q | 388165 | NM_207382 | Hs.498348 | ENSG00000189136 |
| | | | family member 2 pseudogene 1 | | | | |
| UBL7-AS1 | 4.09 | 3.42 | UBL7 antisense RNA 1 | 440288 | NR_038448 | Hs.611046 | ENSG00000247240 |
| | | | (head to head) | | | | |
| UBOX5 | 2.27 | 2.05 | U-box domain containing 5 | 22888 | NM_001267584 | Hs.654646 | ENSG00000185019 |
| UCKL1-AS1 | 3.97 | 3.53 | UCKL1 antisense RNA 1 | 100113386 | NR_027287 | Hs.551552 | |
| UGDH-AS1 | 4.44 | 3.36 | UGDH antisense RNA 1 | 100885776 | NR_047679 | Hs.640769 | ENSG00000249348 |
| UGGT1 | 2.1 | 1.94 | UDP-glucose glycoprotein | 56886 | NM_001025777 | Hs.743306 | ENSG00000136731 |
| | | | glucosyltransferase 1 | | | | |
| UGT8 | 4.93 | 3.72 | UDP glycosyltransferase 8 | 7368 | NM_001128174 | Hs.144197 | ENSG00000174607 |
| UPK1B | 4.09 | 3.31 | uroplakin 1B | 7348 | NM_006952 | Hs.271580 | ENSG00000114638 |
| USP49 | 2.46 | 2.25 | ubiquitin specific peptidase 49 | 25862 | NM_001286554 | Hs.593575 | ENSG00000164663 |
| USP54 | 2.37 | 2.16 | ubiquitin specific peptidase 54 | 159195 | NM_152586 | Hs.657355 | ENSG00000166348 |
| UTP11L | 3.22 | 2.35 | UTP11-like, U3 small nucleolar | 51118 | NM_016037 | Hs.472038 | ENSG00000183520 |
| | | | ribonucleoprotein (yeast) | | | | |
| UTS2B | 4.79 | 3.78 | urotensin 2B | 257313 | NM_198152 | Hs.518492 | ENSG00000188958 |
| VSIG1 | 2.55 | 2.09 | V-set and immunoglobulin | 340547 | NM_001170553 | Hs.177164 | ENSG00000101842 |
| | | | domain containing 1 | | | | |
| VSTM4 | 4.19 | 3.25 | V-set and transmembrane | 196740 | NM_001031746 | Hs.522928 | ENSG00000165633 |
| | | | domain containing 4 | | | | |
| WDR11-AS1 | 4.3 | 3.3 | WDR11 antisense RNA 1 | 283089 | NR_033850 | Hs.568750 | ENSG00000227165 |
| WDR45 | 2.27 | 1.9 | WD repeat domain 45 | 11152 | NM_001029896 | Hs.632807 | ENSG00000196998 |
| WDR92 | 2.37 | 1.67 | WD repeat domain 92 | 116143 | NM_001256476 | Hs.631877 | ENSG00000243667 |
| WFDC8 | 4.12 | 3.11 | WAP four-disulfide core domain 8 | 90199 | NM_130896 | Hs.116128 | ENSG00000158901 |
| WNT7B | 3.91 | 3.4 | wingless-type MMTV integration site | 7477 | NM_058238 | Hs.512714 | ENSG00000188064 |
| | | | family, member 7B | | | | |
| XIAP | 2.32 | 1.93 | X-linked inhibitor of apoptosis, E3 | 331 | NM_001167 | Hs.356076 | ENSG00000101966 |
| | | | ubiquitin protein ligase | | | | |
| XKR9 | 4.97 | 3.6 | XK, Kell blood group complex | 389668 | NM_001011720 | Hs.458938 | ENSG00000221947 |
| | | | subunit-related family, member 9 | | | | |
| XPNPEP3 | 2.73 | 2.35 | X-prolyl aminopeptidase 3, | 63929 | NM_001204827 | Hs.529163 | ENSG00000196236 |
| | | | mitochondrial | | | | |
| XRCC2 | 3.95 | 3.39 | X-ray repair complementing defective | 7516 | NM_005431 | Hs.647093 | ENSG00000196584 |
| | | | repair in Chinese hamster cells 2 | | | | |
| ZBTB8A | 3.97 | 3.28 | zinc finger and BTB domain | 653121 | NM_001040441 | Hs.546479 | ENSG00000160062 |
| | | | containing 8A | | | | |
| ZC3H12D | 2.26 | 2.3 | zinc finger CCCH-type | 340152 | NM_207360 | Hs.632618 | ENSG00000178199 |
| | | | containing 12D | | | | |
| ZFP14 | 2.62 | 1.96 | ZFP14 zinc finger protein | 57677 | NM_001297619 | Hs.35524 | ENSG00000142065 |
| ZFP30 | 2.66 | 2.28 | ZFP30 zinc finger protein | 22835 | NM_014898 | Hs.716719 | ENSG00000120784 |
| ZFP42 | 3.79 | 2.91 | ZFP42 zinc finger protein | 132625 | NM_001304358 | Hs.335787 | ENSG00000179059 |
| ZKSCAN3 | 3.87 | 2.77 | zinc finger with KRAB and | 80317 | NM_001242894 | Hs.380930 | ENSG00000189298 |
| | | | SCAN domains 3 | | | | |
| ZKSCAN7 | 2.64 | 2.17 | zinc finger with KRAB and | 55888 | NM_001288590 | Hs.529512 | ENSG00000196345 |
| | | | SCAN domains 7 | | | | |
| ZMYM5 | 2.23 | 1.95 | zinc finger, MYM-type 5 | 9205 | NM_001039649 | Hs.530988 | ENSG00000132950 |
| ZNF154 | 2.49 | 2.26 | zinc finger protein 154 | 7710 | NM_001085384 | Hs.646378 | ENSG00000179909 |
| ZNF2 | 3.33 | 2.35 | zinc finger protein 2 | 7549 | NM_001017396 | Hs.590916 | ENSG00000275111 |
| ZNF264 | 2.16 | 1.84 | zinc finger protein 264 | 9422 | NM_003417 | Hs.515634 | ENSG00000083844 |
| ZNF286B | 3.02 | 2.56 | zinc finger protein 286B | 729288 | NM_001145045 | Hs.534279 | ENSG00000249459 |
| ZNF34 | 4 | 3.19 | zinc finger protein 34 | 80778 | NM_001286769 | Hs.631854 | ENSG00000196378 |
| ZNF347 | 3.18 | 2.72 | zinc finger protein 347 | 84671 | NM_001172674 | Hs.467239 | ENSG00000197937 |
| ZNF471 | 5.04 | 3.57 | zinc finger protein 471 | 57573 | NM_020813 | Hs.710590 | ENSG00000196263 |
| ZNF483 | 3.18 | 2.91 | zinc finger protein 483 | 158399 | NM_001007169 | Hs.584864 | ENSG00000173258 |
| ZNF490 | 2.8 | 2.42 | zinc finger protein 490 | 57474 | NM_020714 | Hs.655860 | ENSG00000188033 |
| ZNF492 | 3.5 | 2.92 | zinc finger protein 492 | 57615 | NM_020855 | Hs.232108 | ENSG00000229676 |
| ZNF526 | 3.18 | 2.4 | zinc finger protein 526 | 116115 | NM_133444 | Hs.137282 | ENSG00000167625 |
| ZNF527 | 2.89 | 2.38 | zinc finger protein 527 | 84503 | NM_032453 | Hs.590940 | ENSG00000189164 |
| ZNF543 | 2.48 | 2.13 | zinc finger protein 543 | 125919 | NM_213598 | Hs.202544 | ENSG00000178229 |
| ZNF554 | 3.36 | 2.65 | zinc finger protein 554 | 115196 | NM_001102651 | Hs.307043 | ENSG00000172006 |
| ZNF556 | 4.28 | 4.37 | zinc finger protein 556 | 80032 | NM_001300843 | Hs.287433 | ENSG00000172000 |
| ZNF562 | 2.57 | 2.1 | zinc finger protein 562 | 54811 | NM_001130031 | Hs.371107 | ENSG00000171466 |
| ZNF662 | 3.76 | 2.78 | zinc finger protein 662 | 389114 | NM_001134656 | Hs.720173 | ENSG00000182983 |
| ZNF665 | 4.04 | 3.18 | zinc finger protein 665 | 79788 | NM_024733 | Hs.745230 | ENSG00000197497 |
| ZNF677 | 3.48 | 2.9 | zinc finger protein 677 | 342926 | NM_182609 | Hs.20506 | ENSG00000197928 |
| ZNF713 | 3.93 | 3.69 | zinc finger protein 713 | 349075 | NM_182633 | Hs.660834 | ENSG00000178665 |
| ZNF716 | 3.7 | 3.18 | zinc finger protein 716 | 441234 | NM_001159279 | Hs.533121 | ENSG00000182111 |
| ZNF761 | 3.08 | 2.59 | zinc finger protein 761 | 388561 | NM_001008401 | Hs.433293 | ENSG00000160336 |
| ZNF785 | 2.85 | 2.5 | zinc finger protein 785 | 146540 | NM_152458 | Hs.513509 | ENSG00000197162 |
| ZNF793 | 3.98 | 3.26 | zinc finger protein 793 | 390927 | NM_001013659 | Hs.568010 | ENSG00000188227 |
| ZNF814 | 2.58 | 2.29 | zinc finger protein 814 | 730051 | NM_001144989 | Hs.634143 | ENSG00000204514 |
| ZNF818P | 3.18 | 2.52 | zinc finger protein 818, pseudogene | 390963 | NM_001001675 | Hs.444446 | |
| ZNF850 | 3.21 | 2.68 | zinc finger protein 850 | 342892 | NM_001193552 | Hs.406307 | ENSG00000267041 |
| ZNRF3-AS1 | 4.24 | 3.54 | ZNRF3 antisense RNA 1 | 100874123 | NR_046851 | Hs.674708 | ENSG00000177993 |
| ZSCAN22 | 3.74 | 2.67 | zinc finger and SCAN | 342945 | NM_181846 | Hs.388162 | ENSG00000182318 |
| | | | domain containing 22 | | | | |
| ZYG11A | 4.09 | 3.51 | zyg-11 family member A, | 440590 | NM_001004339 | Hs.658458 | ENSG00000203995 |
| | | | cell cycle regulator | | | | |

TABLE 7A
Monocyte Subtype Genes.

| Fold-change | | | Entrez | | | |
|---|---|---|---|---|---|---|
| mono1 vs mono2 | UniqueID | Name | ID | Accession | UGCluster | Ensembl |
| −2.1276596 | NT5DC3 | 5′-nucleotidase domain | 51559 | NM_001031701 | Hs.48428 | ENSG00000111696 |
| | | containing 3 | | | | |
| 2.0400000 | PGLS | 6-phosphogluconolactonase | 25796 | NM_012088 | Hs.466165 | ENSG00000130313 |
| 2.0200000 | ABHD14A | abhydrolase domain | 25864 | NM_015407 | Hs.534400 | ENSG00000248487 |
| | | containing 14A | | | | |
| 2.0600000 | AAAS | achalasia, adrenocortical | 8086 | NM_001173466 | Hs.369144 | ENSG00000094914 |
| | | insufficiency, alacrimia | | | | |
| 1.7100000 | ACP2 | acid phosphatase 2, lysosomal | 53 | NM_001131064 | Hs.532492 | ENSG00000134575 |
| 1.5600000 | ACO2 | aconitase 2, mitochondrial | 50 | NM_001098 | Hs.643610 | ENSG00000100412 |
| 1.6400000 | APEH | acylaminoacyl-peptide | 327 | NM_001640 | Hs.517969 | ENSG00000164062 |
| | | hydrolase | | | | |
| −2.7027027 | ACBD7 | acyl-CoA binding domain | 414149 | NM_001039844 | Hs.644598 | ENSG00000176244 |
| | | containing 7 | | | | |
| −2.1276596 | ACADSB | acyl-CoA dehydrogenase, | 36 | NM_001609 | Hs.81934 | ENSG00000196177 |
| | | short/branched chain | | | | |
| 1.6300000 | AP1M1 | adaptor-related protein | 8907 | NM_001130524 | Hs.71040 | ENSG00000072958 |
| | | complex 1, mu 1 subunit | | | | |
| −2.3809524 | AP1S3 | adaptor-related protein | 130340 | NM_001039569 | Hs.632555 | ENSG00000152056 |
| | | complex 1, sigma 3 subunit | | | | |
| 1.4800000 | AP2B1 | adaptor-related protein | 163 | NM_001030006 | Hs.514819 | ENSG00000006125 |
| | | complex 2, beta 1 subunit | | | | |
| −1.7857143 | AP4S1 | adaptor-related protein | 11154 | NM_001128126 | Hs.293411 | ENSG00000100478 |
| | | complex 4, sigma 1 subunit | | | | |
| −1.7543860 | ADAT1 | adenosine deaminase, tRNA- | 23536 | NM_012091 | Hs.729312 | ENSG00000065457 |
| | | specific 1 | | | | |
| 1.5200000 | ADSL | adenylosuccinate lyase | 158 | NM_000026 | Hs.75527 | ENSG00000239900 |
| 1.5600000 | ARL2BP | ADP-ribosylation factor-like | 23568 | NM_012106 | Hs.632873 | ENSG00000102931 |
| | | 2 binding protein | | | | |
| −2.4390244 | ADRA1A | adrenoceptor alpha 1A | 148 | NM_000680 | Hs.709175 | ENSG00000120907 |
| −1.8181818 | AARS2 | alanyl-tRNA synthetase 2, | 57505 | NM_020745 | Hs.158381 | ENSG00000124608 |
| | | mitochondrial | | | | |
| 1.7400000 | ALDH2 | aldehyde dehydrogenase 2 | 217 | NM_000690 | Hs.604551 | ENSG00000111275 |
| | | family (mitochondrial) | | | | |
| −2.0408163 | ALDH6A1 | aldehyde dehydrogenase 6 | 4329 | NM_001278593 | Hs.293970 | ENSG00000119711 |
| | | family, member A1 | | | | |
| 1.5700000 | AKR1B1 | aldo-keto reductase family 1, | 231 | NM_001628 | Hs.521212 | ENSG00000085662 |
| | | member B1 (aldose | | | | |
| | | reductase) | | | | |
| 1.5800000 | ALKBH5 | AlkB family member 5, RNA | 54890 | NM_017758 | Hs.744130 | ENSG00000091542 |
| | | demethylase | | | | |
| −1.5384615 | ALPK1 | alpha-kinase 1 | 80216 | NM_001102406 | Hs.652825 | ENSG00000073331 |
| −2.0408163 | AASS | aminoadipate-semialdehyde | 10157 | NM_005763 | Hs.156738 | ENSG00000008311 |
| | | synthase | | | | |
| 2.0800000 | AGTRAP | angiotensin II receptor- | 57085 | NM_001040194 | Hs.464438 | ENSG00000177674 |
| | | associated protein | | | | |
| −1.8181818 | ASB11 | ankyrin repeat and SOCS box | 140456 | NM_001012428 | Hs.352183 | ENSG00000165192 |
| | | containing 11, E3 ubiquitin | | | | |
| | | protein ligase | | | | |
| −2.4390244 | ANKRD20A9P | ankyrin repeat domain 20 | 284232 | NR_027995 | Hs.679496 | |
| | | family, member A9, | | | | |
| | | pseudogene | | | | |
| −1.6949153 | ANKRD36B | ankyrin repeat domain 36B | 57730 | NM_020970 | Hs.532921 | ENSG00000196912 |
| 1.3900000 | ANXA11 | annexin A11 | 311 | NM_001157 | Hs.530291 | ENSG00000122359 |
| −1.6393443 | ANP32A-IT1 | ANP32A intronic transcript 1 | 80035 | NM_001040150 | Hs.662150 | |
| −1.3698630 | AAK1 | AP2 associated kinase 1 | 22848 | NM_014911 | Hs.468878 | ENSG00000115977 |
| −3.1250000 | APOBEC3B-AS1 | APOBEC3B antisense RNA 1 | 100874530 | NR_104187 | Hs.626951 | ENSG00000249310 |
| 1.7200000 | APOA1BP | apolipoprotein A-I binding | 128240 | NM_144772 | Hs.528320 | ENSG00000163382 |
| | | protein | | | | |
| −3.8461538 | APOA2 | apolipoprotein A-II | 336 | NM_001643 | Hs.237658 | ENSG00000158874 |
| −1.8181818 | APOL1 | apolipoprotein L, 1 | 8542 | NM_001136540 | Hs.114309 | ENSG00000100342 |
| −2.4390244 | APOL4 | apolipoprotein L, 4 | 80832 | NM_030643 | Hs.115099 | ENSG00000100336 |
| −1.9607843 | ARGFX | arginine-fifty homeobox | 503582 | NM_001012659 | Hs.224976 | ENSG00000186103 |
| −2.8571429 | ARHGEF26-AS1 | ARHGEF26 antisense RNA 1 | 100507524 | NR_037901 | Hs.370221 | ENSG00000243069 |
| −1.8518519 | ARMC9 | armadillo repeat containing 9 | 80210 | NM_001271466 | Hs.471610 | ENSG00000135931 |
| 1.5400000 | ASNA1 | arsA arsenite transporter, | 439 | NM_004317 | Hs.465985 | ENSG00000198356 |
| | | ATP-binding, homolog 1 | | | | |
| | | (bacterial) | | | | |
| −5.2631579 | AS3MT | arsenite methyltransferase | 57412 | NM_020682 | Hs.720370 | ENSG00000214435 |
| −2.0833333 | AIPL1 | aryl hydrocarbon receptor | 23746 | NM_001033054 | Hs.279887 | ENSG00000129221 |
| | | interacting protein-like 1 | | | | |
| 1.5600000 | ASTE1 | asteroid homolog 1 | 28990 | NM_001288950 | Hs.100878 | ENSG00000034533 |
| | | (Drosophila) | | | | |
| −3.4482759 | ASTN2 | astrotactin 2 | 23245 | NM_001184734 | Hs.601562 | ENSG00000148219 |
| −1.4285714 | ATXN7 | ataxin 7 | 6314 | NM_000333 | Hs.476595 | ENSG00000163635 |
| 1.5400000 | ATP5B | ATP synthase, H+ | 506 | NM_001686 | Hs.406510 | ENSG00000110955 |
| | | transporting, mitochondrial | | | | |
| | | F1 complex, beta polypeptide | | | | |
| 2.5900000 | ATP5D | ATP synthase, H+ | 513 | NM_001001975 | Hs.418668 | ENSG00000099624 |
| | | transporting, mitochondrial | | | | |
| | | F1 complex, delta subunit | | | | |
| 2.5100000 | ATP5G2 | ATP synthase, H+ | 517 | NM_001002031 | Hs.524464 | ENSG00000135390 |
| | | transporting, mitochondrial | | | | |
| | | Fo complex, subunit C2 | | | | |
| | | (subunit 9) | | | | |
| −2.2222222 | ATAD3C | ATPase family, AAA domain | 219293 | NM_001039211 | Hs.724767 | ENSG00000215915 |
| | | containing 3C | | | | |
| −2.1739130 | ABCA9 | ATP-binding cassette, sub- | 10350 | NM_080283 | Hs.131686 | ENSG00000154258 |
| | | family A (ABC1), member 9 | | | | |
| −2.8571429 | ABCC9 | ATP-binding cassette, sub- | 10060 | NM_005691 | Hs.732701 | ENSG00000069431 |
| | | family C (CFTR/MRP), | | | | |
| | | member 9 | | | | |
| −1.7241379 | ABCF1 | ATP-binding cassette, sub- | 23 | NM_001025091 | Hs.655285 | ENSG00000204574 |
| | | family F (GCN20), member 1 | | | | |
| −2.5641026 | BBS5 | Bardet-Biedl syndrome 5 | 129880 | NM_152384 | Hs.233398 | ENSG00000163093 |
| −2.3255814 | BNIPL | BCL2/adenovirus E1B 19 kD | 149428 | NM_001159642 | Hs.591473 | ENSG00000163141 |
| | | interacting protein like | | | | |
| −2.0833333 | BCL2L2- | BCL2L2-PABPN1 | 100529063 | NM_001199864 | Hs.707712 | ENSG00000258643 |
| | PABPN1 | readthrough | | | | |
| −2.3809524 | BZRAP1 | benzodiazepine receptor | 9256 | NM_001261835 | Hs.112499 | ENSG00000005379 |
| | | (peripheral) associated | | | | |
| | | protein 1 | | | | |
| −2.2222222 | BHMT2 | betaine-homocysteine S- | 23743 | NM_001178005 | Hs.114172 | ENSG00000132840 |
| | | methyltransferase 2 | | | | |
| −1.7857143 | BVES | blood vessel epicardial | 11149 | NM_001199563 | Hs.221660 | ENSG00000112276 |
| | | substance | | | | |
| −2.0000000 | BMS1P4 | BMS1 pseudogene 4 | 729096 | NR_026592 | Hs.709171 | ENSG00000271816 |
| −2.0000000 | BMS1P5 | BMS1 pseudogene 5 | 399761 | NM_001040053 | Hs.571994 | ENSG00000204177 |
| −1.7857143 | BMS1P6 | BMS1 pseudogene 6 | 642826 | NR_024495 | Hs.463017 | ENSG00000251079 |
| 1.9300000 | BOLA3 | bolA family member 3 | 388962 | NM_001035505 | Hs.61472 | ENSG00000163170 |
| −2.1276596 | BMP7 | bone morphogenetic protein 7 | 655 | NM_001719 | Hs.473163 | ENSG00000101144 |
| 1.8200000 | BCKDHA | branched chain keto acid | 593 | NM_000709 | Hs.433307 | ENSG00000248098 |
| | | dehydrogenase E1, alpha | | | | |
| | | polypeptide | | | | |
| −2.3809524 | BRIP1 | BRCA1 interacting protein C- | 83990 | NM_032043 | Hs.128903 | ENSG00000136492 |
| | | terminal helicase 1 | | | | |
| −2.6315789 | BREA2 | breast cancer estrogen- | 286076 | NM_001024610 | Hs.178095 | ENSG00000181097 |
| | | induced apoptosis 2 | | | | |
| −1.4492754 | BAZ2A | bromodomain adjacent to | 11176 | NM_001300905 | Hs.314263 | ENSG00000076108 |
| | | zinc finger domain, 2A | | | | |
| −1.7543860 | BTN2A1 | butyrophilin, subfamily 2. | 11120 | NM_001197233 | Hs.159028 | ENSG00000112763 |
| | | member A1 | | | | |
| 2.5600000 | C10orf32-ASMT | C10orf32-ASMT readthrough | 100528007 | NR_037644 | Hs.720370 | |
| | | (NMD candidate) | | | | |
| −1.7241379 | C1RL-AS1 | C1RL antisense RNA 1 | 283314 | NR_026947 | Hs.744212 | ENSG00000205885 |
| −2.0408163 | CDH23 | cadherin-related 23 | 64072 | NM_001171930 | Hs.656032 | ENSG00000107736 |
| −2.1739130 | LOC729603 | calcineurin-like EF-hand | 729603 | NR_003288 | Hs.674810 | ENSG00000213073 |
| | | protein 1 pseudogene | | | | |
| −1.8867925 | CAMK1D | calcium/calmodulin- | 57118 | NM_020397 | Hs.600547 | ENSG00000183049 |
| | | dependent protein kinase ID | | | | |
| 1.3600000 | CAD | carbamoyl-phosphate | 790 | NM_004341 | Hs.377010 | ENSG00000084774 |
| | | synthetase 2, aspartate | | | | |
| | | transcarbamylase, and | | | | |
| | | dihydroorotase | | | | |
| −2.1739130 | CHST6 | carbohydrate (N- | 4166 | NM_021615 | Hs.655622 | ENSG00000183196 |
| | | acetylglucosamine 6-O) | | | | |
| | | sulfotransferase 6 | | | | |
| −1.8867925 | CA5B | carbonic anhydrase VB, | 11238 | NM_007220 | Hs.653287 | ENSG00000169239 |
| | | mitochondrial | | | | |
| 1.3200000 | CRTAP | cartilage associated protein | 10491 | NM_006371 | Hs.517888 | ENSG00000170275 |
| 1.6700000 | CASD1 | CAS1 domain containing 1 | 64921 | NM_022900 | Hs.260041 | ENSG00000127995 |
| 1.3600000 | CSNK2A1 | casein kinase 2, alpha 1 | 1457 | NM_001895 | Hs.644056 | ENSG00000101266 |
| | | polypeptide | | | | |
| −1.5873016 | CFLAR | CASP8 and FADD-like | 8837 | NM_001127183 | Hs.390736 | ENSG00000003402 |
| | | apoptosis regulator | | | | |
| 1.8600000 | CAT | catalase | 847 | NM_001752 | Hs.502302 | ENSG00000121691 |
| 1.5900000 | COMT | catechol-O-methyltransferase | 1312 | NM_000754 | Hs.370408 | ENSG00000093010 |
| −1.5873016 | CTNNBL1 | catenin, beta like 1 | 56259 | NM_001281495 | Hs.472667 | ENSG00000132792 |
| 1.3200000 | CD164 | CD164 molecule, sialomucin | 8763 | NM_001142401 | Hs.520313 | ENSG00000135535 |
| −2.3809524 | CD24 | CD24 molecule | 100133941 | NM_001291737 | Hs.644105 | ENSG00000272398 |
| −2.5641026 | CDKN2B-AS1 | CDKN2B antisense RNA 1 | 100048912 | NR_003529 | Hs.493614 | ENSG00000240498 |
| 2.2400000 | CDIPT | CDP-diacylglycerol--inositol | 10423 | NM_001286585 | Hs.121549 | ENSG00000103502 |
| | | 3-phosphatidyltransferase | | | | |
| −2.5641026 | LOC101409256 | cell division cycle 42 | 101409256 | NR_102424 | | |
| | | pseudogene | | | | |
| −1.4492754 | CCAR1 | cell division cycle and | 55749 | NM_001282959 | Hs.49853 | ENSG00000060339 |
| | | apoptosis regulator 1 | | | | |
| 1.8200000 | CREG1 | cellular repressor of E1A- | 8804 | NM_003851 | Hs.5710 | ENSG00000143162 |
| | | stimulated genes 1 | | | | |
| −1.5625000 | CENPC | centromere protein C | 1060 | NM_001812 | Hs.479867 | ENSG00000145241 |
| −2.0408163 | CEP41 | centrosomal protein 41 kDa | 95681 | NM_001257158 | Hs.368315 | ENSG00000106477 |
| −1.2820513 | CERS5 | ceramide synthase 5 | 91012 | NM_001281731 | Hs.270525 | ENSG00000139624 |
| 1.5700000 | CCM2 | cerebral cavernous | 83605 | NM_001029835 | Hs.148272 | ENSG00000136280 |
| | | malformation 2 | | | | |
| 1.8600000 | CLN6 | ceroid-lipofuscinosis, | 54982 | NM_017882 | Hs.584921 | ENSG00000128973 |
| | | neuronal 6, late infantile, | | | | |
| | | variant | | | | |
| −1.3888889 | CHMP1B | charged multivesicular body | 57132 | NM_020412 | Hs.656244 | ENSG00000255112 |
| | | protein 1B | | | | |
| 1.2600000 | CHMP3 | charged multivesicular body | 51652 | NM_001005753 | Hs.591582 | ENSG00000115561 |
| | | protein 3 | | | | |
| −2.3255814 | CCR6 | chemokine (C-C motif) | 1235 | NM_004367 | Hs.46468 | ENSG00000112486 |
| | | receptor 6 | | | | |
| −2.5641026 | CHRM3 | cholinergic receptor, | 1131 | NM_000740 | Hs.7138 | ENSG00000133019 |
| | | muscarinic 3 | | | | |
| −1.8181818 | CHRNB1 | cholinergic receptor, | 1140 | NM_000747 | Hs.330386 | ENSG00000170175 |
| | | nicotinic, beta 1 (muscle) | | | | |
| −2.2727273 | LOC440300 | chondroitin sulfate | 440300 | NR_033738 | Hs.546565 | ENSG00000259295 |
| | | proteoglycan 4 pseudogene | | | | |
| −1.4705882 | CBX5 | chromobox homolog 5 | 23468 | NM_001127321 | Hs.349283 | ENSG00000094916 |
| 2.6700000 | C1orf122 | chromosome 1 open reading | 127687 | NM_001142726 | Hs.532749 | ENSG00000197982 |
| | | frame 122 | | | | |
| −1.8867925 | C1orf174 | chromosome 1 open reading | 339448 | NM_207356 | Hs.103939 | ENSG00000198912 |
| | | frame 174 | | | | |
| −2.9411765 | C1orf229 | chromosome 1 open reading | 388759 | NM_207401 | Hs.456511 | ENSG00000221953 |
| | | frame 229 | | | | |
| −1.4492754 | C1orf27 | chromosome 1 open reading | 54953 | NM_001164245 | Hs.371210 | ENSG00000157181 |
| | | frame 27 | | | | |
| −1.6129032 | C10orf32 | chromosome 10 open reading | 119032 | NM_001136200 | Hs.34492 | ENSG00000166275 |
| | | frame 32 | | | | |
| 1.5400000 | C12orf49 | chromosome 12 open reading | 79794 | NM_024738 | Hs.592011 | ENSG00000111412 |
| | | frame 49 | | | | |
| −1.4492754 | C12orf5 | chromosome 12 open reading | 57103 | NM_020375 | Hs.504545 | ENSG00000078237 |
| | | frame 5 | | | | |
| 1.6400000 | C14orf142 | chromosome 14 open reading | 84520 | NM_032490 | Hs.20142 | ENSG00000170270 |
| | | frame 142 | | | | |
| 2.3400000 | C16orf54 | chromosome 16 open reading | 283897 | NM_175900 | Hs.331095 | ENSG00000185905 |
| | | frame 54 | | | | |
| 1.5100000 | C16orf62 | chromosome 16 open reading | 57020 | NM_001300743 | Hs.654964 | ENSG00000103544 |
| | | frame 62 | | | | |
| −2.0000000 | C17orf75 | chromosome 17 open reading | 64149 | NM_022344 | Hs.655257 | ENSG00000108666 |
| | | frame 75 | | | | |
| −2.0000000 | C17orf77 | chromosome 17 open reading | 146723 | NM_001302809 | Hs.350775 | ENSG00000182352 |
| | | frame 77 | | | | |
| 2.5600000 | C19orf24 | chromosome 19 open reading | 55009 | NM_017914 | Hs.591383 | ENSG00000228300 |
| | | frame 24 | | | | |
| −2.2727273 | C19orf40 | chromosome 19 open reading | 91442 | NM_001300978 | Hs.579899 | ENSG00000131944 |
| | | frame 40 | | | | |
| 2.2300000 | C19orf70 | chromosome 19 open reading | 125988 | NM_205767 | Hs.356626 | ENSG00000174917 |
| | | frame 70 | | | | |
| −2.0000000 | C2orf83 | chromosome 2 open reading | 56918 | NM_001162483 | Hs.283092 | ENSG00000042304 |
| | | frame 83 | | | | |
| −3.1250000 | C2orf91 | chromosome 2 open reading | 400950 | NM_001242815 | Hs.738713 | ENSG00000205086 |
| | | frame 91 | | | | |
| −2.7777778 | C20orf203 | chromosome 20 open reading | 284805 | NM_182584 | Hs.353262 | |
| | | frame 203 | | | | |
| 3.1100000 | C20orf27 | chromosome 20 open reading | 54976 | NM_001039140 | Hs.274422 | ENSG00000101220 |
| | | frame 27 | | | | |
| −2.3809524 | C21orf62 | chromosome 21 open reading | 56245 | NM_001162495 | Hs.517235 | ENSG00000262938 |
| | | frame 62 | | | | |
| −2.0000000 | C3orf33 | chromosome 3 open reading | 285315 | NM_173657 | Hs.350846 | ENSG00000174928 |
| | | frame 33 | | | | |
| −1.8518519 | C4orf19 | chromosome 4 open reading | 55286 | NM_001104629 | Hs.107527 | ENSG00000154274 |
| | | frame 19 | | | | |
| −2.2727273 | C4orf26 | chromosome 4 open reading | 152816 | NM_001206981 | Hs.24510 | ENSG00000174792 |
| | | frame 26 | | | | |
| −1.9607843 | C5orf28 | chromosome 5 open reading | 64417 | NM_022483 | Hs.732093 | ENSG00000151881 |
| | | frame 28 | | | | |
| −1.9230769 | C5orf66 | chromosome 5 open reading | 100996485 | NM_001277348 | | ENSG00000224186 |
| | | frame 66 | | | | |
| 1.9400000 | C6orf1 | chromosome 6 open reading | 221491 | NM_001008703 | Hs.381300 | ENSG00000186577 |
| | | frame 1 | | | | |
| 1.4400000 | C6orf120 | chromosome 6 open reading | 387263 | NM_001029863 | Hs.591375 | ENSG00000185127 |
| | | frame 120 | | | | |
| 1.9100000 | C7orf26 | chromosome 7 open reading | 79034 | NM_001303039 | Hs.487511 | ENSG00000146576 |
| | | frame 26 | | | | |
| 2.9900000 | C9orf16 | chromosome 9 open reading | 79095 | NM_024112 | Hs.522412 | ENSG00000171159 |
| | | frame 16 | | | | |
| −1.6129032 | C9orf3 | chromosome 9 open reading | 84909 | NM_001193329 | Hs.434253 | ENSG00000148120 |
| | | frame 3 | | | | |
| 2.2200000 | C9orf69 | chromosome 9 open reading | 90120 | NM_001256526 | Hs.287411 | ENSG00000238227 |
| | | frame 69 | | | | |
| −2.2222222 | CXorf36 | chromosome X open reading | 79742 | NM_024689 | Hs.98321 | ENSG00000147113 |
| | | frame 36 | | | | |
| −2.1276596 | CXorf56 | chromosome X open reading | 63932 | NM_001170569 | Hs.248572 | ENSG00000018610 |
| | | frame 56 | | | | |
| −1.7543860 | CROCCP3 | ciliary rootlet coiled-coil, | 114819 | NR_023386 | Hs.597881 | ENSG00000080947 |
| | | rootletin pseudogene 3 | | | | |
| 1.6000000 | CMTM3 | CKLF-like MARVEL | 123920 | NM_001048251 | Hs.298198 | ENSG00000140931 |
| | | transmembrane domain | | | | |
| | | containing 3 | | | | |
| −2.0000000 | CKMT2-AS1 | CKMT2 antisense RNA 1 | 100131067 | NR_034121 | Hs.655855 | ENSG00000247572 |
| −1.8181818 | CLSPN | claspin | 63967 | NM_001190481 | Hs.175613 | ENSG00000092853 |
| −1.4705882 | CLINT1 | clathrin interactor 1 | 9685 | NM_001195555 | Hs.644000 | ENSG00000113282 |
| 1.7300000 | CLDN15 | claudin 15 | 24146 | NM_001185080 | Hs.38738 | ENSG00000106404 |
| −1.7543860 | CLDN19 | claudin 19 | 149461 | NM_001123395 | Hs.496270 | ENSG00000164007 |
| 1.5300000 | COPZ1 | coatomer protein complex, | 22818 | NM_001271734 | Hs.505652 | ENSG00000111481 |
| | | subunit zeta 1 | | | | |
| 1.4800000 | CCDC115 | coiled-coil domain containing 115 | 84317 | NM_032357 | Hs.104203 | ENSG00000136710 |
| −1.9230769 | CCDC12 | coiled-coil domain containing 12 | 151903 | NM_001277074 | Hs.631918 | ENSG00000160799 |
| −2.0408163 | CCDC144B | coiled-coil domain containing 144B | 284047 | NR_036647 | Hs.448012 | ENSG00000154874 |
| | | (pseudogene) | | | | |
| −2.0833333 | CCDC148 | coiled-coil domain containing 148 | 130940 | NM_001171637 | Hs.668597 | ENSG00000153237 |
| −2.0000000 | CCBE1 | collagen and calcium binding | 147372 | NM_133459 | Hs.34333 | ENSG00000183287 |
| | | EGF domains 1 | | | | |
| −1.7543860 | COMMD2 | COMM domain containing 2 | 51122 | NM_016094 | Hs.432729 | ENSG00000114744 |
| 1.7000000 | COMMD5 | COMM domain containing 5 | 28991 | NM_001081003 | Hs.631856 | ENSG00000170619 |
| 1.6700000 | C1QBP | complement component 1, q | 708 | NM_001212 | Hs.555866 | ENSG00000108561 |
| | | subcomponent binding protein | | | | |
| −2.7027027 | CRX | cone-rod homeobox | 1406 | NM_000554 | Hs.617342 | ENSG00000105392 |
| 1.7300000 | CPNE1 | copine I | 8904 | NM_001198863 | Hs.246413 | ENSG00000214078 |
| 2.1400000 | CORO1A | coronin, actin binding | 11151 | NM_001193333 | Hs.415067 | ENSG00000102879 |
| | | protein, 1A | | | | |
| −2.0000000 | CXADR | coxsackie virus and | 1525 | NM_001207063 | Hs.627078 | ENSG00000154639 |
| | | adenovirus receptor | | | | |
| −2.1739130 | CRYM-AS1 | CRYM antisense RNA 1 | 400508 | NM_001101368 | Hs.578949 | |
| −2.4390244 | CRYBB2P1 | crystallin, beta B2 | 1416 | NR_033733 | Hs.571835 | ENSG00000100058 |
| | | pseudogene 1 | | | | |
| 1.6400000 | CHTF8 | CTF8, chromosome | 54921 | NM_001002847 | Hs.85962 | ENSG00000168802 |
| | | transmission fidelity factor 8 | | | | |
| | | homolog (S. cerevisiae) | | | | |
| −1.7857143 | CLEC2D | C-type lectin domain family | 29121 | NM_001004419 | Hs.268326 | ENSG00000069493 |
| | | 2, member D | | | | |
| 1.7000000 | CLEC4A | C-type lectin domain family | 50856 | NM_016184 | Hs.504657 | ENSG00000111729 |
| | | 4, member A | | | | |
| −1.8518519 | CWC25 | CWC25 spliceosome- | 54883 | NM_017748 | Hs.406223 | ENSG00000273559 |
| | | associated protein homolog | | | | |
| | | (S. cerevisiae) | | | | |
| −1.3698630 | DMTF1 | cyclin D binding myb-like | 9988 | NM_001142326 | Hs.196129 | ENSG00000135164 |
| | | transcription factor 1 | | | | |
| −1.2987013 | CCNT2 | cyclin T2 | 905 | NM_001241 | Hs.744115 | ENSG00000082258 |
| −2.2727273 | LOC727896 | cysteine and histidine-rich | 727896 | NR_026659 | Hs.673126 | |
| | | domain (CHORD) containing | | | | |
| | | 1 pseudogene | | | | |
| −1.9607843 | CSAD | cysteine sulfinic acid | 51380 | NM_001244705 | Hs.279815 | ENSG00000139631 |
| | | decarboxylase | | | | |
| 2.5200000 | CDA | cytidine deaminase | 978 | NM_001785 | Hs.466910 | ENSG00000158825 |
| 1.8900000 | CYB561D2 | cytochrome b561 family, | 11068 | NM_001291284 | Hs.149443 | ENSG00000114395 |
| | | member D2 | | | | |
| −2.0000000 | COA7 | cytochrome c oxidase | 65260 | NM_023077 | Hs.349905 | ENSG00000162377 |
| | | assembly factor 7 (putative) | | | | |
| −2.9411765 | COX6B2 | cytochrome c oxidase subunit | 125965 | NM_144613 | Hs.550544 | ENSG00000160471 |
| | | VIb polypeptide 2 (testis) | | | | |
| 2.3100000 | COX8A | cytochrome c oxidase subunit | 1351 | NM_004074 | Hs.743989 | ENSG00000176340 |
| | | VIIIA (ubiquitous) | | | | |
| −1.4925373 | CYCS | cytochrome c, somatic | 54205 | NM_018947 | Hs.437060 | ENSG00000172115 |
| 1.6800000 | CYC1 | cytochrome c-1 | 1537 | NM_001916 | Hs.289271 | ENSG00000179091 |
| −1.6393443 | CYP20A1 | cytochrome P450, family 20, | 57404 | NM_020674 | Hs.446065 | ENSG00000119004 |
| | | subfamily A, polypeptide 1 | | | | |
| −1.8867925 | CYP4V2 | cytochrome P450, family 4, | 285440 | NM_207352 | Hs.587231 | ENSG00000145476 |
| | | subfamily V, polypeptide 2 | | | | |
| −1.8181818 | CYP51A1 | cytochrome P450, family 51, | 1595 | NM_000786 | Hs.417077 | ENSG00000001630 |
| | | subfamily A, polypeptide 1 | | | | |
| −1.6129032 | CYTIP | cytohesin 1 interacting protein | 9595 | NM_004288 | Hs.270 | ENSG00000115165 |
| −2.5000000 | CRLF2 | cytokine receptor-like factor 2 | 64109 | NM_001012288 | Hs.287729 | ENSG00000205755 |
| −1.7857143 | CRTAM | cytotoxic and regulatory T | 56253 | NM_001304782 | Hs.159523 | ENSG00000109943 |
| | | cell molecule | | | | |
| 2.1800000 | DBP | D site of albumin promoter | 1628 | NM_001352 | Hs.414480 | ENSG00000105516 |
| | | (albumin D-box) binding | | | | |
| | | protein | | | | |
| 1.5100000 | DDB1 | damage-specific DNA | 1642 | NM_001923 | Hs.290758 | ENSG00000167986 |
| | | binding protein 1, 127 kDa | | | | |
| −1.6393443 | DZIP3 | DAZ interacting zinc finger | 9666 | NM_014648 | Hs.409210 | ENSG00000198919 |
| | | protein 3 | | | | |
| −2.3255814 | DCUN1D2 | DCN1, defective in cullin | 55208 | NM_001014283 | Hs.682987 | ENSG00000150401 |
| | | neddylation 1, domain | | | | |
| | | containing 2 | | | | |
| −1.4084507 | DCAF16 | DDB1 and CUL4 associated | 54876 | NM_017741 | Hs.614787 | ENSG00000163257 |
| | | factor 16 | | | | |
| 1.9500000 | DDT | D-dopachrome tautomerase | 1652 | NM_001084392 | Hs.656723 | ENSG00000099977 |
| −1.8518519 | DDX51 | DEAD (Asp-Glu-Ala-Asp) | 317781 | NM_175066 | Hs.445168 | ENSG00000185163 |
| | | box polypeptide 51 | | | | |
| 1.5500000 | DEDD | death effector domain | 9191 | NM_001039711 | Hs.744092 | ENSG00000158796 |
| | | containing | | | | |
| −1.6666667 | DLEU1 | deleted in lymphocytic | 10301 | NM_005887 | Hs.591229 | ENSG00000176124 |
| | | leukemia 1 (non-protein | | | | |
| | | coding) | | | | |
| −2.2222222 | DLK2 | delta-like 2 homolog | 65989 | NM_001286655 | Hs.337251 | ENSG00000171462 |
| | | (Drosophila) | | | | |
| −1.4492754 | DENND6A | DENN/MADD domain | 201627 | NM_152678 | Hs.91085 | ENSG00000174839 |
| | | containing 6A | | | | |
| −2.2222222 | DSG2 | desmoglein 2 | 1829 | NM_001943 | Hs.412597 | ENSG00000046604 |
| −2.3255814 | DSG3 | desmoglein 3 | 1830 | NM_001944 | Hs.1925 | ENSG00000134757 |
| −1.2987013 | DIABLO | diablo, IAP-binding | 56616 | NM_001278302 | Hs.169611 | ENSG00000184047 |
| | | mitochondrial protein | | | | |
| 1.6600000 | DBI | diazepam binding inhibitor | 1622 | NM_001079862 | Hs.78888 | ENSG00000155368 |
| | | (GABA receptor modulator, | | | | |
| | | acyl-CoA binding protein) | | | | |
| −2.0000000 | DPH3P1 | diphthamide biosynthesis 3 | 100132911 | NM_080750 | | |
| | | pseudogene 1 | | | | |
| 1.6700000 | DPH7 | diphthamide biosynthesis 7 | 92715 | NM_138778 | Hs.292570 | ENSG00000148399 |
| −1.4492754 | DIS3 | DIS3 exosome | 22894 | NM_001128226 | Hs.744104 | ENSG00000083520 |
| | | endoribonuclease and 3′-5′ | | | | |
| | | exoribonuclease | | | | |
| −3.3333333 | DLGAP1-AS2 | DLGAP1 antisense RNA 2 | 84777 | NM_032691 | Hs.659053 | |
| −1.6666667 | DFFA | DNA fragmentation factor, | 1676 | NM_004401 | Hs.484782 | ENSG00000160049 |
| | | 45 kDa, alpha polypeptide | | | | |
| −2.2727273 | DMC1 | DNA meiotic recombinase 1 | 11144 | NM_001278208 | Hs.339396 | ENSG00000100206 |
| −1.8518519 | DNAH17-AS1 | DNAH17 antisense RNA 1 | 100996295 | NR_102401 | Hs.615304 | ENSG00000267432 |
| −1.4285714 | DNAJC21 | DnaJ (Hsp40) homolog, | 134218 | NM_001012339 | Hs.131887 | ENSG00000168724 |
| | | subfamily C, member 21 | | | | |
| −2.5000000 | DNAJC22 | DnaJ (Hsp40) homolog, | 79962 | NM_001304944 | Hs.659300 | ENSG00000178401 |
| | | subfamily C, member 22 | | | | |
| 1.9400000 | DNAJC4 | DnaJ (Hsp40) homolog, | 3338 | NM_005528 | Hs.172847 | ENSG00000110011 |
| | | subfamily C, member 4 | | | | |
| −2.0408163 | DNAJC9-AS1 | DNAJC9 antisense RNA 1 | 414245 | NR_038373 | Hs.661857 | ENSG00000236756 |
| 2.3400000 | DNLZ | DNL-type zinc finger | 728489 | NM_001080849 | Hs.528581 | ENSG00000213221 |
| −1.8518519 | DNM1P46 | DNM1 pseudogene 46 | 196968 | NM_194295 | Hs.567763 | ENSG00000182397 |
| −2.2222222 | DUXA | double homeobox A | 503835 | NM_001012729 | Hs.585857 | ENSG00000258873 |
| −2.5641026 | DYDC1 | DPY30 domain containing 1 | 143241 | NM_001269053 | Hs.407751 | ENSG00000170788 |
| −1.6129032 | DSTYK | dual serine/threonine and | 25778 | NM_015375 | Hs.6874 | ENSG00000133059 |
| | | tyrosine protein kinase | | | | |
| 2.1000000 | DUSP23 | dual specificity phosphatase 23 | 54935 | NM_017823 | Hs.425801 | ENSG00000158716 |
| 1.7200000 | DUSP7 | dual specificity phosphatase 7 | 1849 | NM_001947 | Hs.591664 | ENSG00000164086 |
| 2.2900000 | DYRK1B | dual-specificity tyrosine-(Y)- | 9149 | NM_004714 | Hs.130988 | ENSG00000105204 |
| | | phosphorylation regulated | | | | |
| | | kinase 1B | | | | |
| 1.6400000 | DCTN2 | dynactin 2 (p50) | 10540 | NM_001261412 | Hs.289123 | ENSG00000175203 |
| −2.5641026 | DYNAP | dynactin associated protein | 284254 | NM_173629 | Hs.376146 | ENSG00000178690 |
| 1.5100000 | DNAAF2 | dynein, axonemal, assembly | 55172 | NM_001083908 | Hs.231761 | ENSG00000165506 |
| | | factor 2 | | | | |
| −1.9230769 | DNAL1 | dynein, axonemal, light chain 1 | 83544 | NM_001201366 | Hs.271270 | ENSG00000119661 |
| −1.9607843 | DYX1C1 | dyslexia susceptibility 1 | 161582 | NM_001033559 | Hs.126403 | ENSG00000256061 |
| | | candidate 1 | | | | |
| −1.6393443 | ENTPD4 | ectonucleoside triphosphate | 9583 | NM_001128930 | Hs.444389 | ENSG00000197217 |
| | | diphosphohydrolase 4 | | | | |
| −1.9607843 | EFCAB11 | EF-hand calcium binding | 90141 | NM_001284266 | Hs.123232 | ENSG00000140025 |
| | | domain 11 | | | | |
| −2.0833333 | EGFEM1P | EGF-like and EMI domain | 93556 | NR_021485 | Hs.478158 | ENSG00000206120 |
| | | containing 1, pseudogene | | | | |
| −1.6666667 | ERC1 | ELKS/RAB6-interacting/ | 23085 | NM_001301248 | Hs.601216 | ENSG00000082805 |
| | | CAST family member 1 | | | | |
| −2.1276596 | ELMOD1 | ELMO/CED-12 domain | 55531 | NM_001130037 | Hs.495779 | ENSG00000110675 |
| | | containing 1 | | | | |
| −3.2258065 | ESRG | embryonic stem cell related | 790952 | NR_027122 | Hs.720658 | ENSG00000265992 |
| | | (non-protein coding) | | | | |
| −1.7543860 | EBP | emopamil binding protein | 10682 | NM_006579 | Hs.30619 | ENSG00000147155 |
| | | (sterol isomerase) | | | | |
| −2.2222222 | EMX2OS | EMX2 opposite | 196047 | NR_002791 | Hs.312592 | ENSG00000229847 |
| | | strand/antisense RNA | | | | |
| −1.7543860 | ERVK13-1 | endogenous retrovirus group | 100507321 | NM_001012731 | Hs.406976 | ENSG00000260565 |
| | | K13, member 1 | | | | |
| −2.3809524 | ERVV-1 | endogenous retrovirus group | 147664 | NM_152473 | Hs.44329 | ENSG00000269526 |
| | | V, member 1 | | | | |
| 1.5000000 | ERH | enhancer of rudimentary | 2079 | NM_004450 | Hs.509791 | ENSG00000100632 |
| | | homolog (Drosophila) | | | | |
| 1.5800000 | ECH1 | enoyl CoA hydratase 1, | 1891 | NM_001398 | Hs.196176 | ENSG00000104823 |
| | | peroxisomal | | | | |
| −2.1276596 | ENTPD1-AS1 | ENTPD1 antisense RNA 1 | 728558 | NR_038444 | Hs.538374 | ENSG00000226688 |
| −2.5641026 | EPHA10 | EPH receptor A10 | 284656 | NM_001004338 | Hs.129435 | ENSG00000183317 |
| −2.6315789 | EPPIN | epididymal peptidase inhibitor | 57119 | NM_001302861 | Hs.121084 | ENSG00000101448 |
| −1.8867925 | ECT2 | epithelial cell transforming 2 | 1894 | NM_001258315 | Hs.518299 | ENSG00000114346 |
| −2.0000000 | EMP2 | epithelial membrane protein 2 | 2013 | NM_001424 | Hs.531561 | ENSG00000213853 |
| 1.3500000 | EMC10 | ER membrane protein | 284361 | NM_175063 | Hs.448941 | ENSG00000161671 |
| | | complex subunit 10 | | | | |
| 4.5600000 | EMC6 | ER membrane protein | 83460 | NM_001014764 | Hs.30011 | ENSG00000127774 |
| | | complex subunit 6 | | | | |
| 1.6200000 | EMC8 | ER membrane protein | 10328 | NM_001142288 | Hs.173162 | ENSG00000131148 |
| | | complex subunit 8 | | | | |
| 1.9700000 | ERI3 | ERI1 exoribonuclease family | 79033 | NM_001301698 | Hs.731413 | ENSG00000117419 |
| | | member 3 | | | | |
| 2.3100000 | ETHE1 | ethylmalonic encephalopathy 1 | 23474 | NM_014297 | Hs.7486 | ENSG00000105755 |
| −1.3698630 | EEF1D | eukaryotic translation | 1936 | NM_001130053 | Hs.333388 | ENSG00000104529 |
| | | elongation factor 1 delta | | | | |
| | | (guanine nucleotide exchange | | | | |
| | | protein) | | | | |
| 2.2300000 | EIF4EBP3 | eukaryotic translation | 8637 | NM_003732 | Hs.594084 | ENSG00000243056 |
| | | initiation factor 4E binding | | | | |
| | | protein 3 | | | | |
| −1.5873016 | EIF5 | eukaryotic translation | 1983 | NM_001969 | Hs.433702 | ENSG00000100664 |
| | | initiation factor 5 | | | | |
| −1.3888889 | EWSR1 | EWS RNA-binding protein 1 | 2130 | NM_001163285 | Hs.374477 | ENSG00000182944 |
| −2.0408163 | EXPH5 | exophilin 5 | 23086 | NM_001144763 | Hs.28540 | ENSG00000110723 |
| 1.3800000 | ESYT1 | extended synaptotagmin-like | 23344 | NM_001184796 | Hs.632729 | ENSG00000139641 |
| | | protein 1 | | | | |
| −1.9230769 | FAM83H-AS1 | FAM83H antisense RNA 1 | 100128338 | NR_033849 | Hs.493171 | ENSG00000203499 |
| | | (head to head) | | | | |
| −1.8518519 | FAM114A1 | family with sequence | 92689 | NM_138389 | Hs.476517 | ENSG00000197712 |
| | | similarity 114, member A1 | | | | |
| −2.1739130 | FAM122C | family with sequence | 159091 | NM_001170779 | Hs.269127 | ENSG00000156500 |
| | | similarity 122C | | | | |
| −2.8571429 | FAM133DP | family with sequence | 728066 | NR_034169 | Hs.470311 | |
| | | similarity 133, member A | | | | |
| | | pseudogene | | | | |
| −1.4084507 | FAM177A1 | family with sequence | 283635 | NM_001079519 | Hs.446357 | ENSG00000151327 |
| | | similarity 177, member A1 | | | | |
| −1.9230769 | FAM71F2 | family with sequence | 346653 | NM_001012454 | Hs.445236 | ENSG00000205085 |
| | | similarity 71, member F2 | | | | |
| 1.7700000 | FAM89B | family with sequence | 23625 | NM_001098784 | Hs.731854 | ENSG00000176973 |
| | | similarity 89, member B | | | | |
| 1.6400000 | FAM96A | family with sequence | 84191 | NM_001014812 | Hs.439548 | ENSG00000166797 |
| | | similarity 96, member A | | | | |
| −2.2727273 | FDPSP2 | farnesyl diphosphate synthase | 619190 | NR_003262 | Hs.609978 | ENSG00000233980 |
| | | pseudogene 2 | | | | |
| 1.5000000 | FNTA | farnesyltransferase, CAAX | 2339 | NM_001018676 | Hs.370312 | ENSG00000168522 |
| | | box, alpha | | | | |
| −2.3809524 | FAIM | Fas apoptotic inhibitory | 55179 | NM_001033030 | Hs.173438 | ENSG00000158234 |
| | | molecule | | | | |
| −1.8867925 | FEZ1 | fasciculation and elongation | 9638 | NM_005103 | Hs.224008 | ENSG00000149557 |
| | | protein zeta 1 (zygin I) | | | | |
| 1.9500000 | FBXL14 | F-box and leucine-rich repeat | 144699 | NM_152441 | Hs.367956 | ENSG00000171823 |
| | | protein 14 | | | | |
| −1.8181818 | FBXL18 | F-box and leucine-rich repeat | 80028 | NM_024963 | Hs.623974 | ENSG00000155034 |
| | | protein 18 | | | | |
| −1.4492754 | FBXL20 | F-box and leucine-rich repeat | 84961 | NM_001184906 | Hs.462946 | ENSG00000108306 |
| | | protein 20 | | | | |
| 1.7200000 | FBXW5 | F-box and WD repeat domain | 54461 | NM_018998 | Hs.522507 | ENSG00000159069 |
| | | containing 5 | | | | |
| −2.0408163 | FBXO17 | F-box protein 17 | 115290 | NM_024907 | Hs.531770 | ENSG00000269190 |
| −2.2222222 | FBXO27 | F-box protein 27 | 126433 | NM_178820 | Hs.187461 | ENSG00000161243 |
| −1.6129032 | FBXO44 | F-box protein 44 | 93611 | NM_001014765 | Hs.556006 | ENSG00000132879 |
| 1.3800000 | FEM1A | fem-1 homolog a (C. elegans) | 55527 | NM_018708 | Hs.515082 | ENSG00000141965 |
| 1.6800000 | FDX1L | ferredoxin 1-like | 112812 | NM_001031734 | Hs.654865 | ENSG00000267673 |
| −2.2727273 | FRRS1 | ferric-chelate reductase 1 | 391059 | NM_001013660 | Hs.454779 | ENSG00000156869 |
| −1.8181818 | FGFR1OP | FGFR1 oncogene partner | 11116 | NM_001278690 | Hs.487175 | ENSG00000213066 |
| −2.5641026 | LOC100335030 | FGFR1 oncogene partner 2 | 100335030 | NR_033267 | Hs.687044 | ENSG00000257954 |
| | | pseudogene | | | | |
| −2.5000000 | FGF5 | fibroblast growth factor 5 | 2250 | NM_001291812 | Hs.37055 | ENSG00000138675 |
| −1.9607843 | FGFR2 | fibroblast growth factor | 2263 | NM_000141 | Hs.533683 | ENSG00000066468 |
| | | receptor 2 | | | | |
| −2.1739130 | FBLN1 | fibulin 1 | 2192 | NM_001996 | Hs.24601 | ENSG00000077942 |
| −3.4482759 | FILIP1 | filamin A interacting protein 1 | 27145 | NM_001289987 | Hs.696158 | ENSG00000118407 |
| −2.5641026 | FBLIM1 | filamin binding LIM protein 1 | 54751 | NM_001024215 | Hs.530101 | ENSG00000162458 |
| 1.8300000 | FIS1 | fission 1 (mitochondrial outer | 51024 | NM_016068 | Hs.423968 | ENSG00000214253 |
| | | membrane) homolog (S. cerevisiae) | | | | |
| −2.2222222 | FKBP14 | FK506 binding protein 14, 22 kDa | 55033 | NM_017946 | Hs.390838 | ENSG00000106080 |
| −1.7241379 | FOXJ3 | forkhead box J3 | 22887 | NM_001198850 | Hs.26023 | ENSG00000198815 |
| −1.5151515 | FNBP4 | formin binding protein 4 | 23360 | NM_015308 | Hs.6834 | ENSG00000109920 |
| 1.4400000 | FMNL3 | formin-like 3 | 91010 | NM_175736 | Hs.179838 | ENSG00000161791 |
| −2.0000000 | FOXL2NB | FOXL2 neighbor | 401089 | NM_001040061 | Hs.591303 | ENSG00000206262 |
| −2.1276596 | FRY-AS1 | FRY antisense RNA 1 | 100507099 | NR_103839 | Hs.536364 | ENSG00000237637 |
| −4.0000000 | LOC642236 | FSHD region gene 1 | 642236 | NR_033907 | Hs.529357 | |
| | | pseudogene | | | | |
| −1.9230769 | FUT1 | fucosyltransferase 1 | 2523 | NM_000148 | Hs.69747 | ENSG00000174951 |
| | | (galactoside 2-alpha-L- | | | | |
| | | fucosyltransferase, H blood | | | | |
| | | group) | | | | |
| −2.4390244 | FUT2 | fucosyltransferase 2 (secretor | 2524 | NM_000511 | Hs.579928 | ENSG00000176920 |
| | | status included) | | | | |
| −2.0833333 | FUT6 | fucosyltransferase 6 (alpha | 2528 | NM_000150 | Hs.631846 | ENSG00000156413 |
| | | (1,3) fucosyltransferase) | | | | |
| −2.0408163 | FGD5P1 | FYVE, RhoGEF and PH domain | 100132526 | NR_036481 | Hs.637770 | ENSG00000275340 |
| | | containing 5 pseudogene 1 | | | | |
| 1.5600000 | GPR108 | G protein-coupled receptor 108 | 56927 | NM_001080452 | Hs.167641 | ENSG00000125734 |
| 2.3400000 | GPR141 | G protein-coupled receptor 141 | 353345 | NM_181791 | Hs.688230 | ENSG00000187037 |
| −1.5384615 | GPR18 | G protein-coupled receptor 18 | 2841 | NM_001098200 | Hs.741589 | ENSG00000125245 |
| −2.3255814 | GPR37L1 | G protein-coupled receptor 37 | 9283 | NM_004767 | Hs.132049 | ENSG00000170075 |
| | | like 1 | | | | |
| −2.1276596 | GPR82 | G protein-coupled receptor 82 | 27197 | NM_080817 | Hs.567457 | ENSG00000171657 |
| −1.8181818 | GABPB1-AS1 | GABPB1 antisense RNA 1 | 100129387 | NR_024490 | Hs.659360 | |
| 2.4600000 | GALK1 | galactokinase 1 | 2584 | NM_000154 | Hs.407966 | ENSG00000108479 |
| −1.9607843 | GAL3ST4 | galactose-3-O- | 79690 | NM_024637 | Hs.44856 | ENSG00000197093 |
| | | sulfotransferase 4 | | | | |
| −2.5641026 | GGT8P | gamma-glutamyltransferase 8 | 645367 | NR_003503 | Hs.650223 | |
| | | pseudogene | | | | |
| −2.5000000 | GAS6-AS2 | GAS6 antisense RNA 2 | 100506394 | NR_044993 | Hs.132168 | ENSG00000272695 |
| | | (head to head) | | | | |
| −2.0833333 | GSDMA | gasdermin A | 284110 | NM_178171 | Hs.448873 | ENSG00000167914 |
| 1.7800000 | GMPPA | GDP-mannose | 29926 | NM_013335 | Hs.27059 | ENSG00000144591 |
| | | pyrophosphorylase A | | | | |
| 1.9300000 | GSN | gelsolin | 2934 | NM_000177 | Hs.522373 | ENSG00000148180 |
| −2.3809524 | GEMIN8 | gem (nuclear organelle) | 54960 | NM_001042479 | Hs.592237 | ENSG00000046647 |
| | | associated protein 8 | | | | |
| 1.7100000 | GTF2H4 | general transcription factor | 2968 | NM_001517 | Hs.485070 | ENSG00000213780 |
| | | IIH, polypeptide 4, 52 kDa | | | | |
| 1.5500000 | GTF3C2 | general transcription factor | 2976 | NM_001035521 | Hs.75782 | ENSG00000115207 |
| | | IIIC, polypeptide 2, beta | | | | |
| | | 110 kDa | | | | |
| −2.7027027 | GSG1 | germ cell associated 1 | 83445 | NM_001080554 | Hs.240053 | ENSG00000111305 |
| 1.9500000 | GHDC | GH3 domain containing | 84514 | NM_001142622 | Hs.38039 | ENSG00000167925 |
| −3.2258065 | GLIPR1L2 | GLI pathogenesis-related 1 | 144321 | NM_001270396 | Hs.406728 | ENSG00000180481 |
| | | like 2 | | | | |
| −1.5384615 | GMEB1 | glucocorticoid modulatory | 10691 | NM_006582 | Hs.632373 | ENSG00000162419 |
| | | element binding protein 1 | | | | |
| −2.2727273 | GNE | glucosamine (UDP-N-acetyl)- | 10020 | NM_001128227 | Hs.5920 | ENSG00000159921 |
| | | 2-epimerase/N- | | | | |
| | | acetylmannosamine kinase | | | | |
| 1.4600000 | GLUD1 | glutamate dehydrogenase 1 | 2746 | NM_005271 | Hs.500409 | ENSG00000148672 |
| 1.6100000 | QPCT | glutaminyl-peptide | 25797 | NM_012413 | Hs.79033 | ENSG00000115828 |
| | | cyclotransferase | | | | |
| 2.1100000 | GPX4 | glutathione peroxidase 4 | 2879 | NM_001039847 | Hs.433951 | ENSG00000167468 |
| −2.4390244 | GSTM3 | glutathione S-transferase mu | 2947 | NM_000849 | Hs.2006 | ENSG00000134202 |
| | | 3 (brain) | | | | |
| 1.9800000 | GSTP1 | glutathione S-transferase pi 1 | 2950 | NM_000852 | Hs.523836 | ENSG00000084207 |
| −1.7857143 | GK5 | glycerol kinase 5 (putative) | 256356 | NM_001039547 | Hs.135904 | ENSG00000175066 |
| −2.2727273 | GDPD1 | glycerophosphodiester | 284161 | NM_001165993 | Hs.631744 | ENSG00000153982 |
| | | phosphodiesterase domain | | | | |
| | | containing 1 | | | | |
| 1.4800000 | GYS1 | glycogen synthase 1 (muscle) | 2997 | NM_001161587 | Hs.386225 | ENSG00000104812 |
| −1.9230769 | GPLD1 | glycosylphosphatidylinositol | 2822 | NM_001503 | Hs.533291 | ENSG00000112293 |
| | | specific phospholipase D1 | | | | |
| −2.2727273 | GOSR1 | golgi SNAP receptor complex | 9527 | NM_001007024 | Hs.462680 | ENSG00000108587 |
| | | member 1 | | | | |
| −1.5384615 | GOLGA2 | golgin A2 | 2801 | NM_004486 | Hs.155827 | ENSG00000167110 |
| −2.8571429 | GOLGA6L22 | golgin A6 family-like 22 | 440243 | NM_001271664 | | ENSG00000274404 |
| 1.5600000 | GOLGA7 | golgin A7 | 51125 | NM_001002296 | Hs.654773 | ENSG00000147533 |
| −3.0303030 | GNRHR2 | gonadotropin-releasing | 114814 | NM_057163 | Hs.356873 | ENSG00000211451 |
| | | hormone (type 2) receptor 2, | | | | |
| | | pseudogene | | | | |
| −2.3809524 | GPR1-AS | GPR1 antisense RNA | 101669764 | NR_104359 | Hs.574781 | ENSG00000279220 |
| −1.6129032 | GAB2 | GRB2-associated binding | 9846 | NM_012296 | Hs.429434 | ENSG00000033327 |
| | | protein 2 | | | | |
| −2.5641026 | GRTP1 | growth hormone regulated | 79774 | NM_001286732 | Hs.745043 | ENSG00000139835 |
| | | TBC protein 1 | | | | |
| −2.1276596 | GREB1 | growth regulation by estrogen | 9687 | NM_014668 | Hs.467733 | ENSG00000196208 |
| | | in breast cancer 1 | | | | |
| 1.5400000 | GTPBP6 | GTP binding protein 6 (putative) | 8225 | NM_012227 | Hs.437145 | ENSG00000178605 |
| −1.7543860 | GTPBP10 | GTP-binding protein 10 (putative) | 85865 | NM_001042717 | Hs.593547 | ENSG00000105793 |
| 1.7700000 | GNB2 | guanine nucleotide binding | 2783 | NM_005273 | Hs.185172 | ENSG00000172354 |
| | | protein (G protein), beta | | | | |
| | | polypeptide 2 | | | | |
| −1.8867925 | GNG4 | guanine nucleotide binding | 2786 | NM_001098721 | Hs.159711 | ENSG00000168243 |
| | | protein (G protein), gamma 4 | | | | |
| −1.8181818 | GNL1 | guanine nucleotide binding | 2794 | NM_005275 | Hs.83147 | ENSG00000204590 |
| | | protein-like 1 | | | | |
| −1.9230769 | H1FX-AS1 | H1FX antisense RNA 1 | 339942 | NM_001025468 | Hs.450096 | |
| 1.7800000 | H2AFJ | H2A histone family, member J | 55766 | NM_018267 | Hs.524280 | ENSG00000246705 |
| 2.0200000 | H2AFX | H2A histone family, member X | 3014 | NM_002105 | Hs.477879 | ENSG00000188486 |
| 1.3500000 | HDHD3 | haloacid dehalogenase-like | 81932 | NM_001304509 | Hs.7739 | ENSG00000119431 |
| | | hydrolase domain containing 3 | | | | |
| −1.8518519 | HAUS3 | HAUS augmin-like complex, | 79441 | NM_001303143 | Hs.665869 | ENSG00000214367 |
| | | subunit 3 | | | | |
| 1.6700000 | HS1BP3 | HCLS1 binding protein 3 | 64342 | NM_022460 | Hs.531785 | ENSG00000118960 |
| −2.5641026 | HSP90AB4P | heat shock protein 90 kDa | 664618 | NR_002927 | Hs.670224 | |
| | | alpha (cytosolic), class B | | | | |
| | | member 4, pseudogene | | | | |
| 1.6900000 | HCST | hematopoietic cell signal | 10870 | NM_001007469 | Hs.117339 | ENSG00000126264 |
| | | transducer | | | | |
| −1.7543860 | HHLA2 | HERV-H LTR-associating 2 | 11148 | NM_001282556 | Hs.252351 | ENSG00000114455 |
| −2.2222222 | HHLA3 | HERV-H LTR-associating 3 | 11147 | NM_001031693 | Hs.142245 | ENSG00000197568 |
| −2.3809524 | HES2 | hes family bHLH | 54626 | NM_019089 | Hs.118727 | ENSG00000069812 |
| | | transcription factor 2 | | | | |
| −1.8181818 | HNRNPA1L2 | heterogeneous nuclear | 144983 | NM_001011724 | Hs.447506 | ENSG00000139675 |
| | | ribonucleoprotein A1-like 2 | | | | |
| 1.3800000 | H6PD | hexose-6-phosphate | 9563 | NM_001282587 | Hs.463511 | ENSG00000049239 |
| | | dehydrogenase (glucose 1- | | | | |
| | | dehydrogenase) | | | | |
| −2.4390244 | HMGB3P1 | high mobility group box 3 | 128872 | NR_002165 | Hs.558624 | |
| | | pseudogene 1 | | | | |
| 1.9100000 | HIST1H2AC | histone cluster 1, H2ac | 8334 | NM_003512 | Hs.484950 | ENSG00000180573 |
| 2.5100000 | HIST1H3H | histone cluster 1, H3h | 8357 | NM_003536 | Hs.591778 | ENSG00000278828 |
| −2.0833333 | HCG26 | HLA complex group 26 (non- | 352961 | NR_002812 | Hs.132807 | |
| | | protein coding) | | | | |
| −2.1739130 | HNF1A-AS1 | HNF1A antisense RNA 1 | 283460 | NM_178513 | Hs.612351 | ENSG00000241388 |
| 1.7400000 | HCFC1R1 | host cell factor C1 regulator 1 | 54985 | NM_001002017 | Hs.423103 | ENSG00000103145 |
| | | (XPO1 dependent) | | | | |
| −2.4390244 | HTRA4 | HtrA serine peptidase 4 | 203100 | NM_153692 | Hs.661014 | ENSG00000169495 |
| −2.0000000 | HSD17B13 | hydroxysteroid (17-beta) | 345275 | NM_001136230 | Hs.284414 | ENSG00000170509 |
| | | dehydrogenase 13 | | | | |
| −2.0833333 | HILPDA | hypoxia inducible lipid | 29923 | NM_001098786 | Hs.706124 | ENSG00000135245 |
| | | droplet-associated | | | | |
| −1.8181818 | IBA57 | IBA57, iron-sulfur cluster | 200205 | NM_001010867 | Hs.237017 | ENSG00000181873 |
| | | assembly homolog (S. | | | | |
| | | cerevisiae) | | | | |
| −2.2727273 | IKZF3 | IKAROS family zinc finger 3 | 22806 | NM_001257408 | Hs.371680 | ENSG00000161405 |
| | | (Aiolos) | | | | |
| −1.5151515 | ILF3-AS1 | ILF3 antisense RNA 1 | 147727 | NR_024333 | Hs.631616 | ENSG00000267100 |
| | | (head to head) | | | | |
| −1.9230769 | IRGQ | immunity-related GTPase | 126298 | NM_001007561 | Hs.6217 | ENSG00000167378 |
| | | family, Q | | | | |
| −1.9230769 | IPO5P1 | importin 5 pseudogene 1 | 100132815 | NR_103741 | Hs.629249 | ENSG00000269837 |
| −2.3255814 | INE1 | inactivation escape 1 (non- | 8552 | NM_003669 | Hs.657350 | ENSG00000224975 |
| | | protein coding) | | | | |
| −2.4390244 | IDO1 | indoleamine 2,3-dioxygenase 1 | 3620 | NM_002164 | Hs.840 | ENSG00000131203 |
| −2.2727273 | INMT | indolethylamine N- | 11185 | NM_001199219 | Hs.632629 | ENSG00000241644 |
| | | methyltransferase | | | | |
| −1.9607843 | INHBA-AS1 | INHBA antisense RNA 1 | 285954 | NR_027118 | Hs.656869 | ENSG00000224116 |
| −1.5384615 | ING5 | inhibitor of growth family, | 84289 | NM_032329 | Hs.529172 | ENSG00000168395 |
| | | member 5 | | | | |
| −2.5641026 | INGX | inhibitor of growth family, X- | 27160 | NR_002226 | Hs.721806 | ENSG00000243468 |
| | | linked, pseudogene | | | | |
| 1.3800000 | INPP5E | inositol polyphosphate-5- | 56623 | NM_019892 | Hs.120998 | ENSG00000148384 |
| | | phosphatase, 72 kDa | | | | |
| 1.6500000 | INTS9 | integrator complex subunit 9 | 55756 | NM_001145159 | Hs.162397 | ENSG00000104299 |
| −1.8867925 | ITIH5 | inter-alpha-trypsin inhibitor | 80760 | NM_001001851 | Hs.498586 | ENSG00000123243 |
| | | heavy chain family, member 5 | | | | |
| 1.7000000 | IRF2BP1 | interferon regulatory factor 2 | 26145 | NM_015649 | Hs.515477 | ENSG00000170604 |
| | | binding protein 1 | | | | |
| −2.3809524 | IFNLR1 | interferon, lambda receptor 1 | 163702 | NM_170743 | Hs.221375 | ENSG00000185436 |
| −2.0000000 | IL17RD | interleukin 17 receptor D | 54756 | NM_017563 | Hs.150725 | ENSG00000144730 |
| −1.5625000 | ITSN2 | intersectin 2 | 50618 | NM_006277 | Hs.432562 | ENSG00000198399 |
| −1.9607843 | IFT22 | intraflagellar transport 22 | 64792 | NM_001130820 | Hs.389104 | ENSG00000128581 |
| −2.2222222 | IAPP | islet amyloid polypeptide | 3375 | NM_000415 | Hs.46835 | ENSG00000121351 |
| −2.0000000 | ICA1L | islet cell autoantigen 1, | 130026 | NM_001288622 | Hs.516629 | ENSG00000163596 |
| | | 69 kDa-like | | | | |
| 2.3900000 | JOSD2 | Josephin domain containing 2 | 126119 | NM_001270639 | Hs.467151 | ENSG00000161677 |
| −2.2222222 | JPX | JPX transcript, XIST | 554203 | NR_024582 | Hs.648316 | ENSG00000225470 |
| | | activator (non-protein coding) | | | | |
| 1.6100000 | JTB | jumping translocation breakpoint | 10899 | NM_006694 | Hs.6396 | ENSG00000143543 |
| −2.6315789 | KCNQ1OT1 | KCNQ1 opposite | 10984 | NR_002728 | Hs.604823 | ENSG00000269821 |
| | | strand/antisense transcript 1 | | | | |
| | | (non-protein coding) | | | | |
| −2.2727273 | KDELC2 | KDEL (Lys-Asp-Glu-Leu) | 143888 | NM_153705 | Hs.83286 | ENSG00000178202 |
| | | containing 2 | | | | |
| 1.9100000 | KDELR1 | KDEL (Lys-Asp-Glu-Leu) | 10945 | NM_006801 | Hs.515515 | ENSG00000105438 |
| | | endoplasmic reticulum | | | | |
| | | protein retention receptor 1 | | | | |
| 2.0800000 | KDELR2 | KDEL (Lys-Asp-Glu-Leu) | 11014 | NM_001100603 | Hs.654552 | ENSG00000136240 |
| | | endoplasmic reticulum | | | | |
| | | protein retention receptor 2 | | | | |
| −2.5000000 | KDM4A-AS1 | KDM4A antisense RNA 1 | 100132774 | NR_033827 | Hs.655569 | |
| 1.7100000 | KLHDC3 | kelch domain containing 3 | 116138 | NM_001242872 | Hs.412468 | ENSG00000124702 |
| −2.2222222 | KBTBD12 | kelch repeat and BTB (POZ) | 166348 | NM_207335 | Hs.132087 | ENSG00000187715 |
| | | domain containing 12 | | | | |
| −1.8181818 | KRT8 | keratin 8, type II | 3856 | NM_001256282 | Hs.533782 | ENSG00000170421 |
| −3.4482759 | KIAA0101 | KIAA0101 | 9768 | NM_001029989 | Hs.81892 | ENSG00000166803 |
| −2.8571429 | KIAA1161 | KIAA1161 | 57462 | NM_020702 | Hs.522083 | ENSG00000164976 |
| −2.2222222 | KIAA1456 | KIAA1456 | 57604 | NM_001099677 | Hs.202521 | ENSG00000250305 |
| −2.1739130 | KIAA1614 | KIAA1614 | 57710 | NM_020950 | Hs.734816 | ENSG00000135835 |
| −2.0000000 | KIAA1919 | KIAA1919 | 91749 | NM_153369 | Hs.400572 | ENSG00000173214 |
| −2.3255814 | KIR3DX1 | killer cell immunoglobulin- | 90011 | NM_001047605 | Hs.288520 | ENSG00000104970 |
| | | like receptor, three domains, X1 | | | | |
| −3.2258065 | KLRD1 | killer cell lectin-like receptor | 3824 | NM_001114396 | Hs.562457 | ENSG00000134539 |
| | | subfamily D, member 1 | | | | |
| −1.5625000 | KIN | Kin17 DNA and RNA | 22944 | NM_012311 | Hs.397918 | ENSG00000151657 |
| | | binding protein | | | | |
| −2.2727273 | KIF18B | kinesin family member 18B | 146909 | NM_001080443 | Hs.135094 | ENSG00000186185 |
| −1.3157895 | KIF1B | kinesin family member 1B | 23095 | NM_015074 | Hs.97858 | ENSG00000054523 |
| −2.2222222 | KIF3A | kinesin family member 3A | 11127 | NM_001300791 | Hs.43670 | ENSG00000131437 |
| −2.8571429 | L2HGDH | L-2-hydroxyglutarate | 79944 | NM_024884 | Hs.256034 | ENSG00000087299 |
| | | dehydrogenase | | | | |
| −2.0408163 | LARS2-AS1 | LARS2 antisense RNA 1 | 100885795 | NR_048543 | Hs.641094 | ENSG00000232455 |
| −2.2727273 | LGMN | legumain | 5641 | NM_001008530 | Hs.18069 | ENSG00000100600 |
| −2.0000000 | LRRC2 | leucine rich repeat containing 2 | 79442 | NM_024512 | Hs.657345 | ENSG00000163827 |
| −1.9607843 | LRRC27 | leucine rich repeat containing 27 | 80313 | NM_001143757 | Hs.119897 | ENSG00000148814 |
| −1.3698630 | LRRC37BP1 | leucine rich repeat containing 37B | 147172 | NM_207323 | Hs.568209 | |
| | | pseudogene 1 | | | | |
| −2.1739130 | LRTOMT | leucine rich transmembrane | 220074 | NM_001145307 | Hs.317243 | ENSG00000184154 |
| | | and O-methyltransferase | | | | |
| | | domain containing | | | | |
| 1.5400000 | LILRA2 | leukocyte immunoglobulin- | 11027 | NM_001130917 | Hs.655593 | ENSG00000239998 |
| | | like receptor, subfamily A | | | | |
| | | (with TM domain), member 2 | | | | |
| −2.9411765 | LIFR-AS1 | LIFR antisense RNA 1 | 100506495 | NR_103553 | Hs.657602 | ENSG00000244968 |
| 2.1200000 | LIMD2 | LIM domain containing 2 | 80774 | NM_030576 | Hs.591166 | ENSG00000136490 |
| −2.0408163 | LMX1B | LIM homeobox transcription | 4010 | NM_001174146 | Hs.129133 | ENSG00000136944 |
| | | factor 1, beta | | | | |
| −2.3255814 | L1TD1 | LINE-1 type transposase | 54596 | NM_001164835 | Hs.685462 | ENSG00000240563 |
| | | domain containing 1 | | | | |
| −1.9230769 | LINC01010 | long intergenic non-protein | 154092 | NR_038216 | Hs.223718 | ENSG00000236700 |
| | | coding RNA 1010 | | | | |
| −2.1739130 | LINC01012 | long intergenic non-protein | 100507173 | NR_038292 | Hs.635987 | |
| | | coding RNA 1012 | | | | |
| −2.0833333 | LINC01016 | long intergenic non-protein | 100507584 | NR_038989 | Hs.547104 | ENSG00000249346 |
| | | coding RNA 1016 | | | | |
| −2.7027027 | LINC01057 | long intergenic non-protein | 101928079 | NR_104131 | Hs.596857 | ENSG00000224081 |
| | | coding RNA 1057 | | | | |
| −2.2727273 | LINC01087 | long intergenic non-protein | 101927994 | NR_108087 | Hs.635757 | ENSG00000224559 |
| | | coding RNA 1087 | | | | |
| −2.3255814 | LINC01205 | long intergenic non-protein | 401082 | NM_001145553 | Hs.477089 | ENSG00000228980 |
| | | coding RNA 1205 | | | | |
| −2.2222222 | LINC01207 | long intergenic non-protein | 100505989 | NR_038834 | Hs.328236 | ENSG00000248771 |
| | | coding RNA 1207 | | | | |
| −2.3255814 | LINC01209 | long intergenic non-protein | 101928684 | NR_110819 | Hs.639352 | ENSG00000228308 |
| | | coding RNA 1209 | | | | |
| −2.2222222 | LINC01226 | long intergenic non-protein | 284551 | NR_027085 | Hs.658659 | ENSG00000223907 |
| | | coding RNA 1226 | | | | |
| −2.1739130 | LINC01239 | long intergenic non-protein | 441389 | NR_038977 | | ENSG00000234840 |
| | | coding RNA 1239 | | | | |
| −2.1276596 | LINC01247 | long intergenic non-protein | 101929390 | NR_110251 | Hs.434407 | ENSG00000227007 |
| | | coding RNA 1247 | | | | |
| −1.8867925 | LINC01252 | long intergenic non-protein | 338817 | NR_033890 | Hs.733066 | ENSG00000247157 |
| | | coding RNA 1252 | | | | |
| −1.9607843 | LINC01299 | long intergenic non-protein | 286186 | NR_033893 | Hs.449427 | ENSG00000254081 |
| | | coding RNA 1299 | | | | |
| −2.3809524 | LINC01356 | long intergenic non-protein | 100996702 | NR_103746 | Hs.632431 | ENSG00000215866 |
| | | coding RNA 1356 | | | | |
| −2.1739130 | LINC00294 | long intergenic non-protein | 283267 | NR_015451 | Hs.533701 | |
| | | coding RNA 294 | | | | |
| −1.8181818 | LINC00330 | long intergenic non-protein | 144817 | NR_038433 | Hs.585616 | ENSG00000235097 |
| | | coding RNA 330 | | | | |
| −1.8518519 | LINC00342 | long intergenic non-protein | 150759 | NR_103734 | Hs.503463 | |
| | | coding RNA 342 | | | | |
| −2.3255814 | LINC00346 | long intergenic non-protein | 283487 | NM_178514 | Hs.245390 | ENSG00000255874 |
| | | coding RNA 346 | | | | |
| −2.7027027 | LINC00381 | long intergenic non-protein | 100874151 | NR_047005 | Hs.564552 | ENSG00000226240 |
| | | coding RNA 381 | | | | |
| −2.1739130 | LINC00410 | long intergenic non-protein | 144776 | NR_027039 | Hs.434120 | ENSG00000231674 |
| | | coding RNA 410 | | | | |
| −3.0303030 | LINC00458 | long intergenic non-protein | 100507428 | NR_108062 | Hs.351262 | ENSG00000234787 |
| | | coding RNA 458 | | | | |
| −2.0000000 | LINC00470 | long intergenic non-protein | 56651 | NM_031416 | Hs.541165 | |
| | | coding RNA 470 | | | | |
| −2.5641026 | LINC00485 | long intergenic non-protein | 283432 | NR_033855 | Hs.382110 | ENSG00000258169 |
| | | coding RNA 485 | | | | |
| −3.5714286 | LINC00506 | long intergenic non-protein | 100846978 | NR_047469 | Hs.570649 | |
| | | coding RNA 506 | | | | |
| −3.2258065 | LINC00507 | long intergenic non-protein | 100862680 | NR_046392 | Hs.385496 | ENSG00000256193 |
| | | coding RNA 507 | | | | |
| −2.5000000 | LINC00547 | long intergenic non-protein | 400121 | NR_040244 | Hs.558894 | ENSG00000275226 |
| | | coding RNA 547 | | | | |
| −2.3809524 | LINC00620 | long intergenic non-protein | 285375 | NR_027103 | Hs.319969 | ENSG00000224514 |
| | | coding RNA 620 | | | | |
| −2.4390244 | LINC00649 | long intergenic non-protein | 100506334 | NR_038883 | Hs.532855 | ENSG00000237945 |
| | | coding RNA 649 | | | | |
| −2.5641026 | LINC00652 | long intergenic non-protein | 29075 | NM_014162 | Hs.584899 | ENSG00000179935 |
| | | coding RNA 652 | | | | |
| −2.7777778 | LINC00670 | long intergenic non-protein | 284034 | NR_034144 | Hs.376614 | ENSG00000179136 |
| | | coding RNA 670 | | | | |
| −2.3809524 | LINC00672 | long intergenic non-protein | 100505576 | NR_038847 | Hs.634043 | ENSG00000263874 |
| | | coding RNA 672 | | | | |
| −3.8461538 | LINC00678 | long intergenic non-protein | 101410541 | NR_102708 | Hs.471439 | ENSG00000254934 |
| | | coding RNA 678 | | | | |
| −2.0833333 | LINC00889 | long intergenic non-protein | 158696 | NR_026935 | Hs.558664 | |
| | | coding RNA 889 | | | | |
| −1.9607843 | LINC00907 | long intergenic non-protein | 284260 | NR_046174 | Hs.652819 | ENSG00000267586 |
| | | coding RNA 907 | | | | |
| −2.1739130 | LINC00924 | long intergenic non-protein | 145820 | NR_027132 | Hs.652702 | ENSG00000259134 |
| | | coding RNA 924 | | | | |
| −2.7027027 | LINC00958 | long intergenic non-protein | 100506305 | NR_038904 | Hs.153408 | ENSG00000251381 |
| | | coding RNA 958 | | | | |
| −2.8571429 | LINC00965 | long intergenic non-protein | 349196 | NM_001025473 | Hs.559040 | |
| | | coding RNA 965 | | | | |
| −2.7027027 | LINC00970 | long intergenic non-protein | 101978719 | NR_104091 | Hs.517849 | ENSG00000203601 |
| | | coding RNA 970 | | | | |
| −2.5641026 | LOH12CR2 | loss of heterozygosity, 12, | 503693 | NR_024061 | Hs.67553 | ENSG00000205791 |
| | | chromosomal region 2 (non- | | | | |
| | | protein coding) | | | | |
| 1.9300000 | LRP10 | low density lipoprotein | 26020 | NM_014045 | Hs.525232 | ENSG00000197324 |
| | | receptor-related protein 10 | | | | |
| −2.0408163 | LRRN4CL | LRRN4 C-terminal like | 221091 | NM_203422 | Hs.427449 | ENSG00000177363 |
| −1.6949153 | LUC7L2 | LUC7-like 2 (S. cerevisiae) | 51631 | NM_001244585 | Hs.731488 | ENSG00000146963 |
| 1.9500000 | LYL1 | lymphoblastic leukemia | 4066 | NM_005583 | Hs.46446 | ENSG00000104903 |
| | | associated hematopoiesis | | | | |
| | | regulator 1 | | | | |
| −1.8518519 | LYRM7 | LYR motif containing 7 | 90624 | NM_001293735 | Hs.115467 | ENSG00000186687 |
| 2.1100000 | LYPLA2 | lysophospholipase II | 11313 | NM_007260 | Hs.533479 | ENSG00000011009 |
| −2.7777778 | MAB21L3 | mab-21-like 3 (C. elegans) | 126868 | NM_152367 | Hs.376194 | ENSG00000173212 |
| −2.0000000 | MRO | maestro | 83876 | NM_001127174 | Hs.30495 | ENSG00000134042 |
| 2.1000000 | MDP1 | magnesium-dependent | 145553 | NM_001199821 | Hs.220963 | ENSG00000213920 |
| | | phosphatase 1 | | | | |
| 1.8200000 | MDH1 | malate dehydrogenase 1, | 4190 | NM_001199111 | Hs.526521 | ENSG00000014641 |
| | | NAD (soluble) | | | | |
| 1.9200000 | MDH2 | malate dehydrogenase 2, | 4191 | NM_001282403 | Hs.520967 | ENSG00000146701 |
| | | NAD (mitochondrial) | | | | |
| −1.4492754 | MCTS1 | malignant T cell amplified | 28985 | NM_001137554 | Hs.102696 | ENSG00000232119 |
| | | sequence 1 | | | | |
| −2.1276596 | MAN1B1-AS1 | MAN1B1 antisense RNA 1 | 100289341 | NR_027447 | Hs.593896 | ENSG00000268996 |
| | | (head to head) | | | | |
| −3.8461538 | MANEAL | mannosidase, endo-alpha-like | 149175 | NM_001031740 | Hs.534562 | ENSG00000185090 |
| −1.8181818 | MAP7D3 | MAP7 domain containing 3 | 79649 | NM_001173516 | Hs.446275 | ENSG00000129680 |
| −2.1276596 | MRGPRX3 | MAS-related GPR, member X3 | 117195 | NM_054031 | Hs.380177 | ENSG00000179826 |
| −1.5151515 | MDM4 | MDM4, p53 regulator | 4194 | NM_001204171 | Hs.497492 | ENSG00000 198625 |
| −2.1739130 | MED15P9 | mediator complex subunit 15 | 285103 | NR_033903 | Hs.570106 | ENSG00000223760 |
| | | pseudogene 9 | | | | |
| −2.3255814 | MED18 | mediator complex subunit 18 | 54797 | NM_001127350 | Hs.479911 | ENSG00000130772 |
| −1.3888889 | MED29 | mediator complex subunit 29 | 55588 | NM_017592 | Hs.611541 | ENSG00000063322 |
| −2.0408163 | MLANA | melan-A | 2315 | NM_005511 | Hs.154069 | ENSG00000120215 |
| −2.0833333 | MAGEA10 | melanoma antigen family A10 | 4109 | NM_001011543 | Hs.18048 | ENSG00000124260 |
| −1.9230769 | MREG | melanoregulin | 55686 | NM_018000 | Hs.620391 | ENSG00000118242 |
| −2.0833333 | MS4A10 | membrane-spanning 4-domains, | 341116 | NM_206893 | Hs.591956 | ENSG00000172689 |
| | | subfamily A, member 10 | | | | |
| 1.7600000 | METTL17 | methyltransferase like 17 | 64745 | NM_001029991 | Hs.512693 | ENSG00000165792 |
| −2.0408163 | METTL20 | methyltransferase like 20 | 254013 | NM_001135863 | Hs.740628 | ENSG00000139160 |
| −2.5000000 | METTL21A | methyltransferase like 21A | 151194 | NM_001127395 | Hs.664764 | ENSG00000144401 |
| 1.7000000 | METTL23 | methyltransferase like 23 | 124512 | NM_001080510 | Hs.74655 | ENSG00000181038 |
| −2.1739130 | METTL2A | methyltransferase like 2A | 339175 | NM_001005372 | Hs.381204 | ENSG00000087995 |
| −1.9607843 | METTL2B | methyltransferase like 2B | 55798 | NM_018396 | Hs.433213 | ENSG00000165055 |
| 1.3000000 | METTL4 | methyltransferase like 4 | 64863 | NM_022840 | Hs.126888 | ENSG00000101574 |
| −2.0408163 | METTL8 | methyltransferase like 8 | 79828 | NM_024770 | Hs.135146 | ENSG00000123600 |
| 1.7900000 | MFNG | MFNG O-fucosylpeptide 3- | 4242 | NM_001166343 | Hs.517603 | ENSG00000100060 |
| | | beta-N- | | | | |
| | | acetylglucosaminyltransferase | | | | |
| 6.0100000 | MIR3655 | microRNA 3655 | 100500820 | NR_037428 | | ENSG00000264052 |
| −6.6666667 | MIR5194 | microRNA 5194 | 100847051 | NR_049826 | | ENSG00000264653 |
| 3.3700000 | MIR564 | microRNA 564 | 693149 | NR_030290 | | ENSG00000207783 |
| 2.8700000 | MIR636 | microRNA 636 | 693221 | NR_030366 | | ENSG00000207556 |
| −1.8867925 | MIR6723 | microRNA 6723 | 102465432 | NR_106781 | | ENSG00000278791 |
| 9.2400000 | MIR7845 | microRNA 7845 | 102465835 | NR_106999 | | ENSG00000277590 |
| 1.7900000 | MGST1 | microsomal glutathione S- | 4257 | NM_001260511 | Hs.389700 | ENSG00000008394 |
| | | transferase 1 | | | | |
| −1.7241379 | MICAL3 | microtubule associated | 57553 | NM_001122731 | Hs.528024 | ENSG00000243156 |
| | | monooxygenase, calponin | | | | |
| | | and LIM domain containing 3 | | | | |
| −2.7027027 | MAP1LC3C | microtubule-associated | 440738 | NM_001004343 | Hs.534971 | ENSG00000197769 |
| | | protein 1 light chain 3 gamma | | | | |
| 1.7000000 | MCMBP | minichromosome | 79892 | NM_001256378 | Hs.124246 | ENSG00000197771 |
| | | maintenance complex binding | | | | |
| | | protein | | | | |
| −1.7857143 | MIR210HG | MIR210 host gene | 100506211 | NR_038262 | Hs.446388 | ENSG00000247095 |
| −1.8181818 | MIRLET7BHG | MIRLET7B host gene | 400931 | NM_207477 | Hs.235838 | ENSG00000197182 |
| −2.4390244 | MTFMT | mitochondrial methionyl- | 123263 | NM_139242 | Hs.531615 | ENSG00000103707 |
| | | tRNA formyltransferase | | | | |
| 1.7600000 | MRPL12 | mitochondrial ribosomal | 6182 | NM_002949 | Hs.109059 | ENSG00000262814 |
| | | protein L12 | | | | |
| 2.0900000 | MRPL34 | mitochondrial ribosomal | 64981 | NM_023937 | Hs.515242 | ENSG00000130312 |
| | | protein L34 | | | | |
| 1.4900000 | MRPL37 | mitochondrial ribosomal | 51253 | NM_016491 | Hs.584908 | ENSG00000116221 |
| | | protein L37 | | | | |
| 1.8000000 | MRPL38 | mitochondrial ribosomal | 64978 | NM_032478 | Hs.442609 | ENSG00000204316 |
| | | protein L38 | | | | |
| 1.7300000 | MRPL41 | mitochondrial ribosomal | 64975 | NM_032477 | Hs.44017 | ENSG00000182154 |
| | | protein L41 | | | | |
| 2.0000000 | MRPS12 | mitochondrial ribosomal | 6183 | NM_021107 | Hs.411125 | ENSG00000128626 |
| | | protein S12 | | | | |
| −1.8518519 | MAPK13 | mitogen-activated protein | 5603 | NM_002754 | Hs.178695 | ENSG00000156711 |
| | | kinase 13 | | | | |
| 1.9300000 | MAP2K2 | mitogen-activated protein | 5605 | NM_030662 | Hs.465627 | ENSG00000126934 |
| | | kinase kinase 2 | | | | |
| 1.5600000 | MAP2K5 | mitogen-activated protein | 5607 | NM_001206804 | Hs.114198 | ENSG00000137764 |
| | | kinase kinase 5 | | | | |
| −1.6949153 | MAP3K9 | mitogen-activated protein | 4293 | NM_001284230 | Hs.445496 | ENSG00000006432 |
| | | kinase kinase kinase 9 | | | | |
| 1.8800000 | MZT2A | mitotic spindle organizing | 653784 | NM_001085365 | Hs.655067 | ENSG00000173272 |
| | | protein 2A | | | | |
| 1.4700000 | MLX | MLX, MAX dimerization | 6945 | NM_170607 | Hs.383019 | ENSG00000108788 |
| | | protein | | | | |
| −2.5000000 | MOCS3 | molybdenum cofactor | 27304 | NM_014484 | Hs.159410 | ENSG00000124217 |
| | | synthesis 3 | | | | |
| −2.1739130 | MMD2 | monocyte to macrophage | 221938 | NM_001100600 | Hs.558694 | ENSG00000136297 |
| | | differentiation-associated 2 | | | | |
| −1.9230769 | MORN4 | MORN repeat containing 4 | 118812 | NM_001098831 | Hs.217409 | ENSG00000171160 |
| −1.9607843 | MPV17L | MPV17 mitochondrial | 255027 | NM_001128423 | Hs.720673 | ENSG00000156968 |
| | | membrane protein-like | | | | |
| −2.2222222 | MUC6 | mucin 6, oligomeric | 4588 | NM_005961 | Hs.528432 | ENSG00000184956 |
| | | mucus/gel-forming | | | | |
| −2.3255814 | MMRN2 | multimerin 2 | 79812 | NM_024756 | Hs.524479 | ENSG00000173269 |
| −1.7857143 | MCFD2 | multiple coagulation factor | 90411 | NM_001171506 | Hs.662152 | ENSG00000180398 |
| | | deficiency 2 | | | | |
| 1.4700000 | MUS81 | MUS81 structure-specific | 80198 | NM_025128 | Hs.288798 | ENSG00000172732 |
| | | endonuclease subunit | | | | |
| 2.0500000 | MYPOP | Myb-related transcription | 339344 | NM_001012643 | Hs.515478 | ENSG00000176182 |
| | | factor, partner of profilin | | | | |
| 1.3800000 | MBP | myelin basic protein | 4155 | NM_001025081 | Hs.551713 | ENSG00000197971 |
| −1.7857143 | MYEF2 | myelin expression factor 2 | 50804 | NM_001301210 | Hs.6638 | ENSG00000104177 |
| −2.5000000 | MOG | myelin oligodendrocyte | 4340 | NM_001008228 | Hs.141308 | ENSG00000204655 |
| | | glycoprotein | | | | |
| −1.8181818 | MPZL3 | myelin protein zero-like 3 | 196264 | NM_001286152 | Hs.15396 | ENSG00000160588 |
| −1.8867925 | MYLK3 | myosin light chain kinase 3 | 91807 | NM_182493 | Hs.130465 | ENSG00000140795 |
| 1.4000000 | MYO7B | myosin VIIB | 4648 | NM_001080527 | Hs.154578 | ENSG00000169994 |
| −1.6666667 | NAA16 | N(alpha)-acetyltransferase 16, | 79612 | NM_001110798 | Hs.512914 | ENSG00000172766 |
| | | NatA auxiliary subunit | | | | |
| 1.6400000 | NAGA | N-acetylgalactosaminidase, | 4668 | NM_000262 | Hs.75372 | ENSG00000198951 |
| | | alpha- | | | | |
| −2.0833333 | NWD1 | NACHT and WD repeat | 284434 | NM_001007525 | Hs.406014 | ENSG00000188039 |
| | | domain containing 1 | | | | |
| −1.8867925 | NQO1 | NAD(P)H dehydrogenase, | 1728 | NM_000903 | Hs.406515 | ENSG00000181019 |
| | | quinone 1 | | | | |
| 1.8100000 | NDUFA7 | NADH dehydrogenase | 4701 | NM_005001 | Hs.333427 | ENSG00000267855 |
| | | (ubiquinone) 1 alpha | | | | |
| | | subcomplex, 7, 14.5 kDa | | | | |
| 1.9000000 | NDUFB11 | NADH dehydrogenase | 54539 | NM_001135998 | Hs.521969 | ENSG00000147123 |
| | | (ubiquinone) 1 beta | | | | |
| | | subcomplex, 11, 17.3 kDa | | | | |
| 1.7300000 | NDUFB6 | NADH dehydrogenase | 4712 | NM_001199987 | Hs.493668 | ENSG00000165264 |
| | | (ubiquinone) 1 beta | | | | |
| | | subcomplex, 6, 17 kDa | | | | |
| 1.5100000 | NDUFB7 | NADH dehydrogenase | 4713 | NM_004146 | Hs.532853 | ENSG00000099795 |
| | | (ubiquinone) 1 beta | | | | |
| | | subcomplex, 7, 18 kDa | | | | |
| 1.3800000 | NDUFB8 | NADH dehydrogenase | 4714 | NM_001284367 | Hs.523215 | ENSG00000166136 |
| | | (ubiquinone) 1 beta | | | | |
| | | subcomplex, 8, 19 kDa | | | | |
| 2.0600000 | NDUFC1 | NADH dehydrogenase | 4717 | NM_001184986 | Hs.84549 | ENSG00000109390 |
| | | (ubiquinone) 1, subcomplex | | | | |
| | | unknown, 1, 6 kDa | | | | |
| −1.7857143 | NDUFAF7 | NADH dehydrogenase | 55471 | NM_001083946 | Hs.433466 | ENSG00000003509 |
| | | (ubiquinone) complex I, | | | | |
| | | assembly factor 7 | | | | |
| 1.8500000 | NDUFS7 | NADH dehydrogenase | 374291 | NM_024407 | Hs.211914 | ENSG00000115286 |
| | | (ubiquinone) Fe—S protein 7, | | | | |
| | | 20 kDa (NADH-coenzyme Q | | | | |
| | | reductase) | | | | |
| −1.4492754 | NECAP1 | NECAP endocytosis | 25977 | NM_015509 | Hs.555927 | ENSG00000089818 |
| | | associated 1 | | | | |
| −1.9607843 | NPHS1 | nephrosis 1, congenital, | 4868 | NM_004646 | Hs.122186 | ENSG00000161270 |
| | | Finnish type (nephrin) | | | | |
| −2.6315789 | NBPF20 | neuroblastoma breakpoint | 100288142 | NM_001278267 | Hs.445080 | ENSG00000162825 |
| | | family, member 20 | | | | |
| −2.6315789 | NEXN-AS1 | NEXN antisense RNA 1 | 374987 | NM_001039463 | Hs.632414 | ENSG00000235927 |
| 1.5200000 | NKIRAS2 | NFKB inhibitor interacting | 28511 | NM_001001349 | Hs.632252 | ENSG00000168256 |
| | | Ras-like 2 | | | | |
| −1.9230769 | NMNAT1 | nicotinamide nucleotide | 64802 | NM_001297778 | Hs.633762 | ENSG00000173614 |
| | | adenylyltransferase 1 | | | | |
| −2.2222222 | NEK2 | NIMA-related kinase 2 | 4751 | NM_001204182 | Hs.153704 | ENSG00000117650 |
| 1.7900000 | NIPAL2 | NIPA-like domain containing 2 | 79815 | NM_024759 | Hs.309489 | ENSG00000104361 |
| 1.6200000 | NIPSNAP3A | nipsnap homolog 3A (C. elegans) | 25934 | NM_015469 | Hs.530275 | ENSG00000136783 |
| −1.8518519 | NMT2 | N-myristoyltransferase 2 | 9397 | NM_004808 | Hs.60339 | ENSG00000152465 |
| −1.9607843 | NCMAP | noncompact myelin | 400746 | NM_001010980 | Hs.200253 | ENSG00000184454 |
| | | associated protein | | | | |
| −1.9230769 | NFKBIZ | nuclear factor of kappa light | 64332 | NM_001005474 | Hs.319171 | ENSG00000144802 |
| | | polypeptide gene enhancer in | | | | |
| | | B-cells inhibitor, zeta | | | | |
| −1.8518519 | NUGGC | nuclear GTPase, germinal | 389643 | NM_001010906 | Hs.370129 | ENSG00000189233 |
| | | center associated | | | | |
| −1.8867925 | NPAP1 | nuclear pore associated | 23742 | NM_018958 | Hs.649663 | ENSG00000185823 |
| | | protein 1 | | | | |
| 2.0700000 | NPIPA2 | nuclear pore complex | 642799 | NM_001277324 | | ENSG00000254852 |
| | | interacting protein family, | | | | |
| | | member A2 | | | | |
| −2.0408163 | NRIP3 | nuclear receptor interacting | 56675 | NM_020645 | Hs.523467 | ENSG00000175352 |
| | | protein 3 | | | | |
| −1.4705882 | NR6A1 | nuclear receptor subfamily 6, | 2649 | NM_001278546 | Hs.20131 | ENSG00000148200 |
| | | group A, member 1 | | | | |
| −1.8518519 | NFX1 | nuclear transcription factor, | 4799 | NM_002504 | Hs.413074 | ENSG00000086102 |
| | | X-box binding 1 | | | | |
| 1.2500000 | NUP133 | nucleoporin 133 kDa | 55746 | NM_018230 | Hs.12457 | ENSG00000069248 |
| −2.6315789 | NXN | nucleoredoxin | 64359 | NM_001205319 | Hs.527989 | ENSG00000167693 |
| −2.3255814 | NXNL2 | nucleoredoxin-like 2 | 158046 | NM_001161625 | Hs.734507 | ENSG00000130045 |
| 1.8700000 | NUDT22 | nudix (nucleoside diphosphate | 84304 | NM_001128612 | Hs.656074 | ENSG00000149761 |
| | | linked moiety X)-type motif 22 | | | | |
| 1.9300000 | OCEL1 | occludin/ELL domain containing 1 | 79629 | NM_024578 | Hs.422676 | ENSG00000099330 |
| −2.0408163 | OLAH | oleoyl-ACP hydrolase | 55301 | NM_001039702 | Hs.24309 | ENSG00000152463 |
| −2.0833333 | OR11A1 | olfactory receptor, family 11, | 26531 | NM_013937 | Hs.676010 | ENSG00000204694 |
| | | subfamily A, member 1 | | | | |
| 2.0400000 | OLIG1 | oligodendrocyte transcription | 116448 | NM_138983 | Hs.56663 | ENSG00000184221 |
| | | factor 1 | | | | |
| −2.5000000 | OPHN1 | oligophrenin 1 | 4983 | NM_002547 | Hs.128824 | ENSG00000079482 |
| −1.8518519 | OSMR | oncostatin M receptor | 9180 | NM_001168355 | Hs.120658 | ENSG00000145623 |
| 1.8100000 | OPN3 | opsin 3 | 23596 | NM_001030011 | Hs.409081 | ENSG00000054277 |
| −1.6949153 | OPA3 | optic atrophy 3 (autosomal | 80207 | NM_001017989 | Hs.466945 | ENSG00000125741 |
| | | recessive, with chorea and | | | | |
| | | spastic paraplegia) | | | | |
| 1.8600000 | ORAI3 | ORAI calcium release- | 93129 | NM_152288 | Hs.745104 | ENSG00000175938 |
| | | activated calcium modulator 3 | | | | |
| −2.8571429 | ORC4 | origin recognition complex, | 5000 | NM_001190879 | Hs.558364 | ENSG00000115947 |
| | | subunit 4 | | | | |
| −1.9230769 | OSGEPL1-AS1 | OSGEPL1 antisense RNA 1 | 101409258 | NR_102429 | Hs.738558 | ENSG00000253559 |
| −1.8518519 | OTUD3 | OTU deubiquitinase 3 | 23252 | NM_015207 | Hs.374987 | ENSG00000169914 |
| −2.8571429 | OTUD6A | OTU deubiquitinase 6A | 139562 | NM_207320 | Hs.447381 | ENSG00000189401 |
| −3.2258065 | ODF2L | outer dense fiber of sperm | 57489 | NM_001007022 | Hs.149360 | ENSG00000122417 |
| | | tails 2-like | | | | |
| −1.6666667 | OSBPL2 | oxysterol binding protein-like 2 | 9885 | NM_001001691 | Hs.473254 | ENSG00000130703 |
| 1.5200000 | OSBPL7 | oxysterol binding protein-like 7 | 114881 | NM_017731 | Hs.463320 | ENSG00000006025 |
| −2.5000000 | LOC646214 | p21 protein (Cdc42/Rac)- | 646214 | NR_027053 | Hs.510697 | ENSG00000278673 |
| | | activated kinase 2 pseudogene | | | | |
| −2.1739130 | P2RX5-TAX1BP3 | P2RX5-TAX1BP3 readthrough | 100533970 | NR_037928 | Hs.731607 | |
| | | (NMD candidate) | | | | |
| −1.6393443 | PAF1 | Paf1, RNA polymerase II | 54623 | NM_001256826 | Hs.466714 | ENSG00000006712 |
| | | associated factor, homolog | | | | |
| | | (S. cerevisiae) | | | | |
| −2.5000000 | PTCSC3 | papillary thyroid carcinoma | 100886964 | NR_049735 | Hs.742592 | ENSG00000259104 |
| | | susceptibility candidate 3 | | | | |
| | | (non-protein coding) | | | | |
| −2.5000000 | PARD6G | par-6 family cell polarity | 84552 | NM_032510 | Hs.654920 | ENSG00000178184 |
| | | regulator gamma | | | | |
| −2.0833333 | PNMA2 | paraneoplastic Ma antigen 2 | 10687 | NM_007257 | Hs.591838 | ENSG00000240694 |
| −2.0408163 | PARK2 | parkin RBR E3 ubiquitin | 5071 | NM_004562 | Hs.132954 | ENSG00000185345 |
| | | protein ligase | | | | |
| −2.1276596 | PDDC1 | Parkinson disease 7 domain | 347862 | NM_182612 | Hs.218362 | ENSG00000177225 |
| | | containing 1 | | | | |
| 1.3300000 | PARK7 | parkinson protein 7 | 11315 | NM_001123377 | Hs.419640 | ENSG00000116288 |
| −2.0408163 | LOC100287846 | patched 1 pseudogene | 100287846 | NR_037168 | Hs.21550 | |
| −2.7027027 | PTCHD4 | patched domain containing 4 | 442213 | NM_001013732 | Hs.659409 | ENSG00000244694 |
| 4.0900000 | PDZK1IPl | PDZK1 interacting protein 1 | 10158 | NM_005764 | Hs.431099 | ENSG00000162366 |
| 1.6200000 | PEPD | peptidase D | 5184 | NM_000285 | Hs.36473 | ENSG00000124299 |
| −2.1739130 | PPIL6 | peptidylprolyl isomerase | 285755 | NM_001111298 | Hs.32234 | ENSG00000185250 |
| | | (cyclophilin)-like 6 | | | | |
| −2.0833333 | PPIEL | peptidylprolyl isomerase E- | 728448 | NR_003929 | Hs.472508 | ENSG00000182109 |
| | | like pseudogene | | | | |
| −1.4925373 | PER2 | period circadian clock 2 | 8864 | NM_003894 | Hs.58756 | ENSG00000132326 |
| 1.8600000 | PRDX3 | peroxiredoxin 3 | 10935 | NM_001302272 | Hs.523302 | ENSG00000165672 |
| 1.8600000 | PRDX4 | peroxiredoxin 4 | 10549 | NM_006406 | Hs.83383 | ENSG00000123131 |
| −1.8867925 | PXMP4 | peroxisomal membrane | 11264 | NM_007238 | Hs.654857 | ENSG00000101417 |
| | | protein 4, 24 kDa | | | | |
| −1.3698630 | PHF12 | PHD finger protein 12 | 57649 | NM_001033561 | Hs.444173 | ENSG00000109118 |
| −1.7543860 | PHACTR4 | phosphatase and actin | 65979 | NM_001048183 | Hs.225641 | ENSG00000204138 |
| | | regulator 4 | | | | |
| 1.3900000 | PIGY | phosphatidylinositol glycan | 84992 | NM_001042616 | Hs.26136 | ENSG00000255072 |
| | | anchor biosynthesis, class Y | | | | |
| −1.7857143 | PLCXD1 | phosphatidylinositol-specific | 55344 | NM_018390 | Hs.522568 | ENSG00000182378 |
| | | phospholipase C, X domain | | | | |
| | | containing 1 | | | | |
| 1.6000000 | PISD | phosphatidylserine | 23761 | NM_014338 | Hs.420559 | ENSG00000241878 |
| | | decarboxylase | | | | |
| −2.1276596 | PDE4C | phosphodiesterase 4C, | 5143 | NM_000923 | Hs.132584 | ENSG00000105650 |
| | | cAMP-specific | | | | |
| −2.3809524 | PDE6A | phosphodiesterase 6A, | 5145 | NM_000440 | Hs.567314 | ENSG00000132915 |
| | | cGMP-specific, rod, alpha | | | | |
| −3.4482759 | PGM5P2 | phosphoglucomutase 5 | 595135 | NR_002836 | Hs.571593 | ENSG00000277778 |
| | | pseudogene 2 | | | | |
| −2.0833333 | PLA2G4E | phospholipase A2, group IVE | 123745 | NM_001080490 | Hs.668060 | ENSG00000188089 |
| −1.7241379 | PHAX | phosphorylated adaptor for | 51808 | NM_032177 | Hs.555731 | ENSG00000164902 |
| | | RNA export | | | | |
| 1.3900000 | PYURF | PIGY upstream reading frame | 100996939 | NM_032906 | Hs.26136 | ENSG00000145337 |
| −2.0408163 | PNN | pinin, desmosome associated | 5411 | NM_002687 | Hs.409965 | ENSG00000100941 |
| | | protein | | | | |
| −2.2222222 | PLEKHA5 | pleckstrin homology domain | 54477 | NM_001143821 | Hs.188614 | ENSG00000052126 |
| | | containing, family A member 5 | | | | |
| −1.9230769 | PLEKHG2 | pleckstrin homology domain | 64857 | NM_022835 | Hs.631574 | ENSG00000090924 |
| | | containing, family G (with | | | | |
| | | RhoGef domain) member 2 | | | | |
| −2.5000000 | PABPC1P2 | poly(A) binding protein, | 728773 | NR_026904 | Hs.334462 | ENSG00000198526 |
| | | cytoplasmic 1 pseudogene 2 | | | | |
| −1.4492754 | PCGF3 | polycomb group ring finger 3 | 10336 | NM_006315 | Hs.144309 | ENSG00000185619 |
| −1.5625000 | POLE3 | polymerase (DNA directed), | 54107 | NM_001278255 | Hs.108112 | ENSG00000148229 |
| | | epsilon 3, accessory subunit | | | | |
| −1.7543860 | POLH | polymerase (DNA directed), eta | 5429 | NM_001291969 | Hs.655467 | ENSG00000170734 |
| 1.4800000 | POLD4 | polymerase (DNA-directed), | 57804 | NM_001256870 | Hs.523829 | ENSG00000175482 |
| | | delta 4, accessory subunit | | | | |
| 1.3500000 | POLR2D | polymerase (RNA) II (DNA | 5433 | NM_004805 | Hs.715348 | ENSG00000144231 |
| | | directed) polypeptide D | | | | |
| 1.4700000 | PTBP1 | polypyrimidine tract binding | 5725 | NM_002819 | Hs.172550 | ENSG00000011304 |
| | | protein 1 | | | | |
| −1.8518519 | PBDC1 | polysaccharide biosynthesis | 51260 | NM_001300888 | Hs.370100 | ENSG00000102390 |
| | | domain containing 1 | | | | |
| −1.5384615 | KCMF1 | potassium channel | 56888 | NM_020122 | Hs.654968 | ENSG00000176407 |
| | | modulatory factor 1 | | | | |
| −2.3809524 | KCNJ5 | potassium channel, inwardly | 3762 | NM_000890 | Hs.444595 | ENSG00000120457 |
| | | rectifying subfamily J, | | | | |
| | | member 5 | | | | |
| −2.2222222 | KCNA7 | potassium channel, voltage | 3743 | NM_031886 | Hs.306973 | ENSG00000104848 |
| | | gated shaker related | | | | |
| | | subfamily A, member 7 | | | | |
| −2.2727273 | POU2AF1 | POU class 2 associating | 5450 | NM_006235 | Hs.654525 | ENSG00000110777 |
| | | factor 1 | | | | |
| −2.5000000 | POU5F1 | POU class 5 homeobox 1 | 5460 | NM_001173531 | Hs.249184 | ENSG00000204531 |
| 1.6600000 | PQLC3 | PQ loop repeat containing 3 | 130814 | NM_001282710 | Hs.274415 | ENSG00000162976 |
| −1.8518519 | PRDM7 | PR domain containing 7 | 11105 | NM_001098173 | Hs.406695 | ENSG00000126856 |
| −1.7857143 | PRPF3 | pre-mRNA processing factor 3 | 9129 | NM_004698 | Hs.11776 | ENSG00000117360 |
| −2.0408163 | PRPF38B | pre-mRNA processing factor 38B | 55119 | NM_018061 | Hs.342307 | ENSG00000134186 |
| 1.3100000 | PRPF8 | pre-mRNA processing factor 8 | 10594 | NM_006445 | Hs.181368 | ENSG00000174231 |
| −2.4390244 | PRICKLE2-AS3 | PRICKLE2 antisense RNA 3 | 100874243 | NR_046702 | Hs.670840 | ENSG00000226017 |
| −2.2222222 | PRKAR2A-AS1 | PRKAR2A antisense RNA 1 | 100506637 | NR_109996 | Hs.634259 | ENSG00000224424 |
| −2.0408163 | PRKX-AS1 | PRKX antisense RNA 1 | 100873944 | NR_046643 | | ENSG00000236188 |
| 1.5600000 | POP7 | processing of precursor 7, | 10248 | NM_005837 | Hs.416994 | ENSG00000172336 |
| | | ribonuclease P/MRP subunit | | | | |
| | | (S. cerevisiae) | | | | |
| −1.9230769 | PFN1P2 | profilin 1 pseudogene 2 | 767846 | NR_003242 | Hs.657186 | ENSG00000270392 |
| 1.3700000 | PDCD6IP | programmed cell death 6 | 10015 | NM_001162429 | Hs.475896 | ENSG00000170248 |
| | | interacting protein | | | | |
| 1.3600000 | PHB2 | prohibitin 2 | 11331 | NM_001144831 | Hs.504620 | ENSG00000215021 |
| −2.0000000 | PRR11 | proline rich 11 | 55771 | NM_018304 | Hs.631750 | ENSG00000068489 |
| −1.5384615 | PRRC2C | proline-rich coiled-coil 2C | 23215 | NM_015172 | Hs.494614 | ENSG00000117523 |
| −2.8571429 | PART1 | prostate androgen-regulated | 25859 | NM_001039499 | Hs.146312 | ENSG00000152931 |
| | | transcript 1 (non-protein | | | | |
| | | coding) | | | | |
| −2.3809524 | PRNCR1 | prostate cancer associated | 101867536 | NR_109833 | Hs.652970 | ENSG00000224722 |
| | | non-coding RNA 1 | | | | |
| −1.8181818 | PCAT19 | prostate cancer associated | 100505495 | NR_040109 | Hs.648878 | ENSG00000267107 |
| | | transcript 19 (non-protein | | | | |
| | | coding) | | | | |
| 1.5700000 | PSMD14 | proteasome (prosome, | 10213 | NM_005805 | Hs.740477 | ENSG00000115233 |
| | | macropain) 26S subunit, non- | | | | |
| | | ATPase, 14 | | | | |
| 1.4300000 | PSMD9 | proteasome (prosome, | 5715 | NM_001261400 | Hs.131151 | ENSG00000110801 |
| | | macropain) 26S subunit, non- | | | | |
| | | ATPase, 9 | | | | |
| 1.7900000 | PSMB5 | proteasome (prosome, | 5693 | NM_001130725 | Hs.422990 | ENSG00000100804 |
| | | macropain) subunit, beta | | | | |
| | | type, 5 | | | | |
| −2.1276596 | PIN4P1 | protein (peptidylprolyl | 728758 | NR_003571 | Hs.658099 | ENSG00000227973 |
| | | cis/trans isomerase) NIMA- | | | | |
| | | interacting, 4 pseudogene 1 | | | | |
| −2.0000000 | PRKXP1 | protein kinase, X-linked, | 441733 | NR_073405 | Hs.12250 | ENSG00000270127 |
| | | pseudogene 1 | | | | |
| 1.5900000 | PPP1R37 | protein phosphatase 1, | 284352 | NM_019121 | Hs.285363 | ENSG00000104866 |
| | | regulatory subunit 37 | | | | |
| 1.8700000 | PPP2CB | protein phosphatase 2, | 5516 | NM_001009552 | Hs.491440 | ENSG00000104695 |
| | | catalytic subunit, beta | | | | |
| | | isozyme | | | | |
| 1.2900000 | PPP3CB | protein phosphatase 3, | 5532 | NM_001142353 | Hs.500067 | ENSG00000107758 |
| | | catalytic subunit, beta | | | | |
| | | isozyme | | | | |
| −1.7857143 | PPM1K | protein phosphatase, | 152926 | NM_152542 | Hs.43744 | ENSG00000163644 |
| | | Mg2+/Mn2+ dependent, 1K | | | | |
| −2.0408163 | PTK6 | protein tyrosine kinase 6 | 5753 | NM_001256358 | Hs.51133 | ENSG00000101213 |
| −1.7241379 | PTPN14 | protein tyrosine phosphatase, | 5784 | NM_005401 | Hs.193557 | ENSG00000152104 |
| | | non-receptor type 14 | | | | |
| 1.4100000 | PTPN9 | protein tyrosine phosphatase, | 5780 | NM_002833 | Hs.445775 | ENSG00000169410 |
| | | non-receptor type 9 | | | | |
| −2.2727273 | PCDH11X | protocadherin 11 X-linked | 27328 | NM_001168360 | Hs.655673 | ENSG00000102290 |
| −2.3809524 | PCDH11Y | protocadherin 11 Y-linked | 83259 | NM_001278619 | Hs.661308 | ENSG00000099715 |
| −2.1739130 | PCDHB9 | protocadherin beta 9 | 56127 | NM_019119 | Hs.662726 | ENSG00000177839 |
| −1.8181818 | PCBD2 | pterin-4 alpha-carbinolamine | 84105 | NM_032151 | Hs.710014 | ENSG00000132570 |
| | | dehydratase/dimerization | | | | |
| | | cofactor of hepatocyte nuclear | | | | |
| | | factor 1 alpha (TCF1) 2 | | | | |
| −1.8867925 | PTGES2-AS1 | PTGES2 antisense RNA 1 | 389791 | NM_001013652 | Hs.632678 | ENSG00000232850 |
| | | (head to head) | | | | |
| −1.7241379 | PPFIBP1 | PTPRF interacting protein, | 8496 | NM_001198915 | Hs.172445 | ENSG00000110841 |
| | | binding protein 1 (liprin beta 1) | | | | |
| −1.9607843 | PTPRG-AS1 | PTPRG antisense RNA 1 | 100506994 | NR_038281 | Hs.656620 | ENSG00000241472 |
| −1.9607843 | LOC100506127 | putative uncharacterized | 100506127 | NM_001013634 | Hs.503319 | ENSG00000179240 |
| | | protein FLJ37770-like | | | | |
| 2.0800000 | PYCARD | PYD and CARD domain | 29108 | NM_013258 | Hs.499094 | ENSG00000103490 |
| | | containing | | | | |
| 2.1000000 | PYGO2 | pygopus family PHD finger 2 | 90780 | NM_138300 | Hs.533597 | ENSG00000163348 |
| −1.5873016 | PDXDC2P | pyridoxal-dependent | 283970 | NM_199134 | Hs.513695 | ENSG00000196696 |
| | | decarboxylase domain | | | | |
| | | containing 2, pseudogene | | | | |
| −1.4925373 | PGPEP1 | pyroglutamyl-peptidase I | 54858 | NM_001300927 | Hs.131776 | ENSG00000130517 |
| −2.0000000 | PDP2 | pyruvate dehyrogenase | 57546 | NM_020786 | Hs.632214 | ENSG00000172840 |
| | | phosphatase catalytic subunit 2 | | | | |
| 1.5100000 | QTRT1 | queuine tRNA- | 81890 | NM_031209 | Hs.631638 | ENSG00000213339 |
| | | ribosyltransferase 1 | | | | |
| −1.2820513 | QTRTD1 | queuine tRNA- | 79691 | NM_001256835 | Hs.477162 | ENSG00000151576 |
| | | ribosyltransferase domain | | | | |
| | | containing 1 | | | | |
| 1.5700000 | RABAC1 | Rab acceptor 1 (prenylated) | 10567 | NM_006423 | Hs.11417 | ENSG00000105404 |
| −1.4925373 | RAB11FIP4 | RAB11 family interacting | 84440 | NM_001303542 | Hs.406788 | ENSG00000131242 |
| | | protein 4 (class II) | | | | |
| −1.7857143 | RAB12 | RAB12, member RAS | 201475 | NM_001025300 | Hs.270074 | ENSG00000206418 |
| | | oncogene family | | | | |
| −2.1276596 | RAB42 | RAB42, member RAS | 115273 | NM_001193532 | Hs.652321 | ENSG00000188060 |
| | | oncogene family | | | | |
| 1.4000000 | RAB5C | RAB5C, member RAS | 5878 | NM_001252039 | Hs.650382 | ENSG00000108774 |
| | | oncogene family | | | | |
| −1.4705882 | RAD1 | RAD1 checkpoint DNA | 5810 | NM_001033673 | Hs.38114 | ENSG00000113456 |
| | | exonuclease | | | | |
| 1.5300000 | RALY | RALY heterogeneous nuclear | 22913 | NM_007367 | Hs.136947 | ENSG00000125970 |
| | | ribonucleoprotein | | | | |
| −2.7027027 | RAMP2-AS1 | RAMP2 antisense RNA 1 | 100190938 | NR_024461 | Hs.655265 | ENSG00000197291 |
| 2.2400000 | RHOG | ras homolog family member G | 391 | NM_001665 | Hs.501728 | ENSG00000177105 |
| −2.7777778 | RASAL2-AS1 | RASAL2 antisense RNA 1 | 100302401 | NR_027982 | Hs.736117 | ENSG00000224687 |
| 1.8500000 | RASL11A | RAS-like, family 11, member A | 387496 | NM_206827 | Hs.192131 | ENSG00000122035 |
| 1.9700000 | RAC2 | ras-related C3 botulinum | 5880 | NM_002872 | Hs.517601 | ENSG00000128340 |
| | | toxin substrate 2 (rho family, | | | | |
| | | small GTP binding protein Rac2) | | | | |
| 1.6400000 | RITA1 | RBPJ interacting and tubulin | 84934 | NM_001286215 | Hs.524762 | ENSG00000139405 |
| | | associated 1 | | | | |
| −1.3888889 | RIPK1 | receptor (TNFRSF)-interacting | 8737 | NM_003804 | Hs.519842 | ENSG00000137275 |
| | | serine-threonine kinase 1 | | | | |
| 2.0100000 | RGS14 | regulator of G-protein | 10636 | NM_006480 | Hs.9347 | ENSG00000169220 |
| | | signaling 14 | | | | |
| 1.4200000 | RGS3 | regulator of G-protein | 5998 | NM_001276260 | Hs.494875 | ENSG00000138835 |
| | | signaling 3 | | | | |
| 1.6700000 | RFX1 | regulatory factor X, 1 | 5989 | NM_002918 | Hs.655215 | ENSG00000132005 |
| | | (influences HLA class II | | | | |
| | | expression) | | | | |
| 1.4800000 | RTN3 | reticulon 3 | 10313 | NM_001265589 | Hs.743229 | ENSG00000133318 |
| −1.8181818 | RFPL1S | RFPL1 antisense RNA 1 | 10740 | NR_002727 | Hs.167750 | ENSG00000225465 |
| −1.7241379 | RHD | Rh blood group, D antigen | 6007 | NM_001127691 | Hs.449968 | ENSG00000187010 |
| −2.0000000 | RHBG | Rh family, B glycoprotein | 57127 | NM_001256395 | Hs.131835 | ENSG00000132677 |
| | | (gene/pseudogene) | | | | |
| −1.2987013 | ARHGAP26 | Rho GTPase activating | 23092 | NM_001135608 | Hs.654668 | ENSG00000145819 |
| | | protein 26 | | | | |
| −2.0833333 | RHBDL2 | rhomboid, veinlet-like 2 | 54933 | NM_001304746 | Hs.524626 | ENSG00000158315 |
| | | (Drosophila) | | | | |
| 2.0000000 | RAVER1 | ribonucleoprotein, PTB- | 125950 | NM_133452 | Hs.744952 | ENSG00000161847 |
| | | binding 1 | | | | |
| 1.6500000 | RPL18A | ribosomal protein L18a | 6142 | NM_000980 | Hs.337766 | ENSG00000105640 |
| 1.6300000 | RPL28 | ribosomal protein L28 | 6158 | NM_000991 | Hs.652114 | ENSG00000108107 |
| 1.5400000 | RPL41 | ribosomal protein L41 | 6171 | NM_001035267 | Hs.112553 | ENSG00000229117 |
| 1.5900000 | RPS12 | ribosomal protein S12 | 6206 | NM_001016 | Hs.546289 | ENSG00000112306 |
| −1.7241379 | RPS15AP10 | ribosomal protein S15a | 728963 | NR_026768 | Hs.675157 | ENSG00000225447 |
| | | pseudogene 10 | | | | |
| 1.9500000 | RPS6KA4 | ribosomal protein S6 kinase, | 8986 | NM_001006944 | Hs.105584 | ENSG00000162302 |
| | | 90 kDa, polypeptide 4 | | | | |
| 1.8700000 | RPLP1 | ribosomal protein, large, P1 | 6176 | NM_001003 | Hs.356502 | ENSG00000137818 |
| −1.6129032 | RRP15 | ribosomal RNA processing | 51018 | NM_016052 | Hs.660109 | ENSG00000067533 |
| | | 15 homolog (S. cerevisiae) | | | | |
| 1.9700000 | RNF126 | ring finger protein 126 | 55658 | NM_017876 | Hs.69554 | ENSG00000070423 |
| 1.5400000 | RNF167 | ring finger protein 167 | 26001 | NM_015528 | Hs.7158 | ENSG00000108523 |
| −2.1276596 | RNF207 | ring finger protein 207 | 388591 | NM_173795 | Hs.716549 | ENSG00000158286 |
| −1.2195122 | RNF216 | ring finger protein 216 | 54476 | NM_019011 | Hs.487458 | ENSG00000011275 |
| −2.5641026 | RNF222 | ring finger protein 222 | 643904 | NM_001146684 | Hs.526550 | ENSG00000189051 |
| −1.9607843 | RBM25 | RNA binding motif protein 25 | 58517 | NM_021239 | Hs.531106 | ENSG00000119707 |
| −2.1276596 | RBM34 | RNA binding motif protein 34 | 23029 | NM_001161533 | Hs.535224 | ENSG00000188739 |
| 1.4100000 | RBM42 | RNA binding motif protein 42 | 79171 | NM_024321 | Hs.5086 | ENSG00000126254 |
| −1.6666667 | RBM43 | RNA binding motif protein 43 | 375287 | NM_198557 | Hs.302442 | ENSG00000184898 |
| −1.8518519 | RBM48 | RNA binding motif protein 48 | 84060 | NM_032120 | Hs.21590 | ENSG00000127993 |
| −1.6129032 | RBMS2 | RNA binding motif, single | 5939 | NM_002898 | Hs.505729 | ENSG00000076067 |
| | | stranded interacting protein 2 | | | | |
| −2.3255814 | RNF144A-AS1 | RNF144A antisense RNA 1 | 386597 | NR_033997 | Hs.559010 | ENSG00000228203 |
| −3.0303030 | ROR1-AS1 | ROR1 antisense RNA 1 | 101927034 | NR_110665 | Hs.680824 | ENSG00000223949 |
| −1.4492754 | SAP30BP | SAP30 binding protein | 29115 | NM_001301839 | Hs.655088 | ENSG00000161526 |
| −1.8867925 | SFMBT2 | Scm-like with four mbt | 57713 | NM_001018039 | Hs.407983 | ENSG00000198879 |
| | | domains 2 | | | | |
| −1.5151515 | SDE2 | SDE2 telomere maintenance | 163859 | NM_152608 | Hs.520192 | ENSG00000143751 |
| | | homolog (S. pombe) | | | | |
| 1.6000000 | SEMA4A | sema domain, | 64218 | NM_001193300 | Hs.408846 | ENSG00000196189 |
| | | immunoglobulin domain (Ig), | | | | |
| | | transmembrane domain (TM) | | | | |
| | | and short cytoplasmic | | | | |
| | | domain, (semaphorin) 4A | | | | |
| 1.7900000 | SEMA4B | sema domain, | 10509 | NM_020210 | Hs.474935 | ENSG00000185033 |
| | | immunoglobulin domain (Ig), | | | | |
| | | transmembrane domain (TM) | | | | |
| | | and short cytoplasmic | | | | |
| | | domain, (semaphorin) 4B | | | | |
| −1.6393443 | SEPT7P2 | septin 7 pseudogene 2 | 641977 | NR_024271 | Hs.723477 | ENSG00000214765 |
| 1.5500000 | SQSTM1 | sequestosome 1 | 8878 | NM_001142298 | Hs.724025 | ENSG00000161011 |
| 1.6700000 | SHMT2 | serine | 6472 | NM_001166356 | Hs.741179 | ENSG00000182199 |
| | | hydroxymethyltransferase 2 | | | | |
| | | (mitochondrial) | | | | |
| 2.0700000 | SPINT1 | serine peptidase inhibitor, | 6692 | NM_001032367 | Hs.233950 | ENSG00000166145 |
| | | Kunitz type 1 | | | | |
| −1.4925373 | SRSF10 | serine/arginine-rich splicing | 10772 | NM_001191005 | Hs.3530 | ENSG00000188529 |
| | | factor 10 | | | | |
| −1.8181818 | SRSF11 | serine/arginine-rich splicing | 9295 | NM_001190987 | Hs.479693 | ENSG00000116754 |
| | | factor 11 | | | | |
| −1.8181818 | SRSF4 | serine/arginine-rich splicing | 6429 | NM_005626 | Hs.469970 | ENSG00000116350 |
| | | factor 4 | | | | |
| −2.2727273 | SAA2 | serum amyloid A2 | 6289 | NM_001127380 | Hs.731376 | ENSG00000134339 |
| −1.7241379 | SMYD4 | SET and MYND domain | 114826 | NM_052928 | Hs.514602 | ENSG00000186532 |
| | | containing 4 | | | | |
| −1.4492754 | SETMAR | SET domain and mariner | 6419 | NM_001243723 | Hs.475300 | ENSG00000170364 |
| | | transposase fusion gene | | | | |
| −1.8867925 | SCML4 | sex comb on midleg-like 4 | 256380 | NM_001286408 | Hs.486109 | ENSG00000146285 |
| | | (Drosophila) | | | | |
| −2.1739130 | STAC2 | SH3 and cysteine rich domain 2 | 342667 | NM_198993 | Hs.145068 | ENSG00000141750 |
| −1.4925373 | SH3BP5-AS1 | SH3BP5 antisense RNA 1 | 100505696 | NR_046084 | Hs.745026 | ENSG00000224660 |
| −2.0408163 | SHANK2-AS3 | SHANK2 antisense RNA 3 | 220070 | NM_145308 | Hs.326766 | ENSG00000171671 |
| −2.0408163 | LOC100420587 | SHC SH2-domain binding | 100420587 | NR_110759 | Hs.569956 | ENSG00000267243 |
| | | protein 1 pseudogene | | | | |
| −3.0303030 | SHISA9 | shisa family member 9 | 729993 | NM_001145204 | Hs.130661 | ENSG00000237515 |
| −2.2222222 | SGOL1 | shugoshin-like 1 (S. pombe) | 151648 | NM_001012409 | Hs.105153 | ENSG00000129810 |
| 1.9100000 | SIGLEC9 | sialic acid binding Ig-like | 27180 | NM_001198558 | Hs.245828 | ENSG00000129450 |
| | | lectin 9 | | | | |
| 1.6700000 | SPCS1 | signal peptidase complex | 28972 | NM_014041 | Hs.11125 | ENSG00000114902 |
| | | subunit 1 homolog (S. | | | | |
| | | cerevisiae) | | | | |
| 1.5600000 | SRP9 | signal recognition particle | 6726 | NM_001130440 | Hs.511425 | ENSG00000143742 |
| | | 9 kDa | | | | |
| 1.7700000 | SSR2 | signal sequence receptor, beta | 6746 | NM_003145 | Hs.74564 | ENSG00000163479 |
| | | (translocon-associated protein | | | | |
| | | beta) | | | | |
| 1.4000000 | STAT5B | signal transducer and | 6777 | NM_012448 | Hs.595276 | ENSG00000173757 |
| | | activator of transcription 5B | | | | |
| 1.9900000 | SMDT1 | single-pass membrane protein | 91689 | NM_033318 | Hs.306083 | ENSG00000183172 |
| | | with aspartate-rich tail 1 | | | | |
| 2.2700000 | SIRT2 | sirtuin 2 | 22933 | NM_001193286 | Hs.466693 | ENSG00000068903 |
| −1.5384615 | SIRT3 | sirtuin 3 | 23410 | NM_001017524 | Hs.716456 | ENSG00000142082 |
| −2.2222222 | SIX4 | SIX homeobox 4 | 51804 | NM_017420 | Hs.97849 | ENSG00000100625 |
| 1.4600000 | SSNA1 | Sjogren syndrome nuclear | 8636 | NM_003731 | Hs.530314 | ENSG00000176101 |
| | | autoantigen 1 | | | | |
| −2.1739130 | SLFNL1-AS1 | SLFNL1 antisense RNA 1 | 100507178 | NR_037868 | Hs.660056 | |
| 2.7900000 | SLMO2-ATP5E | SLMO2-ATP5E readthrough | 100533975 | NR_037929 | Hs.656865 | ENSG00000236105 |
| 2.9700000 | SLX1A | SLX1 structure-specific | 548593 | NM_001014999 | Hs.729791 | ENSG00000132207 |
| | | endonuclease subunit | | | | |
| | | homolog A (S. cerevisiae) | | | | |
| 2.9700000 | SLX1B | SLX1 structure-specific | 79008 | NM_024044 | Hs.728161 | ENSG00000181625 |
| | | endonuclease subunit | | | | |
| | | homolog B (S. cerevisiae) | | | | |
| −2.0000000 | SNIP1 | Smad nuclear interacting | 79753 | NM_024700 | Hs.47232 | ENSG00000163877 |
| | | protein 1 | | | | |
| 3.2400000 | SCARNA20 | small Cajal body-specific | 677681 | NR_002999 | | ENSG00000252577 |
| | | RNA 20 | | | | |
| −2.1739130 | SGSM1 | small G protein signaling | 129049 | NM_001039948 | Hs.474397 | ENSG00000167037 |
| | | modulator 1 | | | | |
| −2.8571429 | SNHG4 | small nucleolar RNA host | 724102 | NR_003141 | Hs.268939 | ENSG00000015479 |
| | | gene 4 | | | | |
| 3.4900000 | SNORD16 | small nucleolar RNA, C/D | 595097 | NR_002440 | Hs.739034 | ENSG00000174444 |
| | | box 16 | | | | |
| 4.9900000 | SNORD56 | small nucleolar RNA, C/D | 26793 | NR_002739 | | ENSG00000229686 |
| | | box 56 | | | | |
| −2.3809524 | SMG1P7 | SMG1 pseudogene 7 | 100506060 | NR_033959 | Hs.655258 | ENSG00000261556 |
| −1.2987013 | SMU1 | smu-1 suppressor of mec-8 | 55234 | NM_018225 | Hs.655351 | ENSG00000122692 |
| | | and unc-52 homolog (C. | | | | |
| | | elegans) | | | | |
| 2.4700000 | SNAI3 | snail family zinc finger 3 | 333929 | NM_178310 | Hs.673548 | ENSG00000185669 |
| 2.2800000 | SCN1B | sodium channel, voltage | 6324 | NM_001037 | Hs.436646 | ENSG00000105711 |
| | | gated, type I beta subunit | | | | |
| −2.2222222 | SLC14A2 | solute carrier family 14 (urea | 8170 | NM_001242692 | Hs.710927 | ENSG00000132874 |
| | | transporter), member 2 | | | | |
| −1.9607843 | SLC15A1 | solute carrier family 15 | 6564 | NM_005073 | Hs.436893 | ENSG00000088386 |
| | | (oligopeptide transporter), | | | | |
| | | member 1 | | | | |
| 1.8100000 | SLC16A5 | solute carrier family 16 | 9121 | NM_001271765 | Hs.592095 | ENSG00000170190 |
| | | (monocarboxylate | | | | |
| | | transporter), member 5 | | | | |
| 1.8800000 | SLC19A1 | solute carrier family 19 | 6573 | NM_001205206 | Hs.84190 | ENSG00000173638 |
| | | (folate transporter), member 1 | | | | |
| 1.3400000 | SLC25A5 | solute carrier family 25 | 292 | NM_001152 | Hs.632282 | ENSG00000005022 |
| | | (mitochondrial carrier; | | | | |
| | | adenine nucleotide | | | | |
| | | translocator), member 5 | | | | |
| 2.1000000 | SLC25A1 | solute carrier family 25 | 6576 | NM_001256534 | Hs.111024 | ENSG00000100075 |
| | | (mitochondrial carrier; citrate | | | | |
| | | transporter), member 1 | | | | |
| −2.3809524 | SLC25A15 | solute carrier family 25 | 10166 | NM_014252 | Hs.646645 | ENSG00000102743 |
| | | (mitochondrial carrier; | | | | |
| | | ornithine transporter) member 15 | | | | |
| 1.8400000 | SLC25A11 | solute carrier family 25 | 8402 | NM_001165417 | Hs.184877 | ENSG00000108528 |
| | | (mitochondrial carrier; | | | | |
| | | oxoglutarate carrier), member 11 | | | | |
| 1.6100000 | SLC25A3 | solute carrier family 25 | 5250 | NM_002635 | Hs.290404 | ENSG00000075415 |
| | | (mitochondrial carrier; | | | | |
| | | phosphate carrier), member 3 | | | | |
| −1.5873016 | SLC25A32 | solute carrier family 25 | 81034 | NM_030780 | Hs.532265 | ENSG00000164933 |
| | | (mitochondrial folate carrier), | | | | |
| | | member 32 | | | | |
| 1.6500000 | SLC25A38 | solute carrier family 25, | 54977 | NM_017875 | Hs.369615 | ENSG00000144659 |
| | | member 38 | | | | |
| 1.4800000 | SLC27A1 | solute carrier family 27 (fatty | 376497 | NM_198580 | Hs.363138 | ENSG00000130304 |
| | | acid transporter), member 1 | | | | |
| −2.8571429 | SLC28A2 | solute carrier family 28 | 9153 | NM_004212 | Hs.367833 | ENSG00000137860 |
| | | (concentrative nucleoside | | | | |
| | | transporter), member 2 | | | | |
| −1.7857143 | SLC31A1 | solute carrier family 31 | 1317 | NM_001859 | Hs.532315 | ENSG00000136868 |
| | | (copper transporter), member 1 | | | | |
| 1.6600000 | SLC35A1 | solute carrier family 35 | 10559 | NM_001168398 | Hs.423163 | ENSG00000164414 |
| | | (CMP-sialic acid transporter), | | | | |
| | | member A1 | | | | |
| 1.6700000 | SLC35A5 | solute carrier family 35, | 55032 | NM_017945 | Hs.237480 | ENSG00000138459 |
| | | member A5 | | | | |
| −2.1739130 | SLC36A2 | solute carrier family 36 | 153201 | NM_181776 | Hs.483877 | ENSG00000186335 |
| | | (proton/amino acid | | | | |
| | | symporter), member 2 | | | | |
| −2.0000000 | SLC38A1 | solute carrier family 38, | 81539 | NM_001077484 | Hs.533770 | ENSG00000111371 |
| | | member 1 | | | | |
| 1.5500000 | SLC39A3 | solute carrier family 39 (zinc | 29985 | NM_144564 | Hs.515046 | ENSG00000141873 |
| | | transporter), member 3 | | | | |
| −1.7857143 | SLC4A8 | solute carrier family 4, | 9498 | NM_001039960 | Hs.4749 | ENSG00000050438 |
| | | sodium bicarbonate | | | | |
| | | cotransporter, member 8 | | | | |
| 2.1500000 | SLC40A1 | solute carrier family 40 (iron- | 30061 | NM_014585 | Hs.643005 | ENSG00000138449 |
| | | regulated transporter), | | | | |
| | | member 1 | | | | |
| −1.7857143 | SLC41A2 | solute carrier family 41 | 84102 | NM_032148 | Hs.577463 | ENSG00000136052 |
| | | (magnesium transporter), | | | | |
| | | member 2 | | | | |
| 1.7100000 | SLC41A3 | solute carrier family 41, | 54946 | NM_001008485 | Hs.573007 | ENSG00000114544 |
| | | member 3 | | | | |
| −2.2727273 | SLC44A4 | solute carrier family 44, | 80736 | NM_001178044 | Hs.335355 | ENSG00000204385 |
| | | member 4 | | | | |
| −2.3809524 | SLC5A5 | solute carrier family 5 | 6528 | NM_000453 | Hs.584804 | ENSG00000105641 |
| | | (sodium/iodide | | | | |
| | | cotransporter), member 5 | | | | |
| −2.0408163 | SLC7A5P2 | solute carrier family 7 (amino | 387254 | NR_002594 | Hs.448808 | |
| | | acid transporter light chain, L | | | | |
| | | system), member 5 pseudogene 2 | | | | |
| −2.1276596 | LOC284379 | solute carrier family 7 | 284379 | NR_002938 | Hs.631571 | ENSG00000268864 |
| | | (cationic amino acid | | | | |
| | | transporter, y+ system), | | | | |
| | | member 3 pseudogene | | | | |
| −1.7241379 | SLC7A14 | solute carrier family 7, | 57709 | NM_020949 | Hs.596660 | ENSG00000013293 |
| | | member 14 | | | | |
| −2.6315789 | SLC9A4 | solute carrier family 9, | 389015 | NM_001011552 | Hs.447686 | ENSG00000180251 |
| | | subfamily A (NHE4, cation | | | | |
| | | proton antiporter 4), member 4 | | | | |
| −1.4925373 | SLC9A7 | solute carrier family 9, | 84679 | NM_001257291 | Hs.91389 | ENSG00000065923 |
| | | subfamily A (NHE7, cation | | | | |
| | | proton antiporter 7), member 7 | | | | |
| 1.9100000 | SORBS3 | sorbin and SH3 domain | 10174 | NM_001018003 | Hs.528572 | ENSG00000120896 |
| | | containing 3 | | | | |
| 1.4800000 | SNX12 | sorting nexin 12 | 29934 | NM_001256185 | Hs.260750 | ENSG00000147164 |
| 1.6700000 | SNX17 | sorting nexin 17 | 9784 | NM_001267059 | Hs.278569 | ENSG00000115234 |
| −2.3255814 | SOX9-AS1 | SOX9 antisense RNA 1 | 400618 | NR_103737 | Hs.657374 | ENSG00000234899 |
| 1.5900000 | SPG21 | spastic paraplegia 21 (autosomal | 51324 | NM_001127889 | Hs.242458 | ENSG00000090487 |
| | | recessive, Mast syndrome) | | | | |
| −1.3888889 | SPAST | spastin | 6683 | NM_014946 | Hs.468091 | ENSG00000021574 |
| −3.2258065 | SPC25 | SPC25, NDC80 kinetochore | 57405 | NM_020675 | Hs.421956 | ENSG00000152253 |
| | | complex component | | | | |
| −1.6666667 | SPDYE5 | speedy/RINGO cell cycle | 442590 | NM_001099435 | Hs.632298 | ENSG00000170092 |
| | | regulator family member E5 | | | | |
| −1.9230769 | SPATS2 | spermatogenesis associated, | 65244 | NM_001293285 | Hs.654826 | ENSG00000123352 |
| | | serine-rich 2 | | | | |
| −2.3809524 | SKA1 | spindle and kinetochore | 220134 | NM_001039535 | Hs.134726 | ENSG00000154839 |
| | | associated complex subunit 1 | | | | |
| 1.5200000 | SF3A2 | splicing factor 3a, subunit 2, | 8175 | NM_007165 | Hs.115232 | ENSG00000104897 |
| | | 66 kDa | | | | |
| −2.0833333 | SREK1 | splicing regulatory | 140890 | NM_001077199 | Hs.49367 | ENSG00000153914 |
| | | glutamine/lysine-rich protein 1 | | | | |
| −1.8867925 | SPRED1 | sprouty-related, EVH1 | 161742 | NM_152594 | Hs.525781 | ENSG00000166068 |
| | | domain containing 1 | | | | |
| −1.9607843 | SRRM2-AS1 | SRRM2 antisense RNA 1 | 100128788 | NR_027274 | Hs.311208 | ENSG00000205913 |
| −2.3255814 | STAU2-AS1 | STAU2 antisense RNA 1 | 100128126 | NR_038406 | Hs.679921 | ENSG00000253302 |
| −1.9230769 | STEAP2 | STEAP family member 2, | 261729 | NM_001040665 | Hs.489051 | ENSG00000157214 |
| | | metalloreductase | | | | |
| −3.1250000 | SCD5 | stearoyl-CoA desaturase 5 | 79966 | NM_001037582 | Hs.379191 | ENSG00000145284 |
| −1.8867925 | STAR | steroidogenic acute regulatory | 6770 | NM_000349 | Hs.521535 | ENSG00000147465 |
| | | protein | | | | |
| −2.0000000 | STRIP2 | striatin interacting protein 2 | 57464 | NM_001134336 | Hs.489988 | ENSG00000128578 |
| 1.9000000 | SDHAF1 | succinate dehydrogenase | 644096 | NM_001042631 | Hs.356460 | ENSG00000205138 |
| | | complex assembly factor 1 | | | | |
| 1.5200000 | SDHB | succinate dehydrogenase | 6390 | NM_003000 | Hs.465924 | ENSG00000117118 |
| | | complex, subunit B, iron | | | | |
| | | sulfur (Ip) | | | | |
| −2.2727273 | SUMO1P3 | SUMO1 pseudogene 3 | 474338 | NR_002190 | Hs.621179 | |
| | | (functional) | | | | |
| 1.5200000 | SUV420H2 | suppressor of variegation 4- | 84787 | NM_032701 | Hs.590982 | ENSG00000133247 |
| | | 20 homolog 2 (Drosophila) | | | | |
| −1.9230769 | SFTPB | surfactant protein B | 6439 | NM_000542 | Hs.512690 | ENSG00000168878 |
| −1.4285714 | SUGP2 | SURP and G patch domain | 10147 | NM_001017392 | Hs.515271 | ENSG00000064607 |
| | | containing 2 | | | | |
| −1.7857143 | SWSAP1 | SWIM-type zinc finger 7 | 126074 | NM_175871 | Hs.631619 | ENSG00000173928 |
| | | associated protein 1 | | | | |
| 1.7300000 | SYTL1 | synaptotagmin-like 1 | 84958 | NM_001193308 | Hs.469175 | ENSG00000142765 |
| −1.4492754 | SS18 | synovial sarcoma | 6760 | NM_001007559 | Hs.129261 | ENSG00000141380 |
| | | translocation, chromosome 18 | | | | |
| −2.0000000 | TAF8 | TAF8 RNA polymerase II, | 129685 | NM_138572 | Hs.520122 | ENSG00000137413 |
| | | TATA box binding protein | | | | |
| | | (TBP)-associated factor, 43 kDa | | | | |
| −1.7543860 | TBC1D24 | TBC1 domain family, member 24 | 57465 | NM_001199107 | Hs.353087 | ENSG00000162065 |
| −2.0833333 | TBCCD1 | TBCC domain containing 1 | 55171 | NM_001134415 | Hs.518469 | ENSG00000113838 |
| 1.8600000 | TGDS | TDP-glucose 4,6-dehydratase | 23483 | NM_001304430 | Hs.12393 | ENSG00000088451 |
| −2.5641026 | TEX101 | testis expressed 101 | 83639 | NM_001130011 | Hs.97978 | ENSG00000131126 |
| −1.3698630 | TTC21B | tetratricopeptide repeat | 79809 | NM_024753 | Hs.310672 | ENSG00000123607 |
| | | domain 21B | | | | |
| 1.7100000 | THAP11 | THAP domain containing 11 | 57215 | NM_020457 | Hs.632200 | ENSG00000168286 |
| −1.8181818 | THEM4 | thioesterase superfamily | 117145 | NM_053055 | Hs.164070 | ENSG00000159445 |
| | | member 4 | | | | |
| 1.6900000 | TREX1 | three prime repair | 11277 | NM_007248 | Hs.707026 | ENSG00000213689 |
| | | exonuclease 1 | | | | |
| 1.3800000 | TBXAS1 | thromboxane A synthase 1 | 6916 | NM_001061 | Hs.520757 | ENSG00000059377 |
| | | (platelet) | | | | |
| −2.1276596 | TIGD1 | tigger transposable element | 200765 | NM_145702 | Hs.211823 | ENSG00000221944 |
| | | derived 1 | | | | |
| −2.6315789 | TLCD2 | TLC domain containing 2 | 727910 | NM_001164407 | Hs.531005 | ENSG00000185561 |
| −2.1739130 | TLR8-AS1 | TLR8 antisense RNA 1 | 349408 | NR_030727 | Hs.685035 | ENSG00000233338 |
| −1.6949153 | THRIL | TNF and HNRNPL related | 102659353 | NR_110375 | Hs.596464 | |
| | | immunoregulatory long non- | | | | |
| | | coding RNA | | | | |
| −1.6129032 | TRAF3IP1 | TNF receptor-associated | 26146 | NM_001139490 | Hs.631898 | ENSG00000204104 |
| | | factor 3 interacting protein 1 | | | | |
| −2.1739130 | TNFAIP8L2- | TNFAIP8L2-SCNM1 | 100534012 | NM_001204848 | Hs.732060 | ENSG00000163156 |
| | SCNM1 | readthrough | | | | |
| 1.4400000 | TOLLIP | toll interacting protein | 54472 | NM_019009 | Hs.368527 | ENSG00000078902 |
| −1.6949153 | TLR10 | toll-like receptor 10 | 81793 | NM_001017388 | Hs.120551 | ENSG00000174123 |
| 1.3900000 | TOR1A | torsin family 1, member A | 1861 | NM_000113 | Hs.534312 | ENSG00000136827 |
| | | (torsin A) | | | | |
| 2.0000000 | TOR2A | torsin family 2, member A | 27433 | NM_001085347 | Hs.444106 | ENSG00000160404 |
| −1.4925373 | TLK2 | tousled-like kinase 2 | 11011 | NM_001112707 | Hs.445078 | ENSG00000146872 |
| −2.3809524 | TRAF3IP2 | TRAF3 interacting protein 2 | 10758 | NM_001164281 | Hs.561514 | ENSG00000056972 |
| −1.8181818 | TRAF3IP2-AS1 | TRAF3IP2 antisense RNA 1 | 643749 | NR_034108 | Hs.486228 | ENSG00000231889 |
| 1.6800000 | TECR | trans-2,3-enoyl-CoA | 9524 | NM_004868 | Hs.515642 | ENSG00000099797 |
| | | reductase | | | | |
| −1.4492754 | TCERG1 | transcription elongation | 10915 | NM_001040006 | Hs.443465 | ENSG00000113649 |
| | | regulator 1 | | | | |
| −1.8518519 | TVP23A | trans-golgi network vesicle | 780776 | NM_001079512 | Hs.371576 | ENSG00000166676 |
| | | protein 23 homolog A (S. | | | | |
| | | cerevisiae) | | | | |
| −1.9230769 | TVP23C | trans-golgi network vesicle | 201158 | NM_001135036 | Hs.164595 | ENSG00000175106 |
| | | protein 23 homolog C (S. | | | | |
| | | cerevisiae) | | | | |
| −1.8518519 | TRPM6 | transient receptor potential | 140803 | NM_001177310 | Hs.272225 | ENSG00000119121 |
| | | cation channel, subfamily M, | | | | |
| | | member 6 | | | | |
| −2.3255814 | TRPV1 | transient receptor potential | 7442 | NM_018727 | Hs.579217 | ENSG00000196689 |
| | | cation channel, subfamily V, | | | | |
| | | member 1 | | | | |
| −1.4084507 | TIMM10B | translocase of inner | 26515 | NM_012192 | Hs.54943 | ENSG00000132286 |
| | | mitochondrial membrane 10 | | | | |
| | | homolog B (yeast) | | | | |
| 2.2500000 | TSPO | translocator protein (18 kDa) | 706 | NM_000714 | Hs.202 | ENSG00000100300 |
| −2.1276596 | TMIGD2 | transmembrane and | 126259 | NM_001169126 | Hs.263928 | ENSG00000167664 |
| | | immunoglobulin domain | | | | |
| | | containing 2 | | | | |
| 1.3600000 | TMBIM6 | transmembrane BAX | 7009 | NM_001098576 | Hs.743965 | ENSG00000139644 |
| | | inhibitor motif containing 6 | | | | |
| −1.8181818 | TPTE2P1 | transmembrane | 646405 | NM_001126062 | Hs.620592 | |
| | | phosphoinositide 3- | | | | |
| | | phosphatase and tensin | | | | |
| | | homolog 2 pseudogene 1 | | | | |
| 1.8200000 | TMEM120A | transmembrane protein 120A | 83862 | NM_031925 | Hs.488835 | ENSG00000189077 |
| −1.9230769 | TMEM120B | transmembrane protein 120B | 144404 | NM_001080825 | Hs.644504 | ENSG00000188735 |
| 2.3900000 | TMEM141 | transmembrane protein 141 | 85014 | NM_032928 | Hs.356744 | ENSG00000244187 |
| 1.9500000 | TMEM14C | transmembrane protein 14C | 51522 | NM_001165258 | Hs.519557 | ENSG00000111843 |
| 1.7600000 | TMEM150A | transmembrane protein 150A | 129303 | NM_001031738 | Hs.591559 | ENSG00000168890 |
| 2.3300000 | TMEM160 | transmembrane protein 160 | 54958 | NM_017854 | Hs.105606 | ENSG00000130748 |
| 1.5400000 | TMEM165 | transmembrane protein 165 | 55858 | NM_018475 | Hs.479766 | ENSG00000134851 |
| 1.6300000 | TMEM179B | transmembrane protein 179B | 374395 | NM_199337 | Hs.381134 | ENSG00000185475 |
| −1.5625000 | TMEM181 | transmembrane protein 181 | 57583 | NM_020823 | Hs.99145 | ENSG00000146433 |
| −3.0303030 | TMEM212 | transmembrane protein 212 | 389177 | NM_001164436 | Hs.642307 | ENSG00000186329 |
| 1.3600000 | TMEM214 | transmembrane protein 214 | 54867 | NM_001083590 | Hs.533934 | ENSG00000119777 |
| 1.6000000 | TMEM230 | transmembrane protein 230 | 29058 | NM_001009923 | Hs.472024 | ENSG00000089063 |
| 2.5700000 | TMEM256 | transmembrane protein 256 | 254863 | NM_152766 | Hs.730789 | ENSG00000205544 |
| −1.5625000 | TMEM38A | transmembrane protein 38A | 79041 | NM_024074 | Hs.436068 | ENSG00000072954 |
| −1.9607843 | TMEM41B | transmembrane protein 41B | 440026 | NM_001165030 | Hs.594563 | ENSG00000166471 |
| 1.5000000 | TMEM43 | transmembrane protein 43 | 79188 | NM_024334 | Hs.517817 | ENSG00000170876 |
| −1.2987013 | TRIM38 | tripartite motif containing 38 | 10475 | NM_006355 | Hs.584851 | ENSG00000112343 |
| −2.3255814 | TRIM45 | tripartite motif containing 45 | 80263 | NM_001145635 | Hs.301526 | ENSG00000134253 |
| −1.9607843 | TPM3P9 | tropomyosin 3 pseudogene 9 | 147804 | NM_001010856 | Hs.433293 | ENSG00000241015 |
| −2.3255814 | TSIX | TSIX transcript, XIST | 9383 | NR_003255 | Hs.529901 | ENSG00000270641 |
| | | antisense RNA | | | | |
| −1.4084507 | TSPYL1 | TSPY-like 1 | 7259 | NM_003309 | Hs.458358 | ENSG00000189241 |
| 1.7700000 | TTLL12 | tubulin tyrosine ligase-like | 23170 | NM_015140 | Hs.517670 | ENSG00000100304 |
| | | family member 12 | | | | |
| −2.4390244 | TUBA3FP | tubulin, alpha 3f, pseudogene | 113691 | NR_003608 | Hs.585006 | ENSG00000161149 |
| 1.5800000 | TUBB | tubulin, beta class I | 203068 | NM_001293212 | Hs.636480 | ENSG00000196230 |
| −1.7241379 | TUFT1 | tuftelin 1 | 7286 | NM_001126337 | Hs.489922 | ENSG00000143367 |
| −2.3255814 | TP53AIP1 | tumor protein p53 regulated | 63970 | NM_001195194 | Hs.160953 | ENSG00000120471 |
| | | apoptosis inducing protein 1 | | | | |
| 2.0000000 | TPRG1L | tumor protein p63 regulated | 127262 | NM_182752 | Hs.20529 | ENSG00000158109 |
| | | 1-like | | | | |
| −2.0000000 | TSG1 | tumor suppressor TSG1 | 643432 | NR_015362 | Hs.509936 | |
| 1.8000000 | TWF2 | twinfilin actin-binding protein 2 | 11344 | NM_007284 | Hs.436439 | ENSG00000247596 |
| −1.8518519 | TAT | tyrosine aminotransferase | 6898 | NM_000353 | Hs.161640 | ENSG00000198650 |
| 1.8200000 | UQCR10 | ubiquinol-cytochrome c | 29796 | NM_001003684 | Hs.284292 | ENSG00000184076 |
| | | reductase, complex III | | | | |
| | | subunit X | | | | |
| 1.4300000 | UBE3C | ubiquitin protein ligase E3C | 9690 | NM_014671 | Hs.118351 | ENSG00000009335 |
| 1.4500000 | USP21 | ubiquitin specific peptidase 21 | 27005 | NM_001014443 | Hs.8015 | ENSG00000143258 |
| −1.4492754 | USP42 | ubiquitin specific peptidase 42 | 84132 | NM_032172 | Hs.31856 | ENSG00000106346 |
| −1.6393443 | USP49 | ubiquitin specific peptidase 49 | 25862 | NM_001286554 | Hs.593575 | ENSG00000164663 |
| 1.7300000 | UBE2D1 | ubiquitin-conjugating enzyme E2D 1 | 7321 | NM_001204880 | Hs.129683 | ENSG00000072401 |
| −2.2222222 | UBE2Q2P1 | ubiquitin-conjugating enzyme E2Q | 388165 | NM_207382 | Hs.498348 | ENSG00000189136 |
| | | family member 2 pseudogene 1 | | | | |
| 1.4900000 | UBL5 | ubiquitin-like 5 | 59286 | NM_001048241 | Hs.534477 | ENSG00000198258 |
| −2.1739130 | UCKL1-AS1 | UCKL1 antisense RNA 1 | 100113386 | NR_027287 | Hs.551552 | |
| 1.6700000 | B3GALT6 | UDP-Gal:betaGal beta 1,3- | 126792 | NM_080605 | Hs.284284 | ENSG00000176022 |
| | | galactosyltransferase | | | | |
| | | polypeptide 6 | | | | |
| −2.9411765 | B3GNT6 | UDP-GlcNAc:betaGal beta- | 192134 | NM_138706 | Hs.352622 | ENSG00000198488 |
| | | 1,3-N- | | | | |
| | | acetylglucosaminyltransferase 6 | | | | |
| −3.7037037 | UGDH-AS1 | UGDH antisense RNA 1 | 100885776 | NR_047679 | Hs.640769 | ENSG00000249348 |
| 1.5300000 | ULK3 | unc-51 like kinase 3 | 25989 | NM_001099436 | Hs.513034 | ENSG00000140474 |
| −2.0833333 | LOC100128233 | uncharacterized LOC100128233 | 100128233 | NR_103769 | Hs.497323 | ENSG00000255002 |
| −2.6315789 | LOC100128288 | uncharacterized LOC100128288 | 100128288 | NR_024447 | Hs.549913 | |
| −2.0408163 | LOC100128398 | uncharacterized LOC100128398 | 100128398 | NR_036508 | Hs.655081 | ENSG00000176593 |
| −2.1276596 | LOC100129940 | uncharacterized LOC100129940 | 100129940 | NM_001292023 | Hs.685856 | ENSG00000197301 |
| −2.6315789 | LOC100130451 | uncharacterized LOC100130451 | 100130451 | NM_001242575 | | |
| −2.1739130 | LOC100131564 | uncharacterized LOC100131564 | 100131564 | NR_034089 | Hs.732666 | ENSG00000223745 |
| −3.0303030 | LOC100131626 | uncharacterized LOC100131626 | 100131626 | NR_046369 | Hs.721614 | |
| −2.4390244 | LOC100132077 | uncharacterized LOC100132077 | 100132077 | NR_033937 | Hs.679111 | ENSG00000232063 |
| −2.1276596 | LOC100190986 | uncharacterized LOC100190986 | 100190986 | NR_024456 | Hs.648439 | |
| −2.3809524 | LOC100287225 | uncharacterized LOC100287225 | 100287225 | NR_040074 | Hs.448920 | ENSG00000227115 |
| −2.0833333 | LOC100379224 | uncharacterized LOC100379224 | 100379224 | NR_033341 | Hs.585869 | ENSG00000186019 |
| 2.2200000 | LOC100505622 | uncharacterized LOC100505622 | 100505622 | NR_038332 | Hs.661761 | ENSG00000254887 |
| −3.2258065 | LOC100505817 | uncharacterized LOC100505817 | 100505817 | NR_038340 | Hs.197042 | ENSG00000261780 |
| −2.0833333 | LOC100506083 | uncharacterized LOC100506083 | 100506083 | NR_039997 | Hs.635008 | ENSG00000261777 |
| −2.2222222 | LOC100506085 | uncharacterized LOC100506085 | 100506085 | NR_037878 | Hs.649173 | ENSG00000248319 |
| −2.0408163 | LOC100506123 | uncharacterized LOC100506123 | 100506123 | NR_040097 | Hs.720604 | ENSG00000230606 |
| −1.7857143 | LOC100506472 | uncharacterized LOC100506472 | 100506472 | NR_040535 | Hs.729080 | |
| −2.0408163 | LOC100506688 | uncharacterized LOC100506688 | 100506688 | NM_001242737 | Hs.532063 | ENSG00000215246 |
| −1.9230769 | LOC100506746 | uncharacterized LOC100506746 | 100506746 | NR_038841 | Hs.657766 | ENSG00000163633 |
| −2.3809524 | LOC100996251 | uncharacterized LOC100996251 | 100996251 | NR_103777 | Hs.382067 | ENSG00000238198 |
| −2.3809524 | LOC100996351 | uncharacterized LOC100996351 | 100996351 | NR_110670 | | ENSG00000267551 |
| −3.0303030 | LOC101926889 | uncharacterized LOC101926889 | 101926889 | NR_109994 | Hs.585997 | |
| −2.2727273 | LOC101926928 | uncharacterized LOC101926928 | 101926928 | NR_110106 | Hs.638788 | ENSG00000258551 |
| −1.7543860 | LOC101926960 | uncharacterized LOC101926960 | 101926960 | NR_104635 | Hs.365692 | |
| −2.0833333 | LOC101927131 | uncharacterized LOC101927131 | 101927131 | NR_110907 | Hs.569654 | ENSG00000262999 |
| −1.9230769 | LOC101927181 | uncharacterized LOC101927181 | 101927181 | NR_108066 | Hs.288853 | ENSG00000175873 |
| −2.0833333 | LOC101927257 | uncharacterized LOC101927257 | 101927257 | NR_109965 | Hs.662725 | ENSG00000232564 |
| −2.3255814 | LOC101927274 | uncharacterized LOC101927274 | 101927274 | NR_110751 | Hs.591168 | ENSG00000249383 |
| −3.4482759 | LOC101927374 | uncharacterized LOC101927374 | 101927374 | NR_110133 | Hs.570644 | |
| −2.3809524 | LOC101927476 | uncharacterized LOC101927476 | 101927476 | NR_110386 | Hs.522607 | ENSG00000236393 |
| −1.3888889 | LOC101927550 | uncharacterized LOC101927550 | 101927550 | NR_110102 | Hs.636663 | ENSG00000242687 |
| −3.2258065 | LOC101927575 | uncharacterized LOC101927575 | 101927575 | NR_110995 | Hs.459826 | ENSG00000227463 |
| −1.7241379 | LOC101927666 | uncharacterized LOC101927666 | 101927666 | NR_110809 | Hs.552237 | ENSG00000266290 |
| −2.2222222 | LOC101927740 | uncharacterized LOC101927740 | 101927740 | NR_109890 | Hs.738721 | ENSG00000245812 |
| −2.5641026 | LOC101927797 | uncharacterized LOC101927797 | 101927797 | NR_109925 | Hs.551743 | ENSG00000224141 |
| −2.3255814 | LOC101927817 | uncharacterized LOC101927817 | 101927817 | NR_110931 | Hs.667942 | |
| −2.7027027 | LOC101927884 | uncharacterized LOC101927884 | 101927884 | NR_110281 | Hs.671110 | ENSG00000231172 |
| −2.2727273 | LOC101928103 | uncharacterized LOC101928103 | 101928103 | NR_110292 | Hs.665619 | ENSG00000229267 |
| −3.2258065 | LOC101928137 | uncharacterized LOC101928137 | 101928137 | NR_110130 | Hs.694666 | ENSG00000258123 |
| −2.4390244 | LOC101928254 | uncharacterized LOC101928254 | 101928254 | NR_110182 | Hs.571236 | ENSG00000219445 |
| −2.4390244 | LOC101928303 | uncharacterized LOC101928303 | 101928303 | NR_110698 | Hs.375067 | ENSG00000236155 |
| −2.2727273 | LOC101928567 | uncharacterized LOC101928567 | 101928567 | NR_110839 | Hs.569757 | ENSG00000237057 |
| −2.0000000 | LOC101928597 | uncharacterized LOC101928597 | 101928597 | NR_110091 | Hs.638942 | ENSG00000246394 |
| −2.3809524 | LOC101928674 | uncharacterized LOC101928674 | 101928674 | NR_110845 | Hs.637297 | ENSG00000266970 |
| −2.9411765 | LOC101928844 | uncharacterized LOC101928844 | 101928844 | NR_110740 | Hs.434577 | ENSG00000267709 |
| −3.0303030 | LOC101928936 | uncharacterized LOC101928936 | 101928936 | NR_110867 | Hs.533080 | |
| −1.8518519 | LOC101929144 | uncharacterized LOC101929144 | 101929144 | NR_110745 | Hs.531631 | ENSG00000261615 |
| −2.5641026 | LOC101929224 | uncharacterized LOC101929224 | 101929224 | NR_110787 | Hs.639369 | ENSG00000260088 |
| −2.3809524 | LOC101929259 | uncharacterized LOC101929259 | 101929259 | NR_120424 | Hs.638490 | |
| −2.5000000 | LOC101929486 | uncharacterized LOC101929486 | 101929486 | NR_109868 | Hs.548761 | ENSG00000233048 |
| −2.7027027 | LOC101929567 | uncharacterized LOC101929567 | 101929567 | NR_110257 | Hs.634706 | ENSG00000236008 |
| −2.3255814 | LOC101929586 | uncharacterized LOC101929586 | 101929586 | NR_120363 | Hs.569426 | ENSG00000259175 |
| −3.0303030 | LOC101929698 | uncharacterized LOC101929698 | 101929698 | NR_110619 | Hs.638392 | ENSG00000277301 |
| −1.9607843 | LOC101929767 | uncharacterized LOC101929767 | 101929767 | NR_110868 | Hs.640892 | ENSG00000267002 |
| −3.0303030 | LOC102467081 | uncharacterized LOC102467081 | 102467081 | NR_104662 | | ENSG00000240535 |
| −2.7027027 | LOC102723769 | uncharacterized LOC102723769 | 102723769 | NR_110761 | Hs.652926 | |
| −2.3255814 | LOC102724927 | uncharacterized LOC102724927 | 102724927 | NR_120311 | Hs.364739 | ENSG00000262185 |
| −2.0000000 | LOC151475 | uncharacterized LOC151475 | 151475 | NR_040038 | Hs.528154 | ENSG00000226125 |
| −2.1276596 | MGC32805 | uncharacterized LOC153163 | 153163 | NR_051996 | Hs.679757 | ENSG00000250328 |
| −2.1739130 | LOC284023 | uncharacterized LOC284023 | 284023 | NR_024349 | Hs.744470 | ENSG00000179859 |
| −2.1276596 | LOC284581 | uncharacterized LOC284581 | 284581 | NR_046097 | | |
| −2.2222222 | LOC284865 | uncharacterized LOC284865 | 284865 | NR_038460 | Hs.638498 | ENSG00000249923 |
| −2.2222222 | LOC284950 | uncharacterized LOC284950 | 284950 | NR_038888 | Hs.570227 | ENSG00000229498 |
| −2.7777778 | LOC286437 | uncharacterized LOC286437 | 286437 | NR_039980 | Hs.656786 | |
| −2.1276596 | LOC339166 | uncharacterized LOC339166 | 339166 | NR_040000 | Hs.736088 | ENSG00000179314 |
| −2.0000000 | LOC339803 | uncharacterized LOC339803 | 339803 | NR_036496 | Hs.252433 | ENSG00000212978 |
| −2.0408163 | LOC389641 | uncharacterized LOC389641 | 389641 | NR_033928 | Hs.591835 | ENSG00000246582 |
| −2.3809524 | FLJ42102 | uncharacterized LOC399923 | 399923 | NM_001001680 | Hs.128191 | ENSG00000172900 |
| −2.2222222 | LOC400958 | uncharacterized LOC400958 | 400958 | NR_036586 | Hs.591565 | ENSG00000237638 |
| −2.3809524 | LOC401052 | uncharacterized LOC401052 | 401052 | NM_001008737 | Hs.662766 | |
| −1.4705882 | LOC401320 | uncharacterized LOC401320 | 401320 | NR_038889 | Hs.561708 | |
| −2.4390244 | FLJ31662 | uncharacterized LOC440594 | 440594 | NR_033966 | Hs.514123 | ENSG00000233907 |
| −2.3809524 | FLJ31104 | uncharacterized LOC441072 | 441072 | NR_102755 | Hs.482141 | ENSG00000227908 |
| −2.9411765 | LOC643406 | uncharacterized LOC643406 | 643406 | NM_175877 | Hs.431161 | ENSG00000230563 |
| −3.0303030 | LOC644919 | uncharacterized LOC644919 | 644919 | NR_109757 | Hs.434414 | |
| −4.3478261 | LOC728752 | uncharacterized LOC728752 | 728752 | NR_036504 | Hs.729762 | ENSG00000267309 |
| −2.0408163 | LOC729732 | uncharacterized LOC729732 | 729732 | NR_047662 | Hs.322761 | |
| −2.1276596 | LOC731424 | uncharacterized LOC731424 | 731424 | NR_037867 | Hs.427740 | |
| −1.4084507 | DKFZP586I1420 | uncharacterized protein | 222161 | NR_002186 | Hs.112423 | ENSG00000235859 |
| | | DKFZp586I1420 | | | | |
| −2.4390244 | FLJ31356 | uncharacterized protein | 403150 | NR_103831 | Hs.562970 | ENSG00000229951 |
| | | FLJ31356 | | | | |
| −1.8518519 | UPK3B | uroplakin 3B | 80761 | NM_030570 | Hs.488861 | ENSG00000243566 |
| −2.2727273 | UTS2B | urotensin 2B | 257313 | NM_198152 | Hs.518492 | ENSG00000188958 |
| 1.6700000 | VPS28 | vacuolar protein sorting 28 | 51160 | NM_016208 | Hs.418175 | ENSG00000160948 |
| | | homolog (S. cerevisiae) | | | | |
| 1.8000000 | VPS51 | vacuolar protein sorting 51 | 738 | NM_013265 | Hs.277517 | ENSG00000149823 |
| | | homolog (S. cerevisiae) | | | | |
| 1.7100000 | VIPR1 | vasoactive intestinal peptide | 7433 | NM_001251882 | Hs.348500 | ENSG00000114812 |
| | | receptor 1 | | | | |
| −1.4705882 | CRK | v-crk avian sarcoma virus | 1398 | NM_005206 | Hs.461896 | ENSG00000167193 |
| | | CT 10 oncogene homolog | | | | |
| 1.4500000 | CRKL | v-crk avian sarcoma virus | 1399 | NM_005207 | Hs.5613 | ENSG00000099942 |
| | | CT 10 oncogene homolog-like | | | | |
| 1.8000000 | MYCL | v-myc avian myclocytomatosis | 4610 | NM_001033081 | Hs.437922 | ENSG00000116990 |
| | | viral oncogene lung carcinoma | | | | |
| | | derived homolog | | | | |
| −2.5641026 | VN1R2 | vomeronasal 1 receptor 2 | 317701 | NM_173856 | Hs.553684 | ENSG00000196131 |
| 1.7500000 | VBP1 | von Hippel-Lindau binding | 7411 | NM_001303543 | Hs.436803 | ENSG00000155959 |
| | | protein 1 | | | | |
| −2.7777778 | VSIG1 | V-set and immunoglobulin | 340547 | NM_001170553 | Hs.177164 | ENSG00000101842 |
| | | domain containing 1 | | | | |
| −2.5000000 | WFDC8 | WAP four-disulfide core | 90199 | NM_130896 | Hs.116128 | ENSG00000158901 |
| | | domain 8 | | | | |
| −1.5151515 | WHAMM | WAS protein homolog | 123720 | NM_001080435 | Hs.377360 | ENSG00000156232 |
| | | associated with actin, golgi | | | | |
| | | membranes and microtubules | | | | |
| 1.8600000 | WDTC1 | WD and tetratricopeptide | 23038 | NM_001276252 | Hs.469154 | ENSG00000142784 |
| | | repeats 1 | | | | |
| −1.7241379 | WDR55 | WD repeat domain 55 | 54853 | NM_017706 | Hs.286261 | ENSG00000120314 |
| 2.3100000 | WDR83OS | WD repeat domain 83 | 51398 | NM_016145 | Hs.108969 | ENSG00000105583 |
| | | opposite strand | | | | |
| −1.6949153 | WDR92 | WD repeat domain 92 | 116143 | NM_001256476 | Hs.631877 | ENSG00000243667 |
| 1.9000000 | WBP1 | WW domain binding protein 1 | 23559 | NM_012477 | Hs.516114 | ENSG00000239779 |
| −3.4482759 | XKR9 | XK, Kell blood group | 389668 | NM_001011720 | Hs.458938 | ENSG00000221947 |
| | | complex subunit-related | | | | |
| | | family, member 9 | | | | |
| −1.7241379 | XIAP | X-linked inhibitor of | 331 | NM_001167 | Hs.356076 | ENSG00000101966 |
| | | apoptosis, E3 ubiquitin | | | | |
| | | protein ligase | | | | |
| −1.8518519 | XRCC2 | X-ray repair complementing | 7516 | NM_005431 | Hs.647093 | ENSG00000196584 |
| | | defective repair in Chinese | | | | |
| | | hamster cells 2 | | | | |
| −1.9230769 | YES1 | YES proto-oncogene 1, Src | 7525 | NM_005433 | Hs.194148 | ENSG00000176105 |
| | | family tyrosine kinase | | | | |
| 1.3300000 | YIPF1 | Yip1 domain family, member 1 | 54432 | NM_018982 | Hs.11923 | ENSG00000058799 |
| 1.7800000 | YIPF4 | Yip1 domain family, member 4 | 84272 | NM_032312 | Hs.468099 | ENSG00000119820 |
| −1.4492754 | YAF2 | YY1 associated factor 2 | 10138 | NM_001012424 | Hs.649195 | ENSG00000015153 |
| 1.3900000 | YY1 | YY1 transcription factor | 7528 | NM_003403 | Hs.388927 | ENSG00000100811 |
| −1.7241379 | ZFP14 | ZFP14 zinc finger protein | 57677 | NM_001297619 | Hs.35524 | ENSG00000142065 |
| −2.0000000 | ZFP30 | ZFP30 zinc finger protein | 22835 | NM_014898 | Hs.716719 | ENSG00000120784 |
| −2.4390244 | ZFP91-CNTF | ZFP91-CNTF readthrough | 386607 | NM_170768 | Hs.524920 | ENSG00000255073 |
| | | (NMD candidate) | | | | |
| 1.8900000 | ZBTB45 | zinc finger and BTB domain | 84878 | NM_032792 | Hs.515662 | ENSG00000119574 |
| | | containing 45 | | | | |
| −1.8181818 | ZSCAN12 | zinc finger and SCAN | 9753 | NM_001039643 | Hs.134816 | ENSG00000158691 |
| | | domain containing 12 | | | | |
| −1.6949153 | ZSCAN2 | zinc finger and SCAN | 54993 | NM_001007072 | Hs.594023 | ENSG00000176371 |
| | | domain containing 2 | | | | |
| −2.3255814 | ZSCAN22 | zinc finger and SCAN | 342945 | NM_181846 | Hs.388162 | ENSG00000182318 |
| | | domain containing 22 | | | | |
| −2.1739130 | ZC3H12D | zinc finger CCCH-type | 340152 | NM_207360 | Hs.632618 | ENSG00000178199 |
| | | containing 12D | | | | |
| −1.5151515 | ZNF117 | zinc finger protein 117 | 51351 | NM_015852 | Hs.250693 | ENSG00000152926 |
| −1.7857143 | ZNF264 | zinc finger protein 264 | 9422 | NM_003417 | Hs.515634 | ENSG00000083844 |
| −1.7543860 | ZNF286A | zinc finger protein 286A | 57335 | NM_001130842 | Hs.585799 | ENSG00000187607 |
| −1.8181818 | ZNF286B | zinc finger protein 286B | 729288 | NM_001145045 | Hs.534279 | ENSG00000249459 |
| −1.8518519 | ZNF326 | zinc finger protein 326 | 284695 | NM_181781 | Hs.306221 | ENSG00000162664 |
| −2.0833333 | ZNF329 | zinc finger protein 329 | 79673 | NM_024620 | Hs.458377 | ENSG00000181894 |
| −1.5384615 | ZNF417 | zinc finger protein 417 | 147687 | NM_001297734 | Hs.567710 | ENSG00000173480 |
| 1.8500000 | ZNF428 | zinc finger protein 428 | 126299 | NM_182498 | Hs.99093 | ENSG00000131116 |
| −1.4084507 | ZNF44 | zinc finger protein 44 | 51710 | NM_001164276 | Hs.296731 | ENSG00000197857 |
| −2.8571429 | ZNF471 | zinc finger protein 471 | 57573 | NM_020813 | Hs.710590 | ENSG00000196263 |
| −2.5641026 | ZNF483 | zinc finger protein 483 | 158399 | NM_001007169 | Hs.660784 | ENSG00000173258 |
| −2.0000000 | ZNF490 | zinc finger protein 490 | 57474 | NM_020714 | Hs.655860 | ENSG00000188033 |
| −1.9607843 | ZNF527 | zinc finger protein 527 | 84503 | NM_032453 | Hs.590940 | ENSG00000189164 |
| −2.0833333 | ZNF528 | zinc finger protein 528 | 84436 | NM_032423 | Hs.662043 | ENSG00000167555 |
| −1.8867925 | ZNF548 | zinc finger protein 548 | 147694 | NM_001172773 | Hs.126905 | ENSG00000188785 |
| −2.0000000 | ZNF554 | zinc finger protein 554 | 115196 | NM_001102651 | Hs.307043 | ENSG00000172006 |
| −1.7857143 | ZNF562 | zinc finger protein 562 | 54811 | NM_001130031 | Hs.371107 | ENSG00000171466 |
| −2.1276596 | ZNF585B | zinc finger protein 585B | 92285 | NM_152279 | Hs.390568 | ENSG00000245680 |
| −1.4084507 | ZNF587 | zinc finger protein 587 | 84914 | NM_001204817 | Hs.642598 | ENSG00000198466 |
| −1.5151515 | ZNF621 | zinc finger protein 621 | 285268 | NM_001098414 | Hs.19977 | ENSG00000172888 |
| −1.2820513 | ZNF655 | zinc finger protein 655 | 79027 | NM_001009956 | Hs.599798 | ENSG00000197343 |
| −2.7777778 | LOC100131257 | zinc finger protein 655 | 100131257 | NR_034022 | Hs.551110 | |
| | | pseudogene | | | | |
| −1.8867925 | ZNF662 | zinc finger protein 662 | 389114 | NM_001134656 | Hs.720173 | ENSG00000182983 |
| −2.2222222 | ZNF665 | zinc finger protein 665 | 79788 | NM_024733 | Hs.745230 | ENSG00000197497 |
| −2.0833333 | ZNF677 | zinc finger protein 677 | 342926 | NM_182609 | Hs.20506 | ENSG00000197928 |
| −2.0000000 | ZNF713 | zinc finger protein 713 | 349075 | NM_182633 | Hs.660834 | ENSG00000178665 |
| −1.5384615 | ZNF714 | zinc finger protein 714 | 148206 | NM_182515 | Hs.729186 | ENSG00000160352 |
| −1.7857143 | ZNF737 | zinc finger protein 737 | 100129842 | NM_001159293 | Hs.515696 | ENSG00000237440 |
| −2.4390244 | ZNF761 | zinc finger protein 761 | 388561 | NM_001008401 | Hs.433293 | ENSG00000160336 |
| −2.2222222 | ZNF793 | zinc finger protein 793 | 390927 | NM_001013659 | Hs.568010 | ENSG00000188227 |
| −2.0000000 | ZNF814 | zinc finger protein 814 | 730051 | NM_001144989 | Hs.634143 | ENSG00000204514 |
| −1.9607843 | ZNF818P | zinc finger protein 818, | 390963 | NM_001001675 | Hs.444446 | ENSG00000269001 |
| | | pseudogene | | | | |
| −2.0408163 | ZNF850 | zinc finger protein 850 | 342892 | NM_001193552 | Hs.406307 | ENSG00000267041 |
| −2.7777778 | ZKSCAN3 | zinc finger with KRAB and | 80317 | NM_001242894 | Hs.380930 | ENSG00000189298 |
| | | SCAN domains 3 | | | | |
| −1.8518519 | ZCCHC4 | zinc finger, CCHC domain | 29063 | NM_024936 | Hs.278945 | ENSG00000168228 |
| | | containing 4 | | | | |
| 1.5800000 | ZFYVE21 | zinc finger, FYVE domain | 79038 | NM_001198953 | Hs.592322 | ENSG00000100711 |
| | | containing 21 | | | | |
| 1.8100000 | ZNHIT1 | zinc finger, HIT-type | 10467 | NM_006349 | Hs.211079 | ENSG00000106400 |
| | | containing 1 | | | | |
| −1.5625000 | ZMAT1 | zinc finger, matrin-type 1 | 84460 | NM_001011657 | Hs.496512 | ENSG00000166432 |
| −1.4705882 | ZSWIM7 | zinc finger, SWIM-type | 125150 | NM_001042697 | Hs.593985 | ENSG00000214941 |
| | | containing 7 | | | | |
| 2.1700000 | ZMYM6NB | ZMYM6 neighbor | 100506144 | NM_001195156 | Hs.533986 | ENSG00000243749 |
| −2.3255814 | ZNRF3-AS1 | ZNRF3 antisense RNA 1 | 100874123 | NR_046851 | Hs.674708 | ENSG00000177993 |
| −1.9230769 | ZYG11A | zyg-11 family member A, cell | 440590 | NM_001004339 | Hs.658458 | ENSG00000203995 |
| | | cycle regulator | | | | |
| −3.2258065 | LOC101928243 | Non-annotated gene | 101928243 | | | |
| −3.1250000 | OLMALINC | Oligodendrocyte Maturation- | 90271 | NR_026762 | | ENSG00000235823 |
| | | Associated Long Intergenic | | | | |
| | | Non-Coding RNA | | | | |
| −2.9411765 | LOC100506385 | long intergenic non-protein | | NR_038885/ | | ENSG00000234380 |
| | | coding RNA 1426 | | NR_038886 | | |
| −2.6315789 | LOC400644 | long intergenic non-protein | | NR_104164 | | ENSG00000266554 |
| | | coding RNA 1443 | | | | |
| −2.4390244 | HECTD2-AS1 | HECTD2 antisense RNA 1 | 100188947 | NR_024467 | | |
| −2.3809524 | LINC01530 | long intergenic non-protein | 729975 | NR_034159 | | |
| | | coding RNA 1530 | | | | |
| −2.3255814 | TCAF2 | TRPM8 Channel Associated | 285966 | | | ENSG00000170379 |
| | | Factor 2 | | | | |
| −2.3255814 | LOC100130954 | long intergenic non-protein | | NR_034016/ | | |
| | | coding RNA 1502 | | NR_034017/ | | |
| | | | | NR_109814 | | |
| −2.3255814 | RRP7B | Ribosomal RNA Processing 7 | 91695 | NR_002184 | | ENSG00000182841 |
| | | Homolog B, Pseudogene | | | | |
| −2.2222222 | P3H4 | Prolyl 3-Hydroxylase Family | 10609 | NM_006455 | | ENSG00000141696 |
| | | Member 4 | | | | |
| −2.1276596 | L3MBTL4-AS1 | L3MBTL4 antisense RNA 1 | 10192715 | | | ENSG00000264707 |
| −2.0408163 | SIRPG-AS1 | SIRPG antisense RNA 1 | 10192901 | NR_110090 | | ENSG00000237914 |
| −1.9607843 | RUNDC3A-AS1 | RUNDC3A antisense RNA 1 | 101926996 | NR_110802 | | ENSG00000267750 |
| −1.8518519 | SLC25A25-AS1 | SLC25A25 antisense RNA 1 | | NR_033374 | | |
| −1.7543860 | LINC01521 | long intergenic non-protein | 54944 | NR_120386 | | ENSG00000213888 |
| | | coding RNA 1521 | | | | |
| −1.6949153 | LOC100506747 | alpha-1,3-mannosyl- | 100506747 | NR_036557 | | ENSG00000234761 |
| | | glycoprotein 4-beta-N- | | | | |
| | | acetylglucosaminyltransferase- | | | | |
| | | like protein LOC641515 | | | | |
| −1.3698630 | RBSN | Rabenosyn, RAB Effector | 64145 | | | ENSG00000131381 |
| 2.1700000 | CPTP | ceramide-1-phosphate | 80772 | NM_001029885 | | ENSG00000224051 |
| | | transfer protein | | | | |

TABLE 7B
Molecular Mechanisms and Pathways Associated with Monocyte Subtype Genes

| UniqueID | Associated Pathways/Mechanism |
|---|---|
| ABCA9 | ABC transporters |
| ABCC9 | ABC transporters |
| RIPK1 | Acetylation and Deacetylation of RelA in The Nucleus, Ceramide Signaling Pathway, HIV- |
| | I Nef: negative effector of Fas and TNF, Induction of apoptosis through DR3 and DR4/5 |
| | Death Receptors, Keratinocyte Differentiation, MAPKinase Signaling Pathway, NF-kB |
| | Signaling Pathway, p38 MAPK Signaling Pathway, SODD/TNFR1 Signaling Pathway, |
| | TNF/Stress Related Signaling, TNFR1 Signaling Pathway, TNFR2 Signaling Pathway, |
| | Apoptosis, Cytosolic DNA-sensing pathway, Hepatitis C, RIG-I-like receptor signaling |
| | pathway, Toll-like receptor signaling pathway |
| RAC2 | Adherens junction, Axon guidance, B cell receptor signaling pathway, Chemokine |
| | signaling pathway, Colorectal cancer, Fc epsilon RI signaling pathway, Fc gamma R- |
| | mediated phagocytosis, Focal adhesion, Leukocyte transendothelial migration, MAPK |
| | signaling pathway, Natural killer cell mediated cytotoxicity, Pancreatic cancer, Pathways in |
| | cancer, Regulation of actin cytoskeleton, VEGF signaling pathway, Viral myocarditis, Wnt |
| | signaling pathway |
| YES1 | Adherens junction, Tight junction |
| GPLD1 | ADP-Ribosylation Factor, Glycosylphosphatidylinositol(GPI)-anchor biosynthesis |
| APOL1 | African trypanosomiasis |
| IDO1 | African trypanosomiasis, Metabolic pathways, Tryptophan metabolism |
| GLUD1 | Alanine, aspartate and glutamate metabolism, Arginine and proline metabolism, D- |
| | Glutamine and D-glutamate metabolism, Metabolic pathways, Nitrogen metabolism, |
| | Proximal tubule bicarbonate reclamation |
| ADSL | Alanine, aspartate and glutamate metabolism, Metabolic pathways, Purine metabolism |
| CAD | Alanine, aspartate and glutamate metabolism, Metabolic pathways, Pyrimidine metabolism |
| BMP7 | ALK in cardiac myocytes, Cytokine-cytokine receptor interaction, Hedgehog signaling |
| | pathway, TGF-beta signaling pathway |
| PLA2G4E | alpha-Linolenic acid metabolism, Arachidonic acid metabolism, Ether lipid metabolism, |
| | Fat digestion and absorption, Fc epsilon RI signaling pathway, Fc gamma R-mediated |
| | phagocytosis, Glycerophospholipid metabolism, GnRH signaling pathway, Linoleic acid |
| | metabolism, Long-term depression, MAPK signaling pathway, Metabolic pathways, |
| | Pancreatic secretion, Toxoplasmosis, Vascular smooth muscle contraction, VEGF signaling |
| | pathway |
| CYCS | Alzheimer's disease, Amyotrophic lateral sclerosis (ALS), Apoptosis, Colorectal cancer, |
| | Huntington's disease, p53 signaling pathway, Parkinson's disease, Pathways in cancer, |
| | Small cell lung cancer, Toxoplasmosis, Viral myocarditis |
| COX6B2 | Alzheimer's disease, Cardiac muscle contraction, Huntington's disease, Metabolic |
| | pathways, Oxidative phosphorylation, Parkinson's disease |
| COX8A | Alzheimer's disease, Cardiac muscle contraction, Huntington's disease, Metabolic |
| | pathways, Oxidative phosphorylation, Parkinson's disease |
| CYC1 | Alzheimer's disease, Cardiac muscle contraction, Huntington's disease, Metabolic |
| | pathways, Oxidative phosphorylation, Parkinson's disease |
| UQCR10 | Alzheimer's disease, Cardiac muscle contraction, Huntington's disease, Metabolic |
| | pathways, Oxidative phosphorylation, Parkinson's disease |
| ATP5B | Alzheimer's disease, Huntington's disease, Metabolic pathways, Oxidative phosphorylation, |
| | Parkinson's disease |
| ATP5D | Alzheimer's disease, Huntington's disease, Metabolic pathways, Oxidative phosphorylation, |
| | Parkinson's disease |
| ATP5G2 | Alzheimer's disease, Huntington's disease, Metabolic pathways, Oxidative phosphorylation, |
| | Parkinson's disease |
| NDUFA7 | Alzheimer's disease, Huntington's disease, Metabolic pathways, Oxidative phosphorylation, |
| | Parkinson's disease |
| NDUFB6 | Alzheimer's disease, Huntington's disease, Metabolic pathways, Oxidative phosphorylation, |
| | Parkinson's disease |
| NDUFB7 | Alzheimer's disease, Huntington's disease, Metabolic pathways, Oxidative phosphorylation, |
| | Parkinson's disease |
| NDUFB8 | Alzheimer's disease, Huntington's disease, Metabolic pathways, Oxidative phosphorylation, |
| | Parkinson's disease |
| NDUFC1 | Alzheimer's disease, Huntington's disease, Metabolic pathways, Oxidative phosphorylation, |
| | Parkinson's disease |
| NDUFS7 | Alzheimer's disease, Huntington's disease, Metabolic pathways, Oxidative phosphorylation, |
| | Parkinson's disease |
| NDUFB11 | Alzheimer's disease, Huntington's disease, Oxidative phosphorylation, Parkinson's disease |
| GMPPA | Amino sugar and nucleotide sugar metabolism, Fructose and mannose metabolism, |
| | Metabolic pathways |
| GNE | Amino sugar and nucleotide sugar metabolism, Metabolic pathways |
| AARS2 | Aminoacyl-tRNA biosynthesis |
| MTFMT | Aminoacyl-tRNA biosynthesis, One carbon pool by folate |
| RAB5C | Amoebiasis, Endocytosis, Phagosome, Vasopressin-regulated water reabsorption |
| MAP2K2 | Angiotensin II mediated activation of JNK Pathway via Pyk2 dependent signaling, Anthrax |
| | Toxin Mechanism of Action, Bioactive Peptide Induced Signaling Pathway, Erk1/Erk2 |
| | Mapk Signaling pathway, fMLP induced chemokine gene expression in HMC-1 cells, |
| | Human Cytomegalovirus and Map Kinase Pathways, Integrin Signaling Pathway, Links |
| | between Pyk2 and Map Kinases, MAPKinase Signaling Pathway, Phosphorylation of |
| | MEK1 by cdk5/p35 down regulates the MAP kinase pathway, Role of b-arrestins in the |
| | activation and targeting of MAP kinases, Role of MAL in Rho-Mediated Activation of |
| | SRF, Roles of b-arrestin-dependent Recruitment of Src Kinases in GPCR Signaling, |
| | Signaling of Hepatocyte Growth Factor Receptor, Acute myeloid leukemia, B cell receptor |
| | signaling pathway, Bladder cancer, Chronic myeloid leukemia, Endometrial cancer, ErbB |
| | signaling pathway, Fc epsilon RI signaling pathway, Gap junction, Glioma, GnRH |
| | signaling pathway, Insulin signaling pathway, Long-term depression, Long-term |
| | potentiation, . . . |
| LGMN | Antigen processing and presentation, Lysosome |
| PSMB5 | Antigen Processing and Presentation, Proteasome |
| DFFA | Apoptotic DNA fragmentation and tissue homeostasis, Caspase Cascade in Apoptosis, FAS |
| | signaling pathway (CD95), Granzyme A mediated Apoptosis Pathway, HIV-I Nef: |
| | negative effector of Fas and TNF, Induction of apoptosis through DR3 and DR4/5 Death |
| | Receptors, Role of Mitochondria in Apoptotic Signaling, TNFR1 Signaling Pathway, |
| | Apoptosis |
| ALDH2 | Arginine and proline metabolism, Ascorbate and aldarate metabolism, beta-Alanine |
| | metabolism, Fatty acid degradation, Glycerolipid metabolism, Glycolysis/ |
| | Gluconeogenesis, Histidine metabolism, Lysine degradation, Metabolic pathways, Pentose |
| | and glucuronate interconversions, Propanoate metabolism, Pyruvate metabolism, |
| | Tryptophan metabolism, Valine, leucine and isoleucine degradation |
| DSG2 | Arrhythmogenic right ventricular cardiomyopathy (ARVC) |
| TBXAS1 | Aspirin Blocks Signaling Pathway Involved in Platelet Activation, Eicosanoid Metabolism, |
| | Arachidonic acid metabolism, Metabolic pathways |
| RGS3 | Axon guidance |
| SEMA4A | Axon guidance |
| SEMA4B | Axon guidance |
| XIAP | B Cell Survival Pathway, Caspase Cascade in Apoptosis, HIV-I Nef: negative effector of |
| | Fas and TNF, Induction of apoptosis through DR3 and DR4/5 Death Receptors, Role of |
| | Mitochondria in Apoptotic Signaling, Apoptosis, Focal adhesion, NOD-like receptor |
| | signaling pathway, Pathways in cancer, Small cell lung cancer, Toxoplasmosis, Ubiquitin |
| | mediated proteolysis |
| RHOG | Bacterial invasion of epithelial cells, Shigellosis |
| GTF2H4 | Basal transcription factors, Nucleotide excision repair |
| POLD4 | Base excision repair, DNA replication, Homologous recombination, Metabolic pathways, |
| | Mismatch repair, Nucleotide excision repair, Purine metabolism, Pyrimidine metabolism |
| POLE3 | Base excision repair, DNA replication, Metabolic pathways, Nucleotide excision repair, |
| | Purine metabolism, Pyrimidine metabolism |
| PPP3CB | BCR Signaling Pathway, Control of skeletal myogenesis by HDAC & calcium/calmodulin- |
| | dependent kinase (CaMK), Effects of calcineurin in Keratinocyte Differentiation, |
| | Endocytotic role of NDK, Phosphins and Dynamin, Fc Epsilon Receptor I Signaling in |
| | Mast Cells, fMLP induced chemokine gene expression in HMC-1 cells, Neuropeptides VIP |
| | and PACAP inhibit the apoptosis of activated T cells, NFAT and Hypertrophy of the heart |
| | (Transcription in the broken heart), Nitric Oxide Signaling Pathway, Regulation of PGC-1a, |
| | Role of MEF2D in T-cell Apoptosis, Signaling Pathway from G-Protein Families, T Cell |
| | Receptor Signaling Pathway, Alzheimer's disease, Amyotrophic lateral sclerosis (ALS), |
| | Apoptosis, Axon guidance, B cell receptor signaling pathway, Calcium signaling pathway, |
| | Long-term potentiation, MAPK signaling pathway, Natural killer cell mediated |
| | cytotoxicity, Oocyte meiosis, Osteoclast differentiation, T cell receptor signaling pathway, |
| | VEGF signaling pathway, Wnt signaling pathway |
| TECR | Biosynthesis of unsaturated fatty acids |
| SCD5 | Biosynthesis of unsaturated fatty acids, PPAR signaling pathway |
| L2HGDH | Butanoate metabolism |
| MYLK3 | Calcium signaling pathway, Focal adhesion, Gastric acid secretion, Regulation of actin |
| | cytoskeleton, Vascular smooth muscle contraction |
| CHRM3 | Calcium signaling pathway, Gastric acid secretion, Neuroactive ligand-receptor interaction, |
| | Pancreatic secretion, Regulation of actin cytoskeleton, Salivary secretion |
| SLC25A5 | Calcium signaling pathway, Huntington's disease, Parkinson's disease |
| ADRA1A | Calcium signaling pathway, Neuroactive ligand-receptor interaction, Salivary secretion, |
| | Vascular smooth muscle contraction |
| PHB2 | CARM1 and Regulation of the Estrogen Receptor |
| ORC4 | CDK Regulation of DNA Replication, Cell cycle |
| CLDN15 | Cell adhesion molecules (CAMs), Hepatitis C, Leukocyte transendothelial migration, Tight |
| | junction |
| CLDN19 | Cell adhesion molecules (CAMs), Hepatitis C, Leukocyte transendothelial migration, Tight |
| | junction |
| PPP2CB | Chagas disease (American trypanosomiasis), Hepatitis C, Long-term depression, mRNA |
| | surveillance pathway, Oocyte meiosis, TGF-beta signaling pathway, Tight junction, Wnt |
| | signaling pathway |
| GNB2 | Chemokine signaling pathway |
| GNG4 | Chemokine signaling pathway |
| CCR6 | Chemokine signaling pathway, Cytokine-cytokine receptor interaction |
| GAB2 | Chronic myeloid leukemia, Fc epsilon RI signaling pathway, Fc gamma R-mediated |
| | phagocytosis, Osteoclast differentiation |
| PER2 | Circadian rhythm |
| ACO2 | Citrate cycle (TCA cycle), Glyoxylate and dicarboxylate metabolism, Metabolic pathways |
| MDH2 | Citrate cycle (TCA cycle), Glyoxylate and dicarboxylate metabolism, Metabolic pathways, |
| | Pyruvate metabolism |
| CRK | CXCR4 Signaling Pathway, Signaling of Hepatocyte Growth Factor Receptor, Bacterial |
| | invasion of epithelial cells, Chemokine signaling pathway, Chronic myeloid leukemia, |
| | ErbB signaling pathway, Fc gamma R-mediated phagocytosis, Focal adhesion, Insulin |
| | signaling pathway, MAPK signaling pathway, Neurotrophin signaling pathway, Pathways |
| | in cancer, Regulation of actin cytoskeleton, Renal cell carcinoma, Shigellosis |
| SHMT2 | Cyanoamino acid metabolism, Glycine, serine and threonine metabolism, Metabolic |
| | pathways, One carbon pool by folate |
| TAT | Cysteine and methionine metabolism, Metabolic pathways, Phenylalanine metabolism, |
| | Phenylalanine, tyrosine and tryptophan biosynthesis, Tyrosine metabolism, Ubiquinone and |
| | other terpenoid-quinone biosynthesis |
| CRLF2 | Cytokine-cytokine receptor interaction, Jak-STAT signaling pathway |
| IFNLR1 | Cytokine-cytokine receptor interaction, Jak-STAT signaling pathway |
| OSMR | Cytokine-cytokine receptor interaction, Jak-STAT signaling pathway |
| PYCARD | Cytosolic DNA-sensing pathway, NOD-like receptor signaling pathway |
| GREB1 | Downregulated of MTA-3 in ER-negative Breast Tumors |
| GSTM3 | Drug metabolism - cytochrome P450, Glutathione metabolism, Metabolism of xenobiotics |
| | by cytochrome P450 |
| MGST1 | Drug metabolism - cytochrome P450, Glutathione metabolism, Metabolism of xenobiotics |
| | by cytochrome P450 |
| CDA | Drug metabolism - other enzymes, Metabolic pathways, Pyrimidine metabolism |
| CSNK2A1 | EGF Signaling Pathway, EPO Signaling Pathway, IGF-1 Signaling Pathway, IL 2 signaling |
| | pathway, IL 6 signaling pathway, Insulin Signaling Pathway, Lissencephaly gene (LIS1) in |
| | neuronal migration and development, Nerve growth factor pathway (NGF), PDGF |
| | Signaling Pathway, TPO Signaling Pathway, WNT Signaling Pathway, Adherens junction, |
| | Ribosome biogenesis in eukaryotes, Tight junction, Wnt signaling pathway |
| SDHB | Electron Transport Reaction in Mitochondria, Alzheimer's disease, Citrate cycle (TCA |
| | cycle), Huntington's disease, Metabolic pathways, Oxidative phosphorylation, Parkinson's |
| | disease |
| CHMP1B | Endocytosis |
| CHMP3 | Endocytosis |
| PDCD6IP | Endocytosis |
| RAB11FIP4 | Endocytosis |
| VPS28 | Endocytosis |
| AP2B1 | Endocytosis, Huntington's disease |
| FGFR2 | Endocytosis, MAPK signaling pathway, Pathways in cancer, Prostate cancer, Regulation of |
| | actin cytoskeleton |
| PARD6G | Endocytosis, Tight junction |
| STAT5B | EPO Signaling Pathway, Growth Hormone Signaling Pathway, IL 2 signaling pathway, IL |
| | 3 signaling pathway, IL-2 Receptor Beta Chain in T cell Activation, IL22 Soluble Receptor |
| | Signaling Pathway, IL-7 Signal Transduction, Inhibition of Cellular Proliferation by |
| | Gleevec, Mechanism of Gene Regulation by Peroxisome Proliferators via PPARa(alpha), |
| | TPO Signaling Pathway, Acute myeloid leukemia, Chemokine signaling pathway, Chronic |
| | myeloid leukemia, ErbB signaling pathway, Jak-STAT signaling pathway, Pathways in |
| | cancer |
| GSN | Erk and PI-3 Kinase Are Necessary for Collagen Binding in Corneal Epithelia, HIV-I Nef: |
| | negative effector of Fas and TNF, Rho cell motility signaling pathway, Fc gamma R- |
| | mediated phagocytosis, Regulation of actin cytoskeleton |
| EIF5 | Eukaryotic protein translation, Regulation of eIF2, RNA transport |
| CFLAR | FAS signaling pathway (CD95), HIV-I Nef: negative effector of Fas and TNF, IL-2 |
| | Receptor Beta Chain in T cell Activation, Induction of apoptosis through DR3 and DR4/5 |
| | Death Receptors, Apoptosis, Chagas disease (American trypanosomiasis) |
| OLAH | Fatty acid biosynthesis, Metabolic pathways |
| ACADSB | Fatty acid degradation, Metabolic pathways, Valine, leucine and isoleucine degradation |
| AKR1B1 | Fructose and mannose metabolism, Galactose metabolism, Glycerolipid metabolism, |
| | Metabolic pathways, Pentose and glucuronate interconversions, Pyruvate metabolism |
| TUBB | Gap junction, Pathogenic Escherichia coli infection, Phagosome |
| SLC9A4 | Gastric acid secretion |
| GPX4 | Glutathione metabolism |
| LYPLA2 | Glycerophospholipid metabolism |
| CDIPT | Glycerophospholipid metabolism, Inositol phosphate metabolism, Metabolic pathways, |
| | Phosphatidylinositol signaling system |
| PISD | Glycerophospholipid metabolism, Metabolic pathways |
| B3GALT6 | Glycosaminoglycan biosynthesis - chondroitin sulfate/dermatan sulfate, |
| | Glycosaminoglycan biosynthesis - heparan sulfate/heparin, Metabolic pathways |
| CHST6 | Glycosaminoglycan biosynthesis - keratan sulfate |
| FUT1 | Glycosphingolipid biosynthesis - globo series, Glycosphingolipid biosynthesis - lacto and |
| | neolacto series, Metabolic pathways |
| FUT2 | Glycosphingolipid biosynthesis - globo series, Glycosphingolipid biosynthesis - lacto and |
| | neolacto series, Metabolic pathways |
| NAGA | Glycosphingolipid biosynthesis - globo series, Lysosome |
| PIGY | Glycosylphosphatidylinositol(GPI)-anchor biosynthesis, Metabolic pathways |
| CD24 | Hematopoietic cell lineage |
| METTL2B | Histidine metabolism, Tyrosine metabolism |
| MUS81 | Homologous recombination |
| XRCC2 | Homologous recombination |
| DNAL1 | Huntington's disease |
| POLR2D | Huntington's disease, Metabolic pathways, Purine metabolism, Pyrimidine metabolism, |
| | RNA polymerase |
| DCTN2 | Huntington's disease, Vasopressin-regulated water reabsorption |
| NQO1 | Hypoxia and p53 in the Cardiovascular system |
| IKZF3 | IL-2 Receptor Beta Chain in T cell Activation |
| CRKL | IL-2 Receptor Beta Chain in T cell Activation, Inhibition of Cellular Proliferation by |
| | Gleevec, Integrin Signaling Pathway, Links between Pyk2 and Map Kinases, Signaling of |
| | Hepatocyte Growth Factor Receptor, Bacterial invasion of epithelial cells, Chemokine |
| | signaling pathway, Chronic myeloid leukemia, ErbB signaling pathway, Fc gamma R- |
| | mediated phagocytosis, Focal adhesion, Insulin signaling pathway, MAPK signaling |
| | pathway, Neurotrophin signaling pathway, Pathways in cancer, Regulation of actin |
| | cytoskeleton, Renal cell carcinoma, Shigellosis |
| INPP5E | Inositol phosphate metabolism, Metabolic pathways, Phosphatidylinositol signaling system |
| ALDH6A1 | Inositol phosphate metabolism, Metabolic pathways, Propanoate metabolism, Valine, |
| | leucine and isoleucine degradation |
| GYS1 | Insulin signaling pathway, Starch and sucrose metabolism |
| SPRED1 | Jak-STAT signaling pathway |
| MAPK13 | Keratinocyte Differentiation, MAPKinase Signaling Pathway, Stathmin and breast cancer |
| | resistance to antimicrotubule agents, Amyotrophic lateral sclerosis (ALS), Chagas disease |
| | (American trypanosomiasis), Epithelial cell signaling in Helicobacter pylori infection, Fc |
| | epsilon RI signaling pathway, GnRH signaling pathway, Hepatitis C, Leishmaniasis, |
| | Leukocyte transendothelial migration, MAPK signaling pathway, Neurotrophin signaling |
| | pathway, NOD-like receptor signaling pathway, Osteoclast differentiation, Progesterone- |
| | mediated oocyte maturation, RIG-I-like receptor signaling pathway, Shigellosis, T cell |
| | receptor signaling pathway, Toll-like receptor signaling pathway, Toxoplasmosis, VEGF |
| | signaling pathway |
| GALK1 | Leloir pathway of galactose metabolism, Amino sugar and nucleotide sugar metabolism, |
| | Galactose metabolism, Metabolic pathways |
| AASS | Lysine biosynthesis, Lysine degradation, Metabolic pathways |
| SETMAR | Lysine degradation |
| SUV420H2 | Lysine degradation |
| AP1M1 | Lysosome |
| AP1S3 | Lysosome |
| AP4S1 | Lysosome |
| CD164 | Lysosome |
| ENTPD4 | Lysosome, Purine metabolism, Pyrimidine metabolism |
| ACP2 | Lysosome, Riboflavin metabolism |
| SLC25A11 | Malate-aspartate shuttle, Shuttle for transfer of acetyl groups from mitochondria to the |
| | cytosol |
| MDH1 | Malate-aspartate shuttle, Shuttle for transfer of acetyl groups from mitochondria to the |
| | cytosol, Citrate cycle (TCA cycle), Glyoxylate and dicarboxylate metabolism, Metabolic |
| | pathways, Proximal tubule bicarbonate reclamation, Pyruvate metabolism |
| DUSP7 | MAPK signaling pathway |
| FGF5 | MAPK signaling pathway, Melanoma, Pathways in cancer, Regulation of actin |
| | cytoskeleton |
| MAP2K5 | MAPKinase Signaling Pathway, Gap junction, MAPK signaling pathway, Neurotrophin |
| | signaling pathway |
| RPS6KA4 | MAPKinase Signaling Pathway, MAPK signaling pathway, Neurotrophin signaling |
| | pathway |
| MAP3K9 | MAPKinase Signaling Pathway, p38 MAPK Signaling Pathway |
| IAPP | Maturity onset diabetes of the young |
| APOA2 | Mechanism of Gene Regulation by Peroxisome Proliferators via PPARa(alpha), PPAR |
| | signaling pathway |
| TGDS | Metabolic pathways |
| B3GNT6 | Metabolic pathways, Mucin type O-Glycan biosynthesis |
| NMNAT1 | Metabolic pathways, Nicotinate and nicotinamide metabolism |
| PGLS | Metabolic pathways, Pentose phosphate pathway |
| H6PD | Metabolic pathways, Pentose phosphate pathway |
| CYP51A1 | Metabolic pathways, Steroid biosynthesis |
| EBP | Metabolic pathways, Steroid biosynthesis |
| COMT | Metabolic pathways, Steroid hormone biosynthesis, Tyrosine metabolism |
| CSAD | Metabolic pathways, Taurine and hypotaurine metabolism |
| BCKDHA | Metabolic pathways, Valine, leucine and isoleucine degradation |
| PNN | mRNA surveillance pathway, RNA transport |
| ULK3 | mTOR signaling pathway, Regulation of autophagy |
| GSTP1 | Multi-Drug Resistance Factors, Drug metabolism - cytochrome P450, Glutathione |
| | metabolism, Metabolism of xenobiotics by cytochrome P450, Pathways in cancer, Prostate |
| | cancer |
| HCST | Natural killer cell mediated cytotoxicity |
| TRPV1 | Neuroactive ligand-receptor interaction |
| TSPO | Neuroactive ligand-receptor interaction |
| VIPR1 | Neuroactive ligand-receptor interaction |
| UBE2D1 | Neuroregulin receptor degradation protein-1 Controls ErbB3 receptor recycling, Protein |
| | processing in endoplasmic reticulum, Ubiquitin mediated proteolysis |
| CA5B | Nitrogen metabolism |
| MFNG | Notch signaling pathway, Other types of O-glycan biosynthesis |
| DDB1 | Nucleotide excision repair, Ubiquitin mediated proteolysis |
| OR11A1 | Olfactory transduction |
| SGOL1 | Oocyte meiosis |
| LILRA2 | Osteoclast differentiation |
| SQSTM1 | Osteoclast differentiation |
| MDM4 | p53 signaling pathway |
| TP53AIP1 | p53 signaling pathway |
| PARK7 | Parkinson's disease |
| PARK2 | Parkinson's disease, Protein processing in endoplasmic reticulum, Ubiquitin mediated |
| | proteolysis |
| ECH1 | Peroxisome |
| MPV17L | Peroxisome |
| PXMP4 | Peroxisome |
| CORO1A | Phagosome |
| DBI | PPAR signaling pathway |
| SLC27A1 | PPAR signaling pathway |
| PSMD14 | Proteasome |
| SLC15A1 | Protein digestion and absorption |
| SPCS1 | Protein export |
| SRP9 | Protein export |
| SSR2 | Protein processing in endoplasmic reticulum |
| PDE4C | Purine metabolism |
| KLRD1 | Ras-Independent pathway in NK cell-mediated cytotoxicity, Antigen processing and |
| | presentation, Graft-versus-host disease, Natural killer cell mediated cytotoxicity |
| OPHN1 | Rho cell motility signaling pathway |
| RPL18A | Ribosome |
| RPL28 | Ribosome |
| RPL41 | Ribosome |
| RPS12 | Ribosome |
| RPLP1 | Ribosome |
| POP7 | Ribosome biogenesis in eukaryotes, RNA transport |
| DIS3 | RNA degradation |
| AAAS | RNA transport |
| GEMIN8 | RNA transport |
| NUP133 | RNA transport |
| PHAX | RNA transport |
| RAD1 | Role of BRCA1, BRCA2 and ATR in Cancer Susceptibility |
| TREX1 | Role of BRCA1, BRCA2 and ATR in Cancer Susceptibility, Cytosolic DNA-sensing |
| | pathway |
| DIABLO | Role of Mitochondria in Apoptotic Signaling |
| CHRNB1 | Role of nicotinic acetylcholine receptors in the regulation of apoptosis, Neuroactive ligand- |
| | receptor interaction |
| INMT | Selenocompound metabolism, Tryptophan metabolism |
| SLC25A1 | Shuttle for transfer of acetyl groups from mitochondria to the cytosol |
| GOSR1 | SNARE interactions in vesicular transport |
| DYRK1B | Sonic Hedgehog (Shh) Pathway |
| CTNNBL1 | Spliceosome |
| CCDC12 | Spliceosome |
| HNRNPA1L2 | Spliceosome |
| PRPF3 | Spliceosome |
| PRPF38B | Spliceosome |
| PRPF8 | Spliceosome |
| RBM25 | Spliceosome |
| SRSF10 | Spliceosome |
| SRSF4 | Spliceosome |
| SF3A2 | Spliceosome |
| TCERG1 | Spliceosome |
| FUT6 | Steps in the Glycosylation of Mammalian N-linked Oligosaccarides, Glycosphingolipid |
| | biosynthesis - lacto and neolacto series, Metabolic pathways |
| MOCS3 | Sulfur relay system |
| H2AFJ | Systemic lupus erythematosus |
| H2AFX | Systemic lupus erythematosus |
| HIST1H2AC | Systemic lupus erythematosus |
| HIST1H3H | Systemic lupus erythematosus |
| CAT | The IGF-1 Receptor and Longevity, Amyotrophic lateral sclerosis (ALS), Metabolic |
| | pathways, Peroxisome, Tryptophan metabolism |
| YY1 | The PRC2 Complex Sets Long-term Gene Silencing Through Modification of Histone Tails |
| TOLLIP | Toll-like receptor signaling pathway |
| UBE3C | Ubiquitin mediated proteolysis |
| KDELR1 | Vibrio cholerae infection |
| KDELR2 | Vibrio cholerae infection |
| CXADR | Viral myocarditis |
| PDE6A | Visual Signal Transduction, Phototransduction, Purine metabolism |
| SLC19A1 | Vitamin digestion and absorption |

TABLE 8
Anti-TL1A and Anti-DR3 Antibodiy Sequences

| SEQ ID | | |
|---|---|---|
| NO | Identifier | Amino Acid Sequence |
| 209 | HCDR1 | GFTFSTYG |
| 210 | HCDR2 | ISGTGRTT |
| 211 | HCDR3 | TKERGDYYYG VFDY |
| 212 | LCDR1 | QTISSW |
| 213 | LCDR2 | AAS |
| 214 | LCDR3 | QQYHRSWT |
| 215 | HC | EVQLLESGGG LVQPGKSLRL SCAVSGFTFS TYGMNWVRQA |
| | Variable | PGKGLEWVSS |
| | | ISGTGRTTYH ADSVQGRFTV SRDNSKNILY LQMNSLRADD |
| | | TAVYFCTKER |
| | | GDYYYGVFDY WGQGTLVTVS S |
| 216 | LC | DIQMTQSPST LSASVGDRVT ITCRASQTIS SWLAWYQQTP |
| | Variable | EKAPKLLIYA |
| | | ASNLQSGVPS RFSGSGSGTE FTLTISSLQP DDFATYYCQQ |
| | | YHRSWTFGQG |
| | | TKVEIT |
| 217 | HCDR1 | GFTFSSYW |
| 218 | HCDR2 | IKEDGSEK |
| 219 | HCDR3 | AREDYDSYYK YGMDV |
| 220 | LCDR1 | QSILYSSNNK NY |
| 221 | LCDR2 | WAS |
| 222 | LCDR3 | QQYYSTPFT |
| 223 | HC | EVQLVESGGG LVQPGGSLRL SCAVSGFTFS SYWMSWVRQA |
| | Variable | PGKGLEWVAN |
| | | IKEDGSEKNY VDSVKGRFTL SSDNAKNSLY LQMNSLRAED |
| | | TAVYYCARED |
| | | YDSYYKYGMD VWGQGTAVIV SS |
| 224 | LC | DIVMTQSPDS LAVSLGERAT INCKSSQSIL YSSNNKNYLA |
| | Variable | WYQQKPGQPP |
| | | KLLIYWASTR ESGVPDRFSG SGSGTDFTLT ISSLQAEDVS |
| | | VYYCQQYYST |
| | | PFTFGPGTKV DIK |
| 225 | HCDR1 | GGSFTGFY |
| 226 | HCDR2 | INHRGNT |
| 227 | HCDR3 | ASPFYDFWSG SDY |
| 228 | LCDR1 | QSLVHSDGNT Y |
| 229 | LCDR2 | KIS |
| 230 | LCDR3 | MQATQFPLT |
| 231 | HC | QVQLQQWGAG LLKPSETLSL TCAVYGGSFT GFYWSWIRQP |
| | Variable | PGKGLEWIGE |
| | | INHRGNTNYN PSLKSRVTMS VDTSKNQFSL NMISVTAADT |
| | | AMYFCASPFY |
| | | DFWSGSDYWG QGTLVTVSS |
| 232 | LC | DIMLTQTPLT SPVTLGQPAS ISCKSSQSLV HSDGNTYLSW |
| | Variable | LQQRPGQPPR |
| | | LLFYKISNRF SGVPDRFSGS GAGTDFTLKI SRVEAEDVGV |
| | | YYCMQATQFP |
| | | LTFGGGTKVE IK |
| 233 | HCDR1 | GY(X1)F(X2)(X3)YGIS; X1 = P, S, D, Q, N; X2 = T, R; X3 = N, T, Y, H |
| 234 | HCDR2 | WIS(X1)YNG(X2)(X3)(X4)YA(X5)(X6)(X7)QG; X1 = T, P, S, A; X2 = N, G, |
| | | V, K, A; X3 = T, K; X4 = H, N; X5 = Q, R; X6 = K, M; X7 = L, H |
| 235 | HCDR3 | ENYYGSG(X1)(X2)R GGMD(X3); X1 = S, A; X2 = Y, P; X3 = V, A, G |
| 236 | HCDR1 | GYDFTYYGIS |
| 237 | HCDR2 | WISTYNGNTH YARMLQG |
| 238 | HCDR3 | ENYYGSGAYR GGMDV |
| 239 | LCDR1 | RASQSVSSYL A |
| 240 | LCDR2 | DASNRAT |
| 241 | LCDR3 | QQRSNWPWT |
| 242 | HC | QVQLVQSGAE VKKPGASVKV SCKASGYDFT YYGISWVRQA |
| | Variable | PGQGLEWMGW |
| | | ISTYNGNTHY ARMLQGRVTM TTDTSTRTAY MELRSLRSDD |
| | | TAVYYCAREN |
| | | YYGSGAYRGG MDVWGQGTTV TVSS |
| 243 | LC | EIVLTQSPAT LSLSPGERAT LSCRASQSVS SYLAWYQQKP |
| | Variable | GQAPRLLIYD |
| | | ASNRATGIPA RFSGSGSGTD FTLTISSLEP EDFAVYYCQQ |
| | | RSNWPWTFGQ |
| | | GTKVEIK |
| 244 | HC | QVQLVQSGAE VKKPGASVKV SCKASGYDFT YYGISWVRQA |
| | | PGQGLEWMGW |
| | | ISTYNGNTHY ARMLQGRVTM TTDTSTRTAY MELRSLRSDD |
| | | TAVYYCAREN |
| | | YYGSGAYRGG MDVWGQGTTV TVSSASTKGP SVFPLAPSSK |
| | | STSGGTAALG |
| | | CLVKDYFPEP VTVSWNSGAL TSGVHTFPAV LQSSGLYSLS |
| | | SVVTVPSSSL |
| | | GTQTYICNVN HKPSNTKVDK KVEPKSCDKT HTCPPCPAPE |
| | | AAGAPSVFLF |
| | | PPKPKDTLMI SRTPEVTCVV VDVSHEDPEV KFNWYVDGVE |
| | | VHNAKTKPRE |
| | | EQYNSTYRVV SVLTVLHQDW LNGKEYKCKV SNKALPAPIE |
| | | KTISKAKGQP |
| | | REPQVYTLPP SREEMTKNQV SLTCLVKGFY PSDIAVEWES |
| | | NGQPENNYKT |
| | | TPPVLDSDGS FFLYSKLTVD KSRWQQGNVF SCSVMHEALH |
| | | NHYTQKSLSL |
| | | SPG |
| 245 | LC | EIVLTQSPAT LSLSPGERAT LSCRASQSVS SYLAWYQQKP |
| | | GQAPRLLIYD |
| | | ASNRATGIPA RFSGSGSGTD FTLTISSLEP EDFAVYYCQQ |
| | | RSNWPWTFGQ |
| | | GTKVEIKRTV AAPSVFIFPP SDEQLKSGTA SVVCLLNNFY |
| | | PREAKVQWKV |
| | | DNALQSGNSQ ESVTEQDSKD STYSLSSTLT LSKADYEKHK |
| | | VYACEVTHQG |
| | | LSSPVTKSFN RGEC |
| 246 | HCDR1 | SRSYYWG |
| 247 | HCDR2 | SIYYNGRTYY NPSLKS |
| 248 | HCDR3 | EDYGDYGAFD I |
| 249 | LCDR1 | RASQGISSAL A |
| 250 | LCDR2 | DASSLES |
| 251 | LCDR3 | QQFNSYPLT |
| 252 | HC | QLQLQESGPG LVKPSETLSL TCTVSGGSIS SRSYYWGWIR |
| | Variable | QPPGKGLEWI |
| | | GSIYYNGRTY YNPSLKSRVT ISVDTSKNQF SLKLSSVTAA |
| | | DTAVYYCARE |
| | | DYGDYGAFDI WGQGTMVTVS S |
| 253 | LC | AIQLTQSPSS LSASVGDRVT ITCRASQGIS SALAWYQQKP |
| | Variable | GKAPKLLIYD |
| | | ASSLESGVPS RFSGSGSGTD FTLTISSLQP EDFATYYCQQ |
| | | FNSYPLTFGG |
| | | GTKVEIK |
| 254 | HCDR1 | TSNMGVV |
| 255 | HCDR2 | HILWDDREY SNPALKS |
| 256 | HCDR3 | MSRNYYGSSYVMDY |
| 257 | LCDR1 | SASSSVNYMH |
| 258 | LCDR2 | STSNLAS |
| 259 | LCDR3 | HQWNNYGT |
| 260 | HC | QVTLKESGPALVKPTQTLTLTCTFSGFSLSTSNMGVVWIRQPPGKALEW |
| | Variable | LAHILWDD |
| | | REYSNPALKSRLTISKDTSKNQVVLTMTNMDPVDTATYYCARMSRNY |
| | | YGSSYVMD YWGQGTLVTVSS |
| 261 | LC | DIQLTQSPSFLSASVGDRVTITCSASSSVNYMHWYQQKPGKAPKLLIYS |
| | Variable | TSNLASGVP |
| | | SRFSGSGSGTEFTLTISSLQPEDFATYYCHQWNNYGTFGQGTKVEIKR |
| 262 | HCDR1 | LYGMN |
| 263 | HCDR1 | NYGMN |
| 264 | HCDR2 | WINTYTGEPTYADDFKG |
| 265 | HCDR3 | DTAMDYAMAY |
| 266 | HCDR3 | DYGKYGDYYAMDY |
| 267 | LCDR1 | KSSQNIVHSDGNTYLE |
| 268 | LCDR1 | RSSQSIVHSNGNTYLD |
| 269 | LCDR2 | KVSNRFS |
| 270 | LCDR3 | FQGSHVPLT |
| 271 | HC | QVQLVQSGSELKKPGASVKVSCKASGYTFTLYGMNWVRQAPGQGLE |
| | Variable | WMG |
| | | WINTYTGEPTYADDFKGRFVFSLDTSVSTAYLQISSLKAEDTAVYYCAR |
| | | DTAMDYAMAYWGQGTLVTVSS |
| 272 | HC | QVQLVQSGSELKKPGASVKVSCKASGYTFTLYGMNWVKQAPGKGLK |
| | Variable | WMG |
| | | WINTYTGEPTYADDFKGRFVFSLDTSVSTAYLQISSLKAEDTAVYFCAR |
| | | DTAMDYAMAYWGQGTLVTVSS |
| 273 | HC | QVQLVQSGSELKKPGASVKVSCKASGYTFTNYGMNWVRQAPGQGLE |
| | Variable | WMG |
| | | WINTYTGEPTYADDFKGRFVFSLDTSVSTAYLQISSLKAEDTAVYYCAR |
| | | DYGKYGDYYAMDYWGQGTLVTVSS |
| 274 | HC | QVQLVQSGSELKKPGASVKVSCKASGYTFTNYGMNWVRQAPGKGLK |
| | Variable | WMG |
| | | WINTYTGEPTYADDFKGRFVFSLDTSVSTAYLQISSLKAEDTAVYFCAR |
| | | DYGKYGDYYAMDYWGQGTLVTVSS |
| 275 | LC | DVVMTQSPLSLPVTLGQPASISCKSSQNIVHSDGNTYLEWFQQRPGQSP |
| | Variable | RRLIYKVSNRFSGVPDRFSGSGSGTDFTLKISRVEAEDVGVYYCFQGSH |
| | | VPLTFGGGTKVEIKR |
| 276 | LC | DVVMTQSPLSLPVTLGQPASISCKSSQNIVHSDGNTYLEWFQQRPGQSP |
| | Variable | RRLIYKVSNRFSGVPDRFSGSGSGTDFTLKISRVEAEDVGVYYCFQGSH |
| | | VPLTFGQGTKVEIKR |
| 277 | LC | DVVMTQTPLSLPVTPGEPASISCKSSQNIVHSDGNTYLEWYLQKPGQSP |
| | Variable | QLLIYKVSNRFSGVPDRFSGSGSGTDFTLKISRVEAEDLGVYYCFQGSH |
| | | VPLTFGGGTKVEIKR |
| 278 | LC | DVVMTQTPLSLPVSLGDQASISCKSSQNIVHSDGNTYLEWYLQKPGQSP |
| | Variable | KVLIYKVSNRFSGVPDRFSGSGSGTDFTLKISRVEAEDLGVYYCFQGSH |
| | | VPLTFGGGTKVEIKR |
| 279 | LC | DVVMTQSPLSLPVTLGQPASISCRSSQSIVHSNGNTYLDWFQQRPGQSP |
| | Variable | RRLIYKVSNRFSGVPDRFSGSGSGTDFTLKISRVEAEDVGVYYCFQGSH |
| | | VPLTFGGGTKVEIKR |
| 280 | LC | DVVMTQSPLSLPVTLGQPASISCRSSQSIVHSNGNTYLDWFQQRPGQSP |
| | Variable | RRLIYKVSNRFSGVPDRFSGSGSGTDFTLKISRVEAEDVGVYYCFQGSH |
| | | VPLTFGQGTKVEIKR |
| 281 | LC | DVVMTQTPLSLPVTPGEPASISCRSSQSIVHSNGNTYLDWYLQKPGQSP |
| | Variable | QLLIYKVSNRFSGVPDRFSGSGSGTDFTLKISRVEAEDLGVYYCFQGSH |
| | | VPLTFGGGTKVEIKR |
| 282 | LC | DVVMTQTPLSLPVSLGDQASISCRSSQSIVHSNGNTYLDWYLQKPGQSP |
| | Variable | KVLIYKVSNRFSGVPDRFSGSGSGTDFTLKINRVEAEDLGVYFCFQGSH |
| | | VPLTFGGGTKLEIKR |
| 283 | HCDR1 | GYTFTSSWMH |
| 284 | HCDR2 | IHPNSGGT |
| 285 | HCDR3 | ARGDYYGYVS WFAY |
| 286 | LCDR1 | QNINVL |
| 287 | LCDR2 | KAS |
| 288 | LCDR3 | QQGQSYPYT |
| 289 | HC | QVQLQQPGSV LVRPGASVKV SCKASGYTFT SSWMHWAKQR |
| | Variable | PGQGLEWIGE |
| | | IHPNSGGTNY NEKFKGKATV DTSSSTAYVD LSSLTSEDSA |
| | | VYYCARGDYY |
| | | GYVSWFAYWG QGTLVTVSS |
| 290 | HC | QVQLVQSGAEVKKPGASVKVSCKASGYTFTSSWMHWARQA |
| | Variable | PGQGLEWIGE |
| | | IHPNSGGTNY AQKFQGRATL TVDTSSSTAY MELSRLRSDD |
| | | TAVYYCARGD |
| | | YYGYVSWFAY WGQGTLVTVS S |
| 291 | HC | QVQLVQSGAE VKKPGASVKV SCKASGYTFT SSWMHWARQA |
| | Variable | PGQGLEWIGE |
| | | IHPNSGGTNY AQKFQGRATM TVDTSISTAY MELSRLRSDD |
| | | TAVYYCARGD |
| | | YYGYVSWFAY WGQGTLVTVS S |
| 292 | HC | QVQLVQSGAE VKKPGASVKV SCKASGYTFT SSWMHWARQA |
| | Variable | PGQGLEWIGE |
| | | IHPNSGGTNY AQKFQGRVTM TVDTSISTAY MELSRLRSDD |
| | | TAVYYCARGD |
| | | YYGYVSWFAY WGQGTLVTVS S |
| 293 | HC | QVQLVQSGAE VKKPGASVKV SCKASGYTFT SSWMHWARQA |
| | Variable | PGQGLEWMGE |
| | | IHPNSGGTNY AQKFQGRVTM TVDTSISTAY MELSRLRSDD |
| | | TAVYYCARGD |
| | | YYGYVSWFAY WGQGTLVTVS S |
| 294 | LC | DIQMNQSPSS LSASLGDTIT ITCHASQNIN VLLSWYQQKP |
| | Variable | GNIPKLLIYK |
| | | ASNLHTGVPS RFSGSGSGTG FTFTISSLQP EDIATYYCQQ |
| | | GQSYPYTFGG |
| | | GTKLEIK |
| 295 | LC | DIQMTQSPSS LSASVGDRVT ITCQASQDIS NYLNWYQQKP |
| | Variable | GKAPKLLIYD |
| | | ASNLETGVPS RFSGSGSGTD FTFTISSLQP EDIATYYCQQ |
| | | YDNLPYTFGQ |
| | | GTKLEIK |
| 296 | LC | DIQMTQSPSS LSASVGDRVT ITCQASQNIN VLLNWYQQKP |
| | Variable | GKAPKLLIYK |
| | | ASNLHTGVPS RFSGSGSGTD FTFTISSLQP EDIATYYCQQ |
| | | GQSYPYTFGQ |
| | | GTKLEIK |
| 297 | LC | DIQMNQSPSS LSASVGDRVT ITCQASQNIN VLLSWYQQKP |
| | Variable | GKAPKLLIYK |
| | | ASNLHTGVPS RFSGSGSGTD FTFTISSLQP EDIATYYCQQ |
| | | GQSYPYTFGQ |
| | | GTKLEIK |
| 298 | HCDR1 | GYTFTSYDIN |
| 299 | HCDR2 | WLNPNSGXTG; X = N, Y |
| 300 | HCDR3 | EVPETAAFEY |
| 301 | LCDR1 | TSSSSDIGA(X1)(X2)GV(X3); X1 = G, A; X2 = L, S, Q; X3 = H, L |
| 302 | LCDR2 | GYYNRPS |
| 303 | LCDR3 | QSXDGTLSAL; X = Y, W, F |
| 304 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 305 | LC | QSVLTQPPSV SGAPGQRVTI SCTSSSSDIG AXXGVXWYQQ |
| | Variable | LPGTAPKLLI |
| | | EGYYNRPSGV PDRFSGSKSG TSASLTITGL LPEDEGDYYC |
| | | QSXDGTLSAL |
| | | FGGGTKLTVL G |
| 306 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 307 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 308 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 309 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSYDGTLSAL |
| | | FGGGTKLTVL G |
| 310 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 311 | LC | QSVLTQPPSV SGAPGQRVTI SCTSSSSDIG AALGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 312 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 313 | LC | QSVLTQPPSV SGAPGQRVTI SCTSSSSDIG AGSGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 314 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 315 | LC | QSVLTQPPSV SGAPGQRVTI SCTSSSSDIG AGQGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 316 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 317 | LC | QSVLTQPPSV SGAPGQRVTI SCTSSSSDIG AGLGVLWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 318 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 319 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 320 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 321 | LC | QSVLTQPPSV SGAPGQRVTI SCTSSSSDIG AGSGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 322 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 323 | LC | QSVLTQPPSV SGAPGQRVTI SCTSSSSDIG AGQGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 324 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 325 | LC | QSVLTQPPSV SGAPGQRVTI SCTSSSSDIG AGLGVLWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 326 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 327 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSFDGTLSAL |
| | | FGGGTKLTVL G |
| 328 | HCDR1 | SYFWS |
| 329 | HCDR2 | YIYYSGNTKYNPSLKS |
| 330 | HCDR3 | ETGSYYGFDY |
| 331 | LCDR1 | RASQSINNYLN |
| 332 | LCDR2 | AASSLQS |
| 333 | LCDR3 | QQSYSTPRT |
| 334 | HC | QVQLQESGPGLVKPSETLSLTCTVSGGSISSYFWSWIRQPPGKGLEWIGY |
| | Variable | IYYSGNTKYNPSLKSRVTISIDTSKNQFSLKLSSVTAADTAVYYCARETG |
| | | SYYGFDYWGQGTLVTVSS |
| 335 | LC | DIQMTQSPSSLSASVGDRVTITCRASQSINNYLNWYQQRPGKAPKLLIY |
| | Variable | AASSLQSGVPSRFSGSGSGTDFTLTISSLQPGDFATYYCQQSYSTPRTFG |
| | | QGTKLEIK |
| 336 | HCDR1 | GYYWN |
| 337 | HCDR2 | EINHAGNTNYNPSLKS |
| 338 | HCDR3 | GYCRSTTCYFDY |
| 339 | LCDR1 | RASQSVRSSYLA |
| 340 | LCDR2 | GASSRAT |
| 341 | LCDR3 | QQYGSSPT |
| 342 | HC | QVQLQQWGAGLLKPSETLSLTCAVHGGSFSGYYWNWIRQPPGKGLEW |
| | Variable | IGEINHAGNTNYNPSLKSRVTISLDTSKNQFSLTLTSVTAADTAVYYCAR |
| | | GYCRSTTCYFDYWGQGTLVTVSS |
| 343 | LC | EIVLTQSPGTLSLSPGERATLSCRASQSVRSSYLAWYQQKPGQAPRLLIY |
| | Variable | GASSRATGIPDRFSGSGSGTDFTLTISRLEPEDFAVYYCQQYGSSPTFGQ |
| | | GTRLEIK |
| 344 | HC | EVQLQQSGAELVKPGASVKLSCTASGFDIQDTYMHWVKQRPEQGLEWI |
| | Variable | GRIDPASGHTKYDPKFQVKATITTDTSSNTAYLQLSSLTSEDTAVYYCS |
| | | RSGGLPDVWGAGTTVTVSS |
| 345 | LC | QIVLSQSPAILSASPGEKVTMTCRASSSVSYMYWYQQKPGSSPKPWIYA |
| | Variable | TSNLASGVPDRFSGSGSGTSYSLTISRVEAEDAATYYCQQWSGNPRTFG |
| | | GGTKLEIK |
| 346 | HCDR1 | GFDIQDTYMH |
| 347 | HCDR2 | RIDPASGHTKYDPKFQV |
| 348 | HCDR3 | SGGLPDV |
| 349 | LCDR1 | RASSSVSYMY |
| 350 | LCDR2 | ATSNLAS |
| 351 | LCDR3 | QQWSGNPRT |
| 352 | HC | QVQLVQSGAEVKKPGASVKLSCKASGFDIQDTYMHWVRQAPGQGLE |
| | Variable | WMGRIDPASGHTKYDPKFQVRVTMTTDTSTSTVYMELSSLRSEDTAVY |
| | | YCSRSGGLPDVWGQGTTVTVSS |
| 353 | LC | EIVLTQSPGTLSLSPGERVTMSCRASSSVSYMYWYQQKPGQAPRPWIYA |
| | Variable | TSNLASGVPDRFSGSGSGTDYTLTISRLEPEDFAVYYCQQWSGNPRTFG |
| | | GGTKLEIK |
| 354 | (CDR- | QVQLVQSGAEVKKPGASVKLSCKASGFDIQDTYMHWVRQAPGQGLE |
| | grafted | WMGRIDPASGHTKYDPKFQVRVTMTRDTSTSTVYMELSSLRSEDTAVY |
| | LC) HC | YCSRSGGLPDVWGQGTTVTVSS |
| | variable | |
| | region | |
| 355 | (CDR- | EIVLTQSPGTLSLSPGERATLSCRASSSVSYMYWYQQKPGQAPRLLIYA |
| | grafted | TSNLASGIPDRFSGSGSGTDFTLTISRLEPEDFAVYYCQQWSGNPRTFGG |
| | LC) HC | GTKLEIK |
| | variable | |
| | region | |
| 356 | (CDR- | QVQLVQSGAEVKKPGASVKVSCKASGFDIQDTYMHWVRQAPGQGLE |
| | grafted | WMGRIDPASGHTKYDPKFQVRVTMTRDTSTSTVYMELSSLRSEDTAVY |
| | HC) HC | YCARSGGLPDVWGQGTTVTVSS |
| | variable | |
| | region | |
| 357 | (CDR- | EIVLTQSPGTLSLSPGERATLSCRASSSVSYMYWYQQKPGQAPRLLIYA |
| | grafted | TSNLASGVPDRFSGSGSGTDYTLTISRLEPEDFAVYYCQQWSGNPRTFG |
| | HC) LC | GGTKLEIK |
| | variable | |
| | region | |
| 358 | HC | EVMLVESGGGLVKPGGSLKLSCAASGFTFTNYAMSWVRQTPEKRLEW |
| | variable | VATITSGGSYIYYLDSVKGRFTISRDNAKSTLYLQMSSLRSEDTAIYNCA |
| | | RRKDGNYYYAMDYWGQGTSVTVSS |
| 359 | HC | EVMLVESGGGLVKPGGSLKLSCAASGFTFTNYAMSWVRQTPEKRLEW |
| | variable | VATITSGGSYIYYLDSVKGRFTISRDNAKSTLYLQMSSLRSEDTAIYYCA |
| | | RRKDGNYYYAMDYWGQGTSVTVSS |
| 360 | HC | EVQLVESGGGLVKPGGSLRLSCAASGFTFTNYAMSWVRQAPGQRLEW |
| | variable | VSTITSGGSYIYYLDSVKGRFTISRDNAKSTLYLQMNSLRAEDTAVYNC |
| | | ARRKDGNYYYAMDYWGQGTTVTVSS |
| 361 | HC | EVQLVESGGGLVKPGGSLRLSCAASGFTFTNYAMSWVRQAPGQRLEW |
| | variable | VSTITSGGSYIYYLDSVKGRFTISRDNAKSTLYLQMNSLRAEDTAVYYC |
| | | ARRKDGNYYYAMDYWGQGTTVTVSS |
| 362 | HC | EVQLLESGGGLVQPGRSLRLSCAASGFTFTNYAMSWVRQAPGQRLEW |
| | variable | LATITSGGSYIYYLDSVKGRFTISRDNSKSTLYLQMGSLRAEDMAVYNC |
| | | ARRKDGNYYYAMDYWGQGTTVTVSS |
| 363 | HC | EVQLLESGGGLVQPGRSLRLSCAASGFTFTNYAMSWVRQAPGQRLEW |
| | variable | LATITSGGSYIYYLDSVKGRFTISRDNSKSTLYLQMGSLRAEDMAVYYC |
| | | ARRKDGNYYYAMDYWGQGTTVTVSS |
| 364 | HC | QVQLVESGGGLIQPGGSLRLSCAASGFTFTNYAMSWVRQARGQRLEW |
| | variable | VSTITSGGSYIYYLDSVKGRFTISRDNSKSTLYMELSSLRSEDTAVYNCA |
| | | RRKDGNYYYAMDYWGQGTTVTVSS |
| 365 | HC | QVQLVESGGGLIQPGGSLRLSCAASGFTFTNYAMSWVRQARGQRLEW |
| | variable | VSTITSGGSYIYYLDSVKGRFTISRDNSKSTLYMELSSLRSEDTAVYYCA |
| | | RRKDGNYYYAMDYWGQGTTVTVSS |
| 366 | HC | QVQLVQSGSELKKPGASVKVSCKASGFTFTNYAMSWVRQAPGKRLEW |
| | variable | VSTITSGGSYIYYLDSVKGRFTISRENAKSTLYLQMNSLRTEDTALYNCA |
| | | RRKDGNYYYAMDYWGQGTTVTVSS |
| 367 | HC | QVQLVQSGSELKKPGASVKVSCKASGFTFTNYAMSWVRQAPGKRLEW |
| | variable | VATITSGGSYIYYLDSVKGRFTISRENAKSTLYLQMNSLRTEDTALYYC |
| | | ARRKDGNYYYAMDYWGQGTTVTVSS |
| 368 | HC | EVQLLQSGAEVKKPGASVKVSCKASGFTFTNYAMSWVRQAPGQRLEW |
| | variable | VATITSGGSYIYYLDSVKGRFTISRDNAKSTLHLQMNSLRAEDTAVYNC |
| | | ARRKDGNYYYAMDYWGQGTTVTVSS |
| 369 | HC | EVQLLQSGAEVKKPGASVKVSCKASGFTFTNYAMSWVRQAPGQRLEW |
| | variable | VATITSGGSYIYYLDSVKGRFTISRDNAKSTLHLQMNSLRAEDTAIYYC |
| | | ARRKDGNYYYAMDYWGQGTTVTVSS |
| 370 | HC | EVMLLQSGAEVKKPGASVKVSCKASGFTFTNYAMSWVRQAPGQRLE |
| | variable | WVATITSGGSYIYYLDSVKGRFTISRDNAKSTLHLQMNSLRAEDTAVY |
| | | YCARRKDGNYYYAMDYWGQGTTVTVSS |
| 371 | LC | DIVLTQSPASLAVSLGQRATISCRASESVDSYGNSFIHWYQQKAGQPPK |
| | variable | LLIYRASNLESGIPARFSGSGSRTDFTLTINPVEADDVATYYCQQSYEDP |
| | | WTFGGGTKLEIK |
| 372 | LC | DIVLTQSPATLSLSPGERATLSCRASESVDSYGNSFIHWYQQKPGQPPKL |
| | variable | LIYRASNLESGIPARFSGSGSRTDFTLTISSLEPEDFAVYYCQQSYEDPWT |
| | | FGGGTKXEIK |
| 373 | LC | DIVLTQSPSSLSASVGDRVTITCRASESVDSYGNSFIHWYQQKPGQPPKL |
| | variable | LIYRASNLESGIPARFSGSGSRTDFTLTISSLQPEDFATYYCQQSYEDPWT |
| | | FGGGTKXEIK |
| 374 | LC | DIVLTQSPDFQSVTPKEKVTITCRASESVDSYGNSFIHWYQQKPGQPPKL |
| | variable | LIYRASNLESGIPARFSGSGSRTDFTLTISSLEAEDAATYYCQQSYEDPW |
| | | TFGGGTKXEIK |
| 375 | LC | DIVLTQTPLSLSVTPGQPASISCRASESVDSYGNSFIHWYQQKPGQPPKL |
| | variable | LIYRASNLESGIPARFSGSGSRTDFTLKISRVEAEDVGVYYCQQSYEDPW |
| | | TFGGGTKXEIK |
| 376 | HCDR1 | TYGMS |
| 377 | HCDR2 | WMNTYSGVTTYADDFKG |
| 378 | HCDR3 | EGYVFDDYYATDY |
| 379 | LCDR1 | RSSQNIVHSDGNTYLE |
| 380 | LCDR2 | KVSNRFS |
| 381 | LCDR3 | FQGSHVPLT |
| 382 | HC | QIQLVQSGPELKKPGETVKISCKASGYTFTTYGMSWVKQAPGKGLKW |
| | Variable | MGWMNTYSGVTTYADDFKGRFAFSLETSASTAYMQIDNLKNEDTATY |
| | | FCAREGYVFDDYYATDYWGQGTSVTVSS |
| 383 | LC | DVLMTQTPLSLPVSLGDQASISCRSSQNIVHSDGNTYLEWYLQKPGQSP |
| | Variable | KLLIYKVSNRFSGVPDRFSGSGSGTDFTLKISRVEAEDLGIYYCFQGSHV |
| | | PLTFGAGTKLELK |
| 384 | HCDR1 | KYDIN |
| 385 | HCDR2 | WIFPGDGRTDYNEKFKG |
| 386 | HCDR3 | YGPAMDY |
| 387 | LCDR1 | RSSQTIVHSNGDTYLD |
| 388 | LCDR2 | KVSNRFS |
| 389 | LCDR3 | FQGSHVPYT |
| 390 | HC | MGWSWVFLFLLSVTAGVHSQVHLQQSGPELVKPGASVKLSCKASGYT |
| | Variable | FTKYDINWVRQRPEQGLEWIGWIFPGDGRTDYNEKFKGKATLTTDKSS |
| | | STAYMEVSRLTSEDSAVYFCARYGPAMDYWGQGTSVTVA S |
| 391 | LC | MKLPVRLLVLMFWIPASSSDVLMTQTPLSLPVSLGDQASISCRSSQTIVH |
| | Variable | SNGDTYLDWFLQKPGQSPKLLIYKVSNRFSGVPDRFSGSGSGTDFTLKIS |
| | | RVEAEDLGVYYCFQGSHVPYTFGGGTKLEIK |

1. A method for processing or analyzing a biological sample from a subject, comprising:
(a) obtaining the biological sample comprising gene expression products, wherein the subject has or is suspected of having Crohn's Disease (CD);
(b) subjecting the biological sample to an assay to yield a data set including data corresponding to gene expression product levels;
(c) in a programmed computer, inputting said data including said gene expression product levels from (b) to a trained algorithm to generate a classification of said sample as positive for a CD-PBmu subtype based detection of an expression profile comprising an increase in the gene expression levels compared to a reference expression profile,
wherein the trained algorithm is trained with a plurality of training samples, and wherein said biological sample is independent of said plurality of training samples; and
(d) electronically outputting a report that identifies the classification of the biological sample as positive for the CD-PBmu subtype,
wherein optionally the biological sample comprises a blood sample or is purified from a blood sample of the subject.

2. The method of claim 1, wherein the gene expression products comprises RNA.

3. The method of claim 1, wherein the assay comprises using one or more of a microarray, sequencing, and qPCR.

4. The method of claim 1, wherein the trained algorithm is trained with one or more datasets of gene expression product levels obtained from the plurality of training samples.

5. The method of claim 1, wherein the gene expression products are expressed from genes comprising two or more of A disintegrin and metalloproteinase with thrombospondin motifs 1 (ADAMTS1), Neutrophil gelatinase-associated lipocalin (LCN2), Disintegrin and metalloproteinase domain-containing protein 28 (ADAM28), Tryptase beta-2 (TPSB2), peptidylprolyl isomerase A pseudogene 30 (PPIAP30), glutamine-fructose-6-phosphate transaminase 2 (GFPT2), KIT proto-oncogene, receptor tyrosine kinase (KIT), phospholipid transfer protein (PLTP), major facilitator superfamily domain containing 2A (MFSD2A), interleukin 22 (IL22), LIM and cysteine rich domains 1 (LMCD1), interleukin 6 (IL6), TBC1 domain family member 9 (TBC1D9), ChaC glutathione specific gamma-glutamylcyclotransferase 1 (CHAC1), selenoprotein P (SEPP1), superoxide dismutase 3 (SOD3), RAB13, member RAS oncogene family (RAB13), lysozyme (LYZ), carboxypeptidase A3 (CPA3), serine dehydratase (SDS), dual specificity tyrosine phosphorylation regulated kinase 3 (DYRK3), DAB adaptor protein 2 (DAB2), TBC1 domain family member 8 (TBC1D8), crystallin alpha B (CRYAB), TBC1 domain family member 3 (TBC1D3), leucine rich repeat containing 32 (LRRC32), serpin family G member 1 (SERPING1), ubiquitin D (UBD), fatty acid binding protein 1 (FABP1), spleen associated tyrosine kinase (SYK), aldolase, fructose-bisphosphate B (ALDOB), semaphorin 6B (SEMA6B), NANOG neighbor homeobox (NANOGNB), dermatan sulfate epimerase (DSE), formyl peptide receptor 3 (FPR3), tenascin XB (TNXB), olfactory receptor family 4 subfamily A member 5 (OR4A5), decorin (DCN), carbohydrate sulfotransferase 15 (CHST15), ADAM like decysin 1 (ADAMDEC1), histidine decarboxylase (HDC), RRAD, Ras related glycolysis inhibitor and calcium channel regulator (RRAD), complement C1s (C1S), or phospholipase A2 group IIA (PLA2G2A), or a combination thereof.

6. The method of claim 5, wherein the gene expression products are expressed from genes comprising two or more of ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, or UBD, or a combination thereof.

7. The method of claim 1, wherein the increase in the gene expression product levels is at least 2-fold greater than in the reference expression profile, wherein optionally the reference expression profile comprises expression levels of the one or more genes of one or more subjects that do not have CD.

8. (canceled)

9. (canceled)

10. The method of claim 1, further comprising (1) treating the subject by administering to the subject a therapeutic agent or (2) optimizing a therapeutic regimen of the subject comprising increasing or decreasing a dosage amount of a therapeutic agent administered to the subject for the treatment of the CD, based on the CD-PBmu subtype.

11. (canceled)

12. A method of treating Crohn's disease (CD) in a subject, the method comprising administering to the subject a therapeutically effective amount of a therapeutic agent, provided the subject is identified as having a CD-PBmu subtype by: (a) detecting an expression profile comprising an increase in a level of expression of one or more genes in the biological sample, relative to a reference expression profile; and (b) identifying the subject as having a CD-PBmu subtype based upon the expression profile that is detected in (b).

13. The method of claim 12, wherein the one or more genes comprises ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPING1, UBD, FABP1, SYK, ALDOB, SEMA6B, NANOGNB, DSE, FPR3, TNXB, OR4A5, DCN, CHST15, ADAMDEC1, HDC, RRAD, C1S, or PLA2G2A, or a combination thereof.

14. The method of claim 13, wherein the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, or UBD, or a combination thereof.

15. The method of claim 14, wherein (1) the one or more genes comprises at least 10 of the one or more genes, or (2) the one or more genes comprises between about 10-27 of the one or more genes.

16. (canceled)

17. The method of claim 12, wherein the increase in the level of expression of the one or more genes in the biological sample is at least 2-fold greater than in the reference expression profile, wherein optionally the reference expression profile comprises expression levels of the one or more genes of one or more subjects that do not have CD.

18. (canceled)

19. The method of claim 12, wherein detecting the expression profile comprises detecting the increase in the level of expression of the one or more genes by:
(a) contacting the biological sample with a nucleic acid primer and/or detectable nucleic acid probe; and
(b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes.

20. A method of determining a Crohn's Disease (CD) subtype in a subject having CD, the method comprising:
(a) measuring a level of expression of one or more genes from Table 1A in a biological sample obtained from a subject having CD;
(b) detecting an expression profile comprising an increase in the level of expression of the one or more genes in the biological sample, relative to a reference expression profile; and
(b) identifying the subject as having a CD-PBmu subtype based upon the expression profile that is detected in (b),
wherein optionally the biological sample comprises a blood sample or is purified from a blood sample of the subject.

21. The method of claim 20, provided that the one or more genes comprises ADAMTS1, LCN2, ADAM28, TPSB2, PPIAP30, GFPT2, KIT, T PLTP, MFSD2A, IL22, LMCD1, IL6, TBC1D9, CHAC1, SEPP1, SOD3, RAB13, LYZ, CPA3, SDS, DYRK3, DAB2, TBC1D8, CRYAB, TBC1D3, LRRC32, SERPING1, UBD, FABP1, SYK, ALDOB, SEMA6B, NANOGNB, DSE, FPR3, TNXB, OR4A5, DCN, CHST15, ADAMDEC1, HDC, RRAD, C1S, or PLA2G2A or a combination thereof.

22. The method of claim 21, wherein the one or more genes comprises ADAMDEC1, ALDOB, CHST15, C1S, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, or UBD, or a combination thereof.

23. The method of claim 21, wherein the one or more genes comprises at least 10 of the one or more genes.

24. The method of claim 20, wherein the increase in the level of expression of the one or more genes in the biological sample is at least 2-fold greater than in the reference expression profile, wherein optionally the reference expression profile comprises expression levels of the one or more genes of one or more subjects that do not have CD.

25. (canceled)

26. The method of claim 20, wherein measuring a level of expression of one or more genes comprises
(1) utilizing an assay selected from the group consisting of an RNA sequencing method, a microarray method, and quantitative polymerase chain reaction (qPCR), or
(2) (a) contacting the biological sample with a nucleic acid primer and/or detectable nucleic acid probe; and
(b) hybridizing the nucleic acid primer and/or detectable nucleic acid probe to a nucleic acid sequence of the one or more genes that is measured, wherein the detectable nucleic acid probe comprises a nucleic acid sequence comprising at least about 10 contiguous nucleic acids of the one of the one or more genes.

27. (canceled)

28. The method of claim 20, further comprising (1) treating the subject by administering a therapeutic agent to the subject, or (2) optimizing a therapeutic regimen of the subject comprising increasing or decreasing a dosage amount of a therapeutic agent administered to the subject for the treatment of the CD, based on the CD-PBmu subtype.

29. (canceled)

30. (canceled)
